(19) United States
(12) Patent Application Publication
(10) Pub. No.: US 2023/0124862 A1
(43) Pub. Date: Apr. 20, 2023

(54) BISMUTH THIOL COMPOUNDS AND COMPOSITIONS AND METHODS OF TREATING MICROBIAL CO-INFECTIONS

(71) Applicants: Jeffrey W. MILLARD, Bozeman, MT (US); Brett Hugh James BAKER, Bozeman, MT (US); Microbion Corporation, Bozeman, MT (US)

(72) Inventors: Jeffrey W. MILLARD, Bozeman, MT (US); Brett Hugh James BAKER, Bozeman, MT (US)

(21) Appl. No.: 17/914,308

(22) PCT Filed: Mar. 24, 2021
(86) PCT No.: PCT/US2021/023922

§ 371 Date: Sep. 23, 2022

Related U.S. Application Data
(60) Provisional application No. 63/039,850, filed on Jun. 16, 2020.
(60) Provisional application No. 63/000,840, filed on Mar. 27, 2020.
(60) Provisional application No. 62/994,224, filed on Mar. 24, 2020.

(51) Int. Cl. A61K 33/245; A61K 9/00; A61K 45/06; A61P 31/16

(52) U.S. Cl. CPC A61K 33/245; A61K 9/0078; A61K 45/06; A61P 31/16

(57) ABSTRACT

The invention relates to Bismuth thiol compounds such as BisEDT for treating bacterial and fungal infection in patients with viral pulmonary infections such as COVID-19, alone or in combination with other anti-viral drugs.

### CROSS REFERENCE TO RELATED APPLICATIONS

This application claims the benefit of U.S. Provisional Application No. 62/994,224, filed on Mar. 24, 2020, U.S. Provisional Application No. 63/000,840, filed on Mar. 27, 2020, and U.S. Provisional Application No. 63/039,850, filed on Jun. 16, 2020, the contents of each aforementioned application are incorporated by reference in their entireties.

### BACKGROUND

Respiratory viral infections such as influenza and coronavirus are significant causes of respiratory disease. Influenza causes more than 250,000 deaths annually in the industrialized world. The 1918 pandemic is estimated to have killed at least twenty million people worldwide. It was caused by a particular influenza viral strain and was characterized by both rapid transmission and severe symptoms.

Even without a pandemic, influenza infection presents both a health risk and health cost. On average, 5% to 20% of the U.S. population gets influenza (commonly called “the flu”) each year. More than 100,000 people are hospitalized from flu complications, and approximately 36,000 people die. Some people, such as older people, young children, and people with certain health conditions (e.g. immunocompromised people), are at high risk for serious flu complications.

Influenza A and B viruses are responsible for seasonal flu epidemics each year. Over the course of a flu season, different types (A & B) and subtypes of influenza A viruses can circulate through the population and cause illness. A particular problem for treatment strategies is the fact that influenza viruses are constantly changing through a process called “antigenic drift.” Thus, a vaccine that might have been useful last year may be less effective or ineffective this year.

During influenza outbreaks, opportunistic bacteria frequently cause secondary illnesses, including pneumonia, bronchitis, sinusitis, and otitis media. Indeed, pneumococcal pneumonia was the primary cause of death during the 1918 influenza pandemic.

Most recently, the 2019-2021 coronavirus 2019 (COVID-19) pandemic is an ongoing pandemic caused by severe acute respiratory syndrome coronavirus 2 (SARS-CoV-2). The outbreak was first identified in Wuhan, Hubei, China, in December 2019, and was recognized as a pandemic by the World Health Organization (WHO) on Mar. 11, 2020. As of Mar. 15, 2021 more than 120 million cases of COVID-19 have been reported in over 190 countries and territories, resulting in over 2.65 million deaths.

While viral vaccines and antiviral medications are critical to pandemic preparedness, anti-infectives that prevent and treat bacterial co-infections are equally critical. Bacterial co-infections require extended hospital stays, increase hospitalization costs, and are a frequent cause of mortality where viral infection patients might otherwise survive. It is a fact that bacterial superinfections have long been responsible for many, if not the vast majority, of deaths related to respiratory viral pandemics. Bacterial co-infection rates are likely underestimated due to inaccuracy of current diagnostic methodologies (culturing from nasopharyngeal swabs, blood tests), yet have been found to be responsible for at least 30% of pandemic-associated mortality.

While the number of bacterial coinfections in the current COVID-19 pandemic has not yet been defined, published scientific studies are indicating that bacterial coinfections are playing a substantial role in this pandemic, as they have with all pandemics in the last 100 years. There is currently an urgent, ongoing, substantial unmet need for broad-spectrum antibacterial and antifungal drugs for the treatment of pulmonary infections. These infections can occur as primary bacterial or fungal infections, or as infections that are secondary to viral pulmonary infections including those that occur in viral pandemics. Such secondary bacterial and fungal superinfections have been responsible for up to the vast majority of deaths in viral pandemics prior to 1969, and up to 55% of deaths in more recent pandemics. It is critical to identify an antimicrobial compound capable of irradicating these microbial superinfections and which is impervious to the development of antimicrobial-resistance.

Bismuth-1,2-ethanedithiol (BisEDT) has been previously reported as effective for the topical and/or local treatment of antibiotic-resistant and difficult to treat microbial infections. BisEDT and processes for its preparation are disclosed in International Patent Application Nos. PCT/US2010/023108, PCT/US2011/023549, PCT/US2011/047490, PCT/US2019/044489, and PCT/US2019/044495 which are hereby incorporated by reference in their entireties for all purposes, however, an amorphous form of BisEDT has never been previously synthesized despite reports ot the contrary. For example, International Patent Application Nos. PCT/US2010/023108, PCT/US2011/023549, PCT/US2011/047490 described a synthesis of BisEDT under ethanolic solvent conditions, the product of which was believed to be amorphous; however, unexpectedly this form of BisEDT was discovered to be crystalline.

A new amorphous form of a compound may possess physical properties that differ from, and are advantageous over, those of other crystalline or amorphous forms. These include, packing properties such as molar volume, density and hygroscopicity; thermodynamic properties such as melting temperature, vapor pressure and solubility; kinetic properties such as dissolution rate and stability under various storage conditions; surface properties such as surface area, wettability, interfacial tension and shape; mechanical properties such as hardness, tensile strength, compactibility, handling, flow and blend; filtration properties; and improved efficacy and absorption. Variations in any one of these properties may affect the chemical and pharmaceutical processing of a compound as well as its bioavailability and may often render the new form advantageous for pharmaceutical and medical use.

There still remains an unmet need for amorphous forms of BisEDT having good physicochemical properties, desirable bioavailability, and advantageous pharmaceutical parameters. This invention meets those needs.

### SUMMARY

The present disclosure provides methods of treating, managing or lessening the severity of symptoms associated with infections to a primary respiratory viral infection in a subject, the method comprising administering to the subject a bismuth-thiol (BT) composition that comprises BisEDT suspended therein, wherein administering the BT composition is via inhalation, orally or nasally, using an aerosol device.

The present disclosure also provides an aerosol comprising a plurality of dispersed liquid droplets in a gas, said liquid droplets comprising a BT composition comprising BisEDT compound suspended therein and at least one antimicrobial agent; and wherein at least 70% of the liquid droplets have a MMAD from about of from about 0.9 μm to about 3 μm.

The present disclosure also provides a pharmaceutical composition comprising bismuth-thiol (BT) composition that comprises BisEDT suspended therein and at least one antimicrobial agent, wherein the BT composition comprises a plurality of microparticles, wherein the D90 of said microparticles is less than or equal to 1.9 μm.

The present disclosure also provides a kit comprising (1) an aerosol comprising a plurality of dispersed liquid droplets in a gas, said liquid droplets comprising a BT composition comprising BisEDT compound suspended therein; and wherein at least 70% of the liquid droplets have a MMAD from about of from about 0.9 μm to about 3 sim, and (2) at least one antimicrobial agent.

The present disclosure also provides methods of preventing infections to a primary respiratory viral infection in a subject, the method comprising administering to the subject a bismuth-thiol (BT) composition that comprises BisEDT suspended therein, wherein administering the BT composition is via inhalation, orally or nasally, using an aerosol device, wherein the subject.

In some embodiments, the present disclosure provides an amorphous form of bismuth-1,2-ethanedithiol (BisEDT). In some embodiments, the X-ray powder diffraction pattern of amorphous BisEDT does not contain any distinct peaks. In some embodiments, the X-ray powder diffraction pattern of amorphous BisEDT is substantially similar to FIG. 44. In some embodiments, differential scanning calorimetry thermogram of amorphous BisEDT comprises an exothermic peak at about 168° C. In some embodiments, the differential scanning calorimetry thermogram further comprises an endotherm at about 64° C. and/or an endotherm peak at about 112° C. and/or an exotherm peak at about 145° C. In some embodiments, the differential scanning calorimetry thermogram is substantially similar to FIG. 45. In some embodiments, the amorphous BisEDT has a glass transition at about 101° C. In some embodiments, the amorphous form is at least 90% pure. For example, the amorphous form is at least 95% or 98% pure.

In some embodiments, the present disclosure provides a composition comprising an amorphous from of BisEDT. In some embodiments, the composition comprises at least one pharmaceutically acceptable carrier. In some embodiments, the composition comprises BisEDT in a suspension.

In some embodiments, the present disclosure provides a method of treating, managing or lessening the severity of symptoms and infections associated with one or more pulmonary diseases or infections in a subject, the method comprising administering to the subject a bismuth-thiol (BT) composition that comprises amorphous BisEDT suspended therein, wherein administering the BT composition is via inhalation, orally or nasally, using an aerosol device. In some embodiments, the method is treating, managing or lessening the severity of cystic fibrosis (CF) symptoms and infections in a subject.

In some embodiments, the present disclosure provides a method for healing a wound in a subject having a diabetic foot infection, comprising administering the subject a therapeutically effective amount of a composition comprising amorphous BisEDT. In some embodiments, the wound is a diabetic foot ulcer.

In some embodiments, the present disclosure provides a method of making an amorphous form of BisEDT, comprising (a) mixing an acidic aqueous solution that comprises a bismuth salt, with a solvent selected from the group consisting of acetone, acetonitrile, 1,2-dichloroethane, dimethyl sulfoxide, ethyl acetate, isopropanol, methyl tert-butyl ether, and mixtures thereof; (b) combing the product of (a) with a solution of 1,2-ethanedithiol in a solvent selected from the group consisting of acetone, acetonitrile, 1,2-dichloroethane, dimethyl sulfoxide, ethyl acetate, isopropanol, methyl tert-butyl ether, and mixtures thereof, under conditions and for a time sufficient for formation of a precipitate which comprises the amorphous form of BisEDT. In some embodiments, the method further comprises recovering the precipitate to remove impurities. In some embodiments, the bismuth salt is Bi(NO₃)₃. In some embodiments, 1,2-ethanedithiol is at a concentration of from about 1% wt/vol to about 20% wt/vol prior to step (b). In some embodiments, the acidic aqueous solution is prepared by mixing an aqueous suspension of either Bi (III) sub-nitrate or Bi (III) nitrate pentahydrate with an acid under conditions and for a time sufficient to form a substantially clear solution. In some embodiments, the concentration of either Bi (III) sub-nitrate or Bi (III) nitrate pentahydrate in the aqueous solution is from about 100 mg/mL to about 400 mg/mL. In some embodiments, the acid is 70% HNO₃. In some embodiments, the method further comprises adding the clear solution to an acidic solution. In some embodiments, the acidic solution is 5% HNO₃. In some embodiments, step (b) is performed at a temperature ranging from about 20° C. to about 28° C.

In some embodiments, the present disclosure provides a method of treating, managing or lessening the severity of cystic fibrosis (CF) symptoms and infections in a subject, the method comprising administering to the subject a bismuth-thiol (BT) composition that comprises amorphous BisEDT. In some embodiments, the BT composition comprises a plurality of microparticles wherein at least 70%, 80%, or 90% of said microparticles having a volumetric mean diameter (VMD) from about 0.01 μm to about 2.5 μm. In some embodiments, when the BT composition is aerosolized, at least 70%, 80%, or 90% of the aerosolized liquid droplets have a mass median aerodynamic diameter (MMAD) from about 0.03 μm to about 3 μm. In some embodiments, the BT composition comprises BisEDT at a concentration greater than about 0.1 mg/mL, about 0.05% to about 1.0% Tween 80®, about 40 mM to about 250 mM sodium chloride, and optionally about 2 to 20 mM sodium phosphate at about pH. 7.4. In some embodiments, the subject has at least one pulmonary infection containing one or more bacterial pathogens and/or fungal pathogens (as described herein). In some embodiments, the method comprises at least one of: (i) reducing a bacterial biofilm, (ii) impairing growth of a bacterial biofilm, (iii) preventing initial formation of the bacterial biofilm, and/or (iv) preventing reformation of the bacterial biofilm.

In some embodiments, the present disclosure provides an aerosol comprising a plurality of dispersed liquid droplets in a gas, said liquid droplets comprising a BT composition comprising amorphous BisEDT suspended therein; and wherein at least 70% of the liquid droplets have a MMAD from about 0.03 μm to about 3 μm. In some embodiments, prior to aerosolization, the BT composition comprises a plurality of microparticles wherein at least 70%, 80%, or 90% of said microparticles have a VMD from about 0.01 μm to about 2.5 μm. In some embodiments, the droplets further comprise Tween 80 and optionally a buffer at a pH of about 7.4; and/or sodium chloride.

In some embodiments, the present disclosure provides a pharmaceutical composition comprising bismuth-thiol (BT) composition that comprises amorphous BisEDT suspended therein, wherein the BT composition comprises a plurality of microparticles, wherein the D90 of said microparticles is less than or equal to 1.9 μm. In some embodiments, the BT composition comprises a plurality of microparticles, wherein the D90 of said microparticles is less than or equal to about 1.6 μm. In some embodiments, at least 70%4, 80%, or 90% of said microparticles have a volumetric mean diameter from about 0.01 μm to about 2.5 μm.

In some embodiments, the present disclosure provides a method of treating, managing or lessening the severity of symptoms and infections associated with one or more pulmonary diseases or infections in a subject, the method comprising administering to the subject a bismuth-thiol (BT) composition that comprises amorphous BisEDT, wherein the BT composition comprises a plurality of microparticles wherein at least 70% of said microparticles having a volumetric mean diameter from about 0.01 μm to about 2.5 μm, and wherein when the BT composition is aerosolized, at least 70% of the aerosolized liquid droplets have a MMAD from about 0.03 μm to about 3 μm. In some embodiments, one or more pulmonary diseases or infections are not the result of or associated with cystic fibrosis. In some embodiments, the pulmonary infection is bronchiectasis infection, pneumonia, valley fever, allergic bronchopulmonary aspergillosis (ABPA), ventilator acquired pneumonia, hospital acquired pneumonia, community acquired pneumonia, ventilator associated tracheobronchitis, lower respiratory tract infection, non-tuberculous Mycobacteria (NTM), anthrax, legionellosis, pertussis, bronchitis, Bronchiolitis, COPD-associated infection, and post-lung transplantation. In some embodiments, the pulmonary infection is non-tuberculous Mycobacteria (NTM).

In some embodiments, the present disclosure provides a method for healing a wound in a subject having a diabetic foot infection, comprising administering the subject a therapeutically effective amount of a composition comprising amorphous BisEDT, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is healed or substantially healed within 12 weeks of the first administration of the composition. In some embodiments, the wound is a diabetic foot ulcer. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4. In some embodiments, the applied BT composition is present on the surface at a concentration from about 1 μg/cm² to about 1,000,000 μg/cm². In some embodiments, the applied BT composition is present on the surface at a concentration from about 50 μg/cm² to about 100 μg/cm². In some embodiments, the applied BT composition is present on the surface at a concentration greater than about 100 μg/cm². In some embodiments, the BT composition is administered three times per day, two times per day, once daily, every other day, once every three days, three times per week, once every week, once every other week, once every month, or once every other month. In some embodiments, the wound is healed 4 weeks, 8 weeks or 12 weeks after the first administration of the BT composition. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the wound area is from about 0.1 cm² to about 250 cm².

In some embodiments, the present disclosure provides a method for wound size reduction in a subject having a diabetic foot infection, comprising administering to the subject a therapeutically effective amount of a composition comprising amorphous BisEDT, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is reduced in size from about a 1% reduction relative to the original wound size to total elimination of the wound within 12 weeks of the first administration of the composition. In some embodiments, the wound is reduced by about 10%, 20%, 30%, 40%, 50%, 60%, 70%, 80%, or 90%. In some embodiments, the wound is reduced by at least about 50%. In some embodiments, the wound is a diabetic foot ulcer. In some embodiments, the BT composition further comprises about 0.05% to about 1.0% Tween 80®, about 0.05 to 40 mM sodium chloride, optionally about 1% to about 100%6 of methylcellulose, and optionally about 2 to 20 mM sodium phosphate at about pH. 7.4. In some embodiments, the applied BT composition is present on the surface at a concentration from about 1 μg/cm² to about 1,000,000 μg/cm². In some embodiments, the applied BT composition is present on the surface at a concentration from about 50 μg/cm² to about 100 μg/cm². In some embodiments, the applied BT composition is present on the surface at a concentration greater than about 100 μg/cm². In some embodiments, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the wound area is from about 0.1 cm² to about 250 cm². In some embodiments, the wound surface area of said wound is reduced by at least 50% by 12 weeks after the first administration of the BT composition. In some embodiments, the wound surface area of said wound is reduced by at least 50% by 4 weeks after the first administration of the BisEDT composition. In some embodiments, the wound surface area is measured using digital photographs or hand measurement.

In some embodiments, the present disclosure provides a pharmaceutical composition comprising bismuth-thiol (BT) composition that comprises amorphous BisEDT suspended therein, wherein the BT composition comprises a plurality of microparticles, wherein the D90 of said microparticles is less than or equal to 1.9 μm. In some embodiments, the BT composition comprises a plurality of microparticles, wherein the D90 of said microparticles is less than or equal to about 1.6 μm. In some embodiments, the present disclosure provides a method for healing a wound in a subject having a diabetic foot infection, comprising administering the subject a therapeutically effective amount of the composition.

### BRIEF DESCRIPTION OF THE DRAWINGS

FIG. 1 shows a representative inhalation exposure schematic.

FIG. 2 shows an aerosol particle size distribution of a 2.5 mg/mL solution of BisEDT.

FIG. 3 shows an aerosol particle size distribution of a 25 mg/mL solution of BisEDT.

FIG. 4 shows an aerosol particle size distribution of a 50 mg/mL solution of BisEDT.

FIG. 5 shows an aerosol particle size distribution of a 75 mg/mL solution of BisEDT.

FIG. 6 shows an aerosol particle size distribution of a 100 mg/mL solution of BisEDT.

FIG. 7 shows an aerosol particle size distribution of a 100 mg/mL BisEDT in 300 mOsmolality phosphate buffer solution.

FIG. 8 shows an aerosol particle size distribution of a 50 mg/mL BisEDT in 300 mOsmolality phosphate buffer solution.

FIG. 9 shows an aerosol particle size distribution of a 10 mg/mL BisEDT in 300 mOsmolality phosphate buffer solution.

FIG. 10 shows an aerosol particle size distribution of a 2.5 mg/mL BisEDT in 300 mOsmolality phosphate buffer solution.

FIG. 11 shows that inhaled drug delivery of an antibiotic increases lung exposure (B) while reducing systemic exposure (A) of the corresponding side effects.

FIG. 12 shows the results of MIC testing of BisEDT against a variety of clinically relevant CF isolates.

FIG. 13 is a diagram showing the evaluation of cytotoxicity through both LDH release (from the culture medium side) and trans-epithelial electrical resistance (TEER) from the apical/air-exposed side.

FIG. 14 shows the activity of BT compounds against biofilms grown from MR14, which is a multidrug-resistant CF-isolate of Pseudomonas aeruginosa.

FIG. 15 shows the activity of BT compounds against biofilms grown from AG14, which is an aminoglycoside-resistant CF-isolate of Pseudomonas aeruginosa.

FIG. 16 shows the activity of BT compounds against biofilms grown from AU197, which is a CF-isolate of Burkholderia. cenocepacia.

FIG. 17 shows the activity of BT compounds against biofilms grown from AMT0130-8, which represents a CF-isolate of the clinically relevant Mycobacterium abscessus complex (MABSC), which frequently complicates the treatment of CF pulmonary infections

FIG. 18 shows the activity of BT compounds against biofilms grown from AMT0089-5, which is a macrolide-resistant, amikacin-resistant MABSC.

FIG. 19 shows the activity of BT compounds against biofilms grown from ATCC-19977, which is M. abscessus (macrolide-resistant; inducible).

FIG. 20 shows the activity of BT compounds against biofilms grown from MABSC CF isolate.

FIG. 21 shows the activity of BT compounds against biofilms grown from Achromobacter spp.

FIG. 22 shows the activity of BT compounds against biofilms grown from Stenotrophomonas maltophilia.

FIG. 23 shows the activity of BT compounds against biofilms grown from E. coli.

FIG. 24 shows an overview of MucilAir™ which is a fully differentiated model of the human airway epithelia.

FIG. 25 shows the percentage of cytotoxicity (LDH measurement) of BisEDT in solution at 1, 8, 24 and 48 hours exposure. Bars representing 1, 8, 24 and 48 hours are shown from left to right for each concentration.

FIG. 26 shows the effect on tissue integrity of BisEDT in solution at 1, 8, 24 and 48 hours exposure. Bars representing 1, 8, 24 and 48 hours are shown from left to right for each concentration.

FIG. 27 shows the percentage of cytotoxicity (LDH measurement) of solid BisEDT at 1, 8, 24 and 48 hours exposure. Bars representing 1, 8, 24 and 48 hours are shown from left to right for each concentration.

FIG. 28 shows the effect on tissue integrity of solid BisEDT at 1, 8, 24 and 48 hours exposure. Bars representing 1, 8, 24 and 48 hours are shown from left to right for each concentration.

FIG. 29 shows the percentage of cytotoxicity (LDH measurement) of solid BisEDT at 1, 8, 24 and 48 hours exposure. Bars representing 1, 8, 24 and 48 hours are shown from left to right for each concentration.

FIG. 30 shows the effect on tissue integrity of solid BisEDT at 1, 8, 24 and 48 hours exposure. Bars representing 1, 8, 24 and 48 hours are shown from left to right for each concentration.

FIG. 31 shows the effect of sputum on the bacterial killing activity of tobramycin. Time is shown in hours.

FIG. 32 shows that the bactericidal activity of BisEDT appears to be partially inhibited by CF patient sputum. Time is shown in hours.

FIG. 33 shows that the bactericidal activity of BisBDT appears to be partially inhibited by CF patient sputum. Time is shown in hours.

FIG. 34 shows a graph of lung tissue BisEDT concentration vs. time after a single 100 μg/kg lung deposited dose in rats.

FIG. 35 shows whole blood BisEDT concentration vs. time (100 μg/kg TV or 100 μg/kg inhalation or 250 μg/kg oral dose).

FIG. 36 shows rat blood BisEDT vs time after single inhalation dose (μg/kg lung deposited).

FIG. 37 shows rat lung BisEDT concentration (ng/g) at sacrifice (24 or 30 hours after single inhaled dose).

FIG. 38 shows Particle Size Distribution for vehicle.

FIG. 39 shows Particle Size Distribution for Tobramycin.

FIG. 40 shows Particle Size Distribution for BisEDT.

FIG. 41 shows an example schematic diagram of the dog exposure system.

FIG. 42 shows rat efficacy figures showing cumulative (total) administered dose (lung deposited) at days 3 and 5.

FIG. 43 shows a representative checkerboard assay where each compound is tested alone (Column 12 and Row H) and in combination at varying ratios of drug concentration.

FIG. 44 shows an XRPD pattern of amorphous BisEDT.

FIG. 45 shows a DSC thermogram for amorphous BisEDT.

FIG. 46 shows prevention of mortality in respiratory viral pandemics requires antibacterial drugs to address bacterial superinfections.

FIG. 47 shows rat lung tissue and blood BisEDT levels versus dose, 24 to 30 hours after a single inhalation dose demonstrating much higher levels in the lungs as well as near linear dose deposition (and exposure) from the formulation & device.

FIG. 48 shows single-dose rat PK results showing lung tissue levels versus time (top frame) and blood levels versus time (lower frame) after a single dose via 3 routes of administration.

FIG. 49 shows BisEDT in whole blood following inhalation exposure.

FIG. 50 shows comparison of measured lung concentration at Day 14 and Day 21 post exposure.

FIG. 51 shows measured lung tissue concentration at Day 14 necropsy versus the lung deposited dose (Day 0 and Day 7 Average).

FIG. 52 shows pulmonary bacterial burden by treatment group and study day.

FIG. 53 shows severe COVID-19 patients (or non-surviving COVID-19 patients) and presence of associated biomarkers indicating bacterial superinfection.

FIG. 54 shows potentially explosive clinical course relating to viral and underlying host (chronic inflammation) factors.

FIG. 55 shows the relationship between COVID-19 severity and pre-existing host biofilms/chronic inflammation as a risk factor.

FIG. 56 shows biofilm eradication effect of BisEDT on Multidrug-resistant Pseudomonas aeruginosa: strain MR14 (6 log reduction at 0.25 ug/mL).

FIG. 57 shows biofilm eradication effect of BisEDT on Aminoglycoside-resistant Pseudomonas aeruginosa: strain AGR1 (7 log reduction at 2.5 ug/mL).

FIG. 58 shows biofilm eradication effect of BisEDT on Aminoglycoside-resistant Burkholderia cenocepacia: strain AU197 (6 log reduction at 2.5 ug/mL).

FIG. 59 shows biofilm eradication effect of BisEDT on M. abscessus complex: strain AMT0130-8 (6 log reduction at 2.5 ug/mL).

FIG. 60 shows Escherichia coli ATCC 25922 macromolecular synthesis inhibition by pravibismane.

FIG. 61 shows Escherichia coli ATCC 25922 and E. coli tolC mutant (MMX 121) RNA inhibition by pravibismane at higher concentrations.

FIG. 62 shows a temperature cycling DSC thermogram for amorphous BisEDT.

FIG. 63 shows a temperature modulated DSC thermogram for amorphous BisEDT.

### DETAILED DESCRIPTION

The present disclosure is based on the surprising discovery that bismuth thiol compounds such as BisEDT are effective for treating bacterial and fungal infection in patients with viral pulmonary infections such as COVID-19, alone or in combination with other anti-viral drugs.

As described herein, the present disclosure describes bismuth thiol compounds (e.g. BisEDT) and their ability to reduce COVID-19 mortality and morbidity. Patients most severely affected by respiratory viral infections in pandemics, inclusive of COVID-19, may derive substantial benefit from the targeted, inhaled administration of BisEDT.

BisEDT is the first drug candidate from a new class of anti-infective drugs (bismanes), with an extremely relevant, broad-spectrum of activity which includes potent activity against an extremely broad spectrum of relevant, multidrug-resistant pathogens. BisEDT has a unique mechanism of action which is not only capable of overcoming bacteria and their biofilms, but is also anticipated to prevent the production of bacterial toxins known as superantigens. Importantly, BisEDT has also been shown to be comparatively impervious to development of antibiotic-resistance. In human clinical studies evaluating pravibismane in the treatment of orthopedic infections and moderate to severe diabetic foot ulcers, BisEDT has been demonstrated to be very safe and well-tolerated. In addition, in vivo (preclinical) animal studies to date have demonstrated both the safety and tolerability of inhaled pravibismane, and also the efficacy of inhaled pravibismane in the treatment of pulmonary infection.

Many COVID-19 patients develop acute respiratory distress syndrome (ARDS), which leads to pulmonary edema and lung failure, and have liver, heart, and kidney damages. These symptoms are associated with a cytokine storm, manifesting elevated serum levels of IL-1 b, IL-2, IL-7, IL-8, IL-9, IL-10, IL-17, G-CSF, GMCSF, IFN-gamma, TNF-αt, IP10, MCP1, MIP1A and MIP1B. A safe therapeutic drug with the potential to a) prevent and treat biofilm dispersal, b) prevent and treat bacterial superinfections, and c) prevent bacterial superantigen production, could prevent mortality associated with COVID-19, as well as other respiratory viral infections. To this end, BisEDT is anticipated be locally effective, while avoiding the systemic side effects and toxicities that are associated with many systemically administered antibiotics. Further, BisEDT is expected to prevent and to treat biofilms and bacteria in the respiratory tract, and through its mechanism of action, is likely capable of preventing superantigen production and secretion. As a result, it is believed that BisEDT will be efficacious in the treatment and prevention of many chronic inflammatory conditions, including chronic respiratory tract infections, cardiovascular disease including atherosclerosis, G.I. tract inflammatory diseases, and neurodegenerative diseases such as Alzheimer's disease.

Inhaled BisEDT is anticpated to become a critically important tool for the treatment of primary bacterial and fungal infections of the lungs, and also for the treatment of the secondary bacterial and/or fungal pulmonary infections that complicate viral pulmonary infections during viral epidemics and pandemics.

### Biomarkers Implicate Bacterial Coinfections (Superinfections) and Bacterial Superantigens in COVID-19

In the absence of definitive post-mortem data, certain readily available biomarkers/blood tests have been investigated in recent years which may discriminate between bacterial infections or coinfections, and those that are solely viral in nature. Several biomarkers have been consistently associated with worse outcomes in the COVID-19 pandemic, including elevated neutrophil-lymphocyte ratio (NLR), IL-6 and procalcitonin, all of which are strongly associated with primary or secondary bacterial infections. NLR, for instance, is elevated in approximately 80% of severe COVID-19 patients. Based on these biomarkers, it is believed that the majority of the deaths in the current COVID-19 pandemic are due to secondary bacterial infections. Awareness of the profound contribution of bacterial superinfections to the mortality associated with COVID-19 should therefore influence the treatment of severe cases of COVID-19, particularly if, as one study suggests, coronavirus infections tend to predispose to Gram-negative secondary bacterial infections. The arsenal of antibiotics to combat Gram-negative infections is extremely limited, particularly those that are carbapenem-resistant or multidrug-resistant. Due to difficulty in testing for presence of specific bacterial species, empiric use of broad-spectrum antibiotics with strong activity against Pseudomonas, Acinetobacter, and Klebsiella may therefore be critical. However, considering the source of these bacteria are either seeded into the lungs from the upper respiratory tract, or from the intensive care unit (ICU) environment as multidrug-resistant (MDR) nosocomial pathogens, the likelihood is that a broad spectrum of bacteria could be involved. Accordingly, this consideration is urgent.

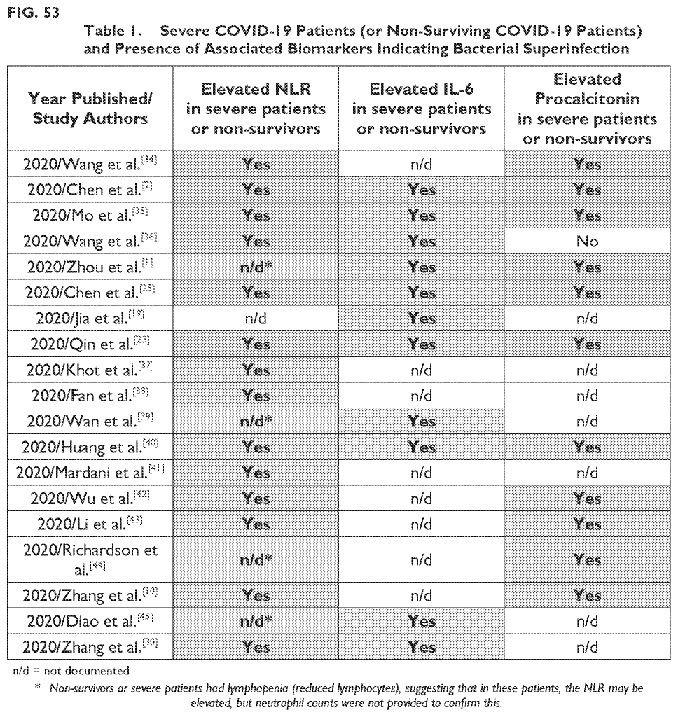

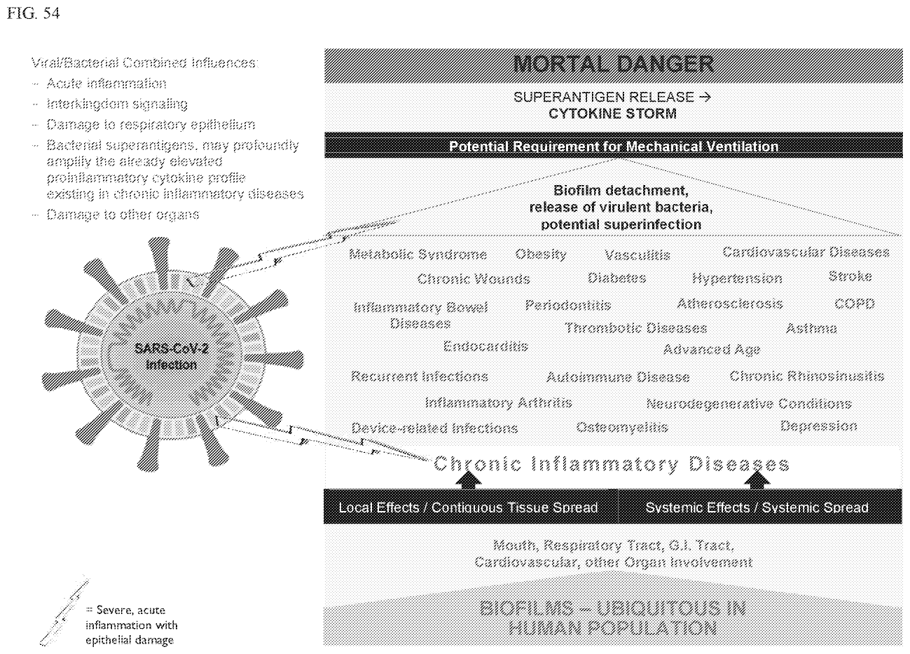

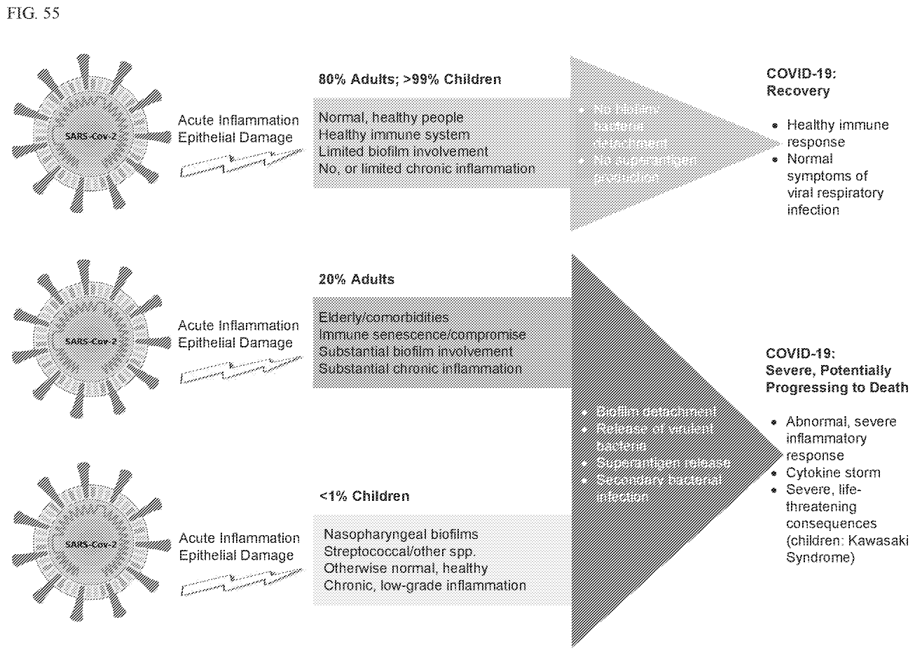

FIG. 53 represents a survey of currently available literature indicating that elevated values of the NLR, IL-6, and procalcitonin are reliably present in those patients that are either categorized as severe patients, or those that died from COVID-19. NLR was elevated in all of the 14 studies in which neutrophil and lymphocyte levels were reported (14 of the 19 studies listed). Of the remaining 5 studies, 4 studies documented lymphopenia (decreased lymphocytes) even though neutrophil levels were not reported (if these 4 lymphopenia-only reported studies were to be included, the total result would be that 18 of 19 studies demonstrated increased NLR). Elevated IL-6 levels were also described in all of the studies listed in FIG. 53, in which IL-6 levels were reported 11 of the 19 studies listed). Elevated procalcitonin levels were reported in all but one of the listed studies in which procalcitonin levels were reported (11 of the 19 studies listed). Every study with data on severe infections and/or non-survivors listed in FIG. 53 demonstrated elevated levels of one, two, or all three of biomarkers that are used to differentiate bacterial coinfections from purely viral infections. To confirm these finding with respect to NLR, two very recent studies published on COVID-19 both identified NLR as being highly predictive of COVID-19 disease severity. In addition, elevated levels of IL-6 were found to be a stable indicator of poor outcome in patients with severe COVID-19 with pneumonia and ARDS. The available biomarker data, in studies in which it was collected, consistently indicates a very strong likelihood that many, if not the majority of the most severe cases of COVID-19, are being caused by viral-bacterial superinfections. Secondary bacterial pulmonary infections (superinfections), opportunistically complicating primary respiratory viral infections, have long been responsible for many, if not the vast majority, of deaths related to respiratory viral pandemics (in pandemics over the past two decades, they have been found to be responsible for 30%-55% of pandemic mortality).

Taken together and combined with the biomarker profile in FIG. 53, these biomarkers and cytokines form a fact pattern consistent with active bacterial involvement: In the COVID-19 reports in which IL-2, IL-10, and interferon-gamma were measured, there were consistently elevated levels of these cytokines. As a result of the remarkable potency of bacterial superantigens, 1L-2, IL-10, and interferon-gamma are elevated in the presence of even the smallest (picomolar) concentrations of bacterial superantigens, and ultimately elicit proinflammatory cytokines that can induce fever, hypotension, multi-organ failure, and lethal shock. IL-17 is another very important cytokine that has been demonstrated to be involved in COVID-19. IL-17 is the key cytokine for protection against bacterial infections and fungi, infections that are controlled first by neutrophils. Thus, any disease in which neutrophils are involved suggests the contribution of the IL-17 pathway. Indeed, this pathway has been shown to be highly activated in patients affected by COVID-19. The elevated numbers of neutrophils in COVID-19 are commensurate with the elevations in IL-17; given the critical role that IL-17 and neutrophils play in bacterial and fungal infections, this again is highly suggestive of the role that bacteria and fungi are responsible for in COVID-19.

Role of Interkingdom Signaling and other Virally-Induced Triggers in Biofilm Dispersal from the Nasopharynx. Bacterial Superinfections, Superantigens. and Induction of Cytokine Storm

The mucosal epithelium, particularly that of the nasopharynx, is a key anatomical site of biofilms with important life-long health implications. Influenced by aging, genetics, lifestyle, the species of bacteria (and potentially fungi) involved, as well as other factors, it is a tissue to which biofilms slowly exert an increasingly insidious influence, by generating/spreading chronic inflammatory and infectious conditions, both locally and systemically. Importantly, the nasal mucosa is also central to COVID-19 as the initial site of attachment and infection of the SARS-CoV-2 virus, causing damage to the mucosa and stimulating an acute inflammatory response. With these two infectious and inflammatory circumstances combined, the mucosa is ‘ground zero’ for the intersection of chronic and acute inflammatory phenomena, with devastating consequences in COVID-19 patients who have pre-existing chronic inflammatory comorbidities.

The capacity of S. pneumoniae, a bacterium widely considered to be part of the normal flora of the nasopharynx which normally colonizes up to 95% of the human population, to establish biofilms on mucosal surfaces, has been identified as a key step in the pathogenesis of pneumococcal pneumonia and other pneumococcal infections, which are responsible for killing up to 2 million people per year.

The pathogenic manifestation of bacterial biofilms exerts a potent, proinflammatory immunomodulating effect, stimulating the increased expression of tumor necrosis factor-α (TNF-α), interleukin-1β(IL-1β), interleukin-6 (IL-6), IL-8, monocyte chemoattractive protein-1, interferon-λ induced protein 10 (IP-10), and intercellular adhesion molecule-1 (ICAM-1 and sICAM-1). The presence of bacterial biofilms in the upper airways is known to cause chronic inflammatory effects (e.g. periodontitis, chronic rhinosinusitis) with well-established local and systemic chronic inflammatory and infectious sequelae. Biofilm-producing bacteria have been shown to have a more potent pro-inflammatory effect on the lungs than non-biofilm-producing bacteria; the presence of biofilms is known to contribute to cytokine storms and other immune system-mediated tissue damage in the lungs.

Viruses can trigger biofilms to undergo ‘biofilm dispersal’ (the spread and activation of bacterial cells from biofilms). This virus-triggered dispersal/activation of bacteria from bacterial biofilms has been referred to as ‘interkingdom signaling’. Respiratory viruses are known, for instance, to trigger the dispersal of S. aureus and S. pneumoniae biofilms from nasal and bronchial epithelia, thus seeding the lungs with bacterial pathogens of increased virulence, causing secondary bacterial pneumonia. Infection with Influenza A has been demonstrated to influence commensal pneumococcal (S. pneumoniae) biofilms to release/disperse highly virulent S. pneumoniae diplococci having the capacity to cause pulmonary infections. Furthermore, through a distinct mechanism, Influenza A Virus (IAV) neuraminidase, which is similar in structure to S. pneumoniae neuraminidase, has been shown to synergistically promote respiratory infection by S. pneumoniae. S. aureus biofilms have also been shown to additionally disperse from the nasal cavity into the lungs by other virally-induced triggers, including fever, which resulted in a four-fold increase in biofilm dispersal. Recent studies have shown that changes in the nasopharyngeal environment caused by concomitant virus infection, changes in the microflora, inflammation, or other host assaults trigger active release of pneumococci from biofilms. These dispersed bacteria have distinct phenotypic properties and transcriptional profiles different from both biofilm and broth-grown, planktonic bacteria, resulting in a significantly increased virulence in vivo. Bacterial biofilms are an important source of eluting superantigens. Superantigens are exotoxins produced by bacteria in order to evade host immune response. Superantigens confer virulence by short-circuiting normal antigen presentation, leading to massive release of inflammatory cytokines. In addition, the host experiences massive T cell activation, expansion, and subsequent anergy as well as B cell activation. In effect, host immunity is debilitated, and pathogenic bacteria persist.

Superantigens target a large fraction of T cell pools to set in motion a cytokine storm. Superantigens are able to bind to the T cell receptor as well as to the major histocompatibility complex (MHC) Class II molecules present on antigen-presenting cells, thus cross-linking the two T-cell surface molecules resulting in the activation those T cells with concomitant secretion of cytokines, and bypassing the normal antigen processing step normally carried out by antigen presenting cells (APCs), which activates a much narrower population of T-cells through a more specific, controlled, and restricted activation pathway. Superantigens are able to ‘superactivate’ a large proportion of accessible T cells, leading to a massive “cytokine storm” and hyperinflammation and, under certain circumstances, to organ failure. Concurrent with the secretion of cytokines including IFN-λ, TNF-α and IL-2, the superactivated T cells are rendered useless in terms of immune activity in a manner consistent with exhaustion, becoming ‘anergic’ (losing functional abilities) and activating pathways leading to programmed cell death, including PD-1. In a study of 221 COVID-19 patients from Wuhan, both CD4+ and CD8+ T cells were found to be significantly reduced in severely affected patients, and T lymphocytes from these patients were also found to have a significantly increased expression of the programmed cell death marker, PD-1. This study also indicated that, of those patients whose final outcome was known at the time of the publication of the article, 100%6 of those who died were suffering from bacterial (55.6%) or fungal (44.4%) coinfections. While certain viruses are also capable of elaborating superantigens, this has not been documented in coronaviruses, including SARS, SARS-CoV-2, and MERS.

Mucosa-associated invariant T lymphocytes (MAIT) are activated by superantigens in extremely large numbers; MAIT cells comprise 1-10% of T cells in systemic circulation, up to 10% of T lymphocytes in the intestines, and about 45% of liver-associated T lymphocytes. Other T lymphocyte lineages are also susceptible to such activation. Superantigens act by triggering a massive and uncontrolled immunologic response activating as many as 20-30% of the T cell population in affected individuals, compared to the 0.001% that is activated in a normal antigen-specific immune response. This represents an up to 30,000-fold increase in T cell activation as compared to a normal adaptive immune response, generating a massive cytokine storm. MAIT lymphocytes have been identified as the most powerful source of pro-inflammatory cytokines after exposure to Superantigens.

Stimulation of chronic inflammation, acute inflammation, and cytokine storm is a fundamental pattern of bacterial/host interactive behavior, with direct relevance to COVID-19. When mucosal tissues in the upper respiratory tract become acutely inflamed and damaged, the biofilms which are adhered to mucosal tissues in those areas, disperse; bacterial cells and fragments of biofilms are spread along a contiguous epithelial route from the initial site of attachment (in the mouth and nasopharynx), to the lungs. The combination of several factors to trigger this dispersal, as well as the relative extent of these triggers, is likely what controls the likelihood of biofilm dispersal.

Chronic inflammation manifests as pre-existing chronic inflammatory conditions. The biofilms, which in the absence of a viral respiratory infection, had remained adherent to these tissues in a comparatively quiescent form. Even in the setting of a chronic inflammatory host response, these biofilms had established a delicate, yet slowly progressive pathogenic equilibrium with the host. By generating a physiological environment conducive to an extremely rapid, widespread, and profound expansion of inflammation and immune system stimulation, chronic inflammation predisposes afflicted patients to much greater risk of development of serious or fatal COVID-19.

It is notable that Kawasaki disease (KD) in children has been increasingly reported in relation to COVID-19. Superantigens are believed to play a causal role in KD. Up to 90%/6 of children, particularly in resource-constrained conditions, carry streptococcal biofilms in their nasopharynx. The presence of such nasopharyngeal bacteria and bacterial biofilms is related to acute and chronic middle ear infections and sinus infections in children, as well as to associated acute and/or chronic inflammation. Beyond increasing bacterial adherence and pathogenicity, biofilms have been found to be integral in the pathogenesis of otitis media (i.e. the inflammation of the area behind the tympanic membrane). Biofilms have been demonstrated to contribute to the inability of the host immune system to clear bacteria during chronic otitis media. The inflammatory mediators generated during otitis media can penetrate from the middle to inner ear potentially leading to hearing loss. Thus again, pre-existing chronic inflammation, in the case of chronic otitis media, or chronic sinus infections, even in a small proportion of susceptible children, may predispose to a greater risk of a rapid and very substantial over-reaction of the immune system, leading to cytokine storm and the Kawasaki syndrome-like illness that has been observed in children with COVID-19.

Overall, it is believed that biofilms, can actively contribute to the pathogenic overexpression of cytokines responsible for the phenomenon of cytokine storm. Considering the known pathophysiology and epidemiology of COVID-19, the long and consistent history of bacterial involvement in the mortality associated with viral epidemics, and ability of bacterial superantigens to explain most of the diverse manifestations of COVID-19, it is justified to investigate relevant therapeutic measures.

### Bacterial Superantigens: Lymphopenia and Lymphocyte Anergy

COVID-19 is characterized by pneumonia, lymphopenia, exhausted lymphocytes and a cytokine storm. It is believed that the consistent lymphopenia observed in severe COVID-19 patients is due to cytokine-induced lymphocyte trafficking into infected tissues, and/or sequestration of T-lymphocytes in the lungs. Exhausted lymphocytes, loss of lymphocyte function, cytokine storm and decreased lymphocyte numbers due to activation of programmed cell death are all associated with lymphocyte anergy, and all are findings that are highly consistent with immune system stimulation by superantigens, which are produced by both bacterial biofilms as well as the opportunistic bacteria that are released from biofilms, or that colonize the upper respiratory tract. Bacterial and superantigen involvement in the pathophysiology of COVID-19 is not just likely; it is at this time, with respect to the observed clinical characteristics of COVID-19, the most consistent, comprehensive, literature-supported explanation for lymphopenia, cytokine storm, and host susceptibilities to the worst outcomes.

### Chronic Inflammatory Diseases and Susceptibility to COVID-19:

Infection with SARS-CoV-2 and the ensuing disease process, COVID-19, poses a particular risk to people living with preexisting conditions that impair the immune response, or that predispose to rapid amplification from a clinical circumstance of the elevated cytokines and other immune factors related to chronic inflammation, to an acute, severely escalated pro-inflammatory response. Chronic inflammation and chronic inflammatory diseases including aging, diabetes, obesity, cardiovascular disease, hypertension, metabolic syndrome and others, comprise the specific populations that have been clearly identified as being most susceptible to the development of the most severe manifestations of COVID-19.

Patients having COVID-19 with preexisting chronic inflammation are at a high risk of ARDS and mortality. Advanced age combines dysfunction of major organs, especially cardiac functions. During the metabolic syndrome linked to obesity and diabetes, there is a massive storage of cytokines in adipose tissue. The main proinflammatory cytokines Interleukin-6 (IL-6), Tumor Necrosis Factor (TNF), and IL-17 play a major role in inflammation, including both chronic and acute inflammation. Accordingly, those suffering from chronic inflammation and chronic inflammatory diseases already demonstrate elevated levels of circulating proinflammatory cytokines, thus placing these individuals at increased risk that additional expression of such proinflammatory cytokines will become exaggerated upon further acute inflammatory insult.

This and other chronically stimulated pathogen/immune system diseases result in development chronic, slowly progressive series of inflammatory and/or metabolic diseases, characterized by low-grade, chronic inflammatory responses and insulin-resistance (think aging, diabetes, obesity, metabolic syndrome, atherosclerosis, hypertension, chronic kidney disease, COPD, cardiovascular and neurodegenerative diseases, and others). With respect to COVID-19, this underlying presence of low-grade, chronic inflammatory comorbidities is a recognized risk factor for the most severe manifestations of COVID-19, including death.

### Aging and Chronic Inflammation:

The microbiome of the upper respiratory tract changes throughout one's life, but in those over the age of 65, the differences in the diversity of bacteria between the various anatomical regions of the upper respiratory tract (that are otherwise present) are diminished, and Streptococci become established as a more significant part of the microbiome, accompanied by Staphylococci, and the anaerobes Prevotella and Veillonella.

Opportunistic secondary bacterial infections prosper in damaged lungs. Intensified systemic inflammation with aging can cause dysfunction in extra-pulmonary organs and tissues such as cardiovascular, musculoskeletal, neuropathologic, hepatic, and renal complications.

In other words, chronic inflammatory diseases beget chronic inflammatory diseases, in part due to the local and systemic effects of increasing levels of cytokines, other immune mediators, and cellular immunity, and in part due to the (often) underlying local and systemic spread of bacterial biofilms. No one single chronic inflammatory condition is the progenitor of all other chronic inflammatory diseases—but the one chronic inflammatory disease that perhaps comes closest to fitting this role is periodontitis. It is important to recognize that microbial biofilms (typically bacterial biofilms, or bacterial/fungal biofilms), are central to the etiology and pathophysiology of periodontitis, as well as many other chronic inflammatory conditions.

### Periodontitis:

The mouth houses a very substantial microbiome, with many of the bacterial cells resident in microbial biofilms, both in normal healthy people and in those suffering from periodontitis, gingivitis, and other oral infectious/inflammatory conditions. Biofilms are well known to house bacteria with characteristically, comparatively very low metabolic rates.

In healthy conditions, the microbial composition of dental plaque biofilm is balanced and stable. When this balance is disrupted by inflammation, microbial homeostasis breaks down and periodontal disease occurs . . . Pathobionts, a specific class of microbes, exemplified by the oral microbe Porphyromonas gingivalis, live mostly under the radar in their human hosts, in a cooperative relationship with the indigenous microbiota. Dendritic cells (DCs), mucosal immune sentinels, often remain undisturbed by such microbes and do not alert adaptive immunity to danger. However, if and when inflammation starts to occur in the mouth, such as in the case of poor oral hygiene, the once-friendly bacteria begin to act in ways that are more clearly pathogenic to the host triggering increased human (host) immune/inflammatory responses (including secretion of up to profound levels of cytokines—with associated pathophysiological consequences in the mouth, and potentially one or more of the respiratory tract, G.I. tract, and the rest of the human body, including (and via) the circulatory system.

At distant peripheral sites, comorbid diseases including atherosclerosis, Alzheimer's disease, macular degeneration, chronic kidney disease, and others are reportedly induced. Periodontitis (PD) is a chronic, dysbiotic inflammatory disease.

### Periodontitis and Relationship to Obesity, Metabolic Syndrome, Diabetes, Hypertension, and Atherosclerosis:

There is a positive association between prevalent periodontal disease and obesity. Obesity is a metabolic condition characterized by chronic, non-resolving low grade inflammation associated with elevated levels of proinflammatory molecules, including TNF-αt, CRP, IL-1β, monocyte chemoattractant protein-1/chemokine (C-C motif) ligand 2 (MCP-1/CCL2), and IL-6. Low-grade chronic systemic inflammation, common in people with obesity, is associated with the development of atherosclerosis, type 2 diabetes, and hypertension, well known comorbidities that adversely affect the outcomes of patients with COVID-19.

Persistent inflammation involves many proinflammatory factors that are inter-related; they are known to impact each other and if the underlying inflammation does not resolve, they mutually stimulate each other in a progressive vicious cycle. This low-grade chronic inflammation is characterized by a variety of chronic diseases, including cardiovascular disease, diabetes, hypertension, nonalcoholic fatty liver disease, hypercholesterolemia, asthma, arthritis, some cancers, and general poor health condition. During obesity, adipose tissue synthesizes and releases a large number of hormones and cytokines that alter the metabolic processes, with a profound influence on endothelial dysfunction, a situation associated with the formation of atherosclerotic plaque. Endothelial cells respond to inflammation and stimulation of MCP-1, which is described as the activation of adhesion molecules leading to proliferation and transmigration of leukocytes, which facilitates their increase in atherogenic and thromboembolic potentials. Endothelial dysfunction forms the cornerstone of this discussion, as it has been considered as the initiator in the progression of cardiovascular diseases in obesity. Obesity-induced inflammation is a multifaceted condition affecting many organs, including skeletal muscle, adipose tissue, liver, brain, heart, and pancreas.

Metabolic syndrome is a group of conditions defined by the presence of obesity, dyslipidemia, hypertension, and dysglycemia leading to an increased risk of diabetes and cardiovascular disease.

### Periodontitis and COPD:

Periodontitis is associated with respiratory diseases, including COPD, asthma, and pneumonia. Like COPD, CF patients afflicted by progressive, chronic infections caused by biofilm-dominated multi-species biofilms, and in both COPD and CF, pulmonary exacerbations are associated with progressively compromised lung function.

### Periodontitis and Alzheimer's Disease:

In addition to the systemic influence of periodontitis on conditions such as metabolic syndrome, atherosclerosis and cardiovascular disease, Alzheimer's disease is an example of the systemic spread of bacteria/bacterial biofilms from the mouth to influence distant sites.

### Role of Biofilms in Ventilator-Associated Pneumonia and Bacterial Superinfections

Ventilator-associated pneumonia (VAP) poses a dire risk to COVID-19 patients requiring mechanical ventilation. Even before COVID-19, rates of mortality associated with mechanical ventilation were reportedly up to 76%. Mortality rates for mechanically ventilated patients in COVID-19, for those patients whose outcomes are known, have been reported as up to 88.1%. Bacterial biofilms from the hospital environment, the patients' nasopharynx, and from the ventilator itself, represent primary causes of VAP.

### Definitions

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the verb “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

Unless specifically stated or obvious from context, as used herein, the term “or” is understood to be inclusive. Unless specifically stated or obvious from context, as used herein, the terms “a” “an”, and “the” are understood to be singular or plural.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

An “alkyl” group or “alkane” is a straight chained or branched non-aromatic hydrocarbon which is completely saturated. Typically, a straight chained or branched alkyl group has from 1 to about 20 carbon atoms, e.g. from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C1-C6 straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl can include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Exemplary substituted alkyls are described below. Cycloalkyls can be further substituted with alkyls, alkenyls, alkoxys, alkylthios, aminoalkyls, carbonyl-substituted alkyls, —CF3, —CN, and the like.

The term “Cx-y” when used in conjunction with a chemical moiety, such as, acyl, acyloxy, alkyl, alkenyl, alkynyl, or alkoxy is meant to include groups that contain from x to y carbons in the chain. For example, the term “Cx-yalkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C0 alkyl indicates a hydrogen where the group is in a terminal position, a bond if internal. The terms “C2-yalkenyl” and “C2-yalkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and can be represented by the general formula alkylS-.

The terms “amine” and “amino” are art-recognized and refer to both unsubstituted and substituted amines and salts thereof, e.g., a moiety that can be represented by

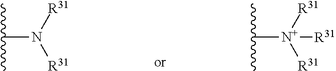

wherein each R³¹ independently represents a hydrogen or a hydrocarbyl group, or two R³¹ are taken together with the N atom to which they are attached complete a heterocycle having from 4 to 8 atoms in the ring structure. The term “aminoalkyl”, as used herein, refers to an alkyl group substituted with an amino group.

The term “aryl” as used herein include substituted or unsubstituted single-ring aromatic groups in which each atom of the ring is carbon. In some embodiments, the ring is a 5- to 7-membered ring, e.g. a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “bismuth” refers to the 83^{rd} element of the periodic table, or atoms or ions thereof. Bismuth can occur in the metallic state or in the ionized state, such as in the III or V oxidation state. Bismuth ions can form complexes with anions, either to make bismuth salts, or to form complex anions which are then further complexed with one or more additional cation(s). Bismuth can also form covalent bonds to other atoms, such as sulfur.

The term “Pravibismane” refers to a BisEDT composition suitable for inhalation by nebulization.

As disclosed herein, a “bismuth-thiol compound” or “BT compound” is a compound that has a bismuth atom covalently bound to one, two or three other sulfur atoms present on one or more thiol compounds. The term “thiol” refers to a carbon-containing compound, or fragment thereof, containing an —SH group and can be represented by the general formula R—SH. These thiol compounds include compounds with one, two, three or more S atoms. Thiol compounds can have other functionality, such as alkyl, hydroxyl, carbocyclyl, heterocyclyl, aryl, heteroaryl, amino, and other substituents. Thiol compounds having two or more S atoms can chelate the bismuth atom, such that two S atoms from the same molecule covalently bond with the bismuth atom. Exemplary bismuth-thiol compounds are shown below:

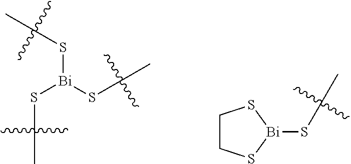

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle can be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle can be selected from saturated, unsaturated and aromatic rings. In an exemplary embodiment, an aromatic ring, e.g., phenyl, can be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” can be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl can be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl can be selected from saturated, unsaturated and aromatic rings. A “cycloalkenyl” group is a cyclic hydrocarbon containing one or more double bonds.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, for example 5- to 7-membered rings, e.g. 5- to 6-membered rings, whose ring structures include at least one heteroatom, for example one to four heteroatoms, e.g. one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, for example, 3- to 10-membered rings, more e.g. 3- to 7-membered rings, whose ring structures include at least one heteroatom, e.g. one to four heteroatoms, e.g. one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but can optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, for example, six or fewer. A “lower alkyl”, for example, refers to an alkyl group that contains ten or fewer carbon atoms, e.g. six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, e.g. from 5 to 7.

The term “N-oxide” refers to a zwitterionic group containing a nitrogen atom in the +1 oxidaton state bound to an oxygen atom in the −1 oxidation state. An non-limiting example of an N-oxide is pyridium N-oxide shown below. As used herein, the term “N-oxide” encompasses substituents of other groups having this functionality.

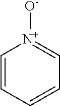

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen can have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “thioalkyl”, as used herein, refers to an alkyl group substituted with a thiol group. A “thiol compound” as discussed above can include a thioalkyl as a substituent on the compound structure. A thiol compound can have, for example, one, two, three or more thioalkyl groups.

The term “thioether”, as used herein, is equivalent to an ether, wherein the oxygen is replaced with a sulfur.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week.

“Coadministration” refers to the administration of the two agents in any manner in which the pharmacological effects of both agents are manifest in the patient at the same time. Thus, concomitant administration does not require that a single pharmaceutical composition, the same dosage form, or even the same route of administration be used for administration of both agents or that the two agents be administered at precisely the same time. However, in some situations, coadministration will be accomplished most conveniently by the same dosage form and the same route of administration, at substantially the same time.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “managing” includes therapeutic treatments as defined above. Managing includes achieving a steady state level of infection as determined by known methods in the art. The steady state can include evaluation of one or more of the severity of the infection(s), the size and location of the infection(s), the number of different microbial pathogens present in the infection(s), the level of antibiotic tolerant or resistant microbial pathogens, the degree of response to treatment, such as with a BT composition disclosed herein, the degree of biofilm formation and reduction, and the side effects experienced by the subject. During management of an infection, the infection may fluctuate from increasing to lessening in severity, in the amount or extent of infection, amount of side effects experienced by the subject, or other subject outcome indicia. Over a period of time, such as days, month, or years, the degree of management of the infection can be determined by evaluation of the above factors to assess whether the clinical course of infection has improved, is bacteriostatic, or has worsened. In some embodiments, managing an infection include successful treatment of microbial pathogen(s) that are otherwise drug tolerant or drug resistant.

The term “lessen the severity” of infection(s) refers to an improvement in the clinical course of the infection on any measurable basis. Such basis can include measurable indices such as reducing the extent of infection (s), whether the infection(s) are considered acute, the number and identity of microbial pathogens causing the infection(s), the extent of microbial (e.g. bacterial or fungal) biofilms, and side effects experienced by the subject. In some embodiments, lessening the severity of an infection is determined by measurements such as reduction in sputum infection counts (e.g. a reduction in CFU in the sputum). In some embodiments, lessening the severity involves halting a steady decline in outcome to achieve stabilized infection(s), resulting in the subject entering successful management of the infection(s). In other embodiments, lessening the severity can result in substantial to complete treatment of the infection(s). In some embodiments, lessening the severity refers to a lessening of exacerbations associated with the disease or infection (for example by a 1%-99% decrease in exacerbations). In some embodiments, lessening the severity can refer to an increase in lung function (for example by a 1%-99% increase in lung function).

As used herein, the term “exacerbation” refers to an increase in the severity of symptoms during a course of a disease which is mostly associated with a worsening of quality of life.

In some embodiments, lessening the severity of infections and/or symptoms can relate to patient-reported outcomes (“PROSs”). A PRO instrument is defined as any measure of a subject's health status that is elicited from the patient and determines how the patient “feels or functions with respect to his or her health condition. Such symptoms can be observable events, behaviors, or feelings (e.g., ability to walk quickly, lack of appetite, expressions of anger), or unobservable outcomes that are known only to the patient (e.g., perceptions of pain, feelings of depression).

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of infection (e.g. respiratory infection).

A “response” to a method of treatment can include a decrease in or amelioration of negative symptoms, a decrease in the progression of an infection or symptoms thereof, an increase in beneficial symptoms or clinical outcomes, a lessening of side effects, stabilization of the infection, and partial or complete remedy of infection, among others.

“Antibiotic susceptibility or sensitivity” refers to whether a bacteria will be successfully treated by a given antibiotic. Similarly, “Antifungal susceptibility or sensitivity” refers to whether a fungi will be successfully treated by a given antibiotic. Testing for susceptibility can be performed by methods known in the art such as the Kirby-Bauer method, the Stokes method and Agar Broth dilution methods. The effectiveness of an antibiotic in killing the bacteria or preventing bacteria from multiplying can be observed as areas of reduced or stable amount, respectively, of bacterial growth on a medium such as a wafer, agar, or broth culture.

“Antimicrobial tolerance” refers to the ability of a microbe, such as bacteria or fungi, to naturally resist being killed by antibiotics. It is not caused by mutant microbes but rather by microbial cells that exist in a transient, dormant, non-dividing state. Antibiotic or drug tolerance is caused by a small subpopulation of microbial cells termed persisters. Persisters are not mutants, but rather are dormant cells that can survive the antimicrobial treatments that kill the majority of their genetically identical siblings. Persister cells have entered a non- or extremely slow-growing physiological state which makes them insensitive (refractory or tolerant) to the action of antimicrobial drugs. Similarly, “antibiotic tolerance” refers to the ability of a bacteria to naturally resist being killed by antibiotics and “antifungal tolerance” refers to the ability of a fungi to naturally resist being killed by antibiotics.

“Antimicrobial resistance” refers to the ability of a microbe to resist the effects of medication that once could successfully treat the microbe. Microbes resistant to multiple antimicrobials are called multidrug resistant (MDR). Resistance arises through one of three mechanisms: natural resistance in certain types of bacteria, genetic mutation, or by one species acquiring resistance from another. Mutations can lead to drug inactivation, alteration of the drug's binding site, alteration of metabolic pathways and decreasing drug permeability.

As used herein, the terms “antibacterial activity”, “antifungal activity” and “antimicrobial activity”, with reference to a BT composition of the present disclosure, refers to the ability to kill and/or inhibit the growth or reproduction of a particular microorganism. In certain embodiments, antibacterial or antimicrobial activity is assessed by culturing bacteria, e.g., Gram-positive bacteria (e.g., S. aureus), Gram-negative bacteria (e.g., A. baumannii, E. coli, and/or P. aeruginosa) or bacteria not classified as either Gram-positive or Gram-negative, or fungi according to standard techniques (e.g., in liquid culture or on agar plates), contacting the culture with a BT composition of the present disclosure and monitoring cell growth after said contacting. For example, in a liquid culture, bacteria may be grown to an optical density (“OD”) representative of a mid-point in exponential growth of the culture; the culture is exposed to one or more concentrations of one or more BT compounds of the present disclosure, or variants thereof, and the OD is monitored relative to a control culture. Decreased OD relative to a control culture is representative of antibacterial activity (e.g., exhibits lytic killing activity). Similarly, bacterial colonies can be allowed to form on an agar plate, the plate exposed to a BT composition of the present disclosure, or variants thereof, and subsequent growth of the colonies evaluated related to control plates. Decreased size of colonies, or decreased total numbers of colonies, indicate antibacterial activity.

“Biofilm” refers any syntrophic consortium of microorganisms in which cells stick to each other and often also to a surface. These adherent cells become embedded within a slimy extracellular matrix that is composed of extracellular polymeric substances (EPS). Upon formation of biofilms, microbial resistance to antibiotics is up to 1000 times greater compared to that of planktonic bacteria. Bacterial aggregates are clusters of laterally aligned cells can initiate biofilm development, which has a more complex and denser 3-D structure. In some embodiments, the biofilm may comprise one or more species of bacteria (e.g., Pseudomonas aeruginosa and Staphylococcus aureus) and/or one or more different phyla (e.g., bacteria, virus and fungi).

As used herein, the term “microbe” refers to a microorganism and is intended to encompass both an individual organism, and hetero and homogenous populations comprising any number of the organisms. As used herein, the term “microorganism” refers to any of a variety of species or microorganism, including but not limited to, archaea, bacteria, fungi, protozoans, mycoplasma, and parasitic organisms, wherein the term “fungi” is used in reference to eukaryotic organisms such as the molds and yeasts, including dimorphic fungi, and the terms “bacteria” and “bacterium” refers to the various examples as specifically disclosed in the tables and description herein, broadly including prokaryotic organisms within the phyla in the kingdom Procaryotae, the microorganisms including Actinomyces, Chlamydia, Streptomyce, and all cocci, bacilli, spirochetes, spheroplasts, protoplasts, all Gram-negative and Gram-positive “Gram-negative” and “Gram-positive” refer to staining patterns with the Gram-staining process, and all non-pathogenic bacteria and pathogenic bacteria.

The term “pathogen” refers to a biological organism that causes or to which can be at least partially attributed any of a variety of disease states in a host, and include, but are not limited to, archaea, bacteria, fungi, protozoans, mycoplasma, parasites, and viruses.

As used herein, discussion of bacterial or fungal pathogens also encompass any microbe (e.g. bacteria and/or fungi) that contributes to the pathological state in the lungs. This includes both recognized and unrecognized microbes, and may also include bacteria or fungi that are not pathogens, but that simply facilitate the activity and presence of pathogens and their biofilms. As an example, embodiments directed to the inhibition of cell viability or cell growth of planktonic cells of the bacterial or fungal pathogen also extend to the inhibition of cell viability or cell growth of planktonic cells of the bacterial and/or fungal microbes that simply facilitate the activity and presence of pathogens and their biofilms.

“Airway surface” and “pulmonary surface,” as used herein, include pulmonary airway surfaces such as the bronchi and bronchioles, alveolar surfaces, and nasal and sinus surfaces.

“Saline” as used herein refers to a solution comprised of, consisting of, or consisting essentially of sodium chloride in water. Saline can be hypertonic, isotonic, or hypotonic. In some embodiments, saline can comprise sodium chloride in an amount of from about 0.1% to about 40% by weight, or any range therein, such as, but not limited to, about 0.1% to about 10%, about 0.5% to about 15%, about 1% to about 20%, about 5% to about 25%, about 10% to about 40%, or about 15% to about 35% by weight (in mg/100 mL). In certain embodiments, sodium chloride is included in a solution in an amount of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% by weight (in mg/100 mL), or any range therein.

“Hypertonic saline” as used herein refers to a solution comprised of, consisting of, or consisting essentially of greater than 0.9 wt % sodium chloride in water. In general, the sodium chloride is included in the solution in an amount of from about 0.9% to about 40% by weight, or any range therein, such as, but not limited to, about 1% to about 15%, about 5% to about 20%, about 5% to about 25%, about 10% to about 40%, or about 15% to about 35% by weight. In certain embodiments, sodium chloride is included in the solution in an amount of about 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40% by weight, or any range therein.

“Hypotonic saline” as used herein refers to a solution comprised of, consisting of, or consisting essentially of less than 0.9 wt % sodium chloride in water. In some embodiments, sodium chloride is included in the solution in an amount of about 0.9%, 0.8%, 0.7%, 0.6%, 0.5%, 0.4%, 0.3%, 0.2%, 0.1% by weight, or any range therein.

“Isotonic saline” as used herein refers to a solution comprised of, consisting of, or consisting essentially of 0.9 wt % sodium chloride in water.

According to some embodiments, saline (e.g., hypertonic saline) can include an excipient. An excipient can be a pharmaceutically acceptable excipient. “Pharmaceutically acceptable” as used herein means that the compound or composition is suitable for administration to a subject to achieve the treatments described herein, without unduly deleterious side effects in light of the severity of the disease and necessity of the treatment. Exemplary excipients include, but are not limited to, a buffer and/or a buffering agent (e.g., an anion, a cation, an organic compound, a salt, etc.). Exemplary buffers include, but are not limited to, carbonic acid/carbonate/bicarbonate-based buffers, disodium hydrogen phthalate/sodium dihydrogen orthophosphate-based buffers, tris(hydroxymethyl)aminomethane/hydrochloric acid-based buffers, barbitone sodium/hydrochloric acid-based buffers, and any combination thereof. Exemplary buffering agents include, but are not limited to, carbonic acid, carbonate, bicarbonate, disodium hydrogen phthalate, sodium dihydrogen orthophosphate, tris(hydroxymethyl)aminomethane, hydrochloric acid, barbitone sodium, dissolved CO₂(e.g., CO₂ formulated at a pH of greater than 6.6), and any combination thereof. In certain embodiments, saline comprises a bicarbonate buffer excipient, such as a bicarbonate anion (HCO₃). In some embodiments, hypertonic saline can include sodium bicarbonate, sodium carbonate, carbonic acid, and/or dissolved CO₂ formulated at a pH of greater than 6.5. Additional ingredients can be included as desired depending upon the particular condition being treated, as discussed further below.

As used herein, the term “volumetric median diameter” or “VMD” of an aerosol is the particle size diameter identified such that half of the mass of the aerosol particles is contained in particles with larger diameter than the VMD, and half of the mass of the aerosol particles is contained in particles with smaller diameter than the VMD. VMD is typically measured by laser diffraction.

“Mass median aerodynamic diameter” or “MMAD” is a measure of the aerodynamic size of a dispersed aerosol particle. The aerodynamic diameter is used to describe an aerosolized particle in terms of its settling behavior, and is the diameter of a unit density sphere having the same settling velocity, generally in air, as the particle in question. The aerodynamic diameter encompasses particle shape, density and physical size of a particle. As used herein, MMAD refers to the midpoint or median of the aerodynamic particle size distribution of an aerosolized particle determined by cascade impaction and/or laser time of flight and/or cascade impactor.

“Mass median diameter” or “MMD” is a measure of mean particle size. Any number of commonly employed techniques can be used for measuring mean particle size.

As used herein, “D90” refers to the 90% value of particle diameter (either the microparticle or aerosolized particle). For example if D90=1 μm, 90% of the particles are smaller than 1 μm. Similarly, “D80” refers to the 80% value of particle diameter (either the microparticle or aerosolized particle), “D70” refers to the 70% value of particle diameter (either the microparticle or aerosolized particle), “D60” refers to the 60% value of particle diameter (either the microparticle or aerosolized particle), “D50” refers to the 50% value of particle diameter (either the microparticle or aerosolized particle), “D40” refers to the 40% value of particle diameter (either the microparticle or aerosolized particle), “D30” refers to the 30% value of particle diameter (either the microparticle or aerosolized particle), “D20” refers to the 20% value of particle diameter (either the microparticle or aerosolized particle), “D10” refers to the 10% value of particle diameter (either the microparticle or aerosolized particle).

As used herein, “monodisperse” refers to a collection of particles (bulk or aerosol dispersion) comprising particles of a substantially uniform MMD and/or MMAD and/or VMD.

As used herein, the term “deposition efficiency” refers to the percentage of the delivered dose that is deposited into the area of interest. Thus, the deposition efficiency of a method and/or system for delivering an aerosolized medicament into the lungs is the amount by mass of the aerosol deposited into the lungs divided by the total amount of the aerosol delivered by the system to the nares.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

As used herein, “superinfection” means infection occurring after, on top, or in addition to an earlier infection, such as a second infection superimposed on an earlier infection.

As used hererin, “superantigen” means an antigen that results in excess activation of the immune system in a subject relative to a non-superantigen, and thus activating higher than 0.001% of the subject's T-cells due to infection of the antigen. For example, a superantigen is known to activate 0.01% to 20% of T-cells in a subject. In other examples, the superantigen is known to activate 0.1% to 20% of T-cells in a subject. In other examples, the superantigen is known to activate 1.0% to 20% of T-cells in a subject.

As used herein, “cytokine storm” means a severe immune reaction in which the body releases an excessive or uncontrolled release of proinflammatory cytokines into the blood too quickly in a subject. Symptoms from a cytokine storm can include a high fever, inflammation (redness and swelling), severe fatigue and nausea, multiple organ failure, and death.

The term “amorphous” can be used in the context of this invention to designate the state of solid substances, in which the components (which can be atoms, ions or molecules), do not exhibit any periodic arrangement over a great range (=long-range order). For example, in the case of amorphous BisEDT, the BisEDT molecules or ions do not exhibit any periodic arrangement over a great range (=long-range order). In amorphous substances, the components are usually not arranged in a totally disordered fashion and completely randomly, but are rather distributed in such a way that a certain regularity and similarity to the crystalline state can be observed with regard to the distance from and orientation towards their closest neighbours (=short-range order). Amorphous substances consequently preferably possess a short-range order, but no long-range order as present in a crystal lattice. The identification and characterization of various morphic or amorphic forms of a pharmaceutically active compound is of great significance in obtaining medicaments with desired properties including a specific dissolution rate, milling property, bulk density, thermal stability or shelf-life.

The term “glass transition temperature” (Tg) is reported to describe the temperature at which amorphous or partially crystalline polymers change from the rigid solid state to the rubbery or more flexible state. In the process, a distinct change in physical parameters, e.g. hardness and elasticity, occurs. Beneath the Tg, a polymer is usually glassy and hard, whereas above the Tg, it changes into a rubber-like to viscous state.

The term “impurity” of a compound, as used herein, means chemicals other than the compound, including, derivatives of the compound, or degradants of the compound, or incompletely reacted reagents of the synthesis that remain with the product compound due to incomplete purification, or that develop over time, such as during stability testing, formulation development of the compound or storage of the compound.

The term “chemical purity” of a compound, as used herein, refers to the purity of a compound from other distinct chemical entities. For example, amorphous BisEDT having 90% chemical purity means that the amorphous form contains less than 10% of molecules or chemical entity different from BisEDTm including synthetic byproducts, residual solvents, or residual organic or inorganic substances.

The term “substantially similar” as used herein with regards to an analytical spectrum, such as XRPD patterns, Raman spectroscopy, etc., means that a spectrum resembles the reference spectrum to a great degree in both the peak locations and their intensity.

The term “substantially free of” as used herein, means free from therapeutically effective amounts of compounds when administered in suggested doses, but may include trace amounts of compounds in non-therapeutically effective amounts.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, the phrase “conjoint administration” refers to any form of administration of two

The term “infection” is used herein in its broadest sense and refers to any infection, such as viral infection or caused by a microorganism bacterial infection, fungal infection or parasitic infection (e.g. protozoa, amoeba or helminths). Examples of such infections can be found in a number of well-known texts such as “Medical Microbiology” (Greenwood, D., Slack, R., Peutherer, J., Churchill Livingstone Press, 2002); “Mims' Pathogenesis of Infectious Disease” (Mims, C., Nash, A., Stephen, J., Academic Press, 2000); “Fields” Virology. (Fields, B N, Knipe D M, Howley, P M, Lippincott Williams and Wilkins, 2001); and “The Sanford Guide To Antimicrobial Therapy,” 26th Edition, JP Sanford et al. (Antimicrobial Therapy, Inc., 1996), which is incorporated by reference herein. The presence of infection in e.g. a diabetic foot wound is defined by clinical signs and symptoms of infection or inflammation, not by the culture of microorganisms, which are always present. However, immediately following resolution of clinical signs and symptoms of a wound infection, most patients will still have the underlying ulcer (e.g. diabetic foot ulcer), which requires continued treatment to facilitate complete wound closure. Of note, however, is that many wound specialists believe that in addition to the clinically defined state of infection, a less clinically apparent pathological state, known as “critical colonization” exists. In this state, a wound may be delayed or arrested in wound healing due to the subclinical presence of a high level of bacteria. This critical colonization, sometimes referred to as a high ‘wound bioburden’, is often polymicrobial and associated with biofilm-producing bacteria; it has been shown to induce, or prolong, the active inflammatory phase of repair, thus preventing a normal wound healing process. The bacterial cells that comprise such biofilms are difficult to recognize because they often exist in a viable, but nonculturable (VBNC), state (Pasquaroli 2013), yet they are adherent to surfaces and are typically more tolerant and resistant than their planktonic counterparts to antibiotics and antiseptics (Costerton 1999, Nguyen 2011). The term “infection” therefore contemplates the clinically defined state of infection as well as “critical colonization.”

The term “wound closure” can encompass healing of a wound wherein sides of the wound are rejoined to form a continuous barrier (e.g., intact skin). In another embodiment, the compositions and methods provided herein promote tissue regeneration. In another embodiment, the compositions and methods provided herein limit scarring of tissues such as glia, tendons, eye tissue, ligament or skin. In some embodiments, “wound closure” refers to complete or substantially complete re-epithelialization. In some embodiments, “wound closure” occurs via secondary intention.

It is to be understood that the term “wound healing” can encompass a regenerative process with the induction of a temporal and spatial healing program comprising wound closure and the processes involved in wound closure. The term “wound healing” can also encompass the processes of granulation, neovascularization, fibroblast, endothelial and epithelial cell migration, extracellular matrix deposition, re-epithelialization, and remodeling. In some embodiments, “wound healing” refers to a wound remaining closed for a sufficient period of time after the initial wound closure (e.g. one day, two days, three days, four days, five days, six days, one week, two weeks, three weeks, or one month). In some embodiments, “wound healing” refers to a wound remaining closed for two weeks after the initial wound closure.

It will be appreciated by a skilled artisan that the term “granulation” can encompass the process whereby small, red, grainlike prominences form on a raw surface (that of wounds or ulcers) as healing agents. Granulation may also include the formation of granulation tissue over the wound. “Granulation tissue” refers to the newly growing tissue material at a wound site formed to heal the wound. The tissue is perfused, fibrous connective tissue including a variety of cell types. The tissue will grow generally from the base of the wound to gradually fill the entire wound space.

It will be appreciated by a skilled artisan that the term “neovascularization” can encompass the new growth of blood vessels with the result that the oxygen and nutrient supply is improved. Similarly, it will be appreciated by the skilled artisan that the term “angiogenesis” may encompass the vascularization process involving the development of new capillary blood vessels. It will also be appreciated that the term “cell migration” refers to the movement of cells (e.g., fibroblast, endothelial, epithelial, etc.) to the wound site.

It is to be understood that the term “extracellular matrix deposition” can encompass the secretion by cells of fibrous elements (e.g., collagen, elastin, reticulin), link proteins (e.g., fibronectin, laminin), and space filling molecules (e.g., glycosaminoglycans). It will be appreciated by the skilled artisan that the term “type I collagen” can encompass the most abundant collagen, which forms large well-organized fibrils having high tensile strength.

It will be appreciated by a skilled artisan that the term “re-epithelialization” can encompass the reformation of epithelium over a denuded surface (e.g., wound).

The term “remodeling” refers to the replacement of and/or devascularization of granulation tissue.

### Methods of Use

In some embodiments, the present disclosure provides methods of preventing infections to a primary respiratory viral infection in a subject, the method comprising administering to the subject a bismuth-thiol (BT) composition that comprises BisEDT suspended therein, wherein administering the BT composition is via inhalation, orally or nasally, using an aerosol device.

In some embodiments, the BT composition is administered by inhalation, either orally or nasally, using an aerosol device, such as a nebulizer. A nebulizer can administer the BT composition topically to the lung tissue, which can include the lung mucosa, the alveoli (e.g. deep lung alveoli), the bronchi and/or the bronchioles. Thus, in some embodiments, the present disclosure provides for administration of the BT composition to the deep lung region of the lung (e.g. the deep lung alveoli). Local topical administration of the BT composition provides several key advantages over systemic antibiotic therapies. The term “systemic” refers to administration of a medication into the circulatory system of the subject such that the majority of the entire body can be exposed. Systemic administration of a medication can occur enterally (absorption through the gastrointestinal tract, e.g. oral administration) or parenterally (absorption through injection or infusion, e.g. intravenously).

In FIG. 11, plasma concentration is depicted for an orally and pulmonary dosed drug. Oral dosing (A) can result in high plasma concentrations that may lead to toxicity and varied inter-patient exposure (variable absorption and variable first-pass, hepatic clearance) or drug-drug interactions. High plasma exposure is necessary to achieve therapeutic exposure in the lungs. In contrast, inhaled dosing for topical lung indications requires a lower total dose to achieve an efficacious MIC, which results in significantly less systemic exposure. Inhaled dosing (B) achieves higher local concentrations in the lung that significantly exceed the MIC of the drug over a long period of time. Due to the delivery of high concentrations of drug directly to the lung, the achieved pulmonary concentrations following inhalation may greatly exceed those achieved by oral dosing. From previous experience with inhaled antibiotics, the lung concentration of drug upon inhalation is >100× greater than upon systemic/oral administration of the same dose.

BT compounds are known broad-spectrum antimicrobial (and anti-biofilm) small molecule drug product for the treatment of chronic, ultimately life-threatening pulmonary infections. Its efficacy extends to Gram-positive, antibiotic-resistant pathogens including methicillin-resistant Staphylococcus aureus (MRSA, including community-associated [CA]-MRSA), methicillin-resistant Staphylococcus epidermidis (MRSE), and vancomycin-resistant Enterococcus (VRE). BT compounds are also potent against Multi-drug-resistant (MDR) Gram-negative pathogens including Pseudomonas aeruginosa, Escherichia co/i, Klebsiella pneumoniae (including, in all of the afore-mentioned bacteria, carbapenem-resistant strains), and Acinetobacter baumannii.

BT compounds have the dual ability to overcome a) a very diversified spectrum of antibiotic resistance profiles (due to evolution/diversification driven by persistence, time and isolation in many different anatomical regions throughout the pulmonary airways), and b) antibiotic-resistant and MDR biofilms.

Disclosed herein are methods of treating, managing or lessening the severity of symptoms associated with infections to a primary respiratory viral infection in a subject, the method comprising administering to the subject a bismuth-thiol (BT) composition that comprises BisEDT suspended therein, wherein administering the BT composition is via inhalation, orally or nasally, using an aerosol device. In some embodiments, the subject has at least one pulmonary infection. In other embodiments, the subject has at least two pulmonary infections and the infections are either concurrent or successive in order. The pulmonary infections could be caused by the same microbial pathogen and be located in two different lungs, or lobes of the lung. In other embodiments, the pulmonary infections could be caused by different microbial pathogens and be located in the same lung, or lobe of the lung. In some embodiments, the pulmonary infection is in one lung, while in others it is present in both lungs. In certain embodiments, the pulmonary infection is in one or more of the three lobes of the right lung. In other embodiments, the pulmonary infection is in one or both of the two lobes of the left lung. Any combination of one or more microbial pathogens, microbial pathogen quantity, and infection location in the lung is contemplated within the term “pulmonary infection”.

In certain embodiments, the pulmonary infection is located in or on the lung mucosa, the bronchi and/or the bronchioles. In other embodiments, the pulmonary infection is located on the surface of or within a bacterial biofilm, aggregated bacteria, a fungal biofilm, or aggregated fungi. In some embodiments, the pulmonary infection is located in the sputum wherein the pulmonary infection involves and is, at least in part, present in the mucous/sputum layers associated with the lungs. In certain embodiments, the bacterial pathogen comprises one or more of gram-positive bacteria and gram-negative bacteria. The bacterial pathogen can comprise one or more of a bacterial biofilm and planktonic bacteria. In some embodiments, the fungal pathogen comprises one or more of a fungal biofilm and planktonic fungi. In certain embodiments, the fungal pathogen is Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, and/or Aspergillus flavus.

In some embodiments, the infection that is treated, managed or lessened is the viral infection. In some embodiments, the infection that is treated, managed or lessened is a bacterial and/or fungal infection that is secondary to the viral infection.

the method comprises at least one of: (i) reducing a biofilm (e.g. bacterial and/or fungal), (ii) impairing growth of a biofilm (e.g. bacterial and/or fungal), (iii) preventing initial formation of the biofilm (e.g. bacterial and/or fungal), and/or (iv) preventing reformation of the biofilm (e.g. bacterial and/or fungal). In other embodiments, the BT composition treats, manages or lessens the severity of the pulmonary infection by one or more of:
prevention of the infection by the bacterial or fungal pathogen; —prevention of elaboration or secretion of exotoxins from the bacterial or fungal pathogen;
reduction of the bacterial or fungal pathogen (e.g. as measure by amount or titer);
inhibition of cell viability or cell growth of planktonic cells (e.g. substantially all of the cells) of the bacterial or fungal pathogen;
inhibition of biofilm formation by the bacterial or fungal pathogen;
inhibition of biofilm viability or biofilm growth of biofilm-form cells (e.g. substantially all of the cells) of the bacterial or fungal pathogen; and
reducing the viscosity of the sputum.

In some embodiments, the bismuth-thiol composition comprises a plurality of microparticles that comprise a bismuth-thiol (BT) compound, substantially all of said microparticles having a volumetric mean diameter of from about 0.4 μm to about 5 μm, and wherein the BT compound comprises bismuth or a bismuth salt and a thiol-containing compound. In some embodiments, the bismuth salt is bismuth nitrate, bismuth subnitrate, or bismuth chloride. In some embodiments, the thiol-containing compound comprises one or more agents selected from 1,2-ethane dithiol, 2,3-dimercaptopropanol, pyrithione, dithioerythritol, 3,4 dimercaptotoluene, 2,3-butanedithiol, 1,3-propanedithiol, 2-hydroxypropanethiol, 1-mercapto-2-propanol, dithioerythritol, dithiothreitol, cysteamine, and alpha-lipoic acid. In some embodiments, at least 60%, 65%, 70, 75%, 80%, 90%, or 95% of the microparticles have a volumetric mean diameter of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the presently disclosed methods, at least 60%, 65%, 70, 75%, 80%, 90%, or 95% of the microparticles have a volumetric mean diameter of from about 0.6 μm to about 2.5 μm. In some embodiments, substantially all of the microparticles have a VMD of from about 0.6 μm to about 2.5 μm. In some embodiments, at least 70% of the aerosolized particles have a MMAD of about 0.9 μm to about 3 μm. In some embodiments, the composition is a suspension of microparticles having a volumetric mean diameter (VMD) from about 0.6 μm to about 2.5 μm and/or a mass median aerodynamic diameter (MMAD) from about 0.9 μm to about 3 μm. In some embodiments, the bismuth-thiol composition comprises a plurality of microparticles that comprise a bismuth-thiol (BT) compound, substantially all of said microparticles having a volumetric mean diameter of from about 0.4 m to about 5 μm, and wherein the BT compound comprises bismuth or a bismuth salt and a thiol-containing compound.

In some embodiments, the BT composition comprises one or more BT compounds selected from

bismuth-2,3-dimercaptopropanol (2:3 molar ratio, BisBAL)
bismuth-dithioerythritol (2:3 molar ratio, BisERY)
bismuth-4-methyl-1,2-benzenedithiol (2:3 molar ratio, BisTOL)
bismuth-2,3-butanedithiol (BisBDT)
bismuth-2,3-butanedithiol, 2-mercaptopyridine N-oxide (2:1:2 molar ratio, BisBDT/PYR)
bismuth-2,3-dimercaptopropanol, 2-mercaptopyridine N-oxide (2:1:2 molar ratio,
BisBAL/PYR)
bismuth-1,2-ethanedithiol, 2-mercaptopyridine N-oxide (2:1:2 molar ratio, BisEDT/PYR)
bismuth-4-methyl-1,2-benzenedithiol, 2-mercaptopyridine N-oxide (2:1:2 molar ratio,
BisTOL/PYR)
bismuth-1,3-propanedithiol, 2-mercaptopyridine N-oxide (2:1:2 molar ratio, BisPDT/PYR)
bismuth-dithioerythritol, 2-mercaptopyridine N-oxide (2:1:2 molar ratio, BisERY/PYR)
bismuth-1-mercapto-2-propanol, 1,2-ethanedithiol (1:1:1 molar ratio, BisHPT/EDT)
bismuth with ethanedithiol and 2-mercaptobenzoimidazole (BisEDT/2MBI (1:1))
bismuth with ethanedithiol and 2-mercaptopyrimidine (BisEDT/SPN (2MPMD) (1:1))
bismuth with ethanedithiol and 3-mercapto-1,2,4-triazole (BisEDT/3MTZ (1:1))
bismuth with ethanedithiol and 1-propane thiol (BisEDT/PT (1:1))
bismuth with ethanedithiol and cysteamine (BisEDT/CSTMN (1:1))
bismuth with ethanedithiol and 3-mercaptopropionic acid (BisEDT/3MPA (1:1))
bismuth with lipoic acid (reduced) (BisALA (BisLipo) (1:1.5))
bismuth with 2-mercaptolpyridine N-oxide and 2-mercaptobenzoimidazole (BisPYR/2MBI
(1:1))
bismuth with 2-mercaptolpyridine N-oxide and cysteamine (BisPYR/CSTMN (1:1))
bismuth with 2,3-dimercapto-1-propanol and 2-mercaptobenzoimidazole (BisBAL/2MBI
(1:1))
bismuth with 2,3-dimercapto-1-propanol and cysteamine (BisBAL/CSTMN (1:1))
bismuth with 3,4 dimercapto toluene and 2-mercaptobenzoimidazole (BisTOL/2MBI (1:1))
bismuth with 3,4 dimercapto toluene and cysteamine (BisTOL/CSTMN (1:1))
bismuth with 2-mercapto pyridine (BisEDT/MPYR)

In some embodiments, the BT composition comprises one or more BT compounds selected from Bis-BAL, Bis-EDT, Bis-dimercaprol, Bis-DTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, Bi-sPyr/EDT, Bis-Pyr/PDT, Bis-Pyr/Tol, Bis-Pyr/Ery, bismuth-1-mercapto-2-propanol, and Bis-EDT/2-hydroxy-1-propanethiol. In other embodiments, the BT compound is selected from one or more of Bis-EDT, Bis-Bal, Bis-Pyr, Bis-Ery, Bis-Tol, Bis-BDT, or Bis-EDT/2-hydroxy-1-propane thiol. As used herein, MB-1B3 (or MB-1-B3) refers to BisEDT; MB-6 refers to BisBDT; MB-8-2 refers to BisBDT/PYR; and MB-11 refers to BisEDT/PYR

In some embodiments, the bismuth thiol compound is BisEDT, which has the following structure:

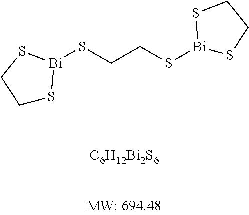

the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.

In some embodiments, if deposited to the deep lung region, the BisEDT compounds have an average half-life of about 4 days.

In some embodiments, the secondary infection is a pulmonary infection comprising one or more bacterial pathogens and/or fungal pathogens. In some embodiments, the pulmonary infection is one or more of bronchiectasis infection, pneumonia, valley fever, allergic bronchopulmonary aspergillosis (ABPA), ventilator-acquired pneumonia, hospital acquired pneumonia, community acquired pneumonia, ventilator associated tracheobronchitis, lower respiratory tract infection, non-tuberculous Mycobacteria, anthrax, legionellosis, pertussis, bronchitis, Bronchiolitis, COPD-associated infection, viral pneumonia, viral bronchiolitis, and post-lung transplantation. In some embodiments, the pulmonary infection is pneumonia or ventilator-acquired pneumonia.

In some embodiments, the one or more pathogens are selected from Haemophilus influenzae, Pseudomonas aeruginosa, Staphylococcus aureus, Staphylococcus warneri Staphylococcus lugdunensis, Staphylococcus epidermidis, Streptococcus milleri/anginous. Streptococcus pyogenes, non-tuberculosis mycobacteria, Mycobacterium tuberculosis, Burkholderia spp., Achromobacter xylosoxidans, Pandoraea sputorum, Stenotrophomonas maltophilia, Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morganii, Inquilinus limosus, Ralstonia mannitolilytica, Pandoraea apista, Pandoraea pnomenusa, Pandoraea sputorum, Bdellovibrio bacteriovorus, Bordetella bronchiseptica, Vampirovibrio chlorellavorus, Actinobacter baumanni, Cupriadidus metallidurans, Cupriavidus pauculus, Cupriavidus respiraculi, Delftia acidivordans, Exophilia dermatitidis, Herbaspirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralstonia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S agalactiae, Bacteroides fragilis, Clostridium difficile, Streptococcus pneumonia, Moraxella catarrhalis, Haemophilus haemolyticus, Haemophilus parainfluenzae, Chlamydophila pneumoniae, Mycoplasma pneumoniae, Atopobium, Sphingomonas, Saccharibacteria, Leptotrichia, Capnocytophaga, Oribacterium, Aquabacterium, Lachnoanaerobaculum, Campylobacter, Acinetobacter; Agrobacterium; Bordetella; Brevundimonas; Chryseobacterium; Delftia; Enterobacter; Klebsiella; Pandoraea; Pseudomonas; Ralstonia, Coccidioides, and Prevotella.

In some embodiments, the respiratory viral infection is one or more of influenza viral infection (e.g., seasonal flu), rhinovirus infection (e.g., common cold), coronavirus infection (e.g., Severe Acute Respiratory Syndrome and common cold), and/or paramyxovirus infection (e.g., measles).

In some embodiments, the coronavirus infection is selected from Severe Acute Respiratory Syndrome-Corona Virus (SARS-CoV), Middle East Respiratory Syndrome virus (CoV-MERS), human HCoV-229E, HCoV-OC43, HCoV-NL63 and HCoV-HKU1. In some embodiments, the coronavirus infection is SARS-CoV (e.g. SARS-CoV-1, SARS-CoV-2). In some embodiments, the SARS-CoV is SARS-CoV-2 (COVID-19). In some embodiments, the respiratory viral infection is an influenza viral infection selected from the group consisting of Influenza A, Influenza B, and Influenza C viral infections. In some embodiments, the Influenza A virus comprises H1N1, H2N2, H3N2, H5N1, H7N7, H1N2, H9N2, H7N2, H7N3, or H10N7 subtypes.

In some embodiments, the BT composition is co-administered with one or more antimicrobial agents. In some embodiments, at least one of the one or more antimicrobial agents is a broad spectrum antiviral agent. In some embodiments, the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Ttraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Tsolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

In some embodiments, the present disclosure provides methods of preventing infections to a primary respiratory viral infection in a subject, the method comprising administering to the subject a bismuth-thiol (BT) composition that comprises BisEDT suspended therein, wherein administering the BT composition is via inhalation, orally or nasally, using an aerosol device, wherein the subject. In some embodiments, the infection is ventilator-acquired pneumonia. In some embodiments, the composition is adminstered to the subject prior to ventilation. In some embodiments, the composition is adminstered to the subject during ventilation. In some embodiments, the composition is adminstered to the subject after ventilation. In some embodiments, the method comprises at least one of: (i) reducing a biofilm (e.g. bacterial and/or fungal), (ii) impairing growth of a biofilm (e.g. bacterial and/or fungal), (iii) preventing initial formation of the biofilm (e.g. bacterial and/or fungal), and/or (iv) preventing reformation of the biofilm (e.g. bacterial and/or fungal).

### Amorphous BisEDT

In some embodiments of the present disclosure, the BisEDT is amorphous BisEDT.

The preferred method of differentiating amorphous BisEDT from crystalline and other non-crystalline forms of BisEDT is X-ray powder diffraction (XRPD). The spectrum of XRPD is typically represented by a diagram plotting the intensity of the peaks versus the location of the peaks, i.e., diffraction angle 2θ (two-theta) in degrees. The XRPD pattern of pure amorphous BisEDT, as illustrated in FIG. 44, can be seen to lack discernible acute peaks. Thus, amorphous BisEDT, according to the present invention, is characterized in providing an X-ray powder diffraction pattern containing one or more broad diffuse halos having very low counts (i.e. see FIG. 44) in contrast to the sharp diffraction peaks characteristic of crystalline materials. The term “broad diffuse halo” is the art recognized term for the ‘humps’ observed in XRPD (see Klug and Alexander, X-ray diffraction procedures: for polycrystalline and amorphous materials, 2nd edition, 1974, John Wiley and Sons, pp 79 1-792). Of course it will be appreciated that a mixture comprising detectable amounts of both crystalline and amorphous BisEDT will exhibit both the characteristic sharp peaks and the diffuse halo(s) on XRPD. This will be evident by an increase in the baseline/background intensities and also a reduction in crystalline peak intensities when compared to the pure crystalline form's XRPD diffractogram. Accordingly, in some embodiments, the X-ray powder diffraction pattern of amorphous BisEDT does not contain any distinct peaks. In some embodiments, the X-ray powder diffraction pattern of amorphous BisEDT is substantially similar to FIG. 44.

In some embodiments, the amorphous form is stable. The term stable as used herein, refers to the tendency to remain substantially in the same physical form for at least a month, preferably at least 6 months, more preferably at least a year, still more preferably at least 3 years, even still more preferably at least 5 years, when stored under ambient conditions (25° C./60% RH) without external treatment. As noted above amorphous forms of many compounds often revert to the crystalline form in a relatively short time period (days/weeks rather than months/years). Substantially the same physical form in this context means that at least 70%, preferably at least 80%/6 and more preferably at least 90% of the amorphous form remains.

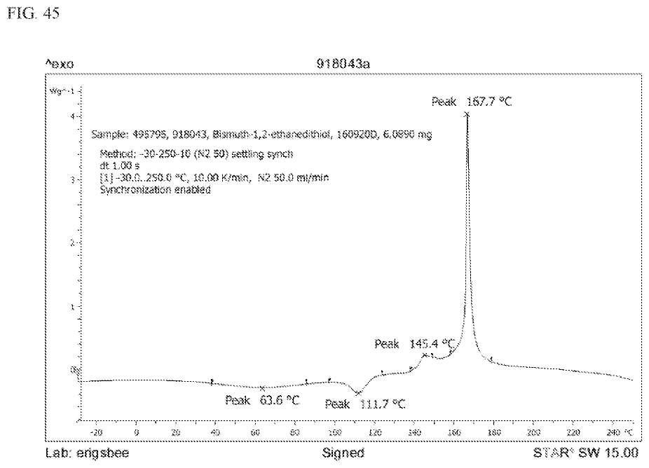

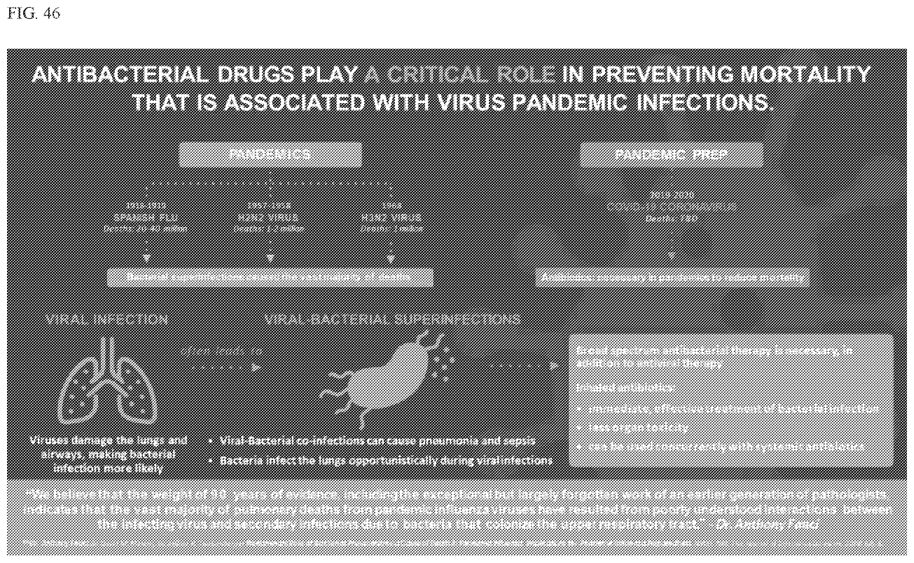

In one embodiment, the amorphous BisEDT is characterized by Differential Scanning Calorimetry (DSC). The DSC thermogram is typically expressed by a diagram plotting the normalized heat flow in units of Watts/gram (“W/g”) versus the measured sample temperature in degree C. The DSC thermogram is usually evaluated for extrapolated onset and end (outset) temperatures, peak temperature, and heat of fusion. In some embodiments, the differential scanning calorimetry thermogram of amorphous BisEDT comprises an exothermic peak at about 168° C., likely related to decomposition. In some embodiments, the differential scanning calorimetry thermogram further comprises an endotherm at about 64° C. and/or an endotherm peak at about 112° C. and/or an exotherm peak at about 145° C. In some embodiments, the differential scanning calorimetry thermogram is substantially similar to FIG. 45.

In some embodiment, amorphous BisEDT has a glass transition point (Tg), measured by Differential Scanning Calorimetry (DSC), modulated DSC, or Thermal Mechanical Analysis (TMA), greater than about 70° C., about 80° C., about 90° C., about 95° C., or 100° C. It is generally regarded that as a rough rule of thumb a Tg of 50° C. or greater above the storage temperature should assure reasonable physical stability to crystallization. Accordingly, in some embodiments the amorphous BisEDT has, when dry, a glass transition point (Tg) measured by Differential Scanning Calorimetry (DSC), modulated DSC, or Thermal Mechanical Analysis (TMA) of 100° C. or more. in some embodiments the amorphous BisEDT has, when dry, a glass transition point (Tg) measured by modulated DSC of 100° C. or more. In some embodiments, the amorphous BisEDT has a glass transition at about 101° C. In some embodiments, the amorphous BisEDT has a glass transition at about 101° C. as measured by modulated DSC.

Thus, according to further embodiments, there is provided amorphous BisEDT, providing an X-ray powder diffraction pattern containing one or more broad diffuse halos having low counts, and possessing, when dry, a glass transition point (Tg) of about 101° C. In some embodiments, the present disclosure provides amorphous BisEDT, providing an X-ray powder diffraction pattern containing one or more broad diffuse halos having low counts, and possessing, when dry, a glass transition point (Tg) measured by modulated DSC of at about 101° C. In some embodiments, the present disclosure provides amorphous BisEDT, providing an X-ray powder diffraction pattern substantially similar to FIG. 44, and possessing, when dry, a glass transition point (Tg) measured by modulated DSC of at about 101° C. In some embodiments, the present disclosure provides amorphous BisEDT, providing an X-ray powder diffraction pattern substantially similar to FIG. 44, and possessing, when dry, a glass transition point (Tg) of at about 101° C.

In some embodiments, the amorphous BisEDT form is at least 60% pure, at least, 70% pure, at least 80% pure, at least 90% pure at least 95% pure, at least 98% pure, or at least 99% pure. For example, the amorphous form in a composition is 60% pure, at least, 70% pure, at least 80% pure, at least 90% pure at least 95% pure, at least 98% pure, or at least 99% pure. In a specific embodiment, the composition is a pharmaceutical composition. Amorphous BisEDT, or the presence of some amorphous BisEDT, can be distinguished from crystalline BisEDT, using a variety of means, including but not limited to X-ray powder diffraction, Raman spectroscopy, solution calorimetry, differential scanning calorimetry, solid state nuclear magnetic resonance spectra (ssNMR) or infra-red spectroscopy. Each of these techniques is well established in the art. Amorphous BisEDT can also be identified based on the morphology of the particles seen under an electron microscope. Furthermore, amorphous BisEDT may be much more soluble than crystalline BisEDT, providing another means of discriminating between the crystalline and amorphous BisEDT forms, or detecting an amount of amorphous form within a BisEDT preparation.

In some embodiments, the amorphous BisEDT of the present invention is substantially free from other forms of BisEDT. Substantially free from other forms of BisEDT shall be understood to mean that amorphous BisEDT contains less than 50%, preferably less than 25%, more preferably less than 10% and still more preferably less than 5% of any other forms of BisEDT, e.g. crystalline BisEDT.

### Methods of Making

In some embodiments, the present disclosure provides a method of making an amorphous form of BisEDT, comprising (a) mixing an acidic aqueous solution that comprises a bismuth salt, with a solvent selected from the group consisting of acetone, acetonitrile, 1,2-dichloroethane, dimethyl sulfoxide, ethyl acetate, isopropanol, methyl tert-butyl ether, and mixtures thereof; (b) combing the product of (a) with a solution of 1,2-ethanedithiol in a solvent selected from the group consisting of acetone, acetonitrile, 1,2-dichloroethane, dimethyl sulfoxide, ethyl acetate, isopropanol, methyl tert-butyl ether, and mixtures thereof, under conditions and for a time sufficient for formation of a precipitate which comprises the amorphous form of BisEDT. No amorphous forms of BisEDT are known or disclosed in the prior art. The previously reported syntheses of BisEDT all were run under various conditions, such as ethanolic solvent conditions that produced crystalline BisEDT. While ethanol and a variety of other common solvents produced crystalline forms of BisEDT, the use of other solvents, such as acetone, acetonitrile, 1,2-dichloroethane, dimethyl sulfoxide, ethyl acetate, isopropanol, and methyl tert-butyl ether were discovered in the present invention to produce amorphous BisEDT.

In some embodiments, the method further comprises recovering the precipitate to remove impurities. The amorphous product may be separated from the solution, e.g. by precipitation, cooling, filtration, concentration, centrifugation, and combinations thereof, optionally followed by washing with a wash solution, preferably a solvent in which amorphous BisEDT has a very low solubility. The amorphous product can be dried to a constant weight, e.g. at +30° C. to +50° C., preferably at reduced pressure, for, e.g. 10 to 48 hours.

In some embodiments, the bismuth salt is Bi(NO₃)₃.

In some embodiments, 1,2-ethanedithiol is at a concentration of from about 1% wt/vol to about 20% wt/vol prior to step (b). For example, 1,2-ethanedithiol is at a concentration of about 1% wt/vol, about 2% wt/vol, about 3% wt/vol, about 4% wt/vol, about 5% wt/vol, about 6% wt/vol, about 7% wt/vol, about 8% wt/vol, about 9% wt/vol, about 10% wt/vol, about 11% wt/vol, about 12% wt/vol, about 13% wt/vol, about 14% wt/vol, about 15% wt/vol, about 16% wt/vol, about 17% wt/vol, about 18% wt/vol, about 19% wt/vol, or about about 20% wt/vol.

In some embodiments, the acidic aqueous solution is prepared by mixing an aqueous suspension of either Bi (III) sub-nitrate or Bi (III) nitrate pentahydrate with an acid under conditions and for a time sufficient to form a substantially clear solution.

In some embodiments, the concentration of either Bi (III) sub-nitrate or Bi (III) nitrate pentahydrate in the aqueous solution is from about 100 mg/mL to about 400 mg/mL. For example, the concentration of either Bi (111) sub-nitrate or Bi (III) nitrate pentahydrate in the aqueous solution is from about 100 mg/mL, about 125 mg/mL, about 150 mg/mL, about 175 mg/mL, about 200 mg/mL, about 225 mg/mL, about 250 mg/mL, about 275 mg/mL, about 300 mg/mL, about 325 mg/mL, about 350 mg/mL, about 375 mg/mL, or about 400 mg/mL.

In some embodiments, the acid is 70% HNO₃. In some embodiments, the acid is 70% HNO₃.

In some embodiments, the method further comprises adding the clear solution to an acidic solution. In some embodiments, the method further comprises adding the clear solution to an acidic solution, wherein the acidic solution is an HNO₃ solution. In some embodiments, the method further comprises adding the clear solution to an acidic solution, wherein the acidic solution is about a 5% HNO₃ solution.

In some embodiments, step (b) is performed at a temperature ranging from about 20° C. to about 28° C. For example, step (b) is performed at a temperature ranging from about 20° C., about 21° C., about 22° C., about 23° C., about 24° C., about 25° C., about 26° C., about 27° C., to about 28° C.

### Combination Treatments

In certain embodiments, compounds disclosed herein can be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

In certain embodiments, conjoint administration of compounds of the disclosure with one or more additional therapeutic agent(s) provides improved efficacy relative to each individual administration of the compound of the disclosure or the one or more additional therapeutic agent(s). In certain such embodiments, the conjoint administration provides an additive effect or synergistic effect, wherein an additive effect refers to the sum of each of the effects of individual administration of the compound of the disclosure and the one or more additional therapeutic agent(s). In some embodiments, the subject receives conjoint administration of a therapy for another disease, disorder, or condition. In some embodiments, the other therapy is a CFTR modulator or bronchodilator.

In some embodiments, the methods of the present disclosure comprise coadministering or conjointly administering to the subject an antibiotic selected from amikacin, tobramycin, gentamicin, piperacillin, mezlocillin, ticarcillin, imipenum, ciprofloxacin, ceftazidime, aztreonam, ticaricillin-clavulanate, dicloxacillin, amoxicillin, ticarcillin-clavulanate, trimethoprim-sulfamethoxazole, cephalexin, piperacillin-tazobactam, linezolid, daptomycin, vancomycin, metronidazole, clindamycin, colistin, tetracycline, levofloxacin, amoxicillin and clavulanic acid (Augmentin®), cloxacillin, dicloxacillin, cefdinir, cefprozil, cefaclor, cefuroxime, erythromycin/sulfisoxazole, erythromycin, clarithromycin, azithromycin, doxycycline, minocycline, tigecycline, imipenem, meripenem, colistimethate/colistin®, methicillin, oxacillin, nafcillin, cabenicillin, azlocillin, piperacillin and tazobactam (Zosyn®), cefepime, ethambutol, rifampin, and meropenem. In some embodiments, the antibiotic is selected from meropenem, ceftazidime, tobramycin, amikacin, aztreonam, ciprofloxacin, colistin, and levofloxacin.

In certain embodiments of the present disclosure, the therapeutic agents that can be conjointly administered with compounds of the disclosure, such as a bismuth-thiol compound, include known antibiotics. In some embodiments, the antibiotic is selected from methicillin, vancomycin, nafcillin, gentamicin, ampicillin, chloramphenicol, doxycycline, colistin amikacin, aztreonam, and tobramycin. In some embodiments, the antibiotic is selected from tobramycin, imipenem, tetracycline, and minocycline. In some embodiments, the antibiotic is administered systemically after revision surgery. In some embodiments, the antibiotic is administered prior to revision surgery. The conjointly administered therapeutic agent, such as an antibiotic, can be administered with any suitable frequency and at any suitable dosage. Such dosage amount and frequency can be determined by those of ordinary skill in the art.

In certain embodiments, BT compounds of the disclosure can be conjointly administered with one or more other BT compounds of the disclosure. Moreover, such combinations can be conjointly administered with other therapeutic agents.

### Pharmaceutical Compositions

In some embodiments, the present disclosure provides a composition comprising bisEDT or an amorphous from of BisEDT.

The compositions and methods of the present disclosure can be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water, physiologically buffered saline, physiologically buffered phosphate, or other solvents or vehicles such as glycols, glycerol, oils such as olive oil, or injectable organic esters. In some embodiments, when such pharmaceutical compositions are for human administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as lyophile for reconstitution, powder, solution, syrup, injection or the like. The composition can also be present in a solution suitable for topical administration.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose, or dextrans; antioxidants, such as ascorbic acid or glutathione; chelating agents; low molecular weight proteins; salts; or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols and sugar alcohols, such as glycerin, sorbitol, mannitol, xylitol, erythritol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances, including salts such as sodium chloride, employed in pharmaceutical formulations.

The formulations can conveniently be presented in unit dosage form and can be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect.

In some embodiments, the BT composition is a powder, spray, ointment, paste, cream, lotion, solution, patch, suspension or gel. In some embodiments, the BT composition is a solution. The BT composition can comprise any suitable concentration of bismuth-thiol compound. In some embodiments, the BT composition is administered as a dosage from about 0.25 mg/mL to about 15 mg/mL, from about 0.4 mg/mL to about 15 mg/mL, from about 0.6 mg/mL to about 15 mg/mL, from about 0.6 mg/mL to about 100 mg/mL, from about 5 mg/mL to about 100 mg/mL, from about 10 mg/mL to about 100 mg/mL, from about 25 mg/mL to about 100 mg/mL, from about 50 mg/mL to about 100 mg/mL, from about 0.8 mg/mL to about 15 mg/mL, from about 1 mg/mL to about 10 mg/mL, from 2.5 mg/mL to about 10 mg/mL, from about 4 mg/mL to about 10 mg/mL, from about 5 mg/mL to about 10 mg/mL, from about 6 mg/mL to about 10 mg/mL, 0.6 mg/mL to about 6 mg/mL, from about 4 mg/mL to about 15 mg/mL, from about 6 mg/mL to about 15 mg/mL, from about 50 μg/mL to about 750 μg/mL, from about 75 μg/mL to about 500 μg/mL, from about 100 μg/mL to about 250 μg/mL, from about 100 μg/mL to about 150 μg/mL, or from about 75 μg/mL to about 150 μg/mL; and/or the total amount of the BT composition administered to the lungs is from about 0.25 mg to about 15 mg, from about 0.4 mg to about 15 mg, from about 0.6 mg to about 15 mg, from about 0.8 mg to about 15 mg, from about 1 mg to about 10 mg, from 2.5 mg to about 10 mg, from about 4 mg to about 10 mg, from about 5 mg to about 10 mg, from about 6 mg to about 10 mg, 0.6 mg to about 6 mg, from about 4 mg to about 15 mg, from about 6 mg to about 15 mg, from about 50 μg to about 750 μg, from about 75 μg to about 500 μg, from about 100 μg to about 250 μg, from about 100 μg to about 150 μg, or from about 75 μg to about 150 μg. In certain embodiments, the BT composition is administered as a dosage from about 0.6 mg/mL to about 6 mg/mL.

In some embodiments, the BT composition is administered three times per day, two times per day, once daily, every other day, once every three days, once every week, once every other week, once monthly, to once every other month. In certain embodiments, the BT composition is administered once daily. In certain embodiments, the BT composition is administered once weekly. In certain embodiments, the BT composition is administered once every other week. In some embodiments, the BT composition is administered chronically in a 4 week on/4 week off dosing schedule. In some embodiments, the BT composition is administered chronically, for example as part of a background therapy. As will be appreciated by a person having ordinary skill in the art, the administration frequency may depend on a number of factors including dose and administration route. For example, if the BT composition is administered via an aerosol administration, a low dose such as 100-1000 μg/mL may be administered once or twice daily; however, a high dose such as 2.5-10 mg/mL may be administered e.g. once or twice a week.

In some embodiments, the BT composition further comprises one or more carriers selected from animal and vegetable fats, oils, waxes, paraffins, starch, tragacanth, cellulose derivatives, polyethylene glycols, silicones, bentonites, silicic acid, polymers, talc, and zinc oxide. In some embodiments, the carrier is methylcellulose. In some embodiments, the carrier is poly(methyl methacrylate).

Compositions can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, by ionizing radiation (gamma photons for example), autoclaving, or by incorporating sterilizing agents in the form of sterile solid compositions that can be dissolved in sterile water, or some other sterile injectable medium immediately before use.

Liquid dosage forms useful for topical administration include pharmaceutically acceptable emulsions, lyophiles for reconstitution, microemulsions, solutions, suspensions, gels, syrups and elixirs. In addition to the active ingredient, the liquid dosage forms can contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (such as cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the topical compositions can also include adjuvants such as wetting agents, emulsifying and suspending agents, and preservative agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound can be mixed under sterile conditions with a pharmaceutically acceptable carrier, and with any preservatives or buffers that can be required.

The ointments, pastes, creams and gels can contain, in addition to an active compound, one or more excipients or carriers, such as animal and vegetable fats, oils, waxes, paraffins, starch, tragacanth, cellulose derivatives, polyethylene glycols, silicones, bentonites, silicic acid, talc, polymers, salts, and zinc oxide, or mixtures thereof. In some embodiments, the BT composition is in the form of an aqueous solution. In some embodiments, the excipient comprises a salt selected from sodium chloride or potassium chloride. In some embodiments, the excipient comprises sodium chloride.

In certain embodiments, the BT composition is a suspension of one or more BT compounds in Tween (e.g. Tween 80) and/or in a buffer (e.g. sodium phosphate buffer). For example, in some embodiments, the BT composition is a suspension of one or more BT compounds in from about 0.1% Tween 80 to about 1.0% Tween 80, including all ranges therebetween. For example, the BT composition is a suspension of one or more BT compounds in about 0.1% Tween 80, about 0.2% Tween 80, about 0.3% Tween 80, about 0.4% Tween 80, about 0.5% Tween 80, about 0.6% Tween 80, about 0.7% Tween 80, about 0.8% Tween 80, about 0.9% Tween 80, or about 1% Tween 80. In some embodiments, the BT composition is a suspension of one or more BT compounds in about 0.5% Tween 80.

In a specific embodiment, the present invention may be a pharmaceutical composition comprising bismuth-thiol (BT) composition that comprises BisEDT suspended therein, wherein the BT composition comprises a plurality of microparticles. In a specific embodiment, the D90 of said microparticles is less than or equal to 4.5 μm, or 4.0 μm, or 3.5 μm, or 3.0 μm, or 2.5 μm, or 2.0 μm, or 1.9 μm, or 1.8 μm, or μm 1.7 μm, or 1.6 μm, or 1.5 μm or any ranges in between. In a specific embodiment, the D90 of said microparticles is less than or equal to 1.9 μm. In another specific embodiment, the D90 of said microparticles is less than or equal to 1.6 μm. In another specific embodiment, the D50 of said microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges in between. In another specific embodiment, the D10 of said microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges in between. In a specific embodiment, the pharmaceutical composition comprising bismuth-thiol (BT) composition comprises BisEDT suspended therein, wherein the BT composition comprises a plurality of microparticles, wherein the D90 of said microparticles is less than or equal to about 1.6 μm. In a specific embodiment, the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4. In another specific embodiment, the compositions described above can be administered to a subject for treating, managing and/or lessening the severity of symptoms and infections associated with one or more pulmonary diseases or infections in a subject or any specific method of treating, managing and/or lessening the severity of symptoms and infections associated with one or more pulmonary diseases described herein.

A variety of buffers may be used in the context of the present disclosure and will be readily apparent to a person having ordinary skill in the art. For example, in some embodiments, suitable buffers include sodium or potassium citrate, citric acid, phosphate buffers such as sodium phosphate, boric acid, sodium bicarbonate and various mixed phosphate buffers including combinations of Na₂HPO₄, NaH₂PO₄ and KH₂PO₄. In some embodiments, sodium phosphate buffer is used. In some embodiments, sodium citrate buffer is used. Changes in lung pH may impact the airway surface liquid environment, improve airway defenses, and alter the disease course. Accordingly, the formulation pH may vary from about 5 to about 10. In some embodiments, the formulation pH is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10. In some embodiments, the formulation pH is about 7.4.

In some embodiments, the BT composition is a suspension of one or more BT compounds in about 0.5% Tween 80 in sodium phosphate buffer at a pH of about 7.4. In some embodiments, the one or more BT compounds are present in the composition at a concentration ranging from about 100 μg/mL to about 1000 mg/mL including all integers and ranges therebetween. For example, in some embodiments, the one or more BT compounds are present in the composition at a concentration ranging from about 100 μg/mL, 200 μg/mL, 300 μg/mL, 400 μg/mL, 500 μg/mL, 600 μg/mL, 700 μg/mL, 800 μg/mL, 900 μg/mL, 1000 μg/mL, 10 mg/mL, 25 mg/mL, 50 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, 525 mg/mL, 550 mg/mL, 575 mg/mL, 600 mg/mL, 625 mg/mL, 650 mg/mL, 675 mg/mL, 700 mg/mL, 725 mg/mL, 750 mg/mL, 775 mg/mL, 800 mg/mL, 825 mg/mL, 850 mg/mL, 875 mg/mL, 900 mg/mL, 925 mg/mL, 950 mg/mL, 975 mg/mL, to about 1000 mg/mL. In some embodiments, the one or more BT compounds are present in the composition at a concentration ranging from about 100 μg/mL to about 1000 μg/mL.

In some embodiments, the composition osmolality may need to be further adjusted with an additive such as NaCl or TDAPS to achieve a desired osmolality. For example, in some embodiments, the osmolality of the composition is adjusted with sodium chloride to an osmolality ranging from about 100 mOsmol/kg to about 500 mOsmol/kg, including all integers and ranges therebetween. In some embodiments, the osmolality of the composition is from about 290 mOsmol/kg to about 310 mOsmol/kg. For example, in some embodiments, the osmolality of the composition is about 290 mOsmol/kg, 291 mOsmol/kg, 292 mOsmol/kg, 293 mOsmol/kg, 294 mOsmol/kg, 295 mOsmol/kg, 296 mOsmol/kg, 297 mOsmol/kg, 298 mOsmol/kg, 299 mOsmol/kg, 300 mOsmol/kg, 301 mOsmol/kg, 302 mOsmol/kg, 303 mOsmol/kg, 304 mOsmol/kg, 305 mOsmol/kg, 306 mOsmol/kg, 307 mOsmol/kg, 308 mOsmol/kg, 309 mOsmol/kg, to about 310 mOsmol/kg. In some embodiments, the osmolality is about 300 mOsmol/kg.

In some embodiments, the BT composition is a suspension of BisEDT in Tween (e.g. Tween 80) in a buffer (e.g. sodium phosphate buffer). In some embodiments, the BT composition is a suspension of BisEDT in about 0.5% Tween 80 in a sodium phosphate buffer at a pH of about 7.4. In some embodiments, the BT composition is a suspension of BisEDT in about 0.5% Tween 80 in a sodium phosphate buffer at a pH of about 7.4, wherein the composition has an osmolality of about 300 mOsmol/kg (e.g. adjusted to 300 mOsmol/kg with sodium chloride). In some embodiments, the BisEDT is present at a concentration of about 100 μg/mL, 250 μg/mL, 500 μg/mL, 750 μg/mL, 1000 μg/mL, 2.5 mg/mL, 10 mg/mL, 25 mg/mL, 50 mg/mL, 75 mg/mL, or about 100 mg/mL.

In some embodiments, the BT composition is a suspension formulation which is intended for pulmonary delivery. For example, the BT composition is a suspension formulation which is ultimately administered by inhalation either orally and/or nasally. Accordingly, in some embodiments, the BT composition is aerosolized by a device such as a nebulized.

Powders and sprays can contain, in addition to an active compound, excipients such as methylcellulose, sodium chloride, PMMA, lactose, talc, silicic acid, aluminum hydroxide, calcium silicates and polyamide powder, dipalmitoylphosphatidylcholine (DPPC), leucine, polyethyleneglycol, or mixtures of these substances. Sprays can additionally contain customary propellants, such as chlorofluorohydrocarbons and volatile unsubstituted hydrocarbons, such as butane and propane.

In some embodiments, the BT composition is administered by inhalation, orally or nasally, using an aerosol device, such as a nebulizer. Known nebulizers, such as PARI IC Plus, can administer the disclosed compositions as an aqueous solution, optionally in buffered saline. The solution can be provided to the subject in the form of an ampule for use in the nebulizer. The nebulizer can be reusable and includes a compressor that provides the formulation over a period of time, such as about 10-15 minutes or longer. Known compressors, such as APRI Vios Air and DeVilbiss Pulmo-aide, are suitable for administration. The nebulizer administers the formulation topically to the lung tissues, such as mucosa, the bronchi and/or the bronchioles, alveoli, deep lung alveoli. The formulation can penetrate lung mucosa and biofilms to reduce the microbial (e.g. bacterial or fungal) biofilm, impair the growth of the microbial (e.g. bacterial or fungal) biofilm, prevent reformation of the microbial (e.g. bacterial or fungal) biofilm, reduce planktonic growth, and/or inhibit planktonic growth.

In other embodiments, a nose-only aerosol device can be used for administration of the formulation.

An exemplary BT composition formulation is a neutral pH, isotonic, buffered aqueous solution of BT compound microparticles with a nonionic surfactant. In certain embodiments, the buffer is a phosphate buffer with added NaCl. In some embodiments, the microparticle size is a D₅₀ of about 1-5 μm. The formulation can be delivered using commercially available compressed air jet nebulizer. In some embodiments, the formulation concentration is about 0.1 μg/mL to about 100 mg/mL.

In some embodiments, the present disclosure provides a pharmaceutical composition comprising bismuth-thiol (BT) composition that comprises BisEDT suspended therein and at least one antimicrobial agent, wherein the BT composition comprises a plurality of microparticles, wherein the D90 of said microparticles is less than or equal to 1.9 μm. In some embodiments, at least 70% of said microparticles having a volumetric mean diameter of from about 0.6 μm to about 2.5 μm. In some embodiments, at least 90% of said microparticles having a volumetric mean diameter of from about 0.6 μm to about 2.5 μm. In some embodiments, at least one antimicrobial agents is a broad spectrum antiviral agent. In some embodiments, the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

In some embodiments, the present disclosure provides an aerosol comprising a plurality of dispersed liquid droplets in a gas, said liquid droplets comprising a BT composition comprising at least one BT compound suspended therein, wherein the BT compound comprises bismuth and/or a bismuth salt and a thiol-containing compound; and wherein at least 60%, 65%, 70, 75%, 80%, 90%, or 95% of the liquid droplets have a mass median aerodynamic diameter (MMAD) from about 0.4 μm to about 5 μm when measured by laser time of flight and/or cascade impactor. In some embodiments, at least 60%, 65%, 70, 75%, 80%, 90%, or 95% of the liquid droplets have a MMAD of from about 0.4 μm to about 7 μm, or from about 0.5 μm to about 5 μm, or from about 0.7 μm to about 4 μm, or from about 0.7 μm to about 3.5 μm, or from about 0.8 μm to about 3.5 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, including all ranges therebetween. In some embodiments, at least 60%, 65%, 70, 75%, 80%, 90%, or 95% of the liquid droplets have a MMAD of from about 0.8 μm to about 1.6 μm, or from about 0.9 μm to about 3.5 μm, or from about 0.9 μm to about 3 μm, or from about 0.9 μm to about 0.4 μm, or from about 1.0 μm to about 2.0 μm, or from about 1.0 μm to about 1.8 μm, and all ranges therebetween.

In some embodiments, the plurality of liquid droplets have a D90 of less than about 10 μm. For example, in some embodiments, the plurality of liquid droplets have a D90 of less than about 10 μm, 9 μm, 8 μm, 7 μm, 6 μm, 5 μm, 4 μm, 3 μm, 2 μm, or about 1 μm. In some embodiments, the plurality of liquid droplets have a D90 of less than about 3 μm. In some embodiments, the plurality of liquid droplets have a D90 ranging from about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the plurality of liquid droplets are dispersed in a continuous gas phase.

In some embodiments, the BT compound of the aerosol comprises bismuth and/or a bismuth salt associated covalently and/or in a coordination complex with one or more thiol-containing compounds. For example, the bismuth salt is bismuth nitrate, bismuth subnitrate, or bismuth chloride. In some embodiments, the thiol-containing compound comprises one or more agents selected from 1,2-ethane dithiol, 2,3-dimercaptopropanol, pyrithione, dithioerythritol, 3,4 dimercaptotoluene, 2,3-butanedithiol, 1,3-propanedithiol, 2-hydroxypropanethiol, 1-mercapto-2-propanol, dithioerythritol, dithiothreitol and alpha-lipoic acid. In some embodiments, the BT composition comprises one or more BT compounds selected from BisBAL, BisEDT, Bis-dimercaprol, BisDTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, BisPyr/EDT, Bis-Pyr/PDT, Bis-PyrfTol, Bis-Pyr/Ery, bismuth-1-mercapto-2-propanol, BisEDT/CSTMN (1:1), BisPYR/CSTMN (1:1), BisBAL/CSTMN (1:1), BisTOL/CSTMN (1:1), and Bis-EDT/2-hydroxy-1-propanethiol. In some embodiments, the BT compound is selected from one or more of Bis-EDT, Bis-Bal, Bis-Pyr, Bis-Ery, Bis-Tol, Bis-BDT, or Bis-EDT/2-hydroxy-1-propane thiol. In some embodiments, the BT compound on the aerosol is BisEDT. In some embodiments, the BT compound of the aerosol is BisBDT or BisBAL.

In some embodiments, the BT compound (e.g. BisEDT) is suspended in the liquid droplet. The BT compounds of the present disclosure have little to no solubility in conventional solvents and aerosol carriers and therefore exist substantially as a suspension of BT particles in the aerosol droplet. For example, in some embodiments, the BT compound (such as BisEDT) is less than 1% soluble in the aerosol carrier and therefore exists primarily (>99%) as a solid.

In some embodiments, the present disclosure provides an aerosol comprising a plurality of dispersed liquid droplets in a gas, said liquid droplets comprising a BT composition comprising BisEDT compound suspended therein and at least one antimicrobial agent; and wherein at least 70% of the liquid droplets have a MMAD from about of from about 0.9 μm to about 3 μm. In some embodiments, prior to aerosolization, the BT composition comprises a plurality of microparticles wherein at least 70% of said microparticles have a VMD of from about 0.6 μm to about 2.5 μm. In some embodiments, at least 90% of the liquid droplets have a MMAD from about of from about 0.9 μm to about 3 μm. In some embodiments, prior to aerosolization, the BT composition comprises a plurality of microparticles wherein at least 90% of said microparticles have a VMD of from about 0.6 μm to about 2.5 μm. In some embodiments, the droplets further comprise Tween 80 (e.g. from about 0.05% to about 1%) and optionally a buffer (e.g. sodium phosphate or sodium citrate) at a pH of about 7.4; and/or sodium chloride. In some embodiments, at least one antimicrobial agents is a broad spectrum antiviral agent. In some embodiments, the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

In some embodiments, the droplets further comprise Tween 80 (e.g. from about 0.05% to about 1%) and optionally a buffer (e.g. sodium phosphate or sodium citrate) at a pH of about 7.4; and/or sodium chloride.

The aerosols of the present disclosure have a very narrow MMAD distribution which is beneficial because of the need to concentrate the particle mass in the target size range, and minimize or eliminate the fraction of the product that is outside of the respirable range or ‘fines’, i.e. particles of typically less than 0.4 μm diameter. The ability to create a narrow droplet size distribution in the appropriate size range provides control of the initial evaporation rate and allows for high deposition efficiency. The limiting factor in terms of the lower limit of particle aerosol droplet size is the BT microparticle size (e.g. the BisEDT microparticle size). An aerosolized droplet cannot be smaller than the BisEDT microparticulate size. As such, the BT microparticle size distribution, as well as the uniformity and consistent reproducibility of the BT microparticulate size distribution, are important beneficial characteristics to support the generation of a safe, effective, and efficient aerosolized BisEDT drug product for inhalation purposes. Accordingly, in some embodiments, the aerosols of the present disclosure effectuate a deposition efficiency of greater than 3%, greater than 5%, greater than 10%, greater than 15%, greater than 20%, greater than 25%, greater than 30%, greater than 35% m greater than 40%, greater than 45%, greater than 50%, greater than 55%, greater than 60%, greater than 65%, greater than 70%, greater than 75%, and greater than 80%. In some embodiments, the deposition efficiency refers to deposition to the deep lung region of lung, for example, to the deep lung alveoli. In some embodiments, the aerosols of the present disclosure effectuate a deposition efficiency upon aerosolization via a nebulizer. For example, the nebulizer is a jet nebulizer. In some embodiments, the jet nebulizer is a Pari LC Plus jet nebulizer or Pari LC SPRINT jet nebulizer. In some embodiments, the nebulizer has an inlet pressure from about 10 to about 40 psig (e.g. 20-25 psig). In some embodiments, the inlet flow is from about 3 L/min to about 8 L/min (e.g. 5.2 L/min). In some embodiments, the exhaust air flow is from about 3 L/min to about 8 L/min (e.g. 5 L/min).

The alveolar region of the lung has a minimal thickness (0.5 μm-2.5 μm) separating the blood flow from the lumen so conventional pulmonary agents that deposit on the alveolar epithelium have extremely short lung residence time due to systemic absorption. Accordingly, conventional pulmonary treatments typically require frequent dosing in order to maintain adequate levels of drug at the tissue level. However, the aerosolized particles of the present disclosure were surprisingly discovered to possess an exceptionally long residence time in the lungs (measured as half-life) and have reduced mucociliary clearance and macrophage uptake relative to conventional pulmonary treatments. Furthermore, the long residence time of the aerosols of the present minimizes systemic activity and associated systemic side effects. Without being bound by any particular theory, it is believed that the aerosolized microparticles dissolve slowly on the lung lumen and the systemic exposure is thus dissolution rate limited. Further, the increased lung residence time results in significant reductions in microbial colony due to the continuous presence of the BT microparticles.

Accordingly, in some embodiments, when the aerosol is deposited to the lung (e.g. to the deep lung alveoli), the BT compounds have an average half-life of at least 2 days. For example, the BT compounds have an average half-life of about 2, 3, 4, or 5 days. In some embodiments, the BT compound is BisEDT. In a specific embodiment, the lung tissue half-life of BisEDT is 30 hrs or more, 40 hrs or more, 50 hrs or more, 60 hrs or more, 70, hrs or more, 80 hrs or more, 90 hrs or more, 100 hrs or more, 110 hrs or more, 125 hrs or more, or 150 hrs or more. In a specific embodiment, the lung tissue half-life is after a single dose via inhalation. In another specific embodiment, lung tissue is from a rat. In another specific embodiment, lung tissue half-life of BisEDT is determined by the ue of protocol as in Example 8 herein.

In another embodiment, the lung tissue half-life of BisEDT is 80 hrs or more when the rat is given a single dose of 100 μg/kg lung using a Pari LC plus jet nebulizer to administer to the rats with the formulations described herein. In another embodiment, the lung tissue half-life of BisEDT is 90 hrs or more. In another embodiment, the lung tissue half-life of BisEDT is 100 hrs or more.

In another embodiment, after delivering the aerosolized composition to a subject, at least 60%, 65%, 70, 75%, 80%, 90%, or 95% of the dose is deposited on the lung, as opposed to the orpharanygeal region and the conducting airways. In a specific embodiment, at least 80% of the dose is deposited on the lung, as opposed to the orpharanygeal region and the conducting airways. In another specific embodiment, at least 90% of the dose is deposited on the lung, as opposed to the orpharanygeal region and the conducting airways.

It was previously unheard of for an aerosolized pulmonary treatment to have aerosol particles with a narrow distribution that effectuate a high deposition efficiency coupled with an exceptionally long lung residence time for continuous treatment and little to no systemic absorption.

In some embodiments, the present disclosure provides a method of treating, managing or lessening the severity of symptoms and infections in a subject having a pulmonary infection, the method comprising administering to the subject a bismuth-thiol (BT) composition that comprises at least one BT compound, wherein the composition is a suspension of microparticles having a volumetric mean diameter (VMD) from about 0.4 μm to about 5 μm and/or a mass median aerodynamic diameter (MMAD) from about 0.4 μm to about 5 μm. In some embodiments, the BT compound is BisEDT. In some embodiments, the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4. For example, in some embodiments, the BT composition comprises BisEDT at a concentration greater than about 0.25 mg/mL, about 0.5% Tween 80®, about 10 mM sodium chloride, and about 10 mM sodium phosphate at about pH 7.4. In another embodiment of the methods herein, the BT composition is administered by aerosolization. In some embodiments, when the aerosol is deposited to the lung (e.g. to the deep lung alveoli), the BT compounds have an average half-life of at least 2 days. For example, the BT compounds have an average half-life of about 2, 3, 4, or 5 days. In some embodiments, the BT compound is BisEDT. In a specific embodiment, the lung tissue half-life of BisEDT is 30 hrs or more, 40 hrs or more, 50 hrs or more, 60 hrs or more, 70, hrs or more, 80 hrs or more, 90 hrs or more, 100 hrs or more, 110 hrs or more, 125 hrs or more, or 150 hrs or more. In a specific embodiment, the lung tissue half-life is after a single dose via inhalation. In another specific embodiment, lung tissue is from a rat. In another specific embodiment, lung tissue half-life of BisEDT is determined by the ue of protocol as in Example 8 herein.

In another embodiment, after delivering the aerosolized composition to a subject, at least 60%, 65%, 70%, 75%, 80%, 90%, or 95% of the dose is deposited on the lung, as opposed to the orpharanygeal region and the conducting airways. In a specific embodiment, at least 80% of the dose is deposited on the lung, as opposed to the orpharanygeal region and the conducting airways. In another specific embodiment, at least 90% of the dose is deposited on the lung, as opposed to the orpharanygeal region and the conducting airways. In a specific embodiment, the subject is a rat. In another specific embodiement, the percent deposition is determined using a Pari LC plus jet nebulizer to administer to the rats with the formulations described herein.

In another embodiment, the lung tissue half-life of BisEDT is 80 hrs or more when the rat is given a single dose of 100 μg/kg lung using a Pari LC plus jet nebulizer to administer to the rats with the formulations described herein. In another embodiment, the lung tissue half-life of BisEDT is 90 hrs or more. In another embodiment, the lung tissue half-life of BisEDT is 100 hrs or more.

In some embodiments, the composition is a suspension of microparticles having a volumetric mean diameter (VMD) from about 0.4 μm to about 5 μm. In some embodiments, at least 60%, 65%, 70%, 75%, 80%, 90%, or 95% of the microparticles have a VMD of from about 0.4 μm to about 5 μm, or from about 0.6 μm to about 2.5 μm, or from about 0.7 μm to about 4 μm, or from about 0.7 μm to about 3.5 μm, or from about 0.7 μm to about 3.0 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the microparticles have a VMD of from about 0.6 μm to about 2.5 μm, or from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm and all ranges therebetween. In some embodiments, the microparticles have a D90 of less than about 10 μm. For example, in some embodiments, the microparticles have a D90 of less than about 10 μm, 9 μm, 8 μm, 7 μm, 6 μm, 5 μm, 4 μm, 3 μm, 2 μm, or about 1 μm. In some embodiments, the microparticles have a D90 of less than about 3 μm. In some embodiments, the microparticles have a D90 ranging from about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the BT composition is aerosolized, wherein the aerosolized liquid droplets have a MMAD from about of from about 0.4 μm to about 5 μm. In some embodiments, at least 60%, 65%, 70%, 75%, 80%, 90%, or 95% of the liquid droplets have a MMAD of from about 0.4 μm to about 7 μm, or from about 0.5 μm to about 5 μm, or from about 0.7 μm to about 4 μm, or from about 0.7 μm to about 3.5 μm, or from about 0.8 μm to about 3.5 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the liquid droplets have a MMAD of from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm and all ranges therebetween. In some embodiments, the plurality of liquid droplets have a D90 of less than about 10 μm. For example, in some embodiments, the plurality of liquid droplets have a D90 of less than about 10 μm, 9 μm, 8 μm, 7 μm, 6 μm, 5 μm, 4 μm, 3 μm, 2 μm, or about 1 μm. In some embodiments, the plurality of liquid droplets have a D90 of less than about 3 μm. In some embodiments, the plurality of liquid droplets have a D90 ranging from about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the plurality of liquid droplets are dispersed in a continuous gas phase.

In some embodiments, the BT compound of the aerosol comprises bismuth and/or a bismuth salt associated covalently and/or in a coordination complex with one or more thiol-containing compounds. For example, the bismuth salt is bismuth nitrate, bismuth subnitrate, or bismuth chloride. In some embodiments, the thiol-containing compound comprises one or more agents selected from 1,2-ethane dithiol, 2,3-dimercaptopropanol, pyrithione, dithioerythritol, 3,4 dimercaptotoluene, 2,3-butanedithiol, 1,3-propanedithiol, 2-hydroxypropanethiol, 1-mercapto-2-propanol, dithioerythritol, dithiothreitol, cysteamine, and alpha-lipoic acid. In some embodiments, the BT composition comprises one or more BT compounds selected from BisBAL, BisEDT, Bis-dimercaprol, BisDTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, BisPyr/EDT, Bis-Pyr/PDT, Bis-Pyr/Tol, Bis-Pyr/Ery, BisEDT/CSTMN (1:1), BisPYR/CSTMN (1:1), BisBAL/CSTMN (1:1), BisTOL/CSTMN (1:1), bismuth-1-mercapto-2-propanol, and Bis-EDT/2-hydroxy-1-propanethiol. In some embodiments, the BT compound is selected from one or more of Bis-EDT, Bis-Bal, Bis-Pyr, Bis-Ery, Bis-Tol, Bis-BDT, or Bis-EDT/2-hydroxy-1-propane thiol. In some embodiments, the BT compound on the aerosol is BisEDT. In some embodiments, the BT compound of the aerosol is BisBDT or BisBAL.

In some embodiments of the presently disclosed compositions, at least 60%, 65%, 70%, 75%, 80%, 90%, or 95% of the microparticles have a volumetric mean diameter of from about 0.6 μm to about 2.5 μm. In some embodiments, substantially all of the microparticles have a VMD of from about 0.6 μm to about 2.5 μm. In some embodiments, at least 70% of the aerosolized particles have a MMAD of about 0.9 μm to about 3 μm. In some embodiments, the composition is a suspension of microparticles having a volumetric mean diameter (VMD) from about 0.6 μm to about 2.5 μm and/or a mass median aerodynamic diameter (MMAD) from about 0.9 μm to about 3 μm. In some embodiments, the bismuth-thiol composition comprises a plurality of microparticles that comprise a bismuth-thiol (BT) compound, substantially all of said microparticles having a volumetric mean diameter of from about 0.4 μm to about 5 μm, and wherein the BT compound comprises bismuth or a bismuth salt and a thiol-containing compound.

In some embodiments, the composition is aerosolized via a nebulizer. For example, the nebulizer is a jet nebulizer or vibrating mesh nebulizer. In some embodiments, the jet nebulizer is a Pari LC Plus jet nebulizer or Pari LC SPRINT jet nebulizer. In some embodiments, the nebulizer has an inlet pressure from about 10 to about 40 psig (e.g. 20-25 psig). In some embodiments, the inlet flow is from about 3 L/min to about 8 L/min (e.g. 5.2 L/min). In some embodiments, the exhaust air flow is from about 3 L/min to about 8 L/min (e.g. 5 L/min).

Examples of suitable aqueous and nonaqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agent scan also be desirable to include isotonic agents, such as sugars, sodium chloride, and the like into the compositions. In addition, prolonged absorption of the injectable pharmaceutical form can be brought about by the inclusion of agents that delay absorption such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, can depend upon crystal size and crystalline form. Alternatively, delayed absorption of a parenterally administered drug form is accomplished by dissolving or suspending the drug in an oil vehicle.

Actual dosage levels of the active ingredients in the pharmaceutical compositions can be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the subject being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount can include, but are not limited to, the severity of the subject's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable dose of an active compound used in the compositions and methods of the disclosure will be that amount of the compound that is the lowest dose effective to produce a therapeutic effect. Such an effective dose will generally depend upon the factors described above.

This disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Kits

In some embodiments, the present disclosure provides a kit comprising (1) an aerosol comprising a plurality of dispersed liquid droplets in a gas, said liquid droplets comprising a BT composition comprising BisEDT compound suspended therein; and wherein at least 70% of the liquid droplets have a MMAD from about of from about 0.9 μm to about 3 μm, and (2) at least one antimicrobial agent. In some embodiments, the at least one antimicrobial agent is formulated for administration orally, parenterally, topically, perorally, internally, intranasally, rectally, vaginally, lingually, or transdermally. In some embodiments, the at least one antimicrobial agents is a broad spectrum antiviral agent. In some embodiments, the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

Methods of Treating Pulmonary Infections

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation.

In healthy individuals, clearance of lung bacteria relies on the concerted action of two anatomic features: (i) the ciliated apical surface of the airway epithelium and (ii) a mucus layer that lines the airway lumen. The airway cilia beat synchronously, creating a steady current that continually moves the mucus layer upward toward the nasopharynx. The mucus layer is biphasic, consisting of an upper, viscous layer that serves to trap particulates and microorganisms and a

lower, more fluid layer in which the cilia beat. When functioning normally, this clearance system traps foreign bodies in the mucus and subsequently carries them to the nasopharynx, where they are expectorated and swallowed.

However, abnormal secretory characteristics of the CF airway cells due to the ion imbalance caused by the mutant CFTR protein alter the viscosity of the airway fluid, such that the normally serous “periciliary” layer becomes thicker, inhibiting escalator action that clears foreign bodies. Bacteria are trapped in the mucous and result in an ongoing infection in the lungs.

CF patients routinely produce sputum from the lungs through coughing, aided by other physical therapies designed to free mucous from the lungs. Many of the organisms that are isolated from CF sputum are pathogens that often benignly colonize the upper respiratory tract (e.g., non-typeable H. influenzae) or the nose (e.g., S. aureus) or are common environmental organisms that behave as pathogens only under certain opportunistic situations (e.g., P. aeruginosa). Different bacteria and the level of infection in the lungs can be determinative of a CF patient's symptoms and outcome. For example, the presence of S. aureus and the absence of P. aeruginosa predicts long term survival in CF patients after the age of 18 years. In addition, the potential for increasing P. aeruginosa colonization as a consequence of suppression of S. aureus infection may be relevant for some patients.

Of all the bacteria that can colonize in the lungs of CF patients, chronic P. aeruginosa airway infection and the accompanying inflammatory response are the major clinical problems for CF patients today. While antibiotic chemotherapy and chemoprophylaxis have reduced the morbidity and early mortality of CF patients from this infection, the intrinsic ability of P. aeruginosa to develop resistance to many commonly used antibiotics probably contributes to the inability to eradicate P. aeruginosa from CF patients' lung and ultimately allows this microbe to be highly problematic for these patients.

CF patients can acquire P. aeruginosa in their respiratory tracts at any time, with most studies indicating that 70 to 80% CF patients are infected by their teen years. P. aeruginosa infection probably initially occurs within the first 3 years of life. After the onset of chronic infection, patients experience episodic exacerbations that can benefit from antibiotic chemotherapy. Infection may result from social contacts or may be hospital acquired, but the diversity of P. aeruginosa clones isolated from CF patients suggests that most clinical isolates originate in the environment. CF patients chronically infected by P. aeruginosa show a steeper lung function decline (expressed as forced expired volume in 1 second (FEV1) decline over time), a higher number of pulmonary exacerbations, more hospital admissions and higher mortality than P. aeruginosa—free patients. The effects of P. aeruginosa are more severe if chronic infection develops early.

P. aeruginosa infections can change over time to develop a mucoid phenotype, which can initiate the chronic-infection stage of cystic fibrosis. The mucoid phenotype results from bacterial production of a polysaccharide known as both alginate and mucoid exopolysaccharide (MEP) and plays an important role in bacterial evasion of the host immune response. The MEP/alginate itself is able to promote bacterial survival in the face of host immune effectors. Alginate overproduction by P. aeruginosa correlates with the onset of significant deterioration in lung function. In addition, P. aeruginosa can grow as a biofilm, which increases bacterial resistance to phagocytic action and antibiotic efficacy.

Bacterial biofilms are a matrix of cells that adhere to each other and often a surface, such as lung mucosa. The bacterial cells become embedded within an extracellular matrix formed from extracellular polymeric substances, such as polysaccharides, proteins, lipids and DNA. Biofilm bacterial cells are physiologically different than planktonic cells in which a large number of genes are differentially regulated. Biofilms can also be more resistant to antibiotics given the shelter provided by the matrix. Biofilms of P. aeruginosa and other bacteria that are present in the lungs of CF patients increase the difficulty of successful infection management and reduction. Combinations of CF-relevant bacteria forming multispecies biofilms containing P. aeruginosa have demonstrated greater resistance, virulence and pathogenicity than comparable single-species biofilms. The presence of such complex biofilms in the lungs of CF patients is considered to be largely responsible for the chronic, persistent nature of these pulmonary infections, which are not only responsible for chronic, ongoing and progressive morbidity, but are also ultimately responsible for mortality in this population.

In addition to P. aeruginosa, other pathogens commonly found in CF patients' lungs include, but are not limited to, Haemophilus influenzae, Staphylococcus aureus, Staphylococcus warneri, Staphylococcus lugdunensis, Staphylococcus epidermidis, Streptococcus milleri/anginous, Streptococcus pyogenes, non-tuberculosis mycobacterium, Mycobacterium tuberculosis, Burkholderia spp., Achromobacter xylosoxidans, Pandoraea sputorum, Stenotrophomonas maltophilia, Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morganii, Inquilinus limosus, Ralstonia mannitolilytica, Pandoraea apista, Pandoraea pnomenusa, Pandoraea sputorum, Bdellovibrio bacteriovorus, Bordetella bronchiseptica, Vampirovibrio chlorellavorus, Actinobacter baumanni, Cupriadidus metallidurans, Cupriavidus pauculus, Cupriavidus respiraculi, Delftia acidivordans, Exophilia dermatitidis, Herbaspirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralstonia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S. agalactiae, Bacteroides fragilis, Clostridium difficile, Streptococcus pneumonia, Moraxella catarrhalis, Haemophilus haemolyticus, Haemophilus parainfluenzae, Chlamydophilia pneumoniae, Mycoplasma pneumoniae, Atopobium spp., Sphingomonas spp., Saccharibacteria spp., Leptotrichia spp., Capnocytophaga, Oribacterium spp., Aquabacterium spp., Lachnoanaerobaculum spp., Campylobacter spp., Acinetobacter spp., Agrobacterium spp., Bordetella spp., Brevundimonas spp., Chryseobacterium spp., Delftia spp., Enterobacter spp., Klebsiella spp., Pandoraea spp., Pseudomonas spp., Ralstonia spp., and Prevotella spp.

Exemplary non-tuberculosis mycobacterium include, but are not limited to, Mycobacterium abscessus, Mycobacterium avium, Mycobacterium intracellulare, Mycobacterium fortuitum, Mycobacterium gordonae, Mycobacterium kansasii, Mycobacterium avium complex (MAC), Mycobacterium abscessus complex (MABSC) Mycobacterium marinum, Mycobacterium terrae and Mycobacterium cheloni.

Exemplary species of Burkholderia include, but are not limited to, Burkholderia cepacia, Burkholderia multivorans, Burkholderia cenocepacia, Burkholderia stabilis, Burkholderia vietnamiensis, Burkholderia dolosa, Burkholderia ambifaria, Burkholderia anthina, Burkholderia pyrrocinia, Burkholderia gladioli, Burkholderia ubonensis, Burkholderia arboris, Burkholderia latens, Burkholderia lata, Burkholderia metallica, Burkholderia seminalis, Burkholderia contaminans, and Burkholderia diffusa.

In some embodiments, the bacterial pathogen is selected from Pseudomonas aeruginosa, multi drug-resistant Pseudomonas aeruginosa, Staphylococcus aureus, multi drug-resistant Staphylococcus aureus, methicillin resistant Staphylococcus aureus, Mycobacterium abscessus, Mycobacterium avium, Burkholderia cepacia, Burkholderia multivorans, Burkholderia cenocepacia, Burkholderia dolosa, Achromobacter xylosoxidans, Stenotrophomonas maltophilia Staphylococcus epidermidis, and Burkholderia vietnamiensis. In certain embodiments, the bacterial pathogen is selected from Haemophilus influenzae, Pseudomonas aeruginosa, and Staphylococcus aureus. In certain embodiments, the bacterial pathogen is selected from biofilms of Pseudomonas aeruginosa, Burkholderia cenocepacia, Burkholderia cepacia complex, Mycobacterium abscessus, Mycobacterium avium, Achromobacter spp., Staphylococcus epidermidis, Stenotrophomonas maltophilia, and Staphylococcus aureus.

In some embodiments, the bacterial pathogen exhibits resistance to one or more antibiotics. Methicillin-resistant S. aureus (MRSA) is an example of a singly resistant strain that is difficult to treat in CF patients and the population at large, while even more challenging multi-drug resistant (MDR) strains can occur in bacteria such as P. aeruginosa and S. aureus. For example, a bacterial pathogen can become resistant to known standards of antibiotic care, including, but not limited to, amikacin, aztreonam, methicillin, vancomycin, nafcillin, gentamicin, ampicillin, chloramphenicol, doxycycline and tobramycin. In some embodiments, the resistant antibiotic is amikacin, aztreonam, or tobramycin.

Long-term, repeated treatment with antibiotics to treat CF-associated infections typically results in development of antibiotic-resistance, characterized by the presence of microbial biofilms. Recent research has repeatedly demonstrated a correlation between multi-drug resistant (MDR) bacteria, and stronger, more prolific biofilm-forming capabilities. Biofilm involvement in the lung is considered highly immunogenic, accelerating structural lung damage. Further, bacteria within biofilms are protected from antibiotics, which increases the minimal inhibitory concentration of such antibiotics. Biofilms tend to reduce the antimicrobial activity of aminoglycosides and beta-lactam antibiotics by both changing the pH of the respiratory mucosa and through the production of beta-lactamase enzymes. The involvement of biofilm-forming bacteria in CF is correlated with decreased lung function and reduced Quality of Life, decreased response to antibiotic therapy, increased exacerbations, and, over time, reduced survival.

In some embodiments, the BT composition is administered by inhalation, either orally or nasally, using an aerosol device, such as a nebulizer. A nebulizer can administer the BT composition topically to the lung tissue, which can include the lung mucosa, the alveoli (e.g. deep lung alveoli), the bronchi and/or the bronchioles. Thus, in some embodiments, the present disclosure provides for administration of the amorphous BisEDT composition to the deep lung region of the lung (e.g. the deep lung alveoli). Local topical administration of the BT composition provides several key advantages over systemic antibiotic therapies. The term “systemic” refers to administration of a medication into the circulatory system of the subject such that the majority of the entire body can be exposed. Systemic administration of a medication can occur enterally (absorption through the gastrointestinal tract, e.g. oral administration) or parenterally (absorption through injection or infusion, e.g. intravenously).

Systemic anti-infective products have several disadvantages relating to the treatment of localized infections, including: (a) difficulty in achieving a therapeutically effective concentration at the site of local infection, particularly in the case of infections associated with topical locations, such as pulmonary airways, (b) frequent unintended toxic effects on organ systems exposed through systemic circulation, and (c) increased generation of antibiotic-resistant bacteria as a result of the widespread exposure of the body's normal flora to the anti-infective agent, (d) resulting in greater likelihood of transmission of such antibiotic-resistant bacteria to others as a result of long term sustained/repeated exposure of the entire body's complement of normal flora and (e) reduced preservation of the beneficial influence of the healthy normal flora throughout the body due to extensive systemic exposure.

Oral dosing can result in high plasma concentrations that may lead to toxicity and varied inter-patient exposure (variable absorption and variable first-pass, hepatic clearance) or drug-drug interactions. When orally dosing to treat a lung infection, high plasma exposure is necessary to achieve therapeutic exposure in the lungs. In contrast, inhaled dosing for topical lung indications requires a lower total dose to achieve an efficacious concentration because the drug is administered directly (topically) to the site of infection, which results in significantly less systemic exposure. Inhaled dosing achieves higher local concentrations in the lung that significantly exceed the MIC of the drug over a long period of time. Due to the delivery of high concentrations of drug directly to the lung, the achieved pulmonary concentrations following inhalation may greatly exceed those achieved by oral dosing. From previous experience with inhaled antibiotics, the lung concentration of drug upon inhalation is >100× greater than upon systemic/oral administration of the same dose.

In some embodiments, one or more of the following symptoms (e.g. cystic fibrosis-related symptoms) is lessened in severity in the subject: cough, wheezing, breathlessness, bronchiectasis, nasal polyps, hemoptysis, respiratory failure, and pulmonary exacerbation. Additional non-cystic fibrosis related infections may also be lessened in severity. Thus, inhaled formulations of the BT compositions disclosed herein provide a more targeted and effective antibiotic treatment than a corresponding formulation administered systemically.

BisEDT is known broad-spectrum antimicrobial (and anti-biofilm) small molecule drug product for the treatment of chronic, ultimately life-threatening pulmonary infections secondary to CF. Its efficacy extends to Gram-positive, antibiotic-resistant pathogens including methicillin-resistant Staphylococcus aureus (MRSA, including community-associated [CA]-MRSA), methicillin-resistant Staphylococcus epidermidis (MRSE), and vancomycin-resistant Enterococcus (VRE). BT compounds are also potent against Multi-drug-resistant (MDR) Gram-negative pathogens including Pseudomonas aeruginosa, Escherichia coli, Klebsiella pneumoniae (including, in all of the afore-mentioned bacteria, carbapenem-resistant strains), and Acinetobacter baumannii.

BisEDT have the dual ability to overcome a) a very diversified spectrum of antibiotic resistance profiles (due to evolution/diversification driven by persistence, time and isolation in many different anatomical regions throughout the pulmonary airways), and b) antibiotic-resistant and MDR biofilms.

Disclosed herein are methods of treating, managing or lessening the severity of cystic fibrosis (CF) symptoms and infections in a subject, the method comprising administering to the subject a bismuth-thiol (BT) composition that comprises amorphous BisEDT. Also disclosed herein are methods of treating, managing or lessening the severity of symptoms and infections associated with one or more pulmonary diseases or infections in a subject, including non-CF associated diseases, the method comprising administering to the subject a bismuth-thiol (BT) composition that comprises amorphous BisEDT. In some embodiments, the subject has at least one pulmonary infection, such as a CF-related pulmonary infection. In other embodiments, the subject has at least two pulmonary infections and the infections are either concurrent or successive in order. The pulmonary infections could be cause by the same microbial pathogen and be located in two different lungs, or lobes of the lung. In other embodiments, the pulmonary infections could be caused by different microbial pathogens and be located in the same lung, or lobe of the lung. In some embodiments, the pulmonary infection is in one lung, while in others it is present in both lungs. In certain embodiments, the pulmonary infection is in one or more of the three lobes of the right lung. In other embodiments, the pulmonary infection is in one or both of the two lobes of the left lung. Any combination of one or more microbial pathogens, microbial pathogen quantity, and infection location in the lung is contemplated within the term “pulmonary infection”. In some embodiments, the pulmonary infection is a bronchiectasis infection, pneumonia, valley fever, allergic bronchopulmonary aspergillosis (ABPA), ventilator acquired pneumonia, hospital acquired pneumonia, community acquired pneumonia, ventilator associated tracheobronchitis, lower respiratory tract infection, non-tuberculous Mycobacteria, anthrax, legionellosis, pertussis, bronchitis, Bronchiolitis, COPD-associated infection, and post-lung transplantation. In some embodiments, the pulmonary infection is a bronchiectasis infection.

In some embodiments, the pulmonary infection contains one or more bacterial or fungal pathogens. In some embodiments, the disclosed methods comprise treating the CF-related pulmonary infection. In some embodiments, the disclosed methods comprise managing the CF-related pulmonary infection. In some embodiments, the disclosed methods comprise lessening the severity of the CF-related pulmonary infection.

In some embodiments, the methods of the present invention may include treating, managing or lessening the severity of symptoms and infections associated with one or more pulmonary diseases or infections in a subject by administering to the subject a bismuth-thiol (BT) composition that comprises amorphous BisEDT

In certain embodiments, the pulmonary infection is located in or on the lung mucosa, the bronchi and/or the bronchioles. In other embodiments, the pulmonary infection is located on the surface of or within a bacterial biofilm, aggregated bacteria, a fungal biofilm, or aggregated fungi. In some embodiments, the pulmonary infection is located in the sputum wherein the pulmonary infection involves and is, at least in part, present in the mucous/sputum layers associated with the lungs. In certain embodiments, the bacterial pathogen comprises one or more of gram-positive bacteria and gram-negative bacteria. The bacterial pathogen can comprise one or more of a bacterial biofilm and planktonic bacteria. In some embodiments, the fungal pathogen comprises one or more of a fungal biofilm and planktonic fungi. In certain embodiments, the fungal pathogen is Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, and/or Aspergillus flavus.

In some embodiments, the method comprises at least one of: (i) reducing the microbial (e.g. bacterial or fungal) biofilm, (ii) impairing growth of the microbial (e.g. bacterial or fungal) biofilm, and (iii) preventing reformation of the microbial (e.g. bacterial or fungal) biofilm. In other embodiments, the amorphous BisEDT manages or lessens the severity of the pulmonary infection by one or more of:
prevention of the infection by the bacterial or fungal pathogen; —prevention of elaboration or secretion of exotoxins from the bacterial or fungal pathogen;
reduction of the bacterial or fungal pathogen (e.g. as measure by amount or titer);
inhibition of cell viability or cell growth of planktonic cells (e.g. substantially all of the cells) of the bacterial or fungal pathogen;
inhibition of biofilm formation by the bacterial or fungal pathogen;
inhibition of biofilm viability or biofilm growth of biofilm-form cells (e.g. substantially all of the cells) of the bacterial or fungal pathogen; and
reducing the viscosity of the sputum.

In some embodiments, the bismuth-thiol composition comprises a plurality of microparticles that comprise amorphous BisEDT, substantially all of said microparticles having a volumetric mean diameter of less than about 5 μm, less than about 4 μm, less than about 3 μm, less than about 2 μm, less than about 1 μm, from about 0.01 μm to about 5 μm, from about 0.1 μm to about 5 μm, from about 0.4 μm to about 5 μm, and wherein the BT compound comprises amorphous BisEDT. In some embodiments, at least 60%, 65%, 70, 75%, 80%, 90%, or 95% of the microparticles have a volumetric mean diameter of less than about 5 μm, less than about 4 μm, less than about 3 μm, less than about 2 μm, less than about 1 μm, from about 0.01 μm to about 5 μm, from about 0.1 μm to about 5 μm, or of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the presently disclosed methods, at least 60%, 65%, 70, 75%, 80%, 90%, or 95% of the microparticles have a volumetric mean diameter of less than about 5 μm, less than about 4 μm, less than about 3 μm, less than about 2 μm, less than about 1 μm, from about 0.01 μm to about 5 μm, from about 0.1 μm to about 5 μm or of from about 0.01 μm to about 2.5 μm. In some embodiments, substantially all of the microparticles have a VMD of from about 0.1 μm to about 2.5 μm or less than about 5 μm, less than about 4 μm, less than about 3 μm, less than about 2 μm, less than about 1 μm, from about 0.01 μm to about 5 μm, or from about 0.1 μm to about 5 μm. In some embodiments, at least 70% of the aerosolized particles have a MMAD of about 0.01 μm to about 3 μm. In some embodiments, the composition is a suspension of microparticles having a volumetric mean diameter (VMD) from about 0.1 μm to about 2.5 μm and/or a mass median aerodynamic diameter (MMAD) from about 0.1 μm to about 3 μm. In some embodiments, the bismuth-thiol composition comprises a plurality of microparticles that comprise amorphous BisEDT, substantially all of said microparticles having a volumetric mean diameter of from about 0.1 μm to about 5 μm, and wherein the BT compound comprises bismuth or a bismuth salt and a thiol-containing compound.

In some embodiments, the present disclosure provides a method of treating, managing or lessening the severity of symptoms and infections associated with one or more pulmonary diseases or infections in a subject, the method comprising administering to the subject a bismuth-thiol (BT) composition that comprises amorphous BisEDT suspended therein, wherein administering the BT composition is via inhalation, orally or nasally, using an aerosol device. In some embodiments, the method is treating, managing or lessening the severity of cystic fibrosis (CF) symptoms and infections in a subject.

In some embodiments, the present disclosure provides a method of treating, managing or lessening the severity of cystic fibrosis (CF) symptoms and infections in a subject, the method comprising administering to the subject a bismuth-thiol (BT) composition that comprises amorphous BisEDT. In some embodiments, the BT composition comprises a plurality of microparticles wherein at least 70%, 80%, or 90% of said microparticles having a volumetric mean diameter (VMD) from about 0.6 μm to about 2.5 μm. In some embodiments, when the BT composition is aerosolized, at least 70%, 80%, or 90% of the aerosolized liquid droplets have a mass median aerodynamic diameter (MMAD) from about 0.9 μm to about 3 μm. In some embodiments, the BT composition comprises BisEDT at a concentration greater than about 0.1 mg/mL, about 0.05% to about 1.0% Tween 80®, about 40 mM to about 250 mM sodium chloride, and optionally about 2 to 20 mM sodium phosphate at about pH. 7.4. In some embodiments, the subject has at least one pulmonary infection containing one or more bacterial pathogens and/or fungal pathogens (as described herein). In some embodiments, the method comprises at least one of: (i) reducing a bacterial biofilm, (ii) impairing growth of a bacterial biofilm, (iii) preventing initial formation of the bacterial biofilm, and/or (iv) preventing reformation of the bacterial biofilm.

### Methods of Treating Wounds

In some embodiments, the present disclosure provides methods for treating a topical wound, comprising administering to a subject in need thereof a therapeutically effective amount of a composition comprising amorphous BisEDT, wherein the composition is applied to the infection (e.g. applied to the surface of the infection).

In some embodiments, the topical wound is a skin ulcer (e.g. a skin ulcer on a lower extremity). In some embodiments, the topical wound is a skin ulcer on a lower extremity, such as the leg and/or foot. In some embodiments, the skin ulcer is one or more of foot ulcer, ischemic ulcer, gangrenous ulcer, venous stasis ulcer, decubitus ulcer, Buruli ulcer, or traumatic ulcer. In some embodiments, the skin ulcer is a foot ulcer. In some embodiments, the topical wound is infected by one or more bacterial and/or fungal pathogens. In some embodiments, the topical wound is infected by bacterial pathogens. In some embodiments, the topical wound is a diabetic foot ulcer. In some embodiments, the diabetic foot ulcer is a diabetic foot infection. In some embodiments, the topical wound is infected with one or more of the following bacterial pathogens: Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis.

Biofilms of S. aureus and other bacteria that are present in the wounds of DFI patients increase the difficulty of successful infection management and reduction. Combinations of DFI-relevant bacteria forming multispecies biofilms containing e.g. S. aureus have demonstrated greater resistance, virulence and pathogenicity than comparable single-species biofilms. The presence of such complex biofilms in the wounds of DFI patients is considered to be largely responsible for the chronic, persistent nature of these infections.

In some embodiments, the bacterial pathogen exhibits resistance to one or more antibiotics. Of particular concern are the methicillin-resistant Staphylococcus aureus strains (MRSA). MRSA remained an uncommon occurrence in hospital setting until the 1990's, when there was an explosion in MRSA prevalence in hospitals. MRSA now is considered endemic to hospitals, especially in the UK (Johnson A P et al. 2001 J. Antimicrobial Chemotherapy 48(1): 143-144). Moreover, MRSA presents a new threat in diabetic foot infections (Retrieved Jan. 17, 2009, from CDC: Centers for Disease Control and Prevention Web site). The ulcers and open sores that can occur in diabetic feet put patients at risk for contracting MRSA, and recent studies show evidence of MRSA impairing healing when present in the diabetic wound (Bowling F L, et al. 2009 Curr Diab Rep 9(6):440-444). See also, Kosinski, M A, et al. 2010. Expert Rev AntiInfect Ther. 8(11):1293-1305. In some embodiments, a bacterial pathogen is resistant to known standards of antibiotic care, including, but not limited to, amikacin, methicillin, vancomycin, nafcillin, gentamicin, metronidazole, Piperacillin/Tazobactam, ampicillin, chloramphenicol, doxycycline, tobramycin, levofloxacin, cephalosporins (e.g. cephalexin, cefoxitin, ceftizoxime, ceftibiprole, ceftazidime, ceftaroline), penicillin/3-lactamase inhibitor combinations (e.g. amoxicillin/clavulanate, ampicillin/sulbactam, piperacillin/tazobactam, and ticarcillin/clavulanate), carbapenems (e.g. imipenem/cilastatin, ertapenem), fluoroquinolones (e.g. ciprofloxacin, moxifloxacin), clindamycin, linezolid, daptomycin, tigecycline, and vancomycin.

Long-term, repeated treatment with antibiotics to treat DFI-associated infections may result in development of antibiotic-resistance, characterized by the presence of microbial biofilms. Recent research has repeatedly demonstrated a correlation between multi-drug resistant (MDR) bacteria, and stronger, more prolific biofilm-forming capabilities. Bacteria within biofilms are protected from antibiotics, which increases the minimal inhibitory concentration of such antibiotics.

The BT compositions (i.e. amorphous BisEDT compositions) of the present disclosure have activity against a plurality of bacterial and fungal strains. In some embodiments, the BT compositions have activity against a plurality of strains including but not limited to Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. Accordingly, some embodiments of the present disclosure provide methods of treating and/or preventing infections associated with Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis in both humans and animals using the BT compositions. In other aspects, the present disclosure provides methods of treating and/or preventing infections associated with related species or strains of these bacteria. In some embodiments, the bacterial infection is an infection associated with diabetic lower extremity infections, such as diabetic foot infections.

Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis are responsible for many severe opportunistic infections, particularly in individuals with compromised immune systems, including diabetic patients. The pharmaceutical compositions of the present disclosure are contemplated for treating and/or preventing any infection associated with Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis or associated with other species or strains of bacteria, including, but not limited to, infections of the skin, infections in and around wounds, chronic ulcers, ulcers associated with burn wounds, post-operative infections, infections associated with catheters and surgical drains, and infections of the blood. In some embodiments, the pharmaceutical compositions of the present disclosure find use in treating and/or preventing bacterial infections associated with areas of non-intact skin, including but not limited to, infections associated with cutaneous ulcers, such as diabetic foot ulcers, skin lesions, vesicles, cysts, blisters, bullae, open sores such as decubitus ulcers (bed sores) and other pressure sores, chronic ulcers, cellulitis and sores associated therewith, erysipelas and lesions associated therewith, wounds, burns and wounds associated therewith, carbuncles, or other conditions where the skin is damaged, cracked, broken, breached, and/or otherwise compromised.

In any of the embodiments described herein, the BT compositions may be used to treat an infection (e.g. DFI) of one or more of the following bacterial pathogens:

Acinetobacter baumanii
Acinetobacter junii
Anaerococcus lactolyticus
Anaerococcus vaginalis
Anaerococcus murdoch
Anaerococcus tetradius
Anaerococcus hydrogenalis
Actinobaculum massiliense
Actinobaculum schaalii
Actinomyces europacus
Actinomyces hominis
Actinomyces neuii
Actinomyces radingae
Alcaligenes faecalis
Abiotrophia paraadiacens
Bacteroides fragilis
Bulleidia extructa
Bilophila wadsworthia
Campylobacter ureolyticus
Citrobacter murliniae
Clostridium saccharogumia
Clostridium novyi
Corynebacterium accolens
Corynebacterium amycolatum
Corynebacterium aurimucosum
Corynebacterium freiburgense
Corynebacterium hansenii
Corynebacterium jeikeium
Corynebacterium mycetoide
Corynebacterium simulans

### Corynebacterium Tuberculostearicum

Corynebacterium xerosis
Corynebacterium striatum
Dermabacter hominis
Dialister invisus
Dialister propionicifaciens
Dialister micraerophilus
Dialister pneumosintes
Delftia acidovorans
Enterobacter aerogenes
Enterobacter cloacae
Enterobacter hormaechei
Enterococcus faecalis
Enterococcus canintestini
Echerichia coli
Escherichia fergusonii
Escherichia vulneris
Enterococcus avium
Enterococcus gallinarum
Enterococcus casseliflavus
Eggerthella lenta
Finegoldia magna
Fusobacterium canifelinum
Fusobacterium nucleatum
Fusobacterium periodontium
Granulicatella adiacens
Gemella morbillorum
Globicatella sanguinis
Haemophilus parainfluenzae
Haemophilus segnis
Helcococcus kunzii
Helcococcus kunzii
Klebsiella oxytoca
Kocuria atrinae
Leclercia adecarboxylata
Mobiluncus curtisii
Moryella indoligenes
Morganella morganii
Negativicoccus succinicivorans
Peptoniphilus harei
Peptoniphilus gorbachii
Peptoniphilus ivorii
Peptoniphilus lacrimalis
Peptoniphilus olsenii
Peptoniphilus asacchrolyticus
Parvimonas micra
Peptococcus niger
Peptostreptococcus anaerobius
Peptostreptococcus stomatis
Porphyromonas asaccharolytica
Porphyromonas bennonis
Porphyromonas somerae
Porphyromonas uenonis
Porphyromonas levii
Prevotella timonensis
Prevotella bergensis
Prevotella buccalis
Prevotella corporis
Prevotella disiens
Prevotella intermedia
Prevotella nanceiensis
Pseudomonas indica
Pseudomonas olitidis
Psychrobacter lutiphocae
Proteus myxofaciens
Proteus hauseri
Providencia rettgeri
Providencia stuartii
Staphylococcus aureus
Staphylococcus epidermidis
Staphylococcus carnosus
Staphylococcus chromogenes
Staphylococcus devriesei
Staphylococcus hominis
Staphylococcus lugdunensis
Serratia nematodiphila
Stenotrophomonas maltophilia
Staphylococcus pettenkoferi
Staphylococcus capitis
Staphylococcus saprophyticus
Streptococcus agalactiae
Streptococcus anginosus
Streptococcus canis
Streptococcus dysgalactiae
Streptococcus infantarius
Streptococcus oralis
Serratia grimesii
Stenotrophomonas pavanii
Salmonella enterica
Trueperella bernardiae
Varibaculum cambriense
Veillonella atypica
Veillonella parvula
Veillonella dispar
Veillonella rogosae
Acinetobacter calcoaceticus
Acinetobacter lwoffi
Anaerococcus prevotii
Bacteroides caccae
Bacteroides distasonis
Bacteroides ovatus
Bacteroides stercoris
Bacteroides thetaiotaomicron
Bacteroides uniformis
Bacteroides vulgatus
Corynebacterium striatum
Clostridium innocuum
Clostridium perfringens
Clostridium ramosum
Pluralibacter gergoviae
Fusobacterium mortiferum
Finegoldia magna
Klebsiella oxytoca
Klebsiella pneumoniae
Pseudomonas aeruginosa
Peptococcus magnus
Prevotella bivia
Prevotella melaninogenica
Porphyromonas asaccharolytica
Peptostreptococcus asaccharolyticus
Peptostreptococcus micros
Parvimonas micra
Proteus mirabilis
Staphylococcus haemolyticus
Staphylococcus simulans

### Staphylococcussaprophyticus

Streptococcus pneumoniae
Streptococcus agalactiae
Streptococcus mitis
Streptococcus milleri
Streptococcus dysgalactiae
Streptococcus canis
Serratia marcescens
Serratia liquefaciens
Stenotrophomonas maltophila
Epidermolysis bullosa

In any of the embodiments described herein, the BT compositions may be used to treat an infection (e.g. DFI) of one or more of the following fungal pathogens: Candida spp., Cladosporium spp., Aspergillus spp., Penicillium spp., Alternaria spp., Pleospora spp., Fusarium spp, Candida lusitaniae, Candida parapsilisis, and Candida albicans.

In some embodiments, the BT compositions of the present disclosure find use in treating chronic ulcers. Chronic ulcers may arise from wounds caused by a variety of factors, especially in patients with impaired blood circulation, for example, caused by cardiovascular issues or external pressure from a bed or a wheelchair. More than 8 million patients are diagnosed with chronic skin ulcers each year in the United States alone (Harsha, A. et al., 2008, Journal of Molecular Medicine, 86(8): 961-969), which costs more than 10 billion dollars per year (Margolis, D J, et al., 2002, Journal of the American Academy of Dermatology 46(3): 381-386). Chronic ulcers may develop in the mouth, throat, stomach, and skin. Chronic skin ulcers include diabetic ulcers, venous ulcers, radiation ulcers, and pressure ulcers, the three major categories of chronic skin ulcers being diabetic ulcers, venous stasis ulcers, and pressure ulcers. Chronic ulcers can cause the loss of the integrity of large portions of the skin, even leading to morbidity and mortality.

In some embodiments, the BT compositions of the present disclosure find use in treating diabetic lower extremity infections, such as diabetic foot infections. Diabetic foot infection is one of the major complications of diabetes mellitus, occurring in about 15% of all diabetic patients and resulting in about 85% of all lower leg amputations. (Brem, et al., J. Clinical Invest., 2007, 117(5). 1219-1222). Diabetes mellitus impedes the normal steps of the wound healing process, such that diabetic foot infections can become associated with non-healing, chronic cutaneous ulcers.

A chronic wound represents a failure of the normal processes of acute wound healing. Wound healing has traditionally been divided into three distinct phases: inflammation, proliferation and remodeling. The inflammatory phase of wound healing begins at the time of injury by forming a clot via a platelet plug, thereby initiating a response from neutrophils and macrophages. Neutrophils initially clear the wound of bacteria and debris by releasing a variety of proteases and reactive oxygen free radicals. Macrophages are then attracted to the wound site by chemoattractants and subsequently release their own chemoattractants to stimulate fibroblasts and more macrophages. During the proliferation phase, fibroblasts initiate epithelialization, angiogenesis, and collagenation. Epithelialization generally occurs from the basement membrane if it remains intact and from the wound margins if not intact. Fibroblasts synthesize type II collagen during this phase and transform into myofibroblasts, which help to stimulate wound contraction. During the remodeling phase, type III collagen begins to be replaced by type I collagen. Collagen is woven into an organized, cross-linked network whose strength approaches 80% of the original uninjured tissue.

There are many factors that can stall the three-phase healing process and convert an acute wound into a chronic wound. These may include a low proliferative capacity of the fibroblasts, downregulation of receptors, reduced growth factors, or the absence of a suitable protein matrix in the dermis. Further, poor perfusion and/or nutrition can cause a wound to halt in the inflammatory phase and lead to excessive build-up of exudate in the wound. A chronic ulcer can be considered to be a non-healing area of non-intact skin, such as an area of non-intact skin that fails to follow the normal processes of wound healing, e.g., as described above, and/or that fails to respond, or fails to respond appropriately, to initial treatment. A chronic ulcer on the skin may be characterized as a wound lesion lasting more than four weeks, without remarkable healing tendency or as a frequently recurrent wound (Fonder, M. et al., 2012, Journal of the American Academy of Dermatology 58(2): 185-206). A chronic wound may appear with red granulation and yellow pus, a dim purple skin around granular tissues, or gray-white and swelling granulation. Standard care procedures for chronic skin ulcer include, e.g., the following: removal of necrotic or infected tissue; establishment of adequate blood circulation; maintenance of a moist wound environment; management of wound infection; wound cleansing; and nutritional support, including blood glucose control for subjects with diabetic ulcers. For example, in the diabetic patient, poor control of blood glucose levels allows bacteria to grow more rapidly in a wound; further still, neural degeneration in diabetes means the condition may not be painful and thus go undetected, at least initially. Chronic ulcers, including diabetic foot ulcers, often become further infected with opportunistic bacteria, leading to exacerbation of the condition. Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis are associated with such infections.

In some embodiments, the pharmaceutical composition of the present disclosure is formulated for use in methods of treating and/or preventing bacterial infections caused by Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some embodiments, the pharmaceutical composition of the present disclosure is formulated for use in methods of treating and/or preventing bacterial infections caused by Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some other embodiments, the pharmaceutical composition of the present disclosure is formulated for use in methods of treating and/or preventing bacterial infections caused by bacteria other than Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis.

In some embodiments, the present disclosure provides methods of treating and/or preventing chronic ulcers, comprising administering to a subject in need thereof a therapeutically or prophylactically effective amount of a pharmaceutical composition of the present disclosure. In some embodiments, administration comprises topical administration to the area of non-intact skin associated with the chronic ulcer. In some embodiments, topical administration follows debridement of the area to be treated

In some embodiments, the present disclosure provides methods of treating and/or preventing diabetic foot infections, comprising administering to a subject in need thereof a therapeutically or prophylactically effective amount of a pharmaceutical composition of present disclosure. In some embodiments, administration comprises topical administration to the area of non-intact skin associated with the diabetic foot infection, e.g., a cutaneous ulcer. In some embodiments, topical administration follows debridement of the area to be treated.

Debridement can be accomplished by a number of approaches. Surgical debridement involves cutting away dead tissues of the wound or other area of non-intact skin. Mechanical debridement uses various methods to loosen and remove wound debris, such as a pressurized irrigation device, a whirlpool water bath, ultrasound, larval maggots, or specialized dressings. Autolytic debridement enhances the body's natural process of recruiting enzymes to break down dead tissue, for example, using an appropriate dressing that keeps the wound moist and clean. Enzymatic debridement uses chemical enzymes and appropriate dressings to further aid in the break down dead tissues at the site of a wound or other area of non-intact skin.

Debridement improves topical treatment because it reduces the bioburden of bacteria present and also opens a time-dependent therapeutic window for topical antimicrobial therapy (TAT) (Wolcott R D, et al. 2010. J Wound Care 19:320-328). Regarding the timing for debridement, early or immediate debridement is preferred to delayed debridement once this treatment option is chosen in the management of a wound. Further, multiple debridements during wound management may be indicated (Wolcott R D, et al. 2009. J Wound Care 18(2):54-6). For example, in some embodiments, debridement precedes topical application of a BT composition of the present disclosure, and is repeated before every administration of the BT composition. In some embodiments, debridement is performed only before every other administration of the BT composition, or only before every 3rd, 4th, 5th, or 6th administration of the BT composition. In some embodiments, debridements are performed less frequently than the application of the BT composition, for example, once a week. Accordingly, if the BT composition is applied daily, the patient will not get debridement every time it is applied. In some embodiments, whether or not wound debridement is performed before topical administration of a BT composition of the present disclosure is within the clinical judgment of a health care practitioner treating the wound, e.g., the physician, physician's assistant, or emergency medical personnel.

The BT compositions of the present disclosure can find use in the treatment, management, control, and/or prevention of infections associated with chronic ulcers, including diabetic foot infections and cutaneous ulcers associated therewith. In other embodiments, BT compositions of the present disclosure find use in the treatment, management, control, and/or prevention of microbial (e.g. bacterial and/or fungal) infections associated with other areas of non-intact skin, such as a cellulitis sore, an erysipelas lesion, a decubitus ulcer, a burn wound, a traumatic wound, and a pressure sore. In some such embodiments, the composition used may be a topical composition, formulated for topical administration, e.g., for direct application to an area of non-intact skin, such as described above.

The BT compositions of the present disclosure also find use in the treatment, management, control, and/or prevention of decubitus ulcers. Decubitus ulcers, also called pressure sores or pressure ulcers, are injuries to the skin and underlying tissues resulting from prolonged pressure on the area. For example, bedsores most often develop on skin that covers bony areas of the body, such as the heel, ankles, hips or buttocks.

Bedsores fall into one of four stages based on their severity. Stage 1 is the beginning stage of a pressure sore while the skin still is intact. The skin may appear red, ashen, bluish or purple, and fails to blanch when touched. Stage II often involves an open wound of non-intact skin. At this stage, the outer layer of skin (epidermis) and part of the underlying layer of skin (dermis) has been damaged or lost. The ulcer may appear as a shallow, pinkish-red, basin-shaped wound. In Stage III, the ulcer is a deep wound, where the loss of skin may expose some amount of fat, and the ulcer has a crater-like appearance. The bottom of the wound also may have some yellowish dead tissue (slough). A Stage IV ulcer exhibits large-scale loss of tissue, where the wound may expose muscle, bone and tendons. The bottom of the wound will likely contain slough or dark, crusty, dead tissue (eschar).

As in the treatment of diabetic foot ulcers, debridement (e.g. of pressure ulcers or bedsores) may be used to remove damaged, dead, or infected tissue from the wound, facilitating proper healing, e.g., as described herein and/or otherwise known in the art. In some embodiments, administration of a pharmaceutical composition of the present disclosure follows debridement. For example, a pharmaceutical composition disclosed herein may be topically administered to a decubitus ulcer following surgical, mechanical, autolytic, or enzymatic debridement thereof.

BT compositions of the present disclosure also find use in the treatment, management, control, and/or prevention of cellulitis and/or erysipelas, including but not limited to sores and lesions associated with cellulitis and erysipelas. Cellulitis and erysipelas are skin infections that develop as a result of bacterial entry via breaches in the protective barrier of the skin. For example, cracks in the skin, cuts, blisters, burns, insect bites, spider bites, tattoos, surgical wounds, intravenous drug injection, or sites of intravenous catheter insertion may provide a means of entry for bacteria. Group A Streptococcus and Staphylococcus are the most common bacteria involved in cellulitis. Cellulitis is observed most frequently among middle-aged and elderly individuals, while erysipelas occurs in young children and the elderly (Ellis Simonsen S M et al. 2006. Epidemiol Infect. 134(2):293; and Eriksson B. et al. 1996 Clin Infect Dis 23:1091). Also, people with immune deficiency, diabetes, alcoholism, fungal infections, and impaired lymphatic drainage are at increased risk. Diabetics are especially prone to cellulitis in the feet, because the disease causes impairment of blood circulation in the legs. The lower extremities are the most common site of infection for both erysipelas and cellulitis (Ellis Simonsen S M et al. 2006. Epidemiol Infect. 134(2):293; Chartier C et al 1996 Int J Dermatol 35:779).

Cellulitis and erysipelas often coexist and generally manifest as areas of skin erythema, edema, and warmth. They differ in that erysipelas involves the upper dermis and superficial lymphatics, whereas cellulitis involves the deeper dermis and subcutaneous fat. Accordingly, erysipelas has more distinctive anatomic features than cellulitis-erysipelas lesions may be raised above the level of surrounding skin with a clear line of demarcation between involved and uninvolved tissue (Bisno A L et al. 1996 N Engl J Med 334:240). The lesion may appear red, swollen, warm, hardened, and/or as a rash similar in consistency to an orange peel. Erysipelas may appear on the face, for example, in a “butterfly” pattern. More severe infections can result in vesicles, bullae, and petechiae, with possible skin necrosis. In addition, patients with erysipelas tend to have acute onset of signs and symptoms with systemic manifestations, including fever and chills.

Patients with cellulitis tend to have a more gradual course of development, with signs and symptoms appearing over a few days' time. Various forms of cellulitis include periorbital cellulitis, abdominal wall cellulitis (in morbidly obese individuals), buccal cellulitis (due to Streptococcus pneumoniae), Ludwig's angina (cellulitis within the submandibular space), and perianal cellulitis (due to group A beta-hemolytic streptococcus) (Barzilai A, et al, 1998 Pediatr Infect Dis J. 17(4):358; Thorsteinsdottir B, et al. 2005 Scand J Infect Dis. 37(8):605). Cellulitis also can result in influenza-like signs and symptoms, with high temperatures and shaking.

In some embodiments, treatment of cellulitis or erysipelas further comprises administration of an antibiotic agent. For example, a pharmaceutical composition according to the present disclosure may be topically administered to an erysipelas lesion, in combination with an antibiotic agent selected from the group consisting of penicillin, clindamycin, and erythromycin. As another example, a pharmaceutical composition according to the present disclosure may be topically administered to a sore associated with cellulitis, in combination with an antibiotic agent selected from the group consisting of flucloxacillin, dicloxacillin, penicillins, ampicillin, and amoxicillin. The antibiotic may be administered orally, intravenously, or topically, e.g., along with topical administration of a BT composition of the present disclosure.

BT compositions of the present disclosure also find use in the treatment, management, control, and/or prevention of infections associated with burn wounds. A burn wound is any area of non-intact skin caused, directly or indirectly, from a burn. A burn is a type of injury to the skin that can be caused by heat, as well as electricity, chemicals, light, radiation or friction. Burns may affect only the skin (epidermal tissue), but in some cases also injure deeper tissues, such as muscle, bone, and blood vessels. Burns can be classified by mechanism of injury, depth, extent and associated injuries, and comorbidities. Burns conventionally are described based on the depth of injury to the dermis, being loosely classified as first, second, third, and fourth degree burns (Walls et al., 2009, Rosen's Emergency Medicine: Expert Consult Premium Edition (Rosen's Emergency Medicine: Concepts & Clinical Practice (2v)). Important characteristics of a burn wound include its cause (thermal, chemical, electrical), anatomic location, depth (full or partial thickness), duration, and extent (percent total body surface area). Patient characteristics that affect burn wound healing include age, nutritional status, underlying medical conditions, and concomitant injury (e.g., head trauma, inhalation injury, bone fractures).

Infections among burn patients are a major problem, with the reported incidence of nosocomial infections varying at 63-240 per 100 patient days and 53-93 per 1000 patient days, mainly depending on the definitions used (Chim H, et al, 2007, Burns 33:1008-1014; and Wibbenmeyer L, et al., 2006, J Burn Care Res 27:152-60). Moreover, bacterial infection of burn wounds are associated with adverse outcomes and mortality. In a series of 175 patients with severe burns, for example, infections preceded multiorgan dysfunction in 83% of patients and were considered the direct cause of death in 36% of patients who did not survive (Fitzwater J, et al., 2003, J Trauma 54:959-66). Burn wounds may become infected from multiple sources. Burn wounds may become initially infected with Gram positive bacteria, mainly staphylococci, that are normal deep inhabitants of the sweat glands and hair follicles exposed by the burn (Sharma B R., 2007, Infect Dis Clin North Am 21:745-59; ix). The moist, vascular burn eschar further may foster microbial growth. Gram negative bacterial infections may result from translocation from the colon, for example, due to reduced mesenteric blood flow at the time of burn and subsequent insults (Herndon D N, et al., 2000, Crit Care Med 28:1682-3). Furthermore, burn patients may develop immune deficits, including impaired cytotoxic T lymphocyte response, myeloid maturation arrest causing neutropenia, impaired neutrophil function, and decreased macrophage production (Sharma B R., 2007, Infect Dis Clin North Am 21:745-59, ix; Gamelli R L, et al., 2000, J Burn Care Rehabil 21:64-9; Hunt J P, et al., 1998, J Surg Res 80:243-51; and Shoup M, et al., 1998, Ann Surg 228:112-22). Finally, burn patients are susceptible to hospital acquired infections, common to other patients in intensive care units, including intravascular catheter related infections and ventilator associated pneumonia, with an overall incidence of infection higher than that of other patients in intensive care units (Chim H, et al., 2007, Burns 33:1008-14; and Wibbenmeyer L, et al., 2006, J Burn Care Res 27:152-60). Indeed, most episodes of bloodstream infection in burn patients after the first week are caused by hospital-type multidrug resistant bacteria (Wibbenmeyer L, et al., 2006, J Burn Care Res 27:152-60; and Ressner R A, et al., 2008, J Am Coll Surg 206:439-44).

Conventional treatment of burns includes debridement and excision, applying dressings the wound, wound closure, skin grafting, fluid resuscitation, management of wound infection such as administering antibiotics, pain control, nutritional support, and/or measures to inhibit excessive scar formation. A burn may be covered with a clean and dry sheet or dressing (such as cling film). Early cooling with cool water, within 30 minutes of the burn, reduces burn depth and pain. Debridement, cleaning, and dressings are important aspects of burn wound care.

In some embodiments, treatment of a burn wound further comprises administration of an antibiotic agent. It has been shown that antibiotic prophylaxis may reduce mortality, bacteraemia, and ventilator associated pneumonia among patients in intensive care units (Silvestri L, et al, 2007, J Hosp Infect 65:187-203; and De Smet A M, et al., 2009, N Engl J Med 360:20-31). In burns patients, the skin is an additional source of infection (Avni T, et al., 2010, BMJ 340: c241). In some embodiments, treatment of a burn wound further comprises administration of an agent for managing pain. A pharmaceutical composition according to the present disclosure may be topically administered to a burn wound, in combination with an agent for pain management selected from the group consisting of a simple analgesic, ibuprofen, acetaminophen, and a narcotic. The antibiotic agent and/or agent for managing pain may be administered orally, intravenously, or topically, e.g., along with topical administration of a BT composition of the present disclosure. One or more other aspects of conventional treatment of burns also may be used in combination with a BT composition of the present disclosure.

In some embodiments, the agent for pain management for use in combination with the BT composition of the present disclosure includes one or more agents selected from the group consisting of: paracetamol (acetaminophen), a non-steroidal anti-inflammatory drug, ibuprofen, ketoprofen, piroxicam, hydrocodone, morphine, hydromorphine, oxymorphone, fentanyl, oxycodone, diamorphine, methadone, buprenorphine, meperidine, pentazocine, dextromoramide, dipipanone, amitriptyline, dilaudid, tapentadol, and methadone. The agent for pain management may include any other agent for pain described herein and/or known in the art.

In some embodiments, the agent for pain management is one that can be applied topically, such as a topical anesthetic agent. A topical anesthetic agent is a local anesthetic agent that is used to numb the surface of a body part, such as any area of the skin, the front of the eyeball, the inside of the nose, ear or throat, the anus, or the genital area. In some embodiments, the agent for pain management for use in combination with a BT composition of the present disclosure includes one or more topical anesthetic agents selected from the group consisting of benzocaine, butamben, dibucaine, lidocaine, oxybuprocaine, pramoxine, prilocaine, proparacaine, proxymetacaine, and tetracaine (amethocaine). Topical anesthetic agents are available in creams, ointments, aerosols, sprays, lotions, and jellies. In further embodiments, the topical anesthetic agent may be used with one or more additional agents for pain management, such as another topical anesthetic agent, or a different agent for pain management, such as any other agent(s) for pain management described herein and/or known in the art.

BT compositions of the present disclosure will comprise a therapeutically and/or prophylactically effective amount of one of more BT compounds (e.g. amorphous BisEDT), as described herein. A therapeutically and/or prophylactically effective amount refers to an amount required to bring about a therapeutic and/or prophylactic benefit, respectively, in a subject receiving said amount. A therapeutically and/or prophylactically effective amount will depend on the particular formulation, route of administration, condition being treated, whether other agents or therapies are used in combination with methods of the present disclosure, and other factors.

In some embodiments of the methods for treating a topical wound, the subject experiences one or more of the following outcomes following the completion of dosing:

the wound is healed or substantially healed within 12 weeks of the first administration of the composition; and/or

the prevention of amputation and/or infection-related surgery; and/or

the wound is closed partially or fully; and/or

the wound is reduced in size from about a 1% reduction relative to the original wound size to total elimination of the wound; and/or

the wound is 30 days old or greater and is healed or substantially healed.

In some embodiments, the subject experiences two or more of the recited outcomes. In some embodiments, the subject experiences three or more of the recited outcomes. In some embodiments, the subject experiences four or more of the recited outcomes. In some embodiments, the subject experiences all of the recited outcomes. In some embodiments of the methods for treating a topical wound, the subject experiences one or more of the following outcomes following the completion of dosing: less reinfection/relapse for the 12 weeks after start of treatment: resolution or improvement in signs and/or symptoms of infection that include redness, swelling, induration, exudate, pain, warmth (at site of infection) or fever; improved quality of life; eradication of insulting pathogens and/or biofilm; reduced need for concurrent systemic antibiotics.

In some embodiments, administration of a therapeutically effective amount of a BT composition, in accordance with the present disclosure, results in improved wound closure (partial or full), such as a reduction in the area of non-intact skin (wound area) compared to the area before initiation of treatment. Wound area can be expressed as a percentage of the initial wound area, at one or more time points after initiation of treatment. For example, in some embodiments, wound area decreases by at least about 20%, at least about 30%, at least about 40%, at least about 50%, at least about 60%, at least about 70%, at least about 80%; or at least about 90% over a course of treatment with a BT composition of the present disclosure. In some specific embodiments, the decrease in wound area occurs at least by day 1 after treatment initiation (t1), day 2 after treatment initiation (t2), day 3 after treatment initiation (t3), day 4 after treatment initiation (t4), day 5 after treatment initiation (t5), day 6 after treatment initiation (t6), day 7 after treatment initiation (t7), day 8 after treatment initiation (t8), day 9 after treatment initiation (t9), day 10 after treatment initiation (t10), day 12 after treatment initiation (t12), day 15 after treatment initiation (t15), day 20 after treatment initiation (t20), day 25 after treatment initiation (t25), or day 30 after treatment initiation (t30).

In some embodiments, administration of a therapeutically effective amount of a BT composition, in accordance with the present disclosure, results in improved wound healing, such as an improvement in ulcer grade based on the PEDIS classification compared to the ulcer grade before initiation of treatment. PEDIS is a routinely used, validated classification system for infections associated with wounds that has been developed by the International Working Group on the Diabetic Foot (IWGDF). IWGDF specifically developed a system for classifying wounds associated with diabetic foot infections that uses the acronym PEDIS, which stands for perfusion, extent (size), depth (tissue loss), infection, sensation (neuropathy). The classification originally was developed as a research tool (Schaper N C., 2004, Diabetes Metab Res Rev 20(Suppl 1):S90-5), and offers a semi-quantitative gradation for the severity of each of the categories. Specifically, PEDIS Grade 1 corresponds to no symptoms or signs of infection; Grade 2 corresponds to a local infection involving only the skin and subcutaneous tissue (without involvement of deeper tissues and without systemic signs), while any erythema involved must be between 0.5 cm and 2 cm; Grade 3 corresponds to a local infection, as described for Grade 2, but involving an erythema of greater than 2 cm or involving structures deeper than skin and subcutaneous tissues (e.g., abscess, osteomyelitis, septic arthritis, fasciitis), but without any systemic inflammatory response signs; and Grade 4 corresponds to a local infection, as described for Grades 2 and 3, but with the signs of systemic inflammatory response syndrome, as manifested by more than two of the following: a temperature >38° C. or <36° C.; a heart rate >90 beats/min; a respiratory rate >20 breaths/min or partial pressure of arterial carbon dioxide <32 mm Hg; and a white blood cell count >12000 or <4000 cells/μL or >10% immature (band) forms (see, e.g., Lipsky, B A, et al., 2012, CID 54:e132-e173). Another classification system has been developed by the IDSA (the Infectious Diseases Society of America), which rates the infection severity of infected wounds, in particular, diabetic foot infections. Specifically, the IDSA rates PEDIS Grades 1-4 as “uninfected”, “mild”, “moderate”, and “severe”, respectively (see, again, Lipsky, B A, et al., 2012, CID 54:e132-e173).

In some embodiments, the PEDIS grade decreases from Grade 4 to Grade 3, Grade 2, or Grade 1, over a course of treatment with a BT composition of the present disclosure. In some embodiments, the PEDIS grade decreases from Grade 3 to Grade 2 or Grade 1, over a course of treatment with a BT composition of the present disclosure. In some embodiments, the PEDIS grade decreases from Grade 2 to Grade 1 over a course of treatment with a BT composition of the present disclosure. In some embodiments, the decrease in ulcer grade occurs by at least day 1 after treatment initiation (t1), day 2 after treatment initiation (t2), day 3 after treatment initiation (t3), day 4 after treatment initiation (t4), day 5 after treatment initiation (t5), day 6 after treatment initiation (t6), day 7 after treatment initiation (t7), day 8 after treatment initiation (t8), day 9 after treatment initiation (t9), day 10 after treatment initiation (t10), day 12 after treatment initiation (t12), day 15 after treatment initiation (t15), day 20 after treatment initiation (t20), day 25 after treatment initiation (t25), day 30 after treatment initiation (t30), day 35 after treatment initiation (t35), day 40 after treatment initiation (t40), day 45 after treatment initiation (t45), day 50 after treatment initiation (t50), day 55 after treatment initiation (t55), day 60 after treatment initiation (t60), day 65 after treatment initiation (t65), day 70 after treatment initiation (t70), day 75 after treatment initiation (t75), day 80 after treatment initiation (t80), or day 85 after treatment initiation (t85). In some embodiments, the decrease in ulcer grade occurs after day 85 after treatment initiation (t85+).

In some embodiments of the methods for treating a topical wound, the BT composition comprises amorphous BisEDT as a suspension of microparticles having a volumetric mean diameter (VMD) from about 0.4 μm to about 5 μm.

In some embodiments of the methods for treating a topical wound, the applied amorphous BisEDT is present on the surface at a concentration greater than about 20 μg/cm². In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments of the methods for treating a topical wound, after administration of the BT composition, one or more of the following occurs: (i) reducing and or dispersing a microbial (e.g. bacterial and/or fungal) biofilm, (ii) impairing growth or formation of a microbial (e.g. bacterial and/or fungal) biofilm, and (iii) preventing reformation or spread of a microbial (e.g. bacterial and/or fungal) biofilm. In some embodiments, the BT composition treats, manages, and/or lessens the severity of the diabetic foot infection by one or both of: (i) prevention of the infection by the bacterial or fungal pathogen; and/or (ii) reduction of the bacterial or fungal pathogen. In some embodiments, the BT composition treats, manages or lessens the severity of the infection by one or more of: (i) prevention of elaboration or secretion of exotoxins from the bacterial or fungal pathogen; (ii) inhibition of cell viability or cell growth of planktonic cells of the bacterial or fungal pathogen; (iii) inhibition of biofilm formation by the bacterial or fungal pathogen; (iv) inhibition of biofilm or microbial pathogen invasiveness to underlying tissues (e.g. subcutaneous tissue); (v) inhibition of biofilm or microbial pathogen pathogenicity to underlying tissues (e.g. subcutaneous tissue); (vi) inhibition of biofilm viability or biofilm growth of biofilm-forming cells of the bacterial or fungal pathogen; and/or (vii) prevents the reformation of biofilm after debridement.

In some embodiments, the infection contains one or more bacterial or fungal pathogens. In some embodiments, the disclosed methods comprise treating the DFI-related infection. In some embodiments, the disclosed methods comprise managing the DFI-related infection. In some embodiments, the disclosed methods comprise lessening the severity of the DFI-related infection.

In some embodiments, the bismuth-thiol composition comprises a plurality of microparticles that comprise amorphous BisEDT, substantially all of said microparticles having a volumetric mean diameter of from about 0.4 μm to about 5 μm. In some embodiments, at least 70% of the microparticles have a volumetric mean diameter of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the methods for treating a topical wound, the administered amorphous BisEDT is present on the surface at a concentration from about 1 μg/cm² to about 1,000,000 μg/cm² (e.g. about 1 μg/cm² to about 10,000 μg/cm²), including all integers and ranges therebetween. In some embodiments, the amorphous BisEDT composition is present on the surface at a concentration from about 50 μg/cm² to about 200 μg/cm². In some embodiments, the applied amorphous BisEDT is present on the surface at a concentration from about 250 μg/cm² to about 5,000 μg/cm². For example, in some embodiments, amorphous BisEDT is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, amorphous BisEDT is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, the present disclosure provides methods of treating or preventing a bacterial infection in a subject in need thereof comprising administering to the subject a therapeutically effective amount of the pharmaceutical composition as described herein (i.e. an amorphous BisEDT-containing composition). In some embodiments, the bacterial infection is an infection by one or more of Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some embodiments, the pharmaceutical composition is administered topically. In some embodiments, the subject is a mammal, e.g., a human. In some embodiments, the bacterial infection is diabetic foot infection. In some embodiments, the diabetic foot infection comprises a cutaneous ulcer. In some embodiments, the bacterial infection is associated with an area of non-intact skin selected from a sore associated with cellulitis, an erysipelas lesion, a burn wound, a chronic ulcer, a decubitus ulcer, and a pressure sore. In some embodiments, the treatment comprises topically administering the pharmaceutical composition to a cutaneous ulcer associated with diabetic foot infection. In some embodiments, administration follows mechanical debridement of the ulcer. In some embodiments, administration comprises use of at least one of a dressing, an instillation device, and a negative pressure wound therapy device.

In some embodiments of the methods for treating a topical wound, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for treating a topical wound, the wound area is from about 0.1 cm² to about 250 cm², including all integers and ranges therebetween. For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for treating a bacterial infection, comprising administering to a subject in need thereof a therapeutically effective amount of a composition comprising amorphous BisEDT, wherein the composition is applied to the infection (e.g. applied to the surface of the infection). In some embodiments, the bacterial infection is a diabetic foot infection. In some embodiments, the bacterial infection comprises one or more of the following bacterial pathogens: Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis.

In some embodiments of the methods for treating a microbial (e.g. bacterial and/or fungal) infection, the infection is associated with a wound (e.g. an ulcer) and the subject experiences one or more of the following outcomes following the completion of dosing:

the wound is healed or substantially healed within 12 weeks (e.g. within 4 weeks) of the first administration of the composition; and/or

the prevention of amputation and/or infection-related surgery, and/or

the wound is closed partially or fully; and/or

the wound is reduced in size from about a 1% reduction relative to the original wound size to total elimination of the wound; and/or

the wound is 30 days old or greater and is healed or substantially healed.

In some embodiments, the subject experiences two or more of the recited outcomes. In some embodiments, the subject experiences three or more of the recited outcomes. In some embodiments, the subject experiences four or more of the recited outcomes. In some embodiments, the subject experiences all of the recited outcomes.

In some embodiments of the methods for treating a microbial (e.g. bacterial and/or fungal) infection, the BT composition is a suspension of microparticles comprising amorphous BisEDT having a volumetric mean diameter (VMD) from about 0.01 μm to about 5 μm. In some embodiments, at least 70% of the microparticles have a volumetric mean diameter of less than about 5 μm, less than about 4 μm, less than about 3 μm, less than about 2 μm, less than about 1 μm, from about 0.01 μm to about 5 μm, from about 0.1 μm to about 5 μm, of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the methods for treating a microbial (e.g. bacterial and/or fungal) infection, the BT composition comprises amorphous BisEDT and which is present on the surface at a concentration greater than about 20 μg/cm².

In some embodiments of the methods for treating a microbial (e.g. bacterial and/or fungal) infection, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments of the methods for treating a microbial (e.g. bacterial and/or fungal) infection, the method comprises at least one of: (i) reducing and or dispersing a microbial (e.g. bacterial and/or fungal) biofilm, (ii) impairing growth or formation of a microbial (e.g. bacterial and/or fungal) biofilm, and (iii) preventing reformation or spread of a microbial (e.g. bacterial and/or fungal) biofilm. In some embodiments, the BT composition treats, manages or lessens the severity of the diabetic foot infection by one or both of: (i) prevention of the infection by the bacterial or fungal pathogen; and (ii) reduction of the bacterial or fungal pathogen. In some embodiments, the BT composition treats, manages or lessens the severity of the infection by one or more of: (i) prevention of elaboration or secretion of exotoxins from the bacterial or fungal pathogen; (ii) inhibition of cell viability or cell growth of planktonic cells of the bacterial or fungal pathogen; (iii) inhibition of biofilm or microbial pathogen formation by the bacterial or fungal pathogen; (iv) inhibition of biofilm invasiveness to underlying tissues (e.g. subcutaneous tissue); (v) inhibition of biofilm or microbial pathogen pathogenicity to underlying tissues (e.g. subcutaneous tissue); (vi) inhibition of biofilm viability or biofilm growth of biofilm-forming cells of the bacterial or fungal pathogen; and/or (vii) prevents the reformation of biofilm after debridement.

In some embodiments of the methods for treating a microbial (e.g. bacterial and/or fungal) infection, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration from about 250 μg/cm² to about 5,000 μg/cm². For example, in some embodiments, the bismuth thiol compound in the BT composition is amorphous BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In another embodiment, the dose volume may range from about 0.01 mL to about 10 mL or any range therein between. In another embodiment, the dose volume may range from about 0.1 mL to about 1 mL or any range therein between. In another embodiment, the minimal dose volume is about 0.1 mL to about 0.5 mL or any range therein between.

In some embodiments of the methods for treating a microbial (e.g. bacterial and/or fungal) infection, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for treating a microbial (e.g. bacterial and/or fungal) infection, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm, about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for healing a wound in a subject having a diabetic foot infection, comprising administering the subject a therapeutically effective amount of a composition comprising amorphous BisEDT, wherein the composition is a suspension of microparticles comprising said BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm, and wherein the composition is applied to the infection (e.g. applied to the surface of the infection) and the wound is healed or substantially healed within 12 weeks of the first administration of the composition. In some embodiments, the BT composition further comprises about 0.05% to about 1.0% Tween 80®, about 0.05 to 40 mM sodium chloride, optionally about 1% to about 10% of methylcellulose (or other polymer such as a carbomer), and optionally about 2 to 20 mM sodium phosphate at about pH. 7.4.

In some embodiments of the methods for healing a wound in a subject having a diabetic foot infection, the applied BT composition is present on the surface at a concentration from about 1 μg/cm2 to about 1,000,000 μg/cm2 (e.g. about 1 μg/cm2 to about 10,000 μg/cm2). In some embodiments, the applied BT composition is present on the surface at a concentration from about 50 μg/cm2 to about 100 μg/cm2. In some embodiments, the applied BT composition is present on the surface at a concentration greater than about 100 μg/cm2 (e.g. as a dosage from about 250 μg/cm2 to about 5,000 μg/cm2). For example, in some embodiments, the bismuth thiol compound in the BT composition is amorphous BisEDT which is present on the surface at a concentration from about 1 μg/cm2 to about 10,000 μg/cm2 or from about 50 μg/cm2 to about 200 μg/cm2 or from about 250 μg/cm2 to about 5,000 μg/cm2. In some embodiments, the BT composition is present on the surface at a concentration of about 1 μg/cm2, about 50 μg/cm2, about 100 μg/cm2, about 150 μg/cm2, about 200 μg/cm2, about 250 μg/cm2, about 500 μg/cm2, about 750 μg/cm2, about 1000 μg/cm2, about 1500 μg/cm2, about 2000 μg/cm2, about 2500 μg/cm2, about 3000 μg/cm2, about 3500 μg/cm2, about 4000 μg/cm2, about 4500 μg/cm2, about 5000 μg/cm2, about 5500 μg/cm2, about 6000 μg/cm2, about 6500 μg/cm2, about 7000 μg/cm2, about 7500 μg/cm2, about 8000 μg/cm2, about 8500 μg/cm2, about 9000 μg/cm2, about 9500 μg/cm2, to about 10,000 μg/cm2.

In some embodiments of the methods for healing a wound in a subject having a diabetic foot infection, the BT composition is administered three times per day, two times per day, once daily, every other day, once every three days, three times per week, once every week, once every other week, once every month, or once every other month. In some embodiments, the wound is healed 4 weeks, 8 weeks or 12 weeks after the first administration of the BT composition. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for healing a wound in a subject having a diabetic foot infection, the wound area is from about 0.1 cm2 to about 250 cm2. For example, the wound area may be about 0.1 cm2, about 0.5 cm2, about 1 cm2, about 5 cm2, about 10 cm2, about 15 cm2, about 20 cm2, about 25 cm2, about 30 cm2, about 35 cm2, about 40 cm2, about 45 cm2, about 50 cm2, about 55 cm2, about 60 cm2, about 65 cm2, about 70 cm2, about 75 cm2, about 80 cm2, about 85 cm2, about 90 cm2, about 95 cm2, about 100 cm2, about 105 cm2, about 110 cm2, about 115 cm2, about 120 cm2, about 125 cm2, about 130 cm2, about 135 cm2, about 140 cm2, about 145 cm2, about 150 cm2, about 155 cm2, about 160 cm2, about 165 cm2, about 170 cm2, about 175 cm2, about 180 cm2, about 185 cm2, about 190 cm2, about 195 cm2, about 200 cm2, about 205 cm2, about 210 cm2, about 215 cm2, about 220 cm2, about 225 cm2, about 230 cm2, about 235 cm2, about 240 cm2, about 245 cm2, or about 250 cm2.

In some embodiments, the present disclosure provides methods for reducing the risk of amputation and/or infection-related surgery in a subject having a diabetic foot infection, comprising administering to the subject a therapeutically effective amount of a composition comprising amorphous BisEDT, wherein the composition is applied to the infection (e.g. applied to the surface of the infection) and the risk of amputation and/or infection-related surgery is reduced from about 1% to about 100% relevant to a similarly situated subject not treated with a therapeutically effective amount of a composition comprising a bismuth-thiol compound. In some embodiments, the composition is a suspension of microparticles comprising said BisEDT wherein at least 70% of the microparticles have a volumetric mean diameter (VMD) from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for reducing the risk of amputation and/or infection-related surgery in a subject having a diabetic foot infection, the applied BT composition is present on the surface at a concentration from about 1 μg/cm2 to about 1,000,000 μg/cm2 (e.g. about 1 μg/cm2 to about 10,000 μg/cm2). In some embodiments, the applied BT composition is present on the surface at a concentration from about 50 μg/cm2 to about 100 μg/cm2. In some embodiments, the applied BT composition is present on the surface at a concentration greater than about 100 μg/cm2 (e.g. as a dosage from about 250 μg/cm2 to about 5,000 μg/cm2). For example, in some embodiments, the bismuth thiol compound in the BT composition is BisEDT which is present on the surface at a concentration from about 1 μg/cm2 to about 10,000 μg/cm2 or from about 50 μg/cm2 to about 200 μg/cm2 or from about 250 μg/cm2 to about 5,000 μg/cm2. In some embodiments, the BT composition is present on the surface at a concentration of about 1 μg/cm2, about 50 μg/cm2, about 100 μg/cm2, about 150 μg/cm2, about 200 μg/cm2, about 250 μg/cm2, about 500 μg/cm2, about 750 μg/cm2, about 1000 μg/cm2, about 1500 μg/cm2, about 2000 μg/cm2, about 2500 μg/cm2, about 3000 μg/cm2, about 3500 μg/cm2, about 4000 μg/cm2, about 4500 μg/cm2, about 5000 μg/cm2, about 5500 μg/cm2, about 6000 μg/cm2, about 6500 μg/cm2, about 7000 μg/cm2, about 7500 μg/cm2, about 8000 μg/cm2, about 8500 μg/cm2, about 9000 μg/cm2, about 9500 μg/cm2, to about 10,000 μg/cm2.

In some embodiments, of the methods for reducing the risk of amputation and/or infection-related surgery in a subject having a diabetic foot infection, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, of the methods for reducing the risk of amputation and/or infection-related surgery in a subject having a diabetic foot infection, the wound area is from about 0.1 cm2 to about 250 cm2. For example, the wound area may be about 0.1 cm2, about 0.5 cm2, about 1 cm2, about 5 cm2, about 10 cm2, about 15 cm2, about 20 cm2, about 25 cm2, about 30 cm2, about 35 cm2, about 40 cm2, about 45 cm2, about 50 cm2, about 55 cm2, about 60 cm2, about 65 cm2, about 70 cm2, about 75 cm2, about 80 cm2, about 85 cm2, about 90 cm2, about 95 cm2, about 100 cm2, about 105 cm2, about 110 cm2, about 115 cm2, about 120 cm2, about 125 cm2, about 130 cm2, about 135 cm2, about 140 cm2, about 145 cm2, about 150 cm2, about 155 cm2, about 160 cm2, about 165 cm2, about 170 cm2, about 175 cm2, about 180 cm2, about 185 cm2, about 190 cm2, about 195 cm2, about 200 cm2, about 205 cm2, about 210 cm2, about 215 cm2, about 220 cm2, about 225 cm2, about 230 cm2, about 235 cm2, about 240 cm2, about 245 cm2, or about 250 cm2.

In some embodiments, the present disclosure provides methods for closing a wound in a subject having a diabetic foot infection, comprising administering to the subject a therapeutically effective amount of a composition comprising amorphous BisEDT. In some embodiments, the composition is a suspension of microparticles comprising said BisEDT wherein at least 70% of the microparticles have a volumetric mean diameter (VMD) from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for closing a wound in a subject having a diabetic foot infection, the applied BT composition is present on the surface at a concentration from about 1 μg/cm2 to about 1,000,000 μg/cm2 (e.g. about 1 μg/cm2 to about 10,000 μg/cm2). In some embodiments, the applied BT composition is present on the surface at a concentration from about 50 μg/cm2 to about 100 μg/cm2. In some embodiments, the applied BT composition is present on the surface at a concentration greater than about 100 μg/cm2 (e.g. as a dosage from about 250 μg/cm2 to about 5,000 μg/cm2). For example, in some embodiments, the bismuth thiol compound in the BT composition is amorphous BisEDT which is present on the surface at a concentration from about 1 μg/cm2 to about 10,000 μg/cm2 or from about 50 μg/cm2 to about 200 μg/cm2 or from about 250 μg/cm2 to about 5,000 μg/cm2. In some embodiments, the BT composition is present on the surface at a concentration of about 1 μg/cm2, about 50 μg/cm2, about 100 μg/cm2, about 150 μg/cm2, about 200 μg/cm2, about 250 μg/cm2, about 500 μg/cm2, about 750 μg/cm2, about 1000 μg/cm2, about 1500 μg/cm2, about 2000 μg/cm2, about 2500 μg/cm2, about 3000 μg/cm2, about 3500 μg/cm2, about 4000 μg/cm2, about 4500 μg/cm2, about 5000 μg/cm2, about 5500 μg/cm2, about 6000 μg/cm2, about 6500 μg/cm2, about 7000 μg/cm2, about 7500 μg/cm2, about 8000 μg/cm2, about 8500 μg/cm2, about 9000 μg/cm2, about 9500 μg/cm2, to about 10,000 μg/cm2.

In some embodiments, of the methods for closing a wound in a subject having a diabetic foot infection, the composition is applied to the infection (e.g. applied to the surface of the infection) and the wound is closed within 12 weeks of the first administration of the composition. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, of the methods for closing a wound in a subject having a diabetic foot infection, the wound area is from about 0.1 cm2 to about 250 cm2. For example, the wound area may be about 0.1 cm2, about 0.5 cm2, about 1 cm2, about 5 cm2, about 10 cm2, about 15 cm2, about 20 cm2, about 25 cm2, about 30 cm2, about 35 cm2, about 40 cm2, about 45 cm2, about 50 cm2, about 55 cm2, about 60 cm2, about 65 cm2, about 70 cm2, about 75 cm2, about 80 cm2, about 85 cm2, about 90 cm2, about 95 cm2, about 100 cm2, about 105 cm2, about 110 cm2, about 115 cm2, about 120 cm2, about 125 cm2, about 130 cm2, about 135 cm2, about 140 cm2, about 145 cm2, about 150 cm2, about 155 cm2, about 160 cm2, about 165 cm2, about 170 cm2, about 175 cm2, about 180 cm2, about 185 cm2, about 190 cm2, about 195 cm2, about 200 cm2, about 205 cm2, about 210 cm2, about 215 cm2, about 220 cm2, about 225 cm2, about 230 cm2, about 235 cm2, about 240 cm2, about 245 cm2, or about 250 cm2.

In some embodiments, the present disclosure provides methods for wound size reduction in a subject having a diabetic foot infection, comprising administering to the subject a therapeutically effective amount of a composition comprising amorphous BisEDT, wherein the composition is applied to the infection (e.g. applied to the surface of the infection) and the wound is reduced in size from about a 1% reduction relative to the original wound size to total elimination of the wound within 12 weeks of the first administration of the composition. In some embodiments, the wound is reduced by about 10%, 20%, 30%, 6, 40%, 50%, 60%, 70%, 80%, or 90%. In some embodiments, the wound is reduced by about 50%.

In some embodiments, of the methods for wound size reduction in a subject having a diabetic foot infection, the composition is a suspension of microparticles comprising said amorphous BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for wound size reduction in a subject having a diabetic foot infection, the applied BT composition is present on the surface at a concentration from about 1 μg/cm2 to about 1,000,000 μg/cm2 (e.g. about 1 μg/cm2 to about 10,000 μg/cm2). In some embodiments, the applied BT composition is present on the surface at a concentration from about 50 μg/cm2 to about 100 μg/cm2. In some embodiments, the applied BT composition is present on the surface at a concentration greater than about 100 μg/cm2 (e.g. as a dosage from about 250 μg/cm2 to about 5,000 μg/cm2). For example, in some embodiments, amorphous BisEDT is present on the surface at a concentration from about 1 μg/cm2 to about 10,000 μg/cm2 or from about 50 μg/cm2 to about 200 μg/cm2 or from about 250 μg/cm2 to about 5,000 μg/cm2. In some embodiments, the BT composition is present on the surface at a concentration of about 1 μg/cm2, about 50 μg/cm2, about 100 μg/cm2, about 150 μg/cm2, about 200 μg/cm2, about 250 μg/cm2, about 500 μg/cm2, about 750 μg/cm2, about 1000 μg/cm2, about 1500 μg/cm2, about 2000 μg/cm2, about 2500 μg/cm2, about 3000 μg/cm2, about 3500 μg/cm2, about 4000 μg/cm2, about 4500 μg/cm2, about 5000 μg/cm2, about 5500 μg/cm2, about 6000 μg/cm2, about 6500 μg/cm2, about 7000 μg/cm2, about 7500 μg/cm2, about 8000 μg/cm2, about 8500 μg/cm2, about 9000 μg/cm2, about 9500 μg/cm2, to about 10,000 μg/cm2.

In some embodiments, of the methods for wound size reduction in a subject having a diabetic foot infection, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, of the methods for wound size reduction in a subject having a diabetic foot infection, the wound area is from about 0.1 cm2 to about 250 cm2. For example, the wound area may be about 0.1 cm2, about 0.5 cm2, about 1 cm2, about 5 cm2, about 10 cm2, about 15 cm2, about 20 cm2, about 25 cm2, about 30 cm2, about 35 cm2, about 40 cm2, about 45 cm2, about 50 cm2, about 55 cm2, about 60 cm2, about 65 cm2, about 70 cm2, about 75 cm2, about 80 cm2, about 85 cm2, about 90 cm2, about 95 cm2, about 100 cm2, about 105 cm2, about 110 cm2, about 115 cm2, about 120 cm2, about 125 cm2, about 130 cm2, about 135 cm2, about 140 cm2, about 45 cm2, about 150 cm2, about 155 cm2, about 160 cm2, about 165 cm2, about 170 cm2, about 175 cm2, about 180 cm2, about 185 cm2, about 190 cm2, about 195 cm2, about 200 cm2, about 205 cm2, about 210 cm2, about 215 cm2, about 220 cm2, about 225 cm2, about 230 cm2, about 235 cm2, about 240 cm2, about 245 cm2, or about 250 cm2. In some embodiments, the wound surface area of said wound is reduced by 50% 12 weeks after the first administration of the BT composition, and the BT composition comprises amorphous BisEDT. In some embodiments, the wound surface area of said wound is reduced by 50% 4 weeks after the first administration of the BisEDT composition. In some embodiments, the wound surface area is measured using digital photographs or hand measurements.

In some embodiments, the present disclosure provides a method for preventing amputation and/or infection-related surgery in a subject having a diabetic foot infection, comprising administering to the subject a therapeutically effective amount of a BT composition. In some embodiments, the BT composition is a suspension of microparticles comprising said amorphous BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.

In some embodiments, of the methods for preventing amputation and/or infection-related surgery in a subject having a diabetic foot infection, the applied BT composition is present on the surface at a concentration from about 1 μg/cm2 to about 1,000,000 μg/cm2 (e.g. about 1 μg/cm2 to about 10,000 μg/cm2). In some embodiments, the applied BT composition is present on the surface at a concentration from about 50 μg/cm2 to about 100 μg/cm2. In some embodiments, the applied BT composition is present on the surface at a concentration greater than about 100 μg/cm2 (e.g. as a dosage from about 250 μg/cm2 to about 5,000 μg/cm2). For example, in some embodiments, the bismuth thiol compound in the BT composition is amorphous BisEDT which is present on the surface at a concentration from about 1 μg/cm2 to about 10,000 μg/cm2 or from about 50 μg/cm2 to about 200 μg/cm2 or from about 250 μg/cm2 to about 5,000 μg/cm2. In some embodiments, the BT composition is present on the surface at a concentration of about 1 μg/cm2, about 50 μg/cm2, about 100 μg/cm2, about 150 μg/cm2, about 200 μg/cm2, about 250 μg/cm2, about 500 μg/cm2, about 750 μg/cm2, about 1000 μg/cm2, about 1500 μg/cm2, about 2000 μg/cm2, about 2500 μg/cm2, about 3000 μg/cm2, about 3500 μg/cm2, about 4000 μg/cm2, about 4500 μg/cm2, about 5000 μg/cm2, about 5500 μg/cm2, about 6000 μg/cm2, about 6500 μg/cm2, about 7000 μg/cm2, about 7500 μg/cm2, about 8000 μg/cm2, about 8500 μg/cm2, about 9000 μg/cm2, about 9500 μg/cm2, to about 10,000 μg/cm2.

In some embodiments, the present disclosure provides methods of treating a wound in a subject, wherein the wound is 30 days old or greater, comprising administering to the subject a therapeutically effective amount of a BT composition. In some embodiments, the subject has a diabetic foot infection. In some embodiments, the BT composition is a suspension of microparticles comprising amorphous BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm. In some embodiments, the BT composition further comprises about 0.05% to about 1.0% Tween 80®, about 0.05 to 40 mM sodium chloride, optionally about 1% to about 1% of methylcellulose, and optionally about 2 to 20 mM sodium phosphate at about pH. 7.4.

In some embodiments, the present disclosure provides methods of treating a wound in a subject, wherein the wound is 30 days old or less, comprising administering to the subject a therapeutically effective amount of a BT composition. In some embodiments, the subject has a diabetic foot infection. In some embodiments, the BT composition is a suspension of microparticles comprising amorphous BisEDT having a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm or from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4. For example, the wound may be about 1 day old, about 2 days old, about 3 days old, about 4 days old, about 5 days old, about 6 days old, about 7 days old, about 8 days old, about 9 days old, about 10 days old, about 11 days old, about 12 days old, about 13 days old, about 14 days old, about 15 days old, about 16 days old, about 17 days old, about 18 days old, about 19 days old, about 20 days old, about 21 days old, about 22 days old, about 23 days old, about 24 days old, about 25 days old, about 26 days old, about 27 days old, about 28 days old, about 29 days old, or about 30 days old.

In some embodiments, of the methods of treating a wound in a subject, wherein the wound is 30 days old or greater, the applied BT composition is present on the surface at a concentration from about 1 μg/cm2 to about 1,000,000 μg/cm2 (e.g. about 1 μg/cm2 to about 10,000 μg/cm2). In some embodiments, the applied BT composition is present on the surface at a concentration from about 50 μg/cm2 to about 100 μg/cm2. In some embodiments, the applied BT composition is present on the surface at a concentration greater than about 100 μg/cm2 (e.g. as a dosage from about 250 μg/cm2 to about 5,000 μg/cm2). For example, in some embodiments, the bismuth thiol compound in the BT composition is amorphous BisEDT which is present on the surface at a concentration from about 1 μg/cm2 to about 10,000 μg/cm2 or from about 50 μg/cm2 to about 200 μg/cm2 or from about 250 μg/cm2 to about 5,000 μg/cm2. In some embodiments, the BT composition is present on the surface at a concentration of about 1 μg/cm2, about 50 μg/cm2, about 100 μg/cm2, about 150 μg/cm2, about 200 μg/cm2, about 250 μg/cm2, about 500 μg/cm2, about 750 μg/cm2, about 1000 μg/cm2, about 1500 μg/cm2, about 2000 μg/cm2, about 2500 μg/cm2, about 3000 μg/cm2, about 3500 μg/cm2, about 4000 μg/cm2, about 4500 μg/cm2, about 5000 μg/cm2, about 5500 μg/cm2, about 6000 μg/cm2, about 6500 μg/cm2, about 7000 μg/cm2, about 7500 μg/cm2, about 8000 μg/cm2, about 8500 μg/cm2, about 9000 μg/cm2, about 9500 μg/cm2, to about 10,000 μg/cm2.

In some embodiments, of the methods of treating a wound in a subject, wherein the wound is 30 days old or less, the applied BT composition is present on the surface at a concentration from about 1 μg/cm2 to about 1,000,000 μg/cm2 (e.g. about 1 μg/cm2 to about 10,000 μg/cm2). In some embodiments, the applied BT composition is present on the surface at a concentration from about 50 μg/cm2 to about 100 μg/cm2. In some embodiments, the applied BT composition is present on the surface at a concentration greater than about 100 μg/cm2 (e.g. as a dosage from about 250 μg/cm2 to about 5,000 μg/cm2). For example, in some embodiments, the bismuth thiol compound in the BT composition is amorphous BisEDT which is present on the surface at a concentration from about 1 μg/cm2 to about 10,000 μg/cm2 or from about 50 μg/cm2 to about 200 μg/cm2 or from about 250 μg/cm2 to about 5,000 μg/cm2. In some embodiments, the BT composition is present on the surface at a concentration of about 1 μg/cm2, about 50 μg/cm2, about 100 μg/cm2, about 150 μg/cm2, about 200 μg/cm2, about 250 μg/cm2, about 500 μg/cm2, about 750 μg/cm2, about 1000 μg/cm2, about 1500 μg/cm2, about 2000 μg/cm2, about 2500 μg/cm2, about 3000 μg/cm2, about 3500 μg/cm2, about 4000 μg/cm2, about 4500 μg/cm2, about 5000 μg/cm2, about 5500 μg/cm2, about 6000 μg/cm2, about 6500 μg/cm2, about 7000 μg/cm2, about 7500 μg/cm2, about 8000 μg/cm2, about 8500 μg/cm2, about 9000 μg/cm2, about 9500 μg/cm2, to about 10,000 μg/cm2.

In some embodiments, of the methods of treating a wound in a subject, wherein the wound is 30 days old or greater, the wound is greater than 2 months old, greater than 3 months old, greater than 4 months old, greater than 5 months old, greater than 6 months old, greater than 7 months old, greater than 8 months old, greater than 9 months old, greater than 10 months old, greater than 11 months old, or greater than 1 year old. In some embodiments, the wound is greater than 2 months old. In some embodiments, the wound is greater than 3 months old.

In any of the embodiments of the methods described herein, the BT composition that is ultimately applied or administered to the subject has a concentration from about 1 μg/mL to about 1,000,000 μg/mL (e.g. about 1 μg/cm2 to about 10,000 μg/cm2). In some embodiments, the BT composition has a concentration from about 50 μg/mL to about 100 μg/mL. In some embodiments, the applied BT the BT composition has a concentration from about 250 μg/mL to about 5,000 μg/mL. For example, in some embodiments, the bismuth thiol compound in the BT composition is BisEDT which has a concentration from about 1 μg/mL to about 10,000 μg/mL or from about 50 μg/mL to about 200 μg/mL or from about 250 μg/mL to about 5,000 μg/mL. In some embodiments, the BT composition has a concentration of about 1 μg/mL, about 50 μg/mL, about 100 μg/mL, about 150 μg/mL, about 200 μg/mL, about 250 μg/mL, about 500 μg/mL, about 750 μg/mL, about 1000 μg/mL, about 1500 μg/mL, about 2000 μg/mL, about 2500 μg/mL, about 3000 μg/mL, about 3500 μg/mL, about 4000 μg/mL, about 4500 μg/mL, about 5000 μg/mL, about 5500 μg/mL, about 6000 μg/mL, about 6500 μg/mL, about 7000 μg/mL, about 7500 μg/mL, about 8000 μg/mL, about 8500 μg/mL, about 9000 μg/mL, about 9500 μg/mL, to about 10,000 μg/mL. In some embodiments of the methods described herein, the BT composition that is ultimately applied or administered to the subject has a concentration greater than about 1,000,000 μg/mL.

In a specific embodiment, the present invention may be a pharmaceutical composition comprising bismuth-thiol (BT) composition that comprises amorphous BisEDT suspended therein, wherein the BT composition comprises a plurality of microparticles. In a specific embodiment, the D90 of said microparticles is less than or equal to 4.5 μm, or 4.0 μm, or 3.5 μm, or 3.0 μm, or 2.5 μm, or 2.0 μm, or 1.9 μm, or 1.8 μm, or μm 1.7 μm, or 1.6 μm, or 1.5 μm or any ranges in between. In a specific embodiment, the D90 of said microparticles is less than or equal to 1.9 μm. In another specific embodiment, the D90 of said microparticles is less than or equal to 1.6 μm. In another specific embodiment, the D50 of said microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges in between. In another specific embodiment, the D10 of said microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges in between. In a specific embodiment, the pharmaceutical composition comprising bismuth-thiol (BT) composition comprises amorphous BisEDT suspended therein, wherein the BT composition comprises a plurality of microparticles, wherein the D90 of said microparticles is less than or equal to about 1.6 μm. In a specific embodiment, the BT composition comprises about 0.05% to about 1.0% Tween 80®, about 0.05 to 40 mM sodium chloride, optionally about 1% to about 10% of methylcellulose (or polymer such as a carbomer), and optionally about 2 to 20 mM sodium phosphate at about pH. 7. In another specific embodiment, the compositions described above can be administered to a subject for treating a topical wound in a subject, or any specific methods described herein. In another specific embodiment, the compositions described above can be administered to a subject for treating a diabetic foot infection or diabetic foot ulcer. In a specific embodiment, the methods may include administering the compositions described above to a subject, wherein the subject experiences one or more of the following outcomes following the completion of dosing of said composition:

the wound is healed or substantially healed within 12 weeks of the first administration of the composition; and/or

the prevention of amputation and/or infection-related surgery, and/or

the wound is closed partially or fully; and/or

the wound is reduced in size from about a 1% reduction relative to the original wound size to total elimination of the wound; and/or

the wound is 30 days old or greater and is healed or substantially healed.

In some embodiments, the present disclosure provides a method for healing a wound in a subject having a diabetic foot infection, comprising administering the subject a therapeutically effective amount of a composition comprising amorphous BisEDT. In some embodiments, the wound is a diabetic foot ulcer.

In some embodiments, the present disclosure provides a method for healing a wound in a subject having a diabetic foot infection, comprising administering the subject a therapeutically effective amount of a composition comprising amorphous BisEDT, wherein the composition is a suspension of microparticles comprising said BisEDT wherein at least 70% of the microparticles have a volumetric mean diameter (VMD) from about 0.4 μm to about 5 μm, and wherein the composition is applied to the infection and the wound is healed or substantially healed within 12 weeks of the first administration of the composition. In some embodiments, the wound is a diabetic foot ulcer. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4. In some embodiments, the applied BT composition is present on the surface at a concentration from about 1 μg/cm2 to about 1,000,000 μg/cm2. In some embodiments, the applied BT composition is present on the surface at a concentration from about 50 μg/cm2 to about 100 μg/cm2. In some embodiments, the applied BT composition is present on the surface at a concentration greater than about 100 μg/cm2. In some embodiments, the BT composition is administered three times per day, two times per day, once daily, every other day, once every three days, three times per week, once every week, once every other week, once every month, or once every other month. In some embodiments, the wound is healed 4 weeks, 8 weeks or 12 weeks after the first administration of the BT composition. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the wound area is from about 0.1 cm2 to about 250 cm2.

In some embodiments, the present disclosure provides a method for wound size reduction in a subject having a diabetic foot infection, comprising administering to the subject a therapeutically effective amount of a composition comprising amorphous BisEDT, wherein the composition is a suspension of microparticles comprising said BisEDT wherein at least 70% of the microparticles have a volumetric mean diameter (VMD) of less than about 5 μm, less than about 4 μm, less than about 3 μm, less than about 2 μm, less than about 1 μm, from about 0.01 μm to about 5 μm, from about 0.1 μm to about 5 μm, or from about 0.4 μm to about 5 μm, and wherein the composition is applied to the infection and the wound is reduced in size from about a 1% reduction relative to the original wound size to total elimination of the wound within 12 weeks of the first administration of the composition. In some embodiments, the wound is reduced by about 10%, 20%, 30%, 40%, 50%, 60%, 70%, 80%, or 90%. In some embodiments, the wound is reduced by at least about 50%. In some embodiments, the wound is a diabetic foot ulcer. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4. In some embodiments, the applied BT composition is present on the surface at a concentration from about 1 μg/cm2 to about 1,000,000 μg/cm2. In some embodiments, the applied BT composition is present on the surface at a concentration from about 50 μg/cm2 to about 100 μg/cm2. In some embodiments, the applied BT composition is present on the surface at a concentration greater than about 100 μg/cm2. In some embodiments, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of about 4 weeks. In some embodiments, the wound area is from about 0.1 cm2 to about 250 cm2. In some embodiments, the wound surface area of said wound is reduced by at least 50% by 12 weeks after the first administration of the BT composition. In some embodiments, the wound surface area of said wound is reduced by at least 50% by 4 weeks after the first administration of the BisEDT composition. In some embodiments, the wound surface area is measured using digital photographs or hand measurement.

### Embodiments

1. A method of treating, managing or lessening the severity of symptoms associated with a respiratory viral infection in a subject, the method comprising administering to the subject a bismuth-thiol (BT) composition that comprises BisEDT suspended therein, wherein administering the BT composition is via inhalation, orally or nasally, using an aerosol device.
2. The method of embodiment 1, wherein the infection that is treated, managed or lessened is the viral infection.
3. The method of embodiment 1, wherein the infection that is treated, managed or lessened is a bacterial and/or fungal infection that is secondary to the viral infection.
4. The method of any one of embodiments 1-3, wherein the BT composition comprises a plurality of microparticles wherein at least 70% of said microparticles having a volumetric mean diameter (VMD) from about 0.6 μm to about 2.5 μm.
5. The method of embodiment 4, wherein at least 80% of said microparticles having a VMD from about 0.6 μm to about 2.5 μm.
6. The method of embodiment 5, wherein at least 90% of said microparticles having a VMD from about 0.6 μm to about 2.5 μm.
7. The method of any one of embodiments 1-6, wherein when the BT composition is aerosolized, at least 70% of the aerosolized liquid droplets have a mass median aerodynamic diameter (MMAD) from about 0.9 μm to about 3 μm.
8. The method of embodiment 7, wherein when the BT composition is aerosolized, at least 80% of the aerosolized liquid droplets have a MMAD from about 0.9 μm to about 3 μm.
9. The method of embodiment 8, wherein when the BT composition is aerosolized, at least 90% of the aerosolized liquid droplets have a MMAD from about 0.9 μm to about 3 μm.
10. The method of any of embodiments 1-9, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.
11. The method of any of embodiments 1-10, wherein if deposited to the deep lung region, the BisEDT compounds have an average half-life of about 4 days.
12. The method of any of embodiments 1-11, wherein the secondary infection is a pulmonary infection comprising one or more bacterial pathogens and/or fungal pathogens.
13. The method of any of embodiments 1-12, wherein the pulmonary infection is one or more of bronchiectasis infection, pneumonia, valley fever, allergic bronchopulmonary aspergillosis (ABPA), ventilator-acquired pneumonia, hospital acquired pneumonia, community acquired pneumonia, ventilator associated tracheobronchitis, lower respiratory tract infection, non-tuberculous Mycobacteria, anthrax, legionellosis, pertussis, bronchitis, Bronchiolitis, COPD-associated infection, viral pneumonia, viral bronchiolitis, and post-lung transplantation.
14. The method of embodiment 13, wherein the pulmonary infection is pneumonia or ventilator-acquired pneumonia.
15. The method of any of embodiments 1-14, wherein the method comprises at least one of; (i) reducing a biofilm (e.g. bacterial and/or fungal), (ii) impairing growth of a biofilm (e.g. bacterial and/or fungal), (iii) preventing initial formation of the biofilm (e.g. bacterial and/or fungal), and/or (iv) preventing reformation of the biofilm (e.g. bacterial and/or fungal).
16. The method of embodiment 10, wherein the one or more pathogens are selected from Haemophilus influenzae, Pseudomonas aeruginosa, Staphylococcus aureus, Staphylococcus warneri Staphylococcus lugdunensis, Staphylococcus epidermidis, Streptococcus milleri/anginous, Streptococcus pyogenes, non-tuberculosis mycobacteria, Mycobacterium tuberculosis, Burkholderia spp., Achromobacter xylosoxidans, Pandoraea sputorum, Stenotrophomonas maltophilia, Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morganii, Inquilinus limosus, Ralstonia mannitolilytica, Pandoraea apista, Pandoraea pnomenusa, Pandoraea sputorum, Bdellovibrio bacteriovorus, Bordetella bronchiseptica, Vampirovibrio chlorellaiorus, Actinobacter baumanni, Cupriadidus metallidurans, Cupriavidus pauculus, Cupriavidus respiraculi, Delftia acidivordans, Exophilia dermatitidis, Herbaspirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralstonia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S. agalactiae, Bacteroides fragilis, Clostridium difficile, Streptococcus pneumonia, Moraxella catarrhalis, Haemophilus haemolyticus, Haemophilus parainfluenzae, Chlamydophilia pneumoniae, Mycoplasma pneumoniae, Atopobium, Sphingomonas, Saccharibacteria, Leptotrichia, Capnocytophaga, Oribacterium, Aquabacterium, Lachnoanaerobaculum, Campylobacter, Acinetobacter; Agrobacterium; Bordetella; Brevundimonas; Chryseobacterium; Delftia; Enterobacter; Klebsiella; Pandoraea; Pseudomonas; Ralstonia, Coccidioides, and Prevotella. 17. The method of any of embodiments 1-16, wherein the respiratory viral infection is one or more of influenza viral infection (e.g., seasonal flu), rhinovirus infection (e.g., common cold), coronavirus infection (e.g., Severe Acute Respiratory Syndrome and common cold), paramyxovirus infection (e.g., measles), and/or acute respiratory distress syndrome.
18. The method of embodiment 17, wherein the coronavirus infection is selected from Severe Acute Respiratory Syndrome-Corona Virus (SARS-CoV), Middle East Respiratory Syndrome virus (CoV-MERS), human HCoV-229E, HCoV-OC43, HCoV-NL63 and HCoV-HKU1.
19. The method of embodiment 18, wherein the coronavirus infection is SARS-CoV (e.g. SARS-CoV-1, SARS-CoV-2).
20. The method of embodiment 18, wherein the SARS-CoV is SARS-CoV-2 (COVID-19).
21. The method of embodiment 17, wherein the respiratory viral infection is an influenza viral infection selected from the group consisting of Influenza A, Influenza B, and Influenza C viral infections.
22. The method of embodiment 22, wherein the Influenza A virus comprises H1N1, H2N2, H3N2, H5N1, H7N7, H1N2, H9N2, H7N2, H7N3, or H10N7 subtypes.
23. The method of any of embodiments 1-21, wherein the BT composition is co-administered with one or more antimicrobial agents.
24. The method of embodiment 23, wherein at least one of the one or more antimicrobial agents is a broad spectrum antiviral agent.
25. The method of any of embodiments 1-23, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).
26. The method of any one of embodiments 1-25, wherein the BisEDT is amorphous BisEDT.
27. The method of embodiment 26, wherein the amorphous BisEDT X-ray powder diffraction pattern does not contain any distinct peaks.
28. The method of embodiment 26 or embodiment 27, wherein the amorphous BisEDT X-ray powder diffraction pattern is substantially similar to FIG. 44.
29. The method according to any one of embodiments 26-28, wherein the amorphous BisEDT differential scanning calorimetry thermogram comprises an exothermic peak at about 168° C.
30. The method according to any one of embodiments 26-29, wherein the amorphous BisEDT differential scanning calorimetry thermogram further comprises an endotherm at about 64° C. and/or an endotherm peak at about 112° C. and/or an exotherm peak at about 145° C.
31. The method according to any one of embodiments 26-30, wherein the amorphous BisEDT differential scanning calorimetry thermogram is substantially similar to FIG. 45.
32. The method according to any one of embodiments 26-31, wherein the amorphous BisEDT has a glass transition at about 101° C.
33. The method according to any one of embodiments 26-32, wherein the amorphous BisEDT is at least 90% pure.
34. An aerosol comprising a plurality of dispersed liquid droplets in a gas, said liquid droplets comprising a BT composition comprising BisEDT compound suspended therein and at least one antimicrobial agent; and

wherein at least 70% of the liquid droplets have a MMAD from about of from about 0.9 μm to about 3 μm.

35. The aerosol of embodiment 34, wherein prior to aerosolization, the BT composition comprises a plurality of microparticles wherein at least 70% of said microparticles have a VMD of from about 0.6 μm to about 2.5 μm.
36. The aerosol of any one of embodiments 34-35, wherein least 90% of the liquid droplets have a MMAD from about of from about 0.9 μm to about 3 μm.
37. The aerosol of any one of embodiments 34-36, wherein prior to aerosolization, the BT composition comprises a plurality of microparticles wherein at least 90% of said microparticles have a VMD of from about 0.6 μm to about 2.5 μm.
38. The aerosol of any one of embodiments 34-37, wherein the droplets further comprise Tween 80 (e.g. from about 0.05% to about 1%) and optionally a buffer (e.g. sodium phosphate or sodium citrate) at a pH of about 7.4; and/or sodium chloride.
39. The aerosol of any one of embodiments 34-38, wherein at least one antimicrobial agents is a broad spectrum antiviral agent.
40. The aerosol of any one of embodiments 34-39, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).
41. A pharmaceutical composition comprising bismuth-thiol (BT) composition that comprises BisEDT suspended therein and at least one antimicrobial agent, wherein the BT composition comprises a plurality of microparticles, wherein the D90 of said microparticles is less than or equal to 1.9 μm.
42. The pharmaceutical composition of embodiment 41, wherein at least 70% of said microparticles having a volumetric mean diameter of from about 0.6 μm to about 2.5 μm.
43. The pharmaceutical composition of embodiment 41 or embodiment 42, wherein at least 90% of said microparticles having a volumetric mean diameter of from about 0.6 μm to about 2.5 μm.
44. The pharmaceutical composition of any one of embodiments 41-43, wherein at least one antimicrobial agents is a broad spectrum antiviral agent.
45. The pharmaceutical composition of any one of embodiments 41-44, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).
46. A kit comprising
(1) an aerosol comprising a plurality of dispersed liquid droplets in a gas, said liquid droplets comprising a BT composition comprising BisEDT compound suspended therein; and wherein at least 700% of the liquid droplets have a MMAD from about of from about 0.9 μm to about 3 μm, and
(2) at least one antimicrobial agent.
47. The kit of embodiment 46, wherein the at least one antimicrobial agent is formulated for administration orally, parenterally, topically, perorally, internally, intranasally, rectally, vaginally, lingually, or transdermally.
48. The kit of embodiment 46 or embodiment 47, wherein the at least one antimicrobial agents is a broad spectrum antiviral agent.
49. The kit of any one of embodiments 46-48, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).
50. A method of preventing infections in a subject, the method comprising administering to the subject a bismuth-thiol (BT) composition that comprises BisEDT suspended therein, wherein administering the BT composition is via inhalation, orally or nasally, using an aerosol device, wherein the subject is connected to a ventilator.
51. The method of embodiment 50, wherein the infection is ventilator-acquired pneumonia.
52. The method of embodiment 50 or 51, further comprising administering the composition to the subject prior to ventilation.
53. The method of embodiment 50 or 51, further comprising administering the composition to the subject during or after ventilation.
54. The method of any one of embodiments 50-53, wherein the method comprises at least one of: (i) reducing a biofilm (e.g. bacterial and/or fungal), (ii) impairing growth of a biofilm (e.g. bacterial and/or fungal), (iii) preventing initial formation of the biofilm (e.g. bacterial and/or fungal), and/or (iv) preventing reformation of the biofilm (e.g. bacterial and/or fungal).
55. A method of preventing, treating, managing or lessening the severity of superinfections and/or superantigen production in a subject, the method comprising administering to the subject a bismuth-thiol (BT) composition that comprises BisEDT suspended therein.
56. The method of embodiment 55, wherein the BT composition is administered via inhalation, orally or nasally, using an aerosol device, or intravenously.
57. The method of embodiment 56, wherein the BT composition is administered via inhalation, orally or nasally, using an aerosol device.
58. The method of embodiment any one of embodiments 55-57, wherein the method is preventing symptoms associated with a later respiratory viral infection in a subject.
59. The method of embodiment 58, wherein the respiratory infection is a SARS-CoV-2 (COVID-19) infection.
60. The method of any one of embodiments 55-59, wherein the subject has a pre-existing health condition.
61. The method of embodiment 56, wherein the pre-existing health condition causes chronic inflammation in the subject.
62. The method of embodiment 56, wherein the pre-existing health condition is a chronic inflammatory disease.
63. The method of embodiment 62, wherein the chronic inflammatory disease is related to one of or more conditions selected from one or more of the group consisting of Metabolic Syndrome, Obesity, Vasculitis, Cardiovascular Diseases, Chronic Wounds, Diabetes, hypertension, stroke, inflammatory bowel disease, periodontitis, atherosclerosis, COPD, endocarditis, thrombotic diseases, asthma, advanced age, recurrent infections, autoimmune diseases, chronic rhinosinusitis, inflammatory arthritis, neurodegenerative conditions, device-related infections, osteomyelitis, and depression.
64. The method of any one of embodiments 55-63, wherein the superantigen is a bacterial superantigen.
65. The method of embodiment 61, were in the superantigens include potent extotoxins secreted by Staphylococcus aureus and/or Streptococcus pyogenes.
66. The method of any one of embodiments 55-65, wherein the subject is infected with more than a 1 picomolar concentration of superantigen.
67. The method of any one of embodiments 55-66, wherein the subject is infected with more than 0.1 micogram (ug) of superantigen.
68. The method of any one of embodiments 55-61, wherein the subject is infected with more than 0.2 ug of superantigen.
69. The method of any one of embodiments 55-61, wherein the method further prevents, manages or lessens the severity of a cytokine storm in a subject.
70. The method of any one of embodiments 55-69, wherein the subject has elevated levels of neutrophil-lymphocyte ratio (NLR), IL-6, IL-17, IL-10, interferon-gamma, TNF-α, IL-2, and/or procalcitonin.
71. The method of any one of embodiments 55-60, wherein the subject has elevated levels of neutrophil-lymphocyte ratio (NLR), IL-6, IL-17, and/or procalcitonin.
72. The method of embodiment 62, wherein the NLR ratio is greater than 5.
73. The method of embodiment 62, wherein the NLR ratio is greater than 7.
74. The method of embodiment 64, wherein the NLR ratios is between 7 and 100.
75. The method of any one of embodiments 55-74, wherein the administration to the subject a bismuth-thiol (BT) composition, treats, inhibits or prevents the dispersal of biofilms in the subject.
76. The method of embodiment 75, wherein the biofilm is a nasopharyngeal biofilm.
77. The method of any one of embodiments 55-76, wherein the subject is older than 60 years of age, 70 years of age, 80 years of age and/or 90 years of age.
78. The method of any one of embodiments 55-77, wherein the BT composition comprises a plurality of microparticles wherein at least 70% of said microparticles having a volumetric mean diameter (VMD) from about 0.6 μm to about 2.5 μm.
79. The method of embodiment 78, wherein at least 80% of said microparticles having a VMD from about 0.6 μm to about 2.5 μm.
80. The method of embodiment 79, wherein at least 90% of said microparticles having a VMD from about 0.6 μm to about 2.5 μm.
81. The method of any one of embodiments 55-77, wherein when the BT composition is aerosolized, at least 70% of the aerosolized liquid droplets have a mass median aerodynamic diameter (MMAD) from about 0.9 μm to about 3 μm.
82. The method of embodiment 81, wherein when the BT composition is aerosolized, at least 80% of the aerosolized liquid droplets have a MMAD from about 0.9 μm to about 3 μm.
83. The method of embodiment 82, wherein when the BT composition is aerosolized, at least 90% of the aerosolized liquid droplets have a MMAD from about 0.9 μm to about 3 μm.
84. The method of any of embodiments 55-83, wherein the BT composition comprises BisEDT at a concentration greater than about 0.1 mg/mL, about 0.05% to about 1.00% Tween 80®, about 0.05 to 40 mM sodium chloride, and optionally about 2 to 20 mM sodium phosphate at about pH. 7.4.
85. A method of preventing, treating, managing or lessening the severity of symptoms associated with a respiratory viral infection in a subject in a subject, the method comprising administering to the subject a bismuth-thiol (BT) composition that comprises BisEDT suspended therein, wherein administering the BT composition is via inhalation, orally or nasally, using an aerosol device.
86. The method of embodiment 85, wherein the respiratory infection is a SARS-CoV-2 (COVID-19) infection.
87. The method of embodiment any one of embodiments 85-86, wherein the method is preventing symptoms associated with a respiratory viral infection in a subject.
88. The method of any one of embodiments 85-87, wherein the subject has a pre-existing health condition.
89. The method of embodiment 88, wherein the pre-existing health condition causes chronic inflammation in the subject.
90. The method of embodiment 88, wherein the pre-existing health condition is a chronic inflammatory disease.
91. The method of embodiment 90, wherein the chronic inflammatory disease is related to one of or more conditions selected from one or more of the group consisting of Metabolic Syndrome, Obesity, Vasculitis, Cardiovascular Diseases, Chronic Wounds, Diabetes, hypertension, stroke, inflammatory bowel disease, periodontitis, atherosclerosis, COPD, endocarditis, thrombotic diseases, asthma, advanced age, recurrent infections, autoimmune diseases, chronic rhinosinusitis, inflammatory arthritis, neurodegenerative conditions, device-related infections, osteomyelitis, and depression.
92. The method of any one of embodiments 85-91, wherein the patient is infected with a secondary infection.
93. The method of embodiment 92, wherein the secondary infection in the subject is prior to the respiratory viral infection.
94. The method of any one of embodiments 92-93, wherein the secondary infection produces a superantigen in the subject.
95. The method of embodiment 94, wherein the superantigen is a bacterial superantigen.
96. The method of embodiment 95, were in the superantigens include potent extotoxins secreted by Staphylococcus aureus and/or Streptococcus pyogenes.
97. The method of any one of embodiments 94-96, wherein the subject is infected with more than a 1 picomolar concentration of bacterial superantigen.
98. The method of any one of embodiments 94-96, wherein the subject is infected with more than 0.1 ug of superantigen.
99. The method of any one of embodiments 94-96, wherein the subject is infected with more than 0.2 ug of superantigen.
100. The method of any one of embodiments 85-99, wherein the method further prevents, manages or lessens the severity of a cytokine storm in a subject.
101. The method of any one of embodiments 85-100, wherein the subject has elevated levels of neutrophil-lymphocyte ratio (NLR), IL-6, IL-17, IL-10, interferon-gamma, TNF-α, IL-2, and/or procalcitonin.
102. The method of any one of embodiments 85-101, wherein the subject has elevated levels of neutrophil-lymphocyte ratio (NLR), IL-6, IL-17, and/or procalcitonin.
103. The method of embodiment 101 or 102, wherein the NLR ratio is greater than 5.
104. The method of embodiment 101 or 102, wherein the NLR ratio is greater than 7.
105. The method of embodiment 101 or 102, wherein the NLR ratios is between 7 and 100.
106. The method of any one of embodiments 85-105, wherein the administration to the subject a bismuth-thiol (BT) composition, treats, inhibits or prevents the dispersal of biofilms in the subject.
107. The method of embodiment 106, wherein the biofilm is a nasopharyngeal biofilm.
108. The method of any one of embodiments 85-107, wherein the subject is older than 60 years of age, 70 years of age, 80 years of age and/or 90 years of age.
109. The method of any one of embodiments 85-108, wherein the BT composition comprises a plurality of microparticles wherein at least 70% of said microparticles having a volumetric mean diameter (VMD) from about 0.6 m to about 2.5 μm.
110. The method of embodiment 109, wherein at least 80% of said microparticles having a VMD from about 0.6 μm to about 2.5 μm.
111. The method of embodiment 109, wherein at least 90% of said microparticles having a VMD from about 0.6 μm to about 2.5 μm.
112. The method of any one of embodiments 85-110, wherein when the BT composition is aerosolized, at least 70% of the aerosolized liquid droplets have a mass median aerodynamic diameter (MMAD) from about 0.9 μm to about 3 μm.
113. The method of embodiment 112, wherein when the BT composition is aerosolized, at least 800% of the aerosolized liquid droplets have a MMAD from about 0.9 μm to about 3 μm.
114. The method of embodiment 112, wherein when the BT composition is aerosolized, at least 90% of the aerosolized liquid droplets have a MMAD from about 0.9 μm to about 3 μm.
115. The method of any of embodiments 85-114, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.
116. A method of preventing, treating, managing or lessening the severity of Kawasaki disease or Kawasaki syndrome-like illness in a subject, the method comprising administering to the subject a bismuth-thiol (BT) composition that comprises BisEDT suspended therein.
117. The method of embodiment 116, wherein the subject has been previously infected or is infected with SARS-CoV-2 (COVID-19).
118. The method of embodiment 116 or 117, wherein the subject is a child.
119. The method of embodiment 116 or 117, wherein the subject is less than 12 years of age.
120. The method of embodiment 119, wherein the subject is less than 5 years of age.
121. The method of any one of embodiments 116-120, wherein the patient is infected with a secondary infection.
122. The method of embodiment 121, wherein the subject is infected with a bacteria that produces a superantigen.
123. The method of embodiment 121, were in the superantigens include potent extotoxins secreted by Staphylococcus aureus and/or Streptococcus pyogenes.
124. The method of any one of embodiments 122-123, wherein the subject is infected with more than a 1 picomolar concentration of superantigen.
125. The method of any one of embodiments 122-124, wherein the subject is infected with more than 0.1 micogram (ug) of superantigen.
126. The method of any one of embodiments 122-125, wherein the subject is infected with more than 0.2 ug of superantigen.
127. The method of any one of embodiments 116-126, wherein the method further prevents, manages or lessens the severity of a cytokine storm in a subject.
128. The method of any one of embodiments 116-127, wherein the subject has elevated levels of neutrophil-lymphocyte ratio (NLR), IL-6, IL-17, IL-10, interferon-gamma, TNF-αt, IL-2, and/or procalcitonin.
129. The method of any one of embodiments 128, wherein the subject has elevated levels of neutrophil-lymphocyte ratio (NLR), IL-6, IL-17, and/or procalcitonin.
130. The method of any one of embodiments 116-129, wherein the BT composition comprises a plurality of microparticles wherein at least 70% of said microparticles having a volumetric mean diameter (VMD) from about 0.6 μm to about 2.5 μm.
131. The method of embodiment 130, wherein at least 80% of said microparticles having a VMD from about 0.6 μm to about 2.5 μm.
132. The method of embodiment 131, wherein at least 90% of said microparticles having a VMD from about 0.6 μm to about 2.5 μm.
133. A method of preventing, treating, managing or lessening the severity of chronic inflammatory and/or metabolic diseases in a subject, the method comprising administering to the subject a bismuth-thiol (BT) composition that comprises BisEDT suspended therein, wherein administering the BT composition is via inhalation, orally or nasally, using an aerosol device.
134. The method of embodiment 133, wherein the subject has a SARS-CoV-2 (COVID-19) infection.
135. The method of embodiment 133-134, wherein the chronic inflammatory disease is related to one of or more conditions selected from one or more of the group consisting of Metabolic Syndrome, Obesity, Vasculitis, Cardiovascular Diseases, Chronic Wounds, Diabetes, hypertension, stroke, inflammatory bowel disease, periodontitis, atherosclerosis, chronic kidney disease, COPD, endocarditis, thrombotic diseases, asthma, aging, recurrent infections, autoimmune diseases, chronic rhinosinusitis, inflammatory arthritis, neurodegenerative conditions (e.g. Alzheimer's Disease), device-related infections, osteomyelitis, depression, chronic respiratory tract infections, chronic otitis media, and/or G.I. tract inflammatory diseases.
136. The method of any one of embodiments 133-135, wherein the patient is infected with a secondary infection.
137. The method of any one of embodiments 136, wherein the secondary infection produces a superantigen in the subject.
138. The method of embodiment 137, wherein the superantigen is a bacterial superantigen.
139. The method of embodiment 138, were in the superantigens include potent extotoxins secreted by Staphylococcus aureus and/or Streptococcus pyogenes.
140. The method of any one of embodiments 136-139, wherein the subject is infected with more than a 1 picomolar concentration of bacterial superantigen.
141. The method of any one of embodiments 136-140, wherein the subject is infected with more than 0.1 ug of superantigen.
142. The method of any one of embodiments 136-140, wherein the subject is infected with more than 0.2 ug of superantigen.
143. The method of any one of embodiments 133-142, wherein the method further prevents, manages or lessens the severity of a cytokine storm in a subject.
144. The method of any one of embodiments 85-100, wherein the subject has elevated levels of IL-1b, IL-2, IL-7, IL-8, IL-9, IL-10, IL-17, G-CSF, GMCSF, IFN-gamma, TNF-α, IP10, MCP1, MIP1A, MIP1B, and/or procalcitonin.
145. The method of any one of embodiments 144, wherein the subject has elevated levels of neutrophil-lymphocyte ratio (NLR), IL-6, IL-17, and/or procalcitonin.
146. The method of embodiment 144 or 145, wherein the NLR ratio is greater than 5.
147. The method of embodiment 144 or 145, wherein the NLR ratio is greater than 7.
148. The method of embodiment 144 or 145, wherein the NLR ratios is between 7 and 100.
149. The method of any one of embodiments 133-148, wherein the administration to the subject a bismuth-thiol (BT) composition, treats, inhibits or prevents the dispersal of biofilms in the subject.
150. The method of embodiment 149, wherein the biofilm is a nasopharyngeal biofilm.
151. The method of any one of embodiments 133-150, wherein the subject is older than 60 years of age, 70 years of age, 80 years of age and/or 90 years of age.
152. The method of any one of embodiments 133-151, wherein the BT composition comprises a plurality of microparticles wherein at least 70% of said microparticles having a volumetric mean diameter (VMD) from about 0.6 μm to about 2.5 μm.
153. The method of embodiment 152, wherein at least 80% of said microparticles having a VMD from about 0.6 μm to about 2.5 μm.
154. The method of embodiment 152, wherein at least 90% of said microparticles having a VMD from about 0.6 μm to about 2.5 μm.
155. The method of any one of embodiments 133-154, wherein when the BT composition is aerosolized, at least 70% of the aerosolized liquid droplets have a mass median aerodynamic diameter (MMAD) from about 0.9 μm to about 3 μm.
156. The method of embodiment 155, wherein when the BT composition is aerosolized, at least 800% of the aerosolized liquid droplets have a MMAD from about 0.9 μm to about 3 μm.
157 The method of embodiment 155, wherein when the BT composition is aerosolized, at least 90% of the aerosolized liquid droplets have a MMAD from about 0.9 μm to about 3 μm.
158. The method of any of embodiments 133-157, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.
159. A method of preventing, treating, managing or lessening the severity of lymphopenia in a subject, the method comprising administering to the subject a bismuth-thiol (BT) composition that comprises BisEDT suspended therein, wherein administering the BT composition is via inhalation, orally or nasally, using an aerosol device.
160. The method of embodiment 159, wherein the BT composition comprises a plurality of microparticles wherein at least 70% of said microparticles having a volumetric mean diameter (VMD) from about 0.6 μm to about 2.5 μm.
161. The method of embodiment 160, wherein at least 80% of said microparticles having a VMD from about 0.6 μm to about 2.5 μm.
162. The method of embodiment 160, wherein at least 90% of said microparticles having a VMD from about 0.6 μm to about 2.5 μm.
163. The method of any one of embodiments 159-162, wherein when the BT composition is aerosolized, at least 70% of the aerosolized liquid droplets have a mass median aerodynamic diameter (MMAD) from about 0.9 μm to about 3 μm.
164. The method of embodiment 163, wherein when the BT composition is aerosolized, at least 80% of the aerosolized liquid droplets have a MMAD from about 0.9 μm to about 3 μm.
165. The method of embodiment 163, wherein when the BT composition is aerosolized, at least 90% of the aerosolized liquid droplets have a MMAD from about 0.9 μm to about 3 μm.
166. The method of any of embodiments 159-165, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.

### Embodiments

167. An amorphous form of bismuth-1,2-ethanedithiol (BisEDT).
168. The amorphous form of BisEDT according to embodiment 167, wherein its X-ray powder diffraction pattern does not contain any distinct peaks.
169. The amorphous form of BisEDT according to embodiment 167 or embodiment 168, wherein its X-ray powder diffraction pattern is substantially similar to FIG. 44.
170. The amorphous form of BisEDT according to any one of embodiments 167-169, wherein its differential scanning calorimetry thermogram comprises an exothermic peak at about 168° C.
171. The amorphous form of BisEDT according to embodiment 170, wherein its differential scanning calorimetry thermogram further comprises an endotherm at about 64° C. and/or an endotherm peak at about 112° C. and/or an exotherm peak at about 145° C.
172. The amorphous form of BisEDT according to any of the preceding embodiments, wherein its differential scanning calorimetry thermogram is substantially similar to FIG. 45.
173. The amorphous form of BisEDT according to any of the preceding embodiments having a glass transition at about 101° C.
174. The amorphous form of any one of embodiments 167-173, wherein the amorphous form is at least 90% pure.
175. The amorphous form of embodiment 174, wherein the amorphous form is at least 95% pure.
176. The amorphous form of embodiment 175, wherein the amorphous form is at least 98% pure.
177. A composition comprising an amorphous from of bismuth-1,2-ethanedithiol (BisEDT).
178. The composition of embodiment 177, wherein the composition comprises at least one pharmaceutically acceptable carrier.
179. The composition of embodiment 177, wherein the composition comprises BisEDT in a suspension.
180. A method of treating, managing or lessening the severity of symptoms and infections associated with one or more pulmonary diseases or infections in a subject, the method comprising administering to the subject a bismuth-thiol (BT) composition that comprises amorphous BisEDT according to any one of embodiments 167-176 suspended therein, wherein administering the BT composition is via inhalation, orally or nasally, using an aerosol device.
181. The method of embodiment 180, wherein the method is treating, managing or lessening the severity of cystic fibrosis (CF) symptoms and infections in a subject.
182. A method for healing a wound in a subject having a diabetic foot infection, comprising administering the subject a therapeutically effective amount of a composition comprising amorphous BisEDT according to any one of embodiments 167-176.
183. The method of embodiment 182, wherein the wound is a diabetic foot ulcer.
184. A method of making an amorphous form of BisEDT according to any one of embodiments 167-176, comprising

(a) mixing an acidic aqueous solution that comprises a bismuth salt, with a solvent selected from the group consisting of acetone, acetonitrile, 1,2-dichloroethane, dimethyl sulfoxide, ethyl acetate, isopropanol, methyl tert-butyl ether, and mixtures thereof;

(b) combing the product of (a) with a solution of 1,2-ethanedithiol in a solvent selected from the group consisting of acetone, acetonitrile, 1,2-dichloroethane, dimethyl sulfoxide, ethyl acetate, isopropanol, methyl tert-butyl ether, and mixtures thereof, under conditions and for a time sufficient for formation of a precipitate which comprises the amorphous form of BisEDT.

185. The method of embodiment 184, further comprising recovering the precipitate to remove impurities.
186. The method of embodiment 184 or 185, wherein the bismuth salt is Bi(NO₃)₃.
187. The method of any one of embodiments 184-186, wherein 1,2-ethanedithiol is at a concentration of from about 1% wt/vol to about 20% wt/vol prior to step (b).
188. The method of any one of embodiments 184-187, wherein the acidic aqueous solution is prepared by mixing an aqueous suspension of either Bi (III) sub-nitrate or Bi (III) nitrate pentahydrate with an acid under conditions and for a time sufficient to form a substantially clear solution.
189. The method of embodiment 188, wherein the concentration of either Bi (III) sub-nitrate or Bi (III) nitrate pentahydrate in the aqueous solution is from about 100 mg/mL to about 400 mg/mL.
190. The method of embodiment 188 or embodiment 189, wherein the acid is 70% HNO₃.
191. The method of any one of embodiments 188-190, further comprising adding the clear solution to an acidic solution.
192. The method of embodiment 191, wherein the acidic solution is 5% HNO₃.
193. The method of any one of embodiments 184-193, wherein step (b) is performed at a temperature ranging from about 20° C. to about 28° C.
194. A method of treating, managing or lessening the severity of cystic fibrosis (CF) symptoms and infections in a subject, the method comprising administering to the subject a bismuth-thiol (BT) composition that comprises amorphous BisEDT according to any one of embodiments 167-176 suspended therein.
195. The method of embodiment 14-15 or 28, wherein the BT composition comprises a plurality of microparticles wherein at least 70% of said microparticles having a volumetric mean diameter (VMD) from about 0.01 μm to about 2.5 μm.
196. The method of embodiment 180, 181, 194, or 195, wherein at least 80% of said microparticles having a VMD from about 0.01 μm to about 2.5 μm.
197. The method of any one of embodiments 180, 181, 194-196, wherein at least 90% of said microparticles having a VMD from about 0.01 μm to about 2.5 μm.
198. The method of any one of embodiments 180, 181, 194-197, wherein when the BT composition is aerosolized, at least 70% of the aerosolized liquid droplets have a mass median aerodynamic diameter (MMAD) from about 0.01 μm to about 3 μm.
199. The method of any one of embodiments 180, 181, 194-198, wherein when the BT composition is aerosolized, at least 80% of the aerosolized liquid droplets have a MMAD from about 0.01 μm to about 3 μm.
200. The method of any one of embodiments 180, 181, 194-199, wherein when the BT composition is aerosolized, at least 90% of the aerosolized liquid droplets have a MMAD from about 0.01 μm to about 3 μm.
201. The method of any one of embodiments 180, 181, 194-200, wherein the BT composition comprises BisEDT at a concentration greater than about 0.1 mg/mL, about 0.05% to about 1.0% Tween 80®, about 0.05 to 250 mM sodium chloride, and optionally about 2 to 20 mM sodium phosphate at about pH. 7.4.
202. The method of any one of embodiment 201, wherein range of sodium chloride is about 100 mM to about 200 mM.
203. The method of any one of embodiments 180, 181, 194-202, wherein the subject has at least one pulmonary infection containing one or more bacterial pathogens and/or fungal pathogens.
204. The method of any one of embodiments 180, 181, 194-203, wherein the method comprises at least one of: (i) reducing a bacterial biofilm, (ii) impairing growth of a bacterial biofilm, (iii) preventing initial formation of the bacterial biofilm, and/or (iv) preventing reformation of the bacterial biofilm.
205. The method of any one of embodiments 180, 181, 194-204, wherein the one or more pathogens are selected from Haemophilus influenzae, Pseudomonas aeruginosa, Staphylococcus aureus, Staphylococcus warneri Staphylococcus lugdunensis. Staphylococcus epidermidis. Streptococcus milleri/anginous, Streptococcus pyogenes, non-tuberculosis mycobacteria, Mycobacterium tuberculosis, Burkholderia spp., Achromobacter xylosoxidans. Pandoraea sputorum, Stenotrophomonas maltophilia, Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morganii, Inquilinus limosus, Ralstonia mannitolilytica, Pandoraea apista, Pandoraea pnomenusa, Pandoraea sputorum, Bdellovibrio bacteriovorus, Bordetella bronchiseptica, Vampirovibrio chlorellavorus, Actinobacter baumanni, Cupriadidus metallidurans, Cupriavidus pauculus, Cupriavidus respiraculi, Delftia acidivordans, Exophilia dermatitidis, Herbaspirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralstonia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S. agalactiae, Bacteroides fragilis, Clostridium difficile, Streptococcus pneumonia, Moraxella catarrhalis, Haemophilus haemolyticus, Haemophilus parainfluenzae, Chlamydophilia pneumoniae, Mycoplasma pneumoniae, Atopobium, Sphingomonas, Saccharibacteria, Leptotrichia, Capnocytophaga, Oribacterium. Aquabacterium, Lachnoanaerobaculum, Campylobacter, Acinetobacter; Agrobacterium; Bordetella; Brevundimonas; Chryseobacterium; Delftia; Enterobacter; Klebsiella; Pandoraea; Pseudomonas; Ralstonia, and Prevotella.
206. The method of any one of embodiments 180, 181, 194-205, wherein the one or more pathogens are non-tuberculosis mycobacteria.
207. An aerosol comprising a plurality of dispersed liquid droplets in a gas, said liquid droplets comprising a BT composition comprising amorphous BisEDT according to any one of embodiments 167-176 suspended therein; and

wherein at least 70% of the liquid droplets have a MMAD from about 0.9 μm to about 3 μm.

208. The aerosol of embodiment 207, wherein prior to aerosolization, the BT composition comprises a plurality of microparticles wherein at least 70% of said microparticles have a VMD from about 0.01 μm to about 2.5 μm.
209. The aerosol of embodiment 207 or embodiment 208, wherein least 80% of the liquid droplets have a MMAD from about 003 μm to about 3 μm.
210. The aerosol of any one of embodiments 207-209, wherein least 90% of the liquid droplets have a MMAD from about 0.03 μm to about 3 μm.
211. The aerosol of any one of embodiments 207-210, wherein prior to aerosolization, the BT composition comprises a plurality of microparticles wherein at least 80% of said microparticles have a VMD from about 0.01 μm to about 2.5 μm.
212. The aerosol of any one of embodiments 207-212, wherein prior to aerosolization, the BT composition comprises a plurality of microparticles wherein at least 90% of said microparticles have a VMD from about 0.01 μm to about 2.5 μm.
213. The aerosol of any one of embodiments 207-212, wherein the droplets further comprise Tween 80 and optionally a buffer at a pH of about 7.4; and/or sodium chloride.
214. The aerosol of any one of embodiments 207-213, wherein a substantial amount of the BisEDT compounds are deposited to the deep lung region.
215. The aerosol of any one of embodiments 207-214, wherein at least 80% of the BisEDT compounds are deposited to the deep lung region.
216. A pharmaceutical composition comprising bismuth-thiol (BT) composition that comprises amorphous BisEDT according to embodiments 167-176 suspended therein, wherein the BT composition comprises a plurality of microparticles, wherein the D90 of said microparticles is less than or equal to 1.9 μm.
217. The pharmaceutical composition of embodiment 217, comprising bismuth-thiol (BT) composition comprises BisEDT suspended therein, wherein the BT composition comprises a plurality of microparticles, wherein the D90 of said microparticles is less than or equal to about 1.6 μm.
218. The pharmaceutical composition of embodiments 216-217, wherein at least 70% of said microparticles having a volumetric mean diameter from about 0.01 μm to about 2.5 μm.
219. The pharmaceutical composition according to any of embodiments 216-218, wherein at least 90% of said microparticles having a volumetric mean diameter from about 0.6 μm to about 2.5 μm.
220. A method of treating, managing or lessening the severity of symptoms and infections associated with one or more pulmonary diseases or infections in a subject, the method comprising administering to the subject a bismuth-thiol (BT) composition that comprises amorphous BisEDT according to nay of embodiments 167-176, wherein the BT composition comprises a plurality of microparticles wherein at least 70% of said microparticles having a volumetric mean diameter from about 0.01 μm to about 2.5 μm, and wherein when the BT composition is aerosolized, at least 70% of the aerosolized liquid droplets have a MMAD from about 0.03 μm to about 3 μm.
221. The method of embodiment 220, wherein the one or more pulmonary diseases or infections are not the result of or associated with cystic fibrosis.
222. The method of embodiment 220-221, wherein the pulmonary infection is bronchiectasis infection, pneumonia, valley fever, allergic bronchopulmonary aspergillosis (ABPA), ventilator acquired pneumonia, hospital acquired pneumonia, community acquired pneumonia, ventilator associated tracheobronchitis, lower respiratory tract infection, non-tuberculous Mycobacteria (NTM), anthrax, legionellosis, pertussis, bronchitis, Bronchiolitis, COPD-associated infection, and post-lung transplantation.
223. The method of embodiment 222, wherein the pulmonary infection is non-tuberculous Mycobacteria (NTM).
224. A method for healing a wound in a subject having a diabetic foot infection, comprising administering the subject a therapeutically effective amount of a composition comprising amorphous BisEDT according to any one of embodiments 167-176, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is healed or substantially healed within 12 weeks of the first administration of the composition.
225. The method of embodiment 224, wherein the wound is a diabetic foot ulcer.
226. The method of embodiment 182-183 or 224-225, wherein the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.
227. The method of any one of embodiments 182-183 or 224-226, wherein the applied BT composition is present on the surface at a concentration from about 1 μg/cm² to about 1,000,000 μg/cm².
228. The method of any one of embodiments 182-183 or 224-227, wherein the applied BT composition is present on the surface at a concentration from about 50 μg/cm² to about 100 μg/cm².
229. The method of any one of embodiments 182-183 or 224-228, wherein the applied BT composition is present on the surface at a concentration greater than about 100 μg/cm².
230. The method of any one of embodiments 182-183 or 224-229, wherein the BT composition is administered three times per day, two times per day, once daily, every other day, once every three days, three times per week, once every week, once every other week, once every month, or once every other month.
231. The method of any one of embodiments 182-183 or 224-230, wherein the wound is healed 4 weeks, 8 weeks or 12 weeks after the first administration of the BT composition.
232. The method of any one of embodiments 182-183 or 224-231, wherein the subject is administered multiple doses of the BT composition daily or weekly for a length of time ranging from about one week to about 12 weeks.
233. The method of any one of embodiments 182-183 or 224-232, wherein the subject is administered multiple doses of the BT composition daily or weekly for a length of about 4 weeks.
234. The method of any one of embodiments 182-183 or 224-233, wherein the wound area is from about 0.1 cm² to about 250 cm².
235. A method for wound size reduction in a subject having a diabetic foot infection, comprising administering to the subject a therapeutically effective amount of a composition comprising amorphous BisEDT according to embodiments 167-176, wherein the composition is a suspension of microparticles comprising said BisEDT wherein at least 70% of the microparticles have a volumetric mean diameter (VMD) from about 0.01 μm to about 5 μm, and wherein the composition is applied to the infection and the wound is reduced in size from about a 1% reduction relative to the original wound size to total elimination of the wound within 12 weeks of the first administration of the composition.
236. The method of any one of embodiments 182-183 or 2350, wherein the wound is reduced by about 10%, 20%, 30%, 40%, 50%, 60%, 70%, 80%, or 90%.
237. The method of embodiment 182-183 or 235-236, wherein the wound is reduced by at least about 50%.
238. The method of any one of embodiments 182-183 or 235-237, wherein the wound is a diabetic foot ulcer.
239. The method of any one of embodiments 182-183 or 235-238, wherein the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7.4.
240. The method of any one of embodiments 182-183 or 235-239, wherein the applied BT composition is present on the surface at a concentration from about 1 μg/cm² to about 1,000,000 μg/cm².
241. The method of any one of embodiments 182-183 or 235-240, wherein the applied BT composition is present on the surface at a concentration from about 50 μg/cm² to about 100 μg/cm².
242. The method of any one of embodiments 182-183 or 235-241, wherein the applied BT composition is present on the surface at a concentration greater than about 100 μg/cm².
243. The method of any one of embodiments 182-183 or 235-242, wherein the BT composition is administered three times per day, two times per day, once daily, every other day, once every three days, three times per week, once every week, once every other week, once every month, or once every other month.
244. The method of any one of embodiments 182-183 or 235-243 wherein the BT composition is administered once daily or three times per week.
245. The method of any one of embodiments 182-183 or 235-244, wherein the subject is administered multiple doses of the BT composition daily or weekly for a length of time ranging from about one week to about 12 weeks.
246. The method of any one of embodiments 182-183 or 235-244, wherein the subject is administered multiple doses of the BT composition daily or weekly for a length of about 4 weeks.
247. The method of any one of embodiments 182-183 or 235-246, wherein the wound area is from about 0.1 cm² to about 250 cm².
248. The method of any one of embodiments 182-183 or 235-247, wherein the wound surface area of said wound is reduced by at least 50% by 12 weeks after the first administration of the BT composition.
249. The method of any one of embodiments 182-183 or 235-248, wherein the wound surface area of said wound is reduced by at least 50% by 4 weeks after the first administration of the BisEDT composition.
250. The method of any one of embodiments 182-183 or 235-249, wherein the wound surface area is measured using digital photographs or hand measurement.
251. A pharmaceutical composition comprising bismuth-thiol (BT) composition that comprises amorphous BisEDT according to any one of embodiments 167-176 suspended therein, wherein the BT composition comprises a plurality of microparticles, wherein the D90 of said microparticles is less than or equal to 1.9 μm.
252. The pharmaceutical composition of embodiment 251, wherein the BT composition comprises a plurality of microparticles, wherein the D90 of said microparticles is less than or equal to about 1.6 μm.
253. A method for healing a wound in a subject having a diabetic foot infection, comprising administering the subject a therapeutically effective amount of a composition of embodiment 251 or embodiment 252.

### EXAMPLES

The following examples are provided to illustrate the present disclosure, and should not be construed as limiting thereof. Additional experimental procedures and details can be found in International Patent Application Nos. PCT/US2010/023108, PCT/US2011/023549, and PCT/US2011/047490, which are hereby incorporated by reference in their entireties for all purposes.

### Example 1: General Synthesis of Bis-EDT

The starting materials and reagents used in preparing these compounds are either available from commercial supplier such as Aldrich Chemical Co., Bachem, etc., or can be made by methods well known in the art. The starting materials and the intermediates and the final products of the reaction can be isolated and purified if desired using conventional techniques, including but not limited to filtration, distillation, crystallization, chromatography, and the like and can be characterized using conventional means, including physical constants and spectral data. Unless specified otherwise, the reactions described herein take place at atmospheric pressure over a temperature range from about −78° C. to about 150° C.

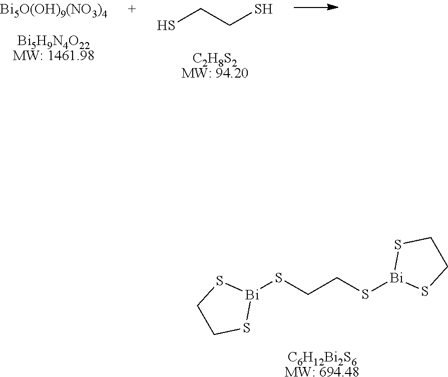

Microparticulate bismuth-1,2-ethanedithiol (Bis-EDT, soluble bismuth preparation) was prepared as follows: To an excess (11.4 L) of 5% aqueous HNO₃ at room temperature in a 15 L polypropylene carboy was slowly added by dropwise addition 0.331 L (˜0.575 moles) of an aqueous Bi(NO) solution (43% Bi(NO₃)₃ (w/w), 5% nitric acid (w/w), 52% water (w/w), Shepherd Chemical Co., Cincinnati, Ohio, product no. 2362; δ˜1.6 g/mL) with stirring, followed by slow addition of absolute ethanol (4 L). Some white precipitate formed but was dissolved by continued stirring. An ethanolic solution (˜1.56 L, ˜0.55 M) of 1,2-ethanedithiol (CAS 540-63-6) was separately prepared by adding, to 1.5 L of absolute ethanol, 72.19 mL (0.863 moles) of 1,2-ethanedithiol using a 60 mL syringe, and then stirring for five minutes. The 1,2-ethanedithiol/EtOH reagent was then slowly added by dropwise addition over the course of five hours to the aqueous Bi(NO₃)₃/HNO₃ solution, with continued stirring overnight. The formed product was allowed to settle as a precipitate for approximately 15 minutes, after which the filtrate was removed at 300 mL/min using a peristaltic pump. The product was then collected by filtration on fine filter paper in a 15-cm diameter Buchner funnel, and washed sequentially with three, 500-mL volumes each of ethanol, USP water, and acetone to obtain BisEDT (694.51 gm/mole) as a yellow amorphous powdered solid. The product was placed in a 500 mL amber glass bottle and dried over CaCl₂ under high vacuum for 48 hours. Recovered material (yield ˜200 g) gave off a thiol-characteristic odor. The crude product was redissolved in 750 mL of absolute ethanol, stirred for 30 min, then filtered and washed sequentially with 3×50 mL ethanol, 2×50 mL acetone, and washed again with 500 mL of acetone. The rewashed powder was triturated in 1M NaOH (500 mL), filtered and washed with 3×220 mL water, 2×50 mL ethanol, and 1×400 mL acetone to afford 156.74 gm of purified BisEDT. Subsequent batches prepared in essentially the same manner resulted in yields of about 78-91%.

The product was characterized as having the structure shown above by analysis of data from ¹H and ¹³C nuclear magnetic resonance (NMR), infrared spectroscopy (IR), ultraviolet spectroscopy (UV), mass spectrometry (MS) and elemental analysis. An HPLC method was developed to determine chemical purity of BisEDT whereby the sample was prepared in DMSO (0.5 mg/mL). The λₘₐₓ was determined by scanning a solution of BisEDT in DMSO between 190 and 600 nm. Isocratic HPLC elution at 1 mL/min was performed at ambient temperature in a mobile phase of 0.1% formic acid in acetonitrile:water (9:1) on a Waters (Millipore Corp., Milford, Mass.) model 2695 chromatograph with UV detector monitoring at 265 nm (λₘₐₓ), 2 μL injection volume, equipped with a YMC Pack PVC Sil NP, 5 μm, 250X4.6 mm inner diameter analytical column (Waters) and a single peak was detected, reflecting chemical purity of 100±0.1%. Elemental analysis was consistent with the structure of BisEDT as shown above.

The dried particulate matter was characterized to assess the particle size properties. Briefly, microparticles were resuspended in 2% Pluronic® F-68 (BASF, Mt. Olive, N.J.) and the suspension was sonicated for 10 minutes in a water bath sonicator at standard setting prior to analysis using a Nanosizer/Zetasizer Nano-S particle analyzer (model ZEN1600 (without zeta-potential measuring capacity), Malvern Instruments, Worcestershire, UK) according to the manufacturer's recommendations. From compiled data of two measurements, microparticles exhibited a unimodal distribution with all detectable events between about 0.6 microns and 4 microns in volumetric mean diameter (VMD) and having a peak VMD at about 1.3 microns.

### Example 2: Preparation of Microparticulate bismuth-1-2-ethanedithiol (Bis-EDT)

Microparticulate bismuth-1,2-ethanedithiol (Bis-EDT) was prepared as follows: Water (25.5 L) and 70%/o nitric acid (1800 mL) were mixed together in a Nalgene reactor. Then, water (2300 mL) was added to an Erlenmeyer flask, followed by bismuth subnitrate (532 g), and the mixture was stirred. To the mixture was added 70% nitric acid (750 mL) to obtain a clear solution. This solution was transferred into the Nalgene reactor and the resulting mixture was stirred for 20 min. Then, 9.5 L of 95% EtOH was added to the reactor in three portions. Separately, 1,2-ethanedithiol, 98%, (229 mL) was added to a bottle followed by two 250 mL EtOH portions with stirring. A further 5 L EtOH was added to the bottle with stirring. The 1,2-ethanedithiol solution was then added to the reactor over about 4 hours while stirring. After stirring for 18 hours, the solids were allowed to settle for 2 hours. EtOH (20 L) was added and the mixture stirred for 24 hours. The solids were allowed to settle for 1.5 hours, then separated by filtration of the mixture, followed by rinsing with EtOH.

To the empty reactor was added 9 L EtOH and the filtered solids, which was stirred for 18 hours. The solids were allowed to settle for 1 hour, then separated by filtration of the mixture, followed by rinsing with EtOH. Next, the empty reactor was charged with 9 L acetone, 99.5%, and the filtered solids, which was stirred for 15 hours. The solids were allowed to settle for 1.5 hours, then separated by filtration of the mixture, followed by rinsing with acetone. Again, the empty reactor was charged with 9 L acetone, 99.5%, and the filtered solids, which was stirred for 1.4 hours. The solids were filtered and air-dried for 69 hours, then vacuum-dried for 4 hours. After mixing the solid, it was sieved through a 10 mesh (2 mm) and then 18 mesh (1 mm) sieve to give BisEDT.

### Example 3: Synthesis of Additional Bismuth Thiol Compounds

The following bismuth thiol compounds can also be prepared according to the methods of Examples 1 and 2:

bismuth-2,3-dimercaptopropanol (2:3 molar ratio, BisBAL)
bismuth-4-methyl-1,2-benzenedithiol (2:3 molar ratio, BisTOL)
bismuth-2,3-butanedithiol (BisBDT)

### Example 4: Formulation(s) for CF Inhalant

Objective: The objective of this study was to develop methods for the nose-only inhalation exposures of BisEDT for rodents. The aerosols were generated from suspension formulations of BisEDT in aqueous media (0.5% Tween 80 in Sodium Phosphate Buffer, pH 7.4). Aerosols were generated with a commercial compressed air jet nebulizer into a rodent nose only inhalation exposure system. Suspension formulations were created by sonication with neat API with 0.5% Tween 80 in Sodium Phosphate Buffer, pH 7.4. This was further refined by adjusting the vehicle to an osmolality of 300 mOsmol/kg. Aerosols were characterized for particle size distribution and aerosol concentration. Aerosol concentration was determined by differential mass and chemical analysis of the aerosol filter samples.

Suspension concentrations between 2.5 and 100 mg/mL were evaluated for one formulation of BisEDT and ranged from 18.5 to 719 μg/L respectively. Further tests were performed with a new formulation of BisEDT and the suspension concentrations of 2.5 mg/mL, 10 mg/mL, 50 mg/mL and 100 mg/mL yielded aerosol concentrations of 18.4, 97.7, 159, and 1300 μg/L, respectively. The particle size increased with suspension concentration from ˜1 μm to 3.5 μm MMAD, which is respirable for rodent inhalation exposures.

To support potential dose range finding studies in rat nose only inhalation exposures are typically recommended for between 30 and 180 minutes. Therefore the pulmonary deposited dose range from these suspension formulations are between 42 μg/kg and 17.5 mg/kg. As appropriate the suspension concentration and/or exposure time can be modulated to adjust pulmonary deposited dose.

Materials and Methods: BisEDT—Two separate lots of Bismuth Ethane Dithiol, BisEDT, were used as received. The initial stages of method development were performed with Lot #ED268-1-11-01 and the final stages were performed with Lot #XL-47-125, which had been shipped in preparation for exposures. The two materials behaved slightly differently, requiring a change in the final determination of exposure times.

Aerosol Methods: Formulation—Suspension formulations of BisEDT were prepared in 0.5% Tween 80 in Sodium Phosphate Buffer, pH 7.4. Suspensions of 2.5, 10, 25, 50, 75 and 100 mg/mL were prepared by sonication with a Covaris sonicator. In later tests, the vehicle was adjusted with sodium chloride to achieve an Osmolality of 300 mOsmol/kg. Suspensions were prepared at 2.5, 50, and 100 mg/mL.

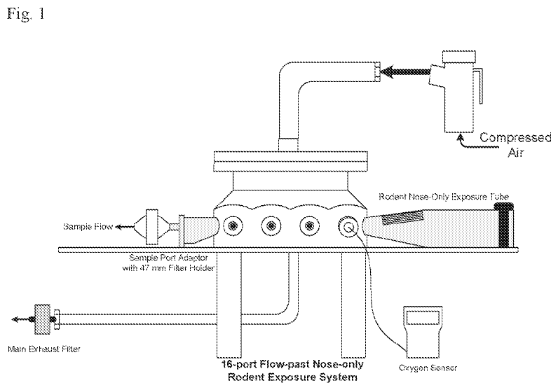

Aerosol Delivery System: FIG. 1 shows a schematic of the aerosol generation and exposure system used during these tests. Aerosol was generated by using a Pari LC Plus compressed air jet nebulizer (Pari Respiratory Equipment Inc., Midlothian, Va.) with an inlet pressure of 20 psi. The aerosols transitioned into a rodent nose-only inhalation exposure system operated with an inlet air flow of ˜8.5 L/min and an exhaust air flow of ˜7.1 L/min. Aerosol generated by the nebulizer was delivered to a one-tier nose-only flow-past inhalation exposure system.

Aerosol concentration was measured at the breathing zone of the exposure system by collection of the aerosol onto 47-mm filters (Whatman GF/A membrane filters). Filter samples were collected at a nominal flowrate of 0.3 L/min. Aerosol mass concentration was determined by differential weight analysis of the filter samples. Filters were then transferred to the analytical chemistry laboratory for analysis using HPLC. Details of the analytical chemistry methods are included in analytical method ACM-1046-0 (Determination of BisEDT in Formulations and Filter Extracts by HPLC-UV).

Particle Size Distribution Measurement: Particle size distribution for exposure was measured by using an In-Tox Mercer 2.0 L/min cascade impactor (In-Tox, LLC, Moriarty, N. Mex.).

Calculation of Deposited Dose: The first two equations were used to calculate the presented aerosol dose and the theoretical deposited dose, respectively. In these calculations the average aerosol concentration (chemistry) along with projected body weights for rats are used.

$${D}_{P} [ mg {kg}^{- 1} ] = \frac{AC [ mg {L}^{- 1} ] RMV [ L {min}^{- 1} ] T [ min ]}{BW [ kg ]}$$

$${D}_{D} [ mg {kg}^{- 1} ] = \frac{AC [ mg {L}^{- 1} ] RMV [ L {min}^{- 1} ] T [ min ] D F}{BW [ kg ]}$$

$$RMV = 0.608 {BW}^{0.852}$$

Where: D_{P}: Presented dose; D_{D}: Deposited Dose; AC: Aerosol

### Exposure Concentration; RMV: Respiratory Minute Volume

(Alexander. D J, et al., 2008. Inhal Toxicol.; 20(13): 1179-89); T:
Exposure time; BW: Body Weight; DF: Pulmonary deposition
fraction (assumed 10%, Tepper et al., 2016 Int J Toxicol; 35(4):376-
392); Time varied between 30 and 180 minutes.

### Results

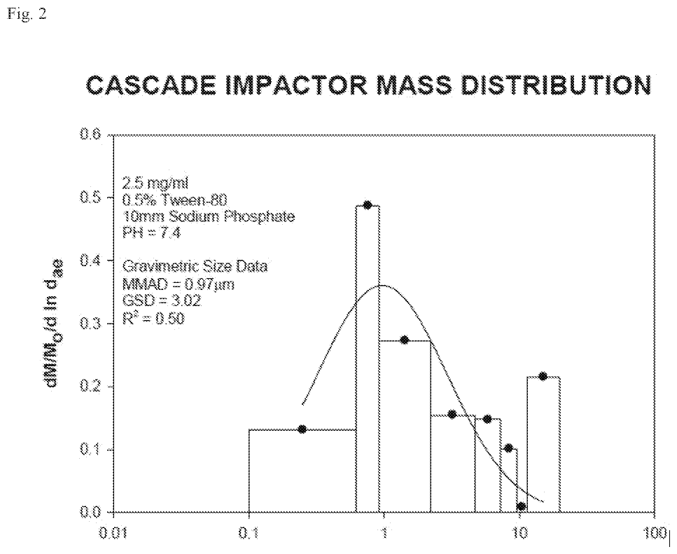

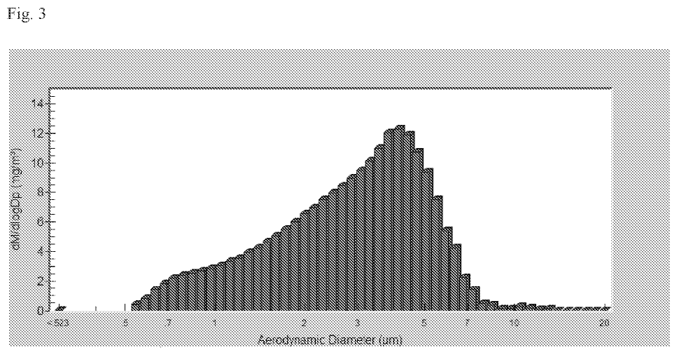

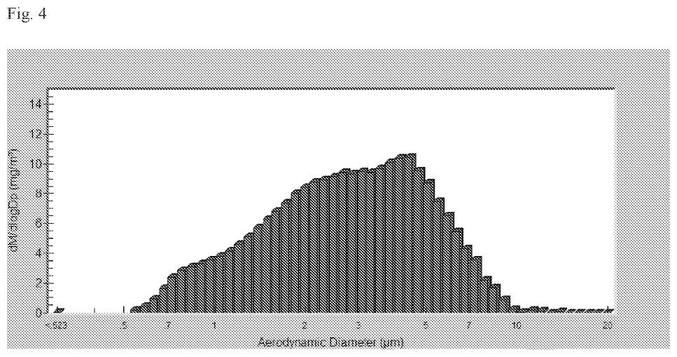

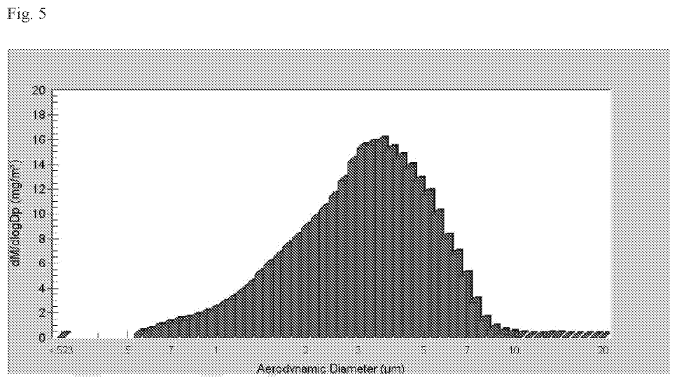

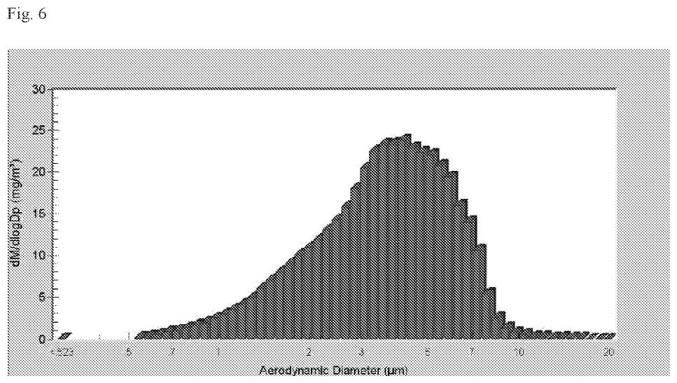

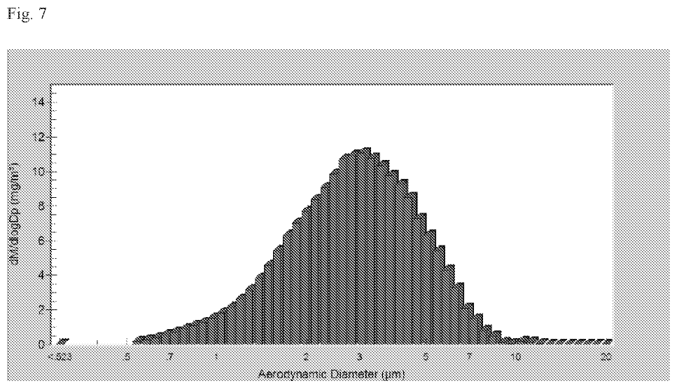

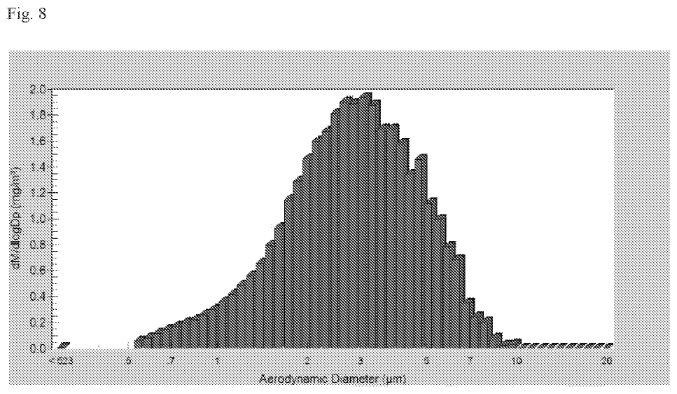

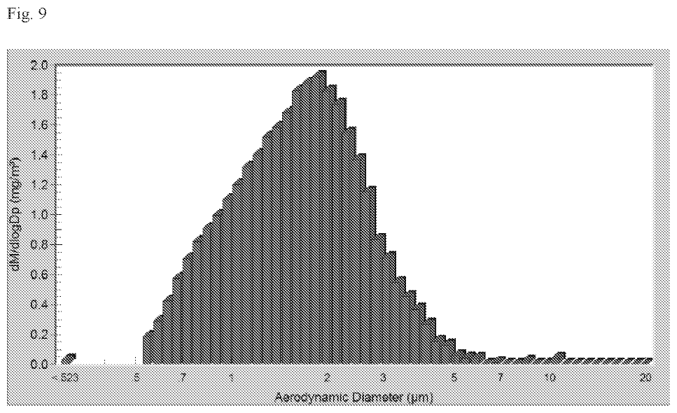

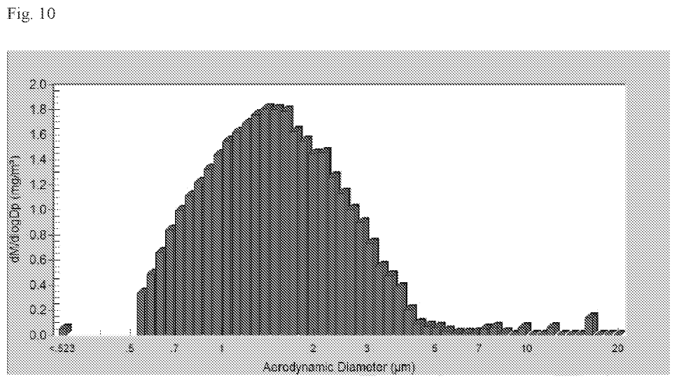

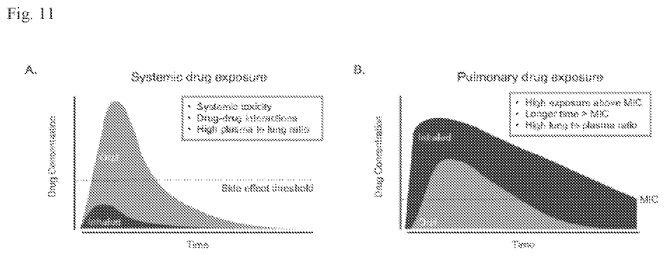

Aerosol Concentration and Particle Size: The average total aerosol concentration, BisEDT aerosol concentration and particle size for the formulations are shown for the first BisEDT formulation (Lot #ED268-1-11-01) in Table 1. An analogous table is shown for the second BisEDT formulation (Lot #XL-47-125) in Table 2. An example histogram of the particle size distribution for the 2.5 mg/mL concentration taken via cascade impactor is shown in FIG. 2. Example histograms of the particle size distributions for the 25, 50, 75, and 100 mg/mL concentrations taken via APS are shown in FIGS. 3, 4, 5, and 6; respectively. Repeated tests for size distributions for the second BisEDT formulation are depicted in FIGS. 7, 8, 9, and 10 for 100 mg/mL, 50 mg/mL, 10 mg/mL, and 2.5 mg/mL respectively.

TABLE 1
Summary of suspension aerosol testing for
BisEDT (Lot # ED268-1-11-01)

| Formulation | Total Aerosol | BisEDT Aerosol | Particle Size | |
|---|---|---|---|---|
| (mg/mL) | Conc. (mg/L) | Conc. (μg/L) | (GSD)* | |
| 1 | | 9.9 | 1.65 (1.6) | μm |
| 2.5 | 0.116 | 18.5 | 0.97 (3.0) | μm |
| 10 | 0.184 | 80.7 | N/A | |
| 25 | 0.441 | 87.9 (64.4)⁺ | 2.98 (1.8) | μm |
| 50 | 0.599 | 156 | 2.83 (1.9) | μm |
| 75 | 0.618 | 530 | 3.16 (1.70) | μm |
| 100 | 0.816 | 719 | 3.53 (1.71) | μm |
| 100* | 0.731 | 568 | 3.60 (1.71) | μm |

*All Particle size data collected with an APS except the 2.5 mg/mL formulation
⁺for one of the filters of the 25 mg/mL solution, chemical extraction yielded a less than expected concentration of BisEDT. 64.4 μg/L is the average value of all extracted concentrations, while 87.9 μg/L is the average excluding this outlier.

TABLE 2
Summary of suspension aerosol testing for BisEDT (Lot
# XL-47-125) using a 300 mOsmol/kg vehicle

| Formulation | Total Aerosol | BisEDT Aerosol | Particle Size |
|---|---|---|---|
| (mg/mL) | Conc. (mg/L) | Conc. (μg/L) | (GSD) |
| 100 | 1.535 | 1300 | 2.90 (1.67) μm |
| 100 | 1.404 | N/A | 2.60 (1.65) μm |
| 50 | 0.711 | 159 | 2.79 (1.69) μm |
| 10 | 0.332 | 97.7 | 1.61 (1.65) μm |
| 2.5 | 0.175 | 18.4 | 1.44 (1.71) μm |

The aerosols in Tables 1 and 2 were generated with a Pari LC Plus® Performance nebulizer. The nebulizer was tested with a 1.2 bar compressor; measured with Malvern MasterSizer X at 50% relative humidity, 0.9 NaCl solution, inspiratory flow of 20 L/min, continuous nebulization at 23° C. with a fill volume of 2.5 mL.

Pulmonary Deposited Dose; Pulmonary deposited doses can be modulated via the aerosol concentration and/or the exposure duration. For a rodent nose-only inhalation exposure, the exposure duration is typically targeted between 30 and 180 minutes. Using the performance of the aerosol and the pulmonary dose equation above, the pulmonary deposited dose range for the first BisEDT formulation would be between 42 μg/kg and 9.6 mg/kg. The second formulation was more efficient in depositing BisEDT and is capable of giving a dose up to 17.5 mg/kg.

Methods for formulation and aerosol generation of BisEDT were developed. They support a pulmonary deposited dose between 42 μg/kg and 17.5 mg/kg. These exposure conditions are appropriate for rodent inhalation exposures.

### Example 5: In Vitro Studies

This Example describes a series of experiments evaluating the suitability and feasibility of the development of bismuth-thiols, particularly BisEDT, as an inhaled drug product for the treatment of CF-related pulmonary infections. A CF-relevant range of infectious bacterial pathogens, including highly resistant strains and biofilm forming strains of such pathogens, were tested for susceptibility to bismuth-thiols. In addition, in vitro evaluations of toxicity against lung airway epithelial cells were conducted.

Standardized microbiological susceptibility testing was carried out with two bismuth-thiol compounds against a range of the most clinically-challenging, highly resistant CF isolates. MIC testing was standardized using ATCC strains of E. coli (25922) and S. aureus (29213).

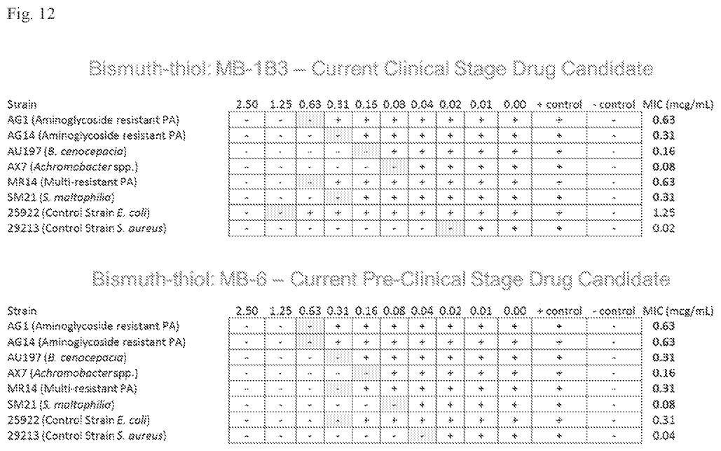

The results of this testing demonstrated BisEDT was consistently potent against all CF isolates tested, including aminoglycoside-resistant and MDR P. aeruginosa, B. cenocepacia, Achromobacter spp., and S. maltophilia, with all test organisms demonstrating MICs of less than 1 μg/mL (see FIG. 12).

To expand upon this, the tested bacteria was expanded to include more Burkholderia spp., as well as to test a wider range of CF isolates and antibiotic-resistant strains.

A range of CF-isolated strains of Mycobacterium avium and Mycobacterium abscessus were included; which allows for understanding of the activity against non-tubercular Mycobacteria (NTM). The results of this study are shown below. Two distinct bismuth-thiol compounds were evaluated, including BisEDT and BisBDT. Mycobacterium avium isolates were found to have the highest MICs, though BisEDT demonstrated lower MICs against these NTM bacteria than did BisBDT. The CF-isolates of greater clinical relevance, including both M. abscessus spp. and Burkholderia spp., were both consistently found to be much more susceptible to BisEDT and BisBDT. This study demonstrated very compelling MIC data against CF-isolates of both NTM and Burkholderia spp., as few commercial antibiotics have this level and spectrum of activity.

TABLE 3A
BisEDT and BisBDT MIC against bacterial strains

| | | | BisEDT | BisBDT |
|---|---|---|---|---|
| Strain | | | MIC | MIC |
| Designation | Species | Strain characteristics | (μg/mL) | (μg/mL) |
| MAC101 | M. avium | MAC reference strain, blood | 2 | 1 |
| | | isolate from HIV patient | | |
| AMT G193-13 | M. avium | MAC CF isolate, macrolide-S | 8 | >16 |
| AMT G119-8 | M. avium | MAC CF isolate, macrolide-S | 8 | >16 |
| BC 5 | B. multivorans | CF isolate, beta lactam-S, | 1 | 2 |
| | (cepacia complex) | TMP/SMX-S, FQ-I | | |
| PC 213 | B. cepcacia complex | CF isolate, beta lactam-S, | 0.25 | 1 |
| | (not further speciated) | TMP/SMX-S, FQ-S | | |
| BC 9 | B. cepcacia complex | CF isolate, beta lactam-S, | 0.5 | 0.5 |
| | (not further speciated) | TMP/SMX-S, FQ-R | | |
| BC 17 | B. cepacia | CF isolate, beta lactam-R, | 8 | 4 |
| | (cepacia complex) | TMP/SMX-R, FQ-R | | |
| BC 11 | B. dolosa | CF isolate, beta lactam-R, | 0.125 | 0.5 |
| | (cepacia complex) | TMP/SMX-S, FQ-R | | |
| ATCC 19977 | M. abscessus abscessus | ATCC type strain | 0.5 | 0.25 |
| AMT 0136-10 | M. abscessus complex | MABSC CF isolate, macrolide-R, | 0.125 | 0.0625 |
| | | amikacin-R | | |
| AMT 0089-5 | M. abscessus complex | MABSC CF isolate, macrolide-R, | 0.25 | 0.125 |
| | | amikacin-R | | |
| AMT 0166-29 | M. abscessus complex | MABSC CF isolate, macrolide-R, | 0.125 | 0.125 |
| | | amikacin-R | | |
| AMT 0157-14 | M. abscessus complex | MABSC CF isolate, macrolide-R, | 0.125 | 0.125 |
| | | amikacin-I | | |
| AMT 0130-8 | M. abscessus complex | MABSC CF isolate, macrolide-S, | 0.125 | 0.0625 |
| | | amikacin-I | | |
| AMT 0153-9 | M. abscessus complex | MABSC CF isolate, macrolide-S, | 0.25 | 0.125 |
| | | amikacin-I | | |
| AMT 0068-40 | M. abscessus complex | MABSC CF isolate, macrolide-S, | 0.25 | 0.125 |
| | | amikacin-1 | | |
| AMT 0119-7 | M. abscessus complex | MABSC CF isolate, macrolide-S, | 0.125 | 0.125 |
| | | amikacin-I | | |
| AMT 0493-2 | M. abscessus complex | MABSC CF isolate, macrolide-S, | 0.5 | 0.5 |
| | | amikacin-R | | |

TABLE 3B
BisEDT and BisBDT MIC against control bacterial strains

| ATCC strains used | | | | |
|---|---|---|---|---|
| to control for | Expected results: goal | | | |
| drug activity | (acceptable range) mcg/mL | | | |
| (see detailed data for measured | | BisEDT | | BisBDT |
| MICs with each assay set-up) | | MIC | | MIC |
| ATCC 27853 | P. aeruginosa | 1-2 | (0.5-4) | NA |
| ATCC 25922 | E. coli | 1 | (0.5-2) | NA |
| ATCC 29213 | S. aureus | 0.25-0.5 | (0.125-1) | 0.25 (0.125- |

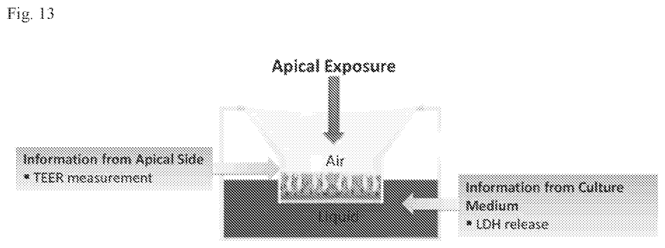

Controlled in vitro evaluation of BisEDT, with respect to potential cytotoxicity on fully differentiated human airway epithelium was carried out. Both solubilized and solid (powder/particulate) forms of BisEDT were evaluated for cytotoxicity by Epithelix, a contract research organization (CRO) specializing in this form of cytotoxicity evaluation utilizing a proprietary, in vitro fully differentiated human airway epithelium test system (MucilAir™). The MucilAir system facilitated evaluation of cytotoxicity through both LDH release (from the culture medium side) and trans-epithelial electrical resistance (TEER) from the apical/air-exposed side as well as pre- and post-exposure microscopic examination for morphological changes (FIG. 13).

With respect to evaluation of six concentrations of solubilized BisEDT, no changes in cellular morphology were noted at four different time points (up to 48 hours), nor were changes noted in LDH release and TEER, indicating that no toxic effects were observed, at all time points even at the highest tested concentration of 30 uM (20.83 ug/mL, which is approximately 20-347 fold higher than the MICs recently derived, against most tested CF bacterial isolates, including M. abscessus (including amikacin- and clarithromycin/macrolide-resistant strains), Burkholderia spp. (including beta-lactam, fluoroquinolone-, and sulfamethoxazole-resistant strains), P. aeruginosa (including multidrug-resistant (MDR) strains), Achromobacter spp., Stenotrophomonas maltophilia, and S. aureus (including MRSA)). The highest tested concentration of 20.83 μg/mL is also approximately 2.5-10 fold higher than the MIC for M. avium, which comparatively, had the highest MICs relative to BisEDT of any tested CF isolates. Additionally, since no hint of toxicity has been demonstrated with even the highest concentration of solubilized BisEDT, it is possible that higher concentrations may also be determined to be non-toxic/safe/well-tolerated.

With respect to evaluation of five concentrations of particulate BisEDT diluted in dextran as a carrier (weight/area), the lower two concentrations did not produce changes in cellular morphology at the four different time points (up to 48 hours), nor were changes noted in LDH release and TEER, indicating that no toxic effects were observed at any of the four time points.

With respect to morphology, the top three concentrations demonstrated changes that were more pronounced in the top two concentrations. With respect to the top three concentrations, no significant effect was noted with respect to TEER at any of these concentrations at the 1 hour time point, but at 8, 24, and 48 hours, TEER, loss of tissue integrity occurred with all three of the highest concentrations. Similarly, no effect on LDH release was noted at any time point with the lower two concentrations, nor at 1 hour for the top three concentrations; but beyond the 1 hour time point, there was a time and dose dependent increase in LDH release and decrease in TEER, indicating cytotoxicity under these conditions. It is notable that in Phase 1 human clinical studies, concentration of up to 2500 μg/cm² (˜8-fold higher than the 333 μg/cm², the highest concentration tested in this in vitro study) was well-tolerated when administered topically humans for 6 hours, and a concentration of up to 75 μg/cm² (intermediate between 33.3 μg/cm² and 333 μg/cm²) was tolerated when administered topically to humans for over 21 days continuous exposure to normal and abraded skin. This indicates that in vitro model conditions may be much more sensitive to the particulate form than in vivo physiologic conditions, thus over-representing the true risk of cytotoxicity of the particulate form. Nevertheless, the highest non-toxic concentrations of both forms of BisEDT tested in this in vitro cytotoxicity model, whether particulate or solubilized, provides 25-345 fold the MIC needed to be effective against a broad spectrum of antibiotic-resistant, difficult to treat CF bacterial pathogens.

The data from this study demonstrates that the solubilized version of BisEDT is safe at sufficient multiples of the anticipated clinical dose.

### Activity of BT Compounds Against (CF-Isolate Biofilms

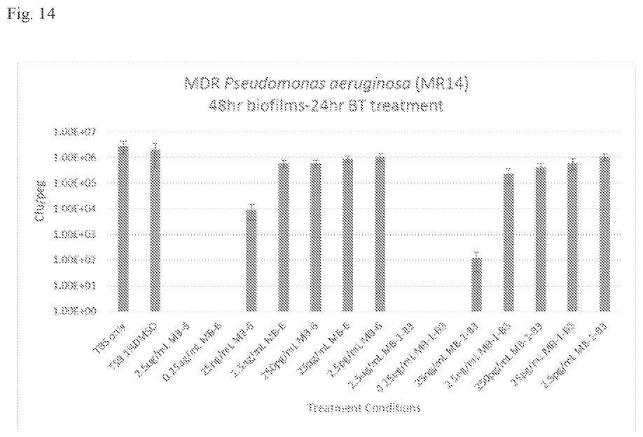

The activity of BT compounds against biofilms grown from CF-isolates was tested. MR14 is a multidrug-resistant (MDR) CF-isolate of Pseudomonas aeruginosa. Reductions in biofilm cell viability of 2 logs (MB-6) to 4 logs (MB-1-B3) occurred at 25 ng/mL (FIG. 14). The bismuth-thiol compounds have previously been reported in the literature to have anti-biofilm effects at subinhibitory concentrations with 24 hour treatment with 0.25 μg/mL. There is nothing comparable in the scientific literature.

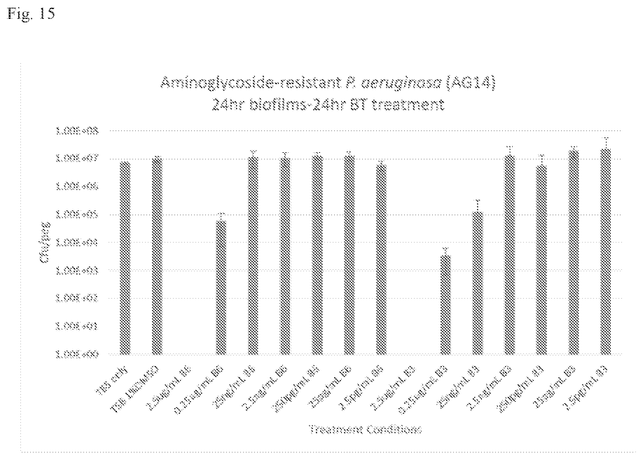

AG14 is an aminoglycoside-resistant CF-isolate of Pseudomonas aeruginosa. Reductions in biofilm cell viability of 2 logs (MB-6) to approximately 3.5 logs (MB-1-B3) occurred at 0.25 μg/mL. Once again, a very advanced level of anti-biofilm activity (a 6 log reduction) with 24 hour treatment occurred with 0.25 μg/mL; this potent level of activity is very likely to be unique to the bismuth-thiol compounds (FIG. 15).

Combined, the results of testing against Pseudomonal biofilms (MR14 and AG14) demonstrate an advanced, possibly unique level of anti-biofilm activity against antibiotic- and multidrug-resistant (MDR) Pseudomonas aeruginosa; this may represent an important new therapeutic activity and clinical strategy in the treatment of pulmonary infections associated with cystic fibrosis.

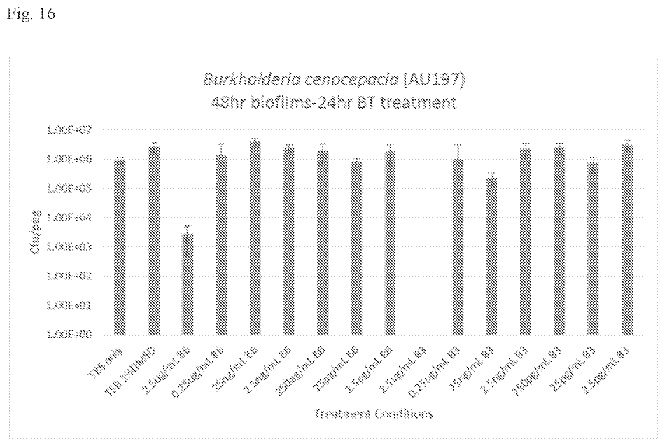

AU197 is a CF-isolate of B. cenocepacia. While in this case, the anti-biofilm activity is not occurring at a subinhibitory level (as with the previous examples of P. aeruginosa), this level of anti-biofilm activity (a 6 log reduction at a concentration of 2.5 μg/mL of BisEDT) is nevertheless extremely potent, and is very likely to be therapeutically achievable (FIG. 16).

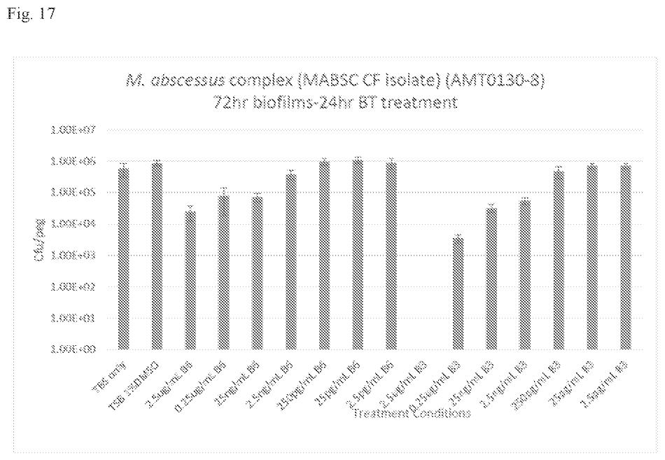

AMT0130-8 represents a CF-isolate of the clinically relevant MABSC, which frequently complicates the treatment of CF pulmonary infections. In this case, while BisBDT demonstrated only very modest reductions in biofilm cell viability, once again, BisEDT demonstrated a 6 log reduction at 2.5 μg/mL, as well as a dose response from 2.5 ng/mL to 2.5 μg/mL. Interestingly, the MIC against this strain was demonstrated to be lower for BisBDT (0.0625 μg/mL) than for BisEDT (0.125 μg/mL), yet the anti-biofilm activity of BisEDT was apparently demonstrated to be much more potent—while it is not surprising to see such differences in activity between distinct bismuth-thiol compounds, this particular MABSC strain was apparently technically difficult to work with (see bullet point notes below figure), which may also have accounted for such (apparent) differences in activity (FIG. 17).

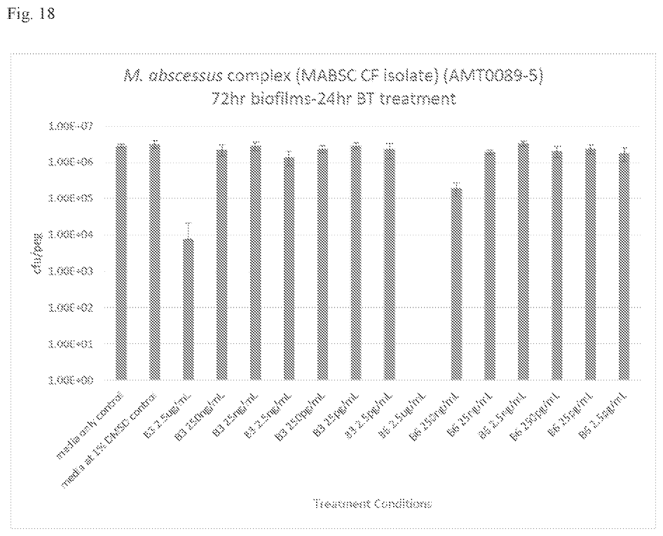

AMT0089-5 is a macrolide-resistant, amikacin-resistant MABSC. The involvement of such antibiotic-resistant strains of MABSC in the pulmonary infections of CF patients is extremely problematic. Here, while BisEDT showed a 2.5 log reduction in viable biofilm cells at a concentration of 2.5 μg/mL, BisBDT was demonstrated to have reduce viable biofilm cells by 6 logs at a concentration of 2.5 μg/mL (FIG. 18).

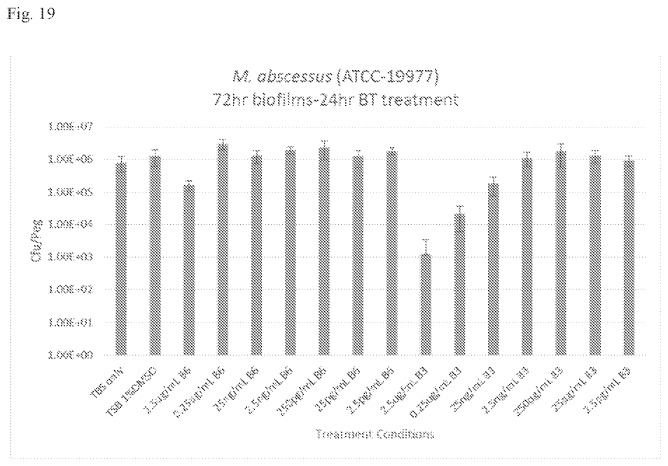

ATCC-19977 is M. abscessus (macrolide-resistant; inducible). A dose response is demonstrated showing a 3 log reduction in viable biofilm cells at 2.5 μg/mL ATCC-19977 is M. abscessus (macrolide-resistant; inducible). A dose response is demonstrated below, with a 3 log reduction in viable biofilm cells at 2.5 μg/mL (FIG. 19).

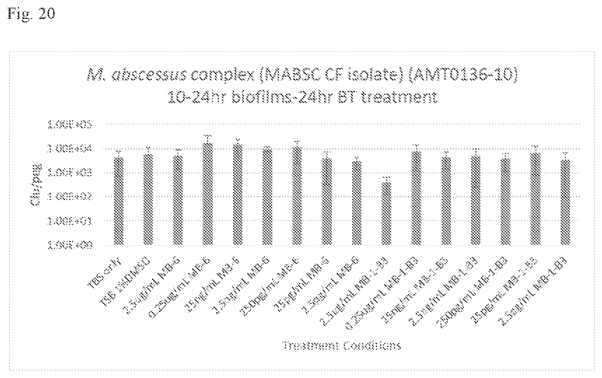

The bismuth-thiols were not observed to be active against biofilm formed by a MABSC CF isolate, though this strain was so slow-growing, a longer exposure to the bismuth-thiol compounds may have been necessary to demonstrate activity (FIG. 20).

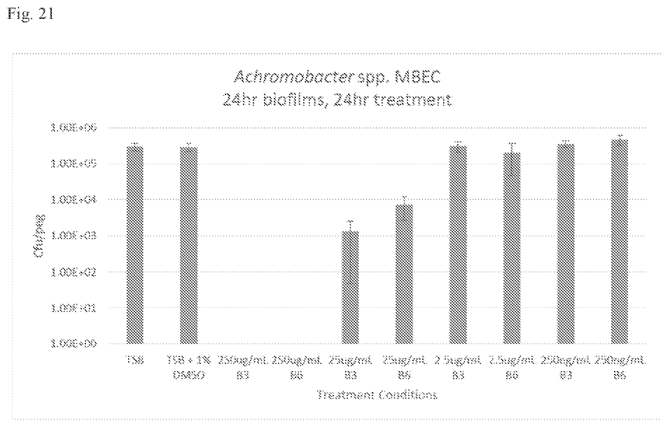

Achromobacter spp. were tested up to concentrations of 250 μg/mL of both BisEDT and BisBDT, which resulted in a 5 log reduction in viable biofilm cells. A dose response is also apparently associated with both compounds (FIG. 21).

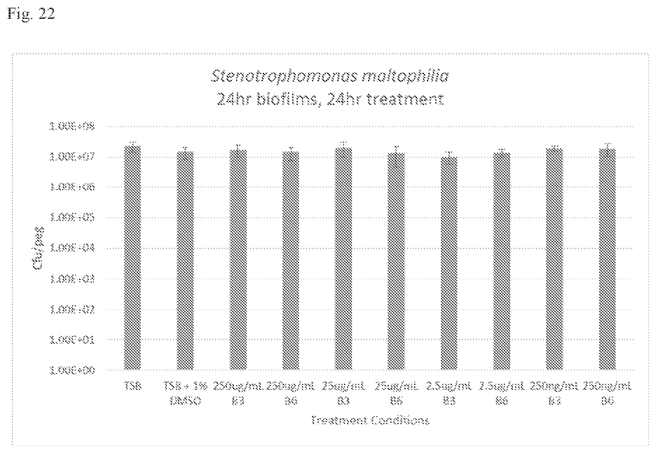

Unfortunately, no activity was apparent for either bismuth-thiol compound against Stenotrophomonas maltophilia (FIG. 22).

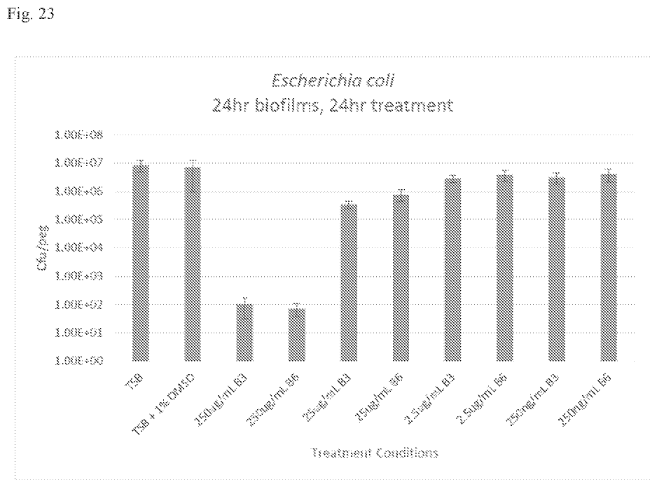

Finally, anti-biofilm activity was also demonstrated at the highest concentrations of both compounds (a 5 log reduction) when tested against E. coli (FIG. 23).

Both BisEDT and BisBDT are demonstrated to have very low MIC values against M. abscessus, MDR P. aeruginosa, Achromobacter spp., and Burkholderia spp. As before, ATCC control strains are utilized to standardize the data. However, in this evaluation, the bismuth-thiols were compared head to head with amikacin and clarithromycin (a clinically important macrolide antibiotic). As can be seen from this data below in Table 4, the bismuth-thiols are notably and consistently more potent than both amikacin and clarithromycin (most dramatically when considering the MABSC strain ATCC 19977, which was induced to be macrolide-resistant).

TABLE 4A
Comparison of conventional antibiotics vs BisEDT and BisBDT activity against bacterial strains
SUMMARY OF MIC RESULTS

| | | Special Strain | MIC | MIC | MIC | MIC |
|---|---|---|---|---|---|---|
| Strain | | characteristics | (mcg/mL) | (mcg/mL) | (mcg/mL) | (mcg/mL) |
| Designation | Species | (if any) | Amikacin | Clarithromycin | Bis-EDT | Bis-BDT |
| ATCC 19977 | M abscessus/massiliense complex | Macrolide resistant | 8 ² | >32 ² | 0.06 ² | <0.03 ² |
| | | (inducible) | | | | |
| AMT0130-8 | M abscessus/massiliense complex | | 16 ² | 1 | 0.06 ² | <0.03 ² |
| AMT153-9 | M abscessus/massiliense complex | | 32 ² | 2 | 0.13 ² | <0.03 ² |
| AMT0068-40 | M abscessus/massiliense complex | | 32 ² | 1 | 0.25 ² | 0.06 ² |
| AMT0119-7 | M abscessus/massiliense complex | | 32 ² | 1 | 0.06 ² | <0.03 ² |
| AMT0493-2 | M abscessus/massiliense complex | Amikacin resistant | >64 ² | 2 | 0.5 ² | 0.125 ² |
| AGR1 | P. aeruginosa | | 16 | na | 1 | 1 |
| AGR14 | P. aeruginosa | Multi-drug resistant | >64 | na | 0.5 | 1 |
| MR14 | P. aeruginosa | Multi-drug resistant | >64 | na | 1 | 1 |
| SM21 | S. maltophilia | | 64 | na | 0.25 | 0.13 |
| AX1 | Achromobacter spp. | | 64 | na | 1 | 1 |
| AX4 | A. xylosoxidans | | >64 | na | 0.2.5 | 0.5 |
| BC5b | B. multivorans (B cepacia complex) | | >64 | na | 0.25 | 1 |
| BC15 | B. cenocepacia (B cepacia complex) | | >64 | na | 2 | 4 |
| BC17 | B. cepacia (B cepacia complex) | | >64 | na | 8 | 8 |
| AU197 | B. cenocepacia (B cepacia complex) | | >64 | na | 0.5 | 4 |

TABLE 4B
BisEDT and BisBDT MIC against control bacterial strains

| ATCC or other strains used to | | | | | | | |
|---|---|---|---|---|---|---|---|
| control for drug activity | | | | | | | |
| (see detailed data for measured | | Expected results (acceptable range) mcg/mL | | | | | |
| MICs with each assay set-up) | | (per CLSI M100-S24 or provided by Microbion) | | | | | |
| Strain | Species | Amikacin | | Clarithromycin | BisEDT | | BisBDT |
| ATCC 29213 | S. aureus | 2 | (1-4) | 0.25 (0.12-0.5) | 0.25-0.5 | (0.13-1) | 0.25 (0.12-0.5) |
| ATCC 25922 | E. coli | 1-2 | (0.5-4) | na | 1 | (0.5-2) | na |

Evaluation of BT compound effect on cytotoxicity on a fully differentiated human airway epithelium (MucilAir™)

The aim of this study is to evaluate the potential local toxic effect of BisEDT on airway epithelium. The project is divided into 2 phases: Study 1: Acute Toxicity testing of BisEDT in solution; Study 2: Acute Toxicity testing of BisEDT as solid.

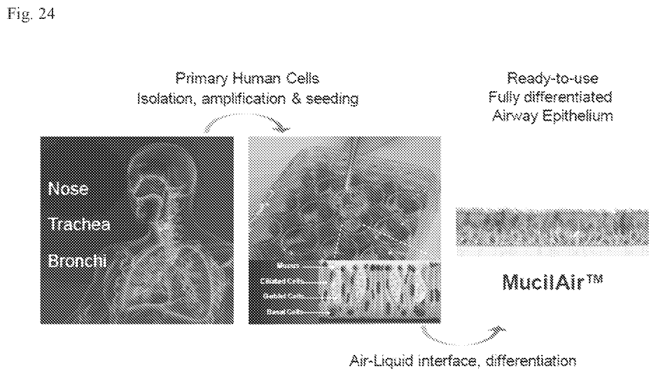

The assay system used in this study is Epithelix's proprietary technology MucilAir™ MucilAir™ is a fully differentiated and ready-to-use 3D model of human airway epithelium, constituted with primary human epithelial cells freshly isolated from nasal, tracheal or bronchial biopsies. MucilAir™ (FIG. 24), is not only morphologically and functionally differentiated, but can also be maintained in a homeostatic state for a long period of time (Huang et al., 2009).

MucilAir™ M is composed of basal cells, ciliated cells and mucus cells. The proportion of these various cell types is preserved compared to what one observes in vivo (Huang et al., 2011). Moreover the epithelia are started from de-differentiated cells. The cells undergo a progressive differentiation with time. After 45 days of culture, the epithelia are fully ciliated, secret mucus and are electrically tight (TEER>200Ω.cm²). The activity of the main epithelial ionic channels, such as CFTR, EnaC, Na/K ATPase, is preserved and the epithelia is shown to respond in a regulated and vectorial manner to the pro-inflammatory stimulus, TNF-α (Huang et al., 2011). A large panel of cytokines, chemokines and metalloproteinases has been detected in MucilAir™ (e.g. 1L-8, IL-6, GM-CSF, MMP-9, GRO-α).

Acute Toxicity Testing of BisEDT in solution

The aim of this phase is to evaluate the potential acute toxicity of BisEDT in solution once applied at the apical surface on a 3D model of fully differentiated human airway epithelium (MucilAir™) after 1, 8, 24 and 48 hours exposure.

TABLE 5
patient information

| Batch | Age of the | Sex of the | Age of the | Special |
|---|---|---|---|---|
| number | patient | patients | culture | comments |
| MD014101 | 38 years | ND | 105 days | Normal donor |

TABLE 6
Test material

| Identification | | | |
|---|---|---|---|
| Name | Concentrations | Vehicle | Solubility |
| BisEDT | 0.001, 0.01, 0.1, | 0.5% DMSO in | OK |
| (MB-1-B3) | 1, 10, 30 μM | Buffered Saline | |

BisEDT (MB-1-B3) was applied on the apical surface of MucilAir™ during 1, 8, 24 and 48 hours (FIG. 13). The effect of 6 concentrations was studied: 0.001, 0.01, 0.1, 1, 10 and 30 μM. The compound was diluted in a buffered saline solution (NaCl 0.9%-1.25 mM CaCl₂-10 mM HEPES) with 0.5% DMSO. 30 μl solution at the selected concentration was applied on the apical surface of MucilAir™. The negative control corresponds to the vehicle solution (0 μM) and untreated cultures. The positive control corresponds to 50 μl to 10% Triton X-100 diluted in a buffered saline solution. The study was run in triplicates.

During the study, inserts were maintained in a C02 incubator (37° C., 5% C02, 100% humidity). The following end-points were determined:
Tissue integrity monitoring: Trans-Epithelial Electrical Resistance (TEER) measurement (quantitative) at D0, D1 and D2.
Cytotoxicity monitoring: LDH test (quantitative) at D0, D1 and D2.
Morphology: cellular and tissue integrity examined under contrast microscope at D0, D1 and D2.

Three days before the experiment, the following quality controls were performed on each epithelium:
Washing: inserts were washed with 200 μl of MucilAir™ culture medium. Washing removes accumulated mucus on the tissue surface which may interfere with the test.
Trans-Epithelial Electrical Resistance (TEER): TEER was measured to verify that all the selected inserts had tight epithelial barriers and the tissue itself was not disrupted prior to application of the test material.
Tissue morphology: each insert was inspected under a conventional inverted microscope to ensure the quality of the epithelia and determine qualitatively that cilia motion was visible. The presence of mucus was detected by the refractive aspect of the apical surface.

### Results

Error bars in the following graphs refer to standard error of the mean (SEM). All comparisons are versus the negative control (vehicle solution, 0 μM).

Before exposure: Each insert was inspected under a conventional inverted microscope to insure the quality of the epithelia. The movement of cilia was clearly visible for all the selected inserts. The presence of mucus was detected by the refractive aspect of the apical surface.

After exposure: No morphology changes were observed for the vehicle control and all tested concentrations and time points.

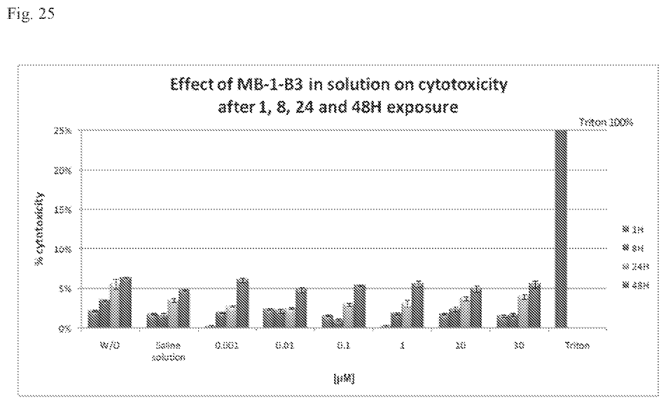

Cytotoxicity assessment: FIG. 25 shows the percentage of cytotoxicity (LDH measurement) at 1, 8, 24 and 48 hours exposure. The control (0 μM, vehicle solution) corresponds to a physiological release of LDH (<5%). LDH release was not altered after exposure to BisEDT at all tested concentrations and at all time-points. Therefore, no toxic effect was observed.

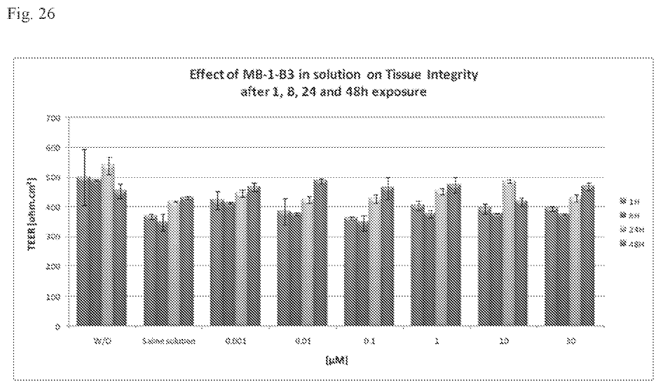

Tissue integrity assessment: FIG. 26 shows the monitoring of TEER at D1. It should be noted that TEER is a dynamic parameter that can be affected by several factors. A decrease of the ionic channel activity can lead to an increase of TEER, and an activation of the ion channels can decrease TEER values. When an epithelium is damaged, a decrease of TEER would be associated with an increase of LDH release. BisEDT didn't show significant effect on TEER at all tested concentrations and time points.

### Acute Toxicity Testing of BisEDT as Solid: 1^{st} Set of Experiments

The aim of this study is to evaluate the potential acute toxicity of BisEDT as solid (at 0.033, 0.33, 3.33, 33.3, 333 μg/cm²) once applied at the apical surface on a 3D model of fully differentiated human airway epithelium (MucilAir™) after 1, 8, 24 and 48 hours exposure.

TABLE 7
Tissues (Patient information)

| Batch | Age of the | Sex of the | Age of the | Special |
|---|---|---|---|---|
| number | patient | patients | culture | comments |
| MD014101 | 38 years | ND | 119 days | Normal donor |

TABLE 8
Test Material

| Identification | | |
|---|---|---|
| Name | Concentrations | Vehicle |
| MB-1-B3 | 0.033, 0.33, 3.33, | Dextran powder (C60) |
| | 33.3, 333 μg/cm² | Ref: Pharmacosmos |
| | | 5510 0060 1007 |

Compound BisEDT was applied on the apical surface of MucilAir™. The effect of 5 concentrations after 1, 8, 24 and 48 hours exposure was studied: 0.033, 0.33, 3.33, 33.3, 333 μg/cm2. The compound was diluted in Dextran powder at the targeted concentration and compressed in order to obtain a tablet. The study was run in triplicates. During the study, inserts were maintained in a CO₂ incubator (37° C., 5% CO₂, 100% humidity). The mucociliary clearance analysis was performed after 1 hour and 24 hours exposure.

Quality control and washing of the apical surface: Three days before the experiment, the following quality controls were performed on each epithelium:
Washing: inserts were washed with 200 μl of MucilAir™ culture medium. Washing removes accumulated mucus on the tissue surface which may interfere with the test.
Trans-Epithelial Electrical Resistance (TEER): TEER was measured to verify that all the selected inserts had tight epithelial barriers and the tissue itself was not disrupted prior to application of the test material.
Tissue morphology: each insert was inspected under a conventional inverted microscope to ensure the quality of the epithelia and determine qualitatively that cilia motion was visible. The presence of mucus was detected by the refractive aspect of the apical surface.

### Results

Error bars in the following graphs refer to standard error of the mean (SEM). All comparisons are versus the negative control (Carrier, Dextran).

Before exposure: Each insert was inspected under a conventional inverted microscope to insure the quality of the epithelia. The movement of cilia was clearly visible for all the selected inserts. The presence of mucus was detected by the refractive aspect of the apical surface.

After exposure: No morphology changes were observed for the non-treated and the vehicle controls.
0.033 and 0.33 μg/cm²: No morphological modifications were observed.
3.33 μg/cm²: The tablets applied apically were poorly dissolved on the epithelia at 24 and 48 h after exposure. Cells were rounded and opaque at the periphery of inserts. Gradually the cells became detached from each other. The appearance of culture medium coming from the basal side on the apical surface was observed.
33.3 and 333 μg/cm²: The tablets applied apically was poorly dissolved on the epithelia at 8, 24 and 48 h after exposure. Cells were rounded and opaque on the inserts. Gradually the cells became detached from each other. The culture medium leaked to the apical side.

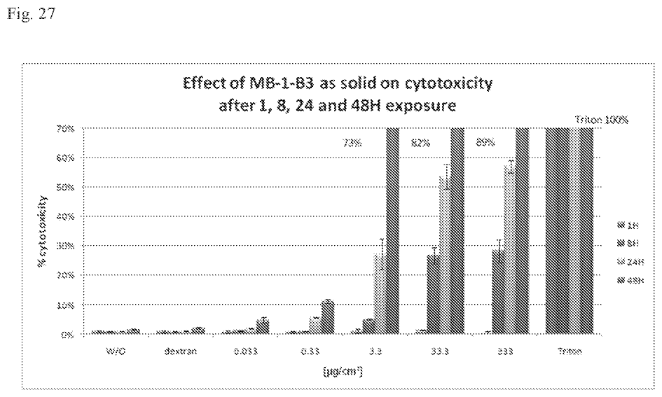

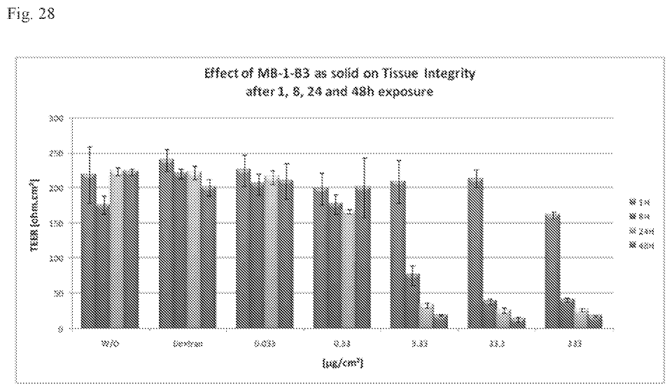

Cytotoxicity assessment: FIG. 27 shows the percentage of cytotoxicity (LDH measurement) at D1. The control (Dextran) corresponds to a physiological release of LDH (<5%). No significant effect on LDH release at 0.033 and 0.33 μg/cm² at all tested time points. A time and dose dependent, cytotoxicity is observed at 3.3; 33.3 and 333 μg/cm².

Tissue integrity assessment: FIG. 28 shows the monitoring of TEER at D1. It should be noted that TEER is a dynamic parameter that can be affected by several factors. A decrease of the ionic channel activity can lead to an increase of TEER, and an activation of the ion channels can decrease TEER values. When an epithelium is damaged, a decrease of TEER would be associated with an increase of LDH release. No significant effect was observed on TEER at 0.033 and 0.33 μg/cm² at all tested time points. After 1 hour exposure, no significant effect on TEER is observed at all tested doses. Loss of tissue integrity is observed at 3.33, 33.3 and 333 μg/cm² after 8, 24 and 48H exposure.

### Acute Toxicity Testing of BisEDT as Solid: 2^{nd} Set of Experiments

The aim of this phase is to evaluate the potential acute toxicity of BisEDT as solid (at 1 μg/cm²) once applied at the apical surface on a 3D model of fully differentiated human airway epithelium (MucilAir™) after 1, 8, 24 and 48 hours exposure.

TABLE 9
Patient Information

| Batch | Age of the | Sex of the | Age of the | Special |
|---|---|---|---|---|
| number | patient | patients | culture | comments |
| MD014101 | 38 years | ND | 125 days | Normal donor |

TABLE 10
Compound Information

| Identification | | |
|---|---|---|
| Name | Concentrations | Vehicle |
| MB-1-B3 | 1 μg/cm² | Dextran, powder (C60) |
| | | Ref: Pharmacosmos |
| | | 5510 0060 1007 |

Testing Strategy and Protocol: Compound BisEDT was applied on the apical surface of MucilAir™. The effect of 1 μg/cm² concentrations after 1, 8, 24 and 48 hours exposure was studied. The compound was diluted in Dextran powder at the targeted concentration and compressed in order to obtain a tablet. The study was run in triplicates. During the study, inserts were maintained in a CO² incubator (37° C., 5% CO2, 100% humidity). The mucociliary clearance analysis was performed after 1 hour and 24 hours exposure.

Quality control and washing of the apical surface: Three days before the experiment, the following quality controls were performed on each epithelium:
Washing: inserts were washed with 200 μl of MucilAir™ culture medium. Washing removes accumulated mucus on the tissue surface which may interfere with the test.
Trans-Epithelial Electrical Resistance (TEER): TEER was measured to verify that all the selected inserts had tight epithelial barriers and the tissue itself was not disrupted prior to application of the test material.
Tissue morphology: each insert was inspected under a conventional inverted microscope to ensure the quality of the epithelia and determine qualitatively that cilia motion was visible. The presence of mucus was detected by the refractive aspect of the apical surface.

### Results

Error bars in the following graphs refer to standard error of the mean (SEM). All comparisons are versus the negative control (Carrier Dextran).

### Morphology

Morphology, Before exposure: Each insert was inspected under a conventional inverted microscope to insure the quality of the epithelia. The movement of cilia was clearly visible for all the selected inserts. The presence of mucus was detected by the refractive aspect of the apical surface.

Morphology, After exposure: No morphology changes were observed for the non-treated and the vehicle controls. 1 μg/cm²: after 1 hour and 8 hour exposure, no important morphological changes were observed. After 24 h exposure, the epithelial cells on 2 inserts out of 3 were dead. After 48 h the epithelial cells on all 3 inserts were dead.

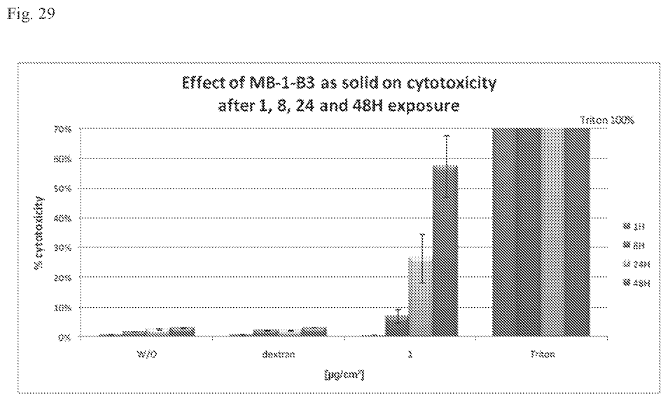

Cytotoxicity assessment: FIG. 29 represents the percentage of cytotoxicity (LDH measurement) at D1. The control (Dextran) corresponds to a physiological release of LDH (<5%). No significant effect on LDH release at 1 μg/cm² after 1 hour exposure. A time- and dose-dependent cytotoxicity is observed at 8, 24 and 48 h after exposure.

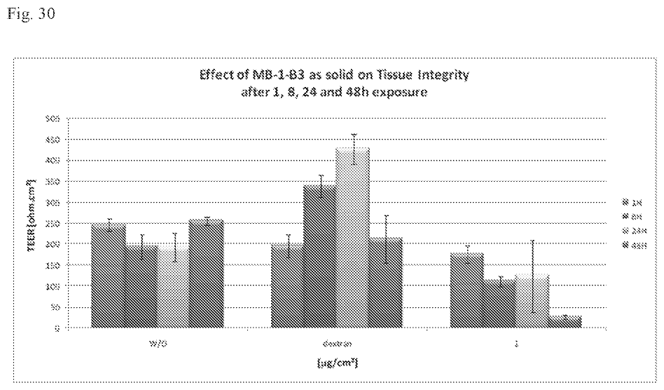

Tissue Integrity Assessment: FIG. 30 represents the monitoring of TEER at D1. It should be noted that TEER is a dynamic parameter that can be affected by several factors. A decrease of the ionic channel activity can lead to an increase of TEER, and an activation of the ion channels can decrease TEER values. When an epithelium is damaged, a decrease of TEER would be associated with an increase of LDH release.

After 1 hour exposure, no significant effect on TEER was observed at 1 μg/cm². Loss of tissue integrity is observed at 1 μg/cm² after 8, 24 and 48H exposure. After 48H exposure, the epithelia were no more tight.

### CONCLUSIONS

The aim of this study is to evaluate the potential local toxic effect of BisEDT on airway epithelium in liquid or solid solution. To evaluate potential effects of BisEDT exposure on fully differentiated human nasal epithelia cultured at the air-liquid interface, two sets of experiments were conducted namely BisEDT in liquid solutions or BisEDT as solid.

In general, within the presented study, BisEDT compound in liquid solution showed no local toxicity on the airway epithelium at different tested concentrations (0.001, 0.01, 0.1, 1, 10, 30 μM) and time exposure (1, 8, 24, and 48 hours). No effect on the morphology of the epithelia, on TEER and LDH release were observed.

Similar results were obtained when the compound was applied as solid at low doses (0.033 and 0.33 μg/cm² for 1, 8, 24, and 48 hour exposures). However, at 1 and 3.33 μg/cm², BisEDT induces cytotoxicity at 24 h and 48 h exposure, demonstrated by an increase of LDH release and a reduction of the TEER value. For higher concentrations (33.3, 333 μg/cm²), strong toxicity was observed with a high amount of LDH released, a very low TEER values and important morphological changes.

### REFERENCES

Huang, S; Caul-Futy, M; “A novel in vitro cell model of the human airway epithelium” 3R-Info-Bulletin No. 41, October 2009.
Huang. S., Wiszniewski, L., & Constant, S. (2011). The use of in vitro 3D cell models in drug development for respiratory diseases. Drug Discovery and Development—Present and Future

### Example 6: Sputum Studies

Bacterial killing curves with BisEDT and BisBDT were performed in the presence of three cystic fibrosis patient sputum in order to determine whether the test compounds are potentially inactivated by sputum. The assay is described in King P. Lomovskaya O, Griffith D C, Burns J L, Dudley M N. In vitro pharmacodynamics of levofloxacin and other aerosolized antibiotics under multiple conditions relevant to chronic pulmonary infection in cystic fibrosis. Antimicrob Agents Chemother. 54:143-8, 2010.

Sputum was collected from cystic fibrosis (CF) patient volunteers without recent antibiotic exposure after appropriate IRB approval. Sputum was sterilized by UV irradiation and sterilization was confirmed by culture. An overnight culture of Pseudomonas aeruginosa strain PA01 was used to inoculate fresh cultures in cation-adjusted Mueller-Hinton broth or cation-adjusted Mueller-Hinton broth plus 10% CF patient sputum. Drugs were added to individual culture tubes with and without sputum at the following final concentrations:

BisEDT: 0.1 μg/mL, 2 μg/mL, and 20 μg/mL

BisBDT: 0.1 μg/mL, 4 μg/mL, and 20 μg/mL

Tobramycin at 1 μg/mL was used as a comparator drug control known to be partially inactivated by patient sputum. Cultures were incubated with shaking at 37° C. and aliquots were removed every hour for quantitation of colony forming units per mL (CFU/mL) by serial dilution and plating on tryptic soy agar for a total of 6 hours. CFU were counted after incubation of the plates overnight at 37° C.

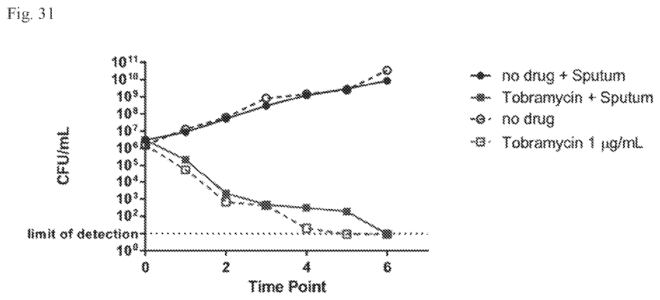

The controls for this assay performed as expected. The growth of PA01 was not obviously inhibited or enhanced by the addition of sterile patient sputum in the absence of additional drug to bacterial cultures (FIG. 31, closed and open circles). As shown, sputum partially inhibits the bacterial killing activity of tobramycin, with approximately 0.5-1 log CFU/mL higher at most time points in cultures with sputum compared to cultures without sputum.

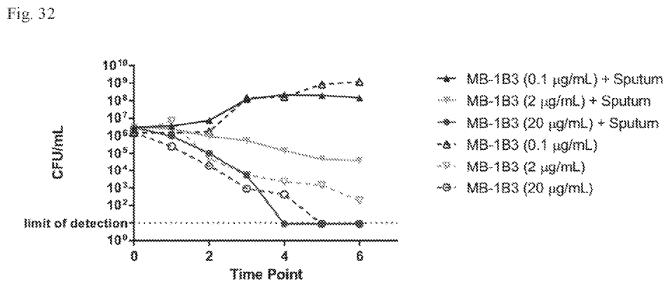

The bactericidal activity of BisEDT appears to be partially inhibited by CF patient sputum based on this assay (FIG. 32). BisEDT is not bactericidal against PA01 at 0.1 μg/mL. With BisEDT at 2 μg/mL, the addition of sputum reduces killing by approximately 1-2 log CFU/mL at 3 to 6 hours. When the concentration of BisEDT is further increased to 20 μg/mL the inhibition of bacterial killing by sputum is less pronounced, with killing in the presence of sputum lagging by only 0.5-1 log CFU/mL behind cultures without sputum at early time points (1-3 hours). Eventually, with this highest dose tested, bacteria in the culture with sputum is reduced below the limit of detection 1 hour earlier than the sample without sputum, suggesting that at higher drug concentrations inhibition of BisEDT by sputum is largely overcome.

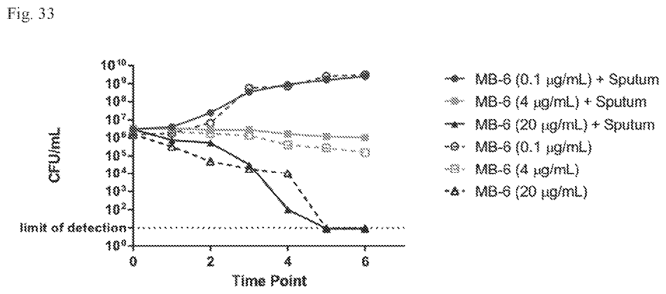

Similarly, the bactericidal activity of BisBDT appears to be partially inhibited by CF patient sputum (FIG. 33). BisBDT at 0.1 μg/mL is not bactericidal against PA01. In the absence of sputum, 4 μg/mL BisBDT demonstrated very slow bactericidal activity against PA01, with killing of only about 1 log over the 6 hour assay; cultures with sputum demonstrated approximately 0.5-0.8 log CFU/mL more surviving at 3-6 hours. At the highest concentration of BisBDT tested, 20 μg/mL, there is an initial lag in killing in PA01 at 1-2 hours with the addition of sputum, but both samples with and without sputum are sterilized below the limit of detection by 5 hours.

Both compounds BisEDT and BisBDT are bactericidal against Pseudomonas aeruginosa strain PA01 in this assay, and this bactericidal activity is partially inhibited in the presence of CF patient sputum. This partial inhibition of bactericidal activity can be overcome by increased concentration of the test compounds. Thus, a higher concentration of bismuth-thiol compound maybe needed in areas where sputum is present compared to bodily compartments without sputum.

### Example 7: In Vitro Activity of Bismuth Thiols and Comparators Against Haemophilus Influenza Clinical Isolates

In this Example, the activity of three Bismuth thiol compounds are evaluated against Haemophilus influenzae, a prevalent pathogen of respiratory disease including pneumonia, otitis media, conjunctivitis, and meningitis.

Test and Control Agents: The test compounds (BisEDT, and analogs BisBDT/PYR and BisEDT/PYR) were shipped from to Micromyx and stored at −20° C. until assayed. The solvent for all test compounds was 100% DMSO, the stock concentration was 2560 μg/mL, and the range tested was 64-0.06 μg/mL. All stock solutions were allowed to stand for at least one hour prior to use to auto-sterilize.

Comparator drugs were provided by Micromyx. Suppliers, lot numbers, diluent, stock concentrations, and test ranges were as follows.

| | | | | Concentration | |
|---|---|---|---|---|---|
| | | | | of Stock | |
| Test/Control | | | | Solution | Test Range |
| Agents | Supplier | Lot No. | Diluent | (μg/mL) | (μg/mL) |
| Azithromycin | USP | JOI240 | DMSO | 2560 | 64-0.06 |
| Ampicillin | Sigma | BCBF0407V | Sorenson | 1280 | 32-0.03 |
| | | | Buffer pH 7.5 | | |
| Cefuroxime | Sigma | 031M0823V | dH₂O | 2560 | 64-0.06 |
| Levofloxacin | Sigma | BCBC2112V | DMSO | 2560 | 64-0.06 |
| | | | | | 1-0.001 |

### Test Organisms

The test organisms were maintained frozen at −80° C. The organisms were originally acquired from the American Type Culture Collection or from clinical laboratories. The isolates were sub-cultured on Chocolate Agar plates (Remel; Lenexa, Kans.) and incubated overnight at 35° C. with 5% C02. H. influenzae ATCC 49247 was tested for the purposes of quality control for the comparator compounds.

Minimal Inhibitory Concentration (MIC) Assay Media: The medium employed for the broth microdilution MIC assay was Haemophilus Test Medium (HTM; Remel; Lenexa, Kans.; Catalog No. R112380; Lot No. 056737) as recommended by the Clinical Laboratory Standards Institute (CLSI; 1).

Broth Dilution Minimal Inhibitory Concentration (MIC) Assay Procedure: MIC values were determined using a broth microdilution method as recommended by CLSI (1, 2). Automated liquid handlers (Multidrop 384, Labsystems, Helsinki, Finland; Biomek 2000 and Biomek F/X, Beckman Coulter, Fullerton Calif.) were used to conduct serial dilutions and make liquid transfers.

The wells of a standard 96-well microdilution plate (Costar 3795; Corning Inc.; Corning, N.Y.) were filled with 150 μL of the appropriate solvent in columns 2-12 on the Multidrop 384. This plate was used to prepare the drug “mother plate” which provided the serial drug dilutions for the replicate “daughter plates”. The Biomek 2000 was used to transfer 150 μl of each stock solution from the wells in Column 1 of the mother plate to make ten subsequent 2-fold serial dilutions. The wells of Column 12 contained no drug and were the organism growth control wells. Each mother plate has the capacity to create a total of 15 daughter plates.

The daughter plates were loaded with 185 μL of HTM using the Multidrop 384. The wells of the daughter plates ultimately contained 185 μL of HTM, 5 μL of drug solution, and 10 μL of inoculum. The daughter plates were prepared on the Biomek F/X instrument which transferred 5 μL of drug solution from each well of the mother plate to each corresponding well of each daughter plate in a single step.

A standardized inoculum of each organism was prepared per CLSI methods (1). The inoculum for each organism was dispensed into sterile reservoirs divided by length (Beckman Coulter), and the Biomek 2000 was used to inoculate the plates. Daughter plates were placed on the Biomek 2000 work surface reversed so that inoculation takes place from low to high drug concentration. The Biomek 2000 delivered 10 μL of standardized inoculum into each well. These dilutions yielded a final cell concentration in the daughter plates of approximately 5.0×105 colony-forming-units/mL.

Plates were stacked three high, covered with a lid on the top plate, placed in plastic bags, and incubated at 35° C. for approximately 24 hr. Following incubation, the microplates were removed from the incubator and viewed from the bottom using a plate viewer. An un-inoculated solubility control plate was observed for evidence of drug precipitation. The MIC was read and recorded as the lowest concentration of drug that inhibited significant visible growth of the organism.

### Results

Examination of the un-inoculated drug solubility plates revealed no precipitation of any of the evaluated agents over the tested concentration range. Against H. influenzae ATCC 49247, evaluated comparators had MICs within the acceptable range for quality control established by CLSI (2) where applicable.

All Bismuth Thiol test compounds were active against all evaluated H. influenzae strains regardless of resistance to other agents, including isolates which were beta-lactamase negative ampicillin-resistant (BLNAR). The MICs of BisEDT and analogs BisEDT/PYR and BisBDT/PYR were all at or below the lowest concentration evaluated in the study (<0.06 μg/mL). This activity was greater than azithromycin (MIC₅₀ and MIC₉₀ of 2 μg/mL), ampicillin (MIC₅₀ of 2 μg/mL and MIC₉₀ of 4 μg/mL), and cefuroxime (MIC₅₀ of 4 μg/mL and MIC₉₀ of 16 μg/mL). Levofloxacin, which was tested down as low as 0.001 μg/mL for purposes of quality control had an MIC₅₀ and MIC₉₀ of 0.015 μg/mL. As the Bismuth Thiol compounds were tested from 0.06-64 μg/mL in this study, and their MICs were <0.06 μg/mL, it is not known whether their activity exceeds that of levofloxacin which was generally had MICs of 0.015 μg/mL.

This study demonstrated a high degree of potency for the tested Bismuth Thiol compounds against H. influenzae at levels exceeding that of ampicillin, azithromycin, and cefuroxime. This activity illustrates that H. influenzae is included as part of the broad spectrum of activity shown in vitro for this class of novel therapeutic agents.

### REFERENCES

1.) Clinical and Laboratory Standards Institute. Methods for Dilution Antimicrobial Susceptibility Tests for Bacteria That Grow Aerobically: Approved Standard-Ninth Edition. Clinical and Laboratory Standards Institute document M07-A9 [ISBN 1-56238-784-7]. Clinical and Laboratory Standards Institute, 940 West Valley Road, Suite 1400, Wayne, Pa. 19087-1898 USA, 2012.
2.) Clinical and Laboratory Standards Institute. Performance Standards for Antimicrobial Susceptibility Testing; Twenty-Second Informational Supplement. CLSI document M100-S22 [ISBN 1-56238-786-3]. Clinical and Laboratory Standards Institute, 940 West Valley Road, Suite 1400, Wayne, Pa. 19087 USA, 2012.

### Example 8: In Vivo Studies

Objectives: The primary objective of this study is to assess the tolerance of BisEDT (Dalton Pharma Services; Lot #ED268-1-11-01 stored at room temperature) following nose only inhalation exposure in F344 rats. Animals will be exposed to differing concentrations (low, middle, high) for up to 180 minutes. Blood will be collected at predetermined timepoints to analyze for systemic presence of BisEDT. At the conclusion of the study (24 hr post exposure) animals will be euthanized and undergo necropsy where lungs will be lavaged and collected for potential, future analysis. Additional respiratory tract related tissues such as the nasal cavity, larynx, pharynx, nasopharynx, trachea, bronchus, and carina will also be collected. Lavage fluid will be analyzed for clinical chemistry and hematology parameters including cell counts and differentials. Liver, kidney, esophagus, stomach, small intestine, and large intestine will also be collected for potential, future analysis.

Animals: Male and female F344 rats provided by Charles River Labs, Wilmington, Mass. were used in this study. The rats were approximately 7-9 weeks old at the time of arrival and 8-10 weeks at study initiation. Body weights of individual animals were ±20% of the group mean. Animals will be uniquely identified by numeric tail markings (made with indelible ink such as a Sharpie®) for body weights, randomization, and treatment administration. Color coded cage cards will also be placed on the cages. A total of forty (40) male and female F344 rats (20 M/20 F) (including spares) were ordered for the study. Thirty six (36) animals were randomized into 3 study groups each consisting of 12 animals per group (6 M/6F). The remaining 4 animals (2 M 12 F) were spares. Upon removal from quarantine, thirty six (36) rats will be randomized into 3 study groups each consisting of 12 animals (6 M/6 F) per group by body weight stratification. Unused spares will be either euthanized or conveyed to another approved study protocol. Prior to the start of exposures, animals will be conditioned to nose-only exposure tubes.

Animal Husbandry: Animals will be housed for a minimum of 7 days, up to 2 per cage in polycarbonate shoebox cages with Alpha Dri or hardwood chip bedding. Caging and bedding were autoclaved. Prior to injection, animals were introduced at least once to restraint tubes that will be used for tail vein dosing. Animal feed was 2016C Harlan Global Certified Rodent Chow, (Harlan Tekland, Madison, Wis.), unlimited access except during study procedures. Each batch of feed wass analyzed for contaminants by the manufacturer and will be used within the manufacturer's designated shelf-life. Animals were provided municipal water (filtered at 5, 1, and 0.2 μm), unlimited access except during study procedures. Only healthy animals were used in this study. A laboratory animal veterinarian or designee visually examined the animals before release from quarantine.

Environmental Conditions: The targeted conditions for animal room environment and photoperiod will be as follows: Temperature: 18-26° C.; Humidity: 30-70%; Light Cycle: 12-h. Light, humidity, and temperature excursions are defined as a sustained reading that falls out of the specified range for more than 3 hours.

Experimental Design: The experimental design for this study was: Group 1: Low dose; inhalation; 6 males and 6 females; blood collection at 0.5, 2 hr and 8 hr post exposure and 24 hr (terminal); and necropsy at 24 hours-post inhalation. Group 2: Mid dose; inhalation; 6 males and 6 females; blood collection at 0.5, 2 hr and 8 hr post exposure and 24 hr (terminal): and necropsy at 24 hours-post inhalation. Group 3: high dose; inhalation; 6 males and 6 females; blood collection at 0.5, 2 hr and 8 hr post exposure and 24 hr (terminal); and necropsy at 24 hours-post inhalation. Animals were weighed and randomized into study groups following the quarantine period. Animals were broken into 3 groups each consisting of 6 M and 6 F per group. Groups were exposed to three different dose levels of BisEDT. The initial exposure was to a formulation concentration of 100 mg/mL for an exposure time of 60 minutes; based on method development this is expected to result in a dose of 3 mg/kg. The maximum duration for exposures was 180 minutes and the maximum formulation concentration was 100 mg/mL.

Following exposure, non-terminal blood collections were performed on 2 animals per sex at 0.5, 2, and 8 hours post exposure. At the conclusion of the study (24 hr post exposure) animals were euthanized and underwent necropsy. The right lung was lavaged, flash frozen, and collected for potential, future analysis. The left lung was flash frozen for potential, future analysis. Additional respiratory tract related tissues such as the nasal cavity, larynx, pharynx, nasopharynx, trachea, bronchus, and carina were collected. Lavage fluid was analyzed for clinical chemistry and hematology parameters including cell counts and differentials. Liver, kidney, esophagus, stomach, small intestine, and large intestine were collected for potential, future analysis. These organ tissue samples were flash frozen and stored pending study completion. The lavage fluid were analyzed for clinical pathology and hematology (including cell counts and differentials). A maximum whole blood collection was also collected at necropsy. Blood samples were analyzed for clinical chemistry and hematology as well as an aliquot snap frozen for potential analysis for BisEDT.

Inhalation Exposure: The initial exposure of BisEDT (Bismuth-1,2-ethanedithiol) was formulated as a solution at 100 mg/mL in suspension in 0.5% Tween 80, 10 mM sodium phosphate, pH 7.4, in NaCl (adjusted to approximately 300 mOsm). Aerosols were generated with a commercial compressed air jet nebulizer, Pad LC Plus, operated with an inlet pressure of 20 psi. A schematic is shown in FIG. 1. The aerosols were transitioned into a rodent nose-only inhalation exposure system. The exposure system was operated with an inlet air flow of −5.2 L/min and an exhaust air flow of −5 L/min. This resulted in 0.31 Lmin to each port which is slightly greater than 1.5× the respiratory minute volume of a rat.

Exposure Concentration and Particle Size Monitoring: Aerosol concentration was monitored at the breathing zone by collection onto a GF/a filter. The filters were analyzed via differential mass and and other methods known in the art. Aerosol particle size was measured using a TSI Aerodynamic Particle Sizer (Model 3321, TSI, Inc., Shoreview, Minn.) or an In-Tox Mercer 2.0 L/min cascade impactor.

Observations and measurements: Observations were documented in Provantis database or the Animal Management System (AMS, LRRI, Albuquerque).

Clinical Observations and Mortality/Morbidity: Detailed clinical observations were recorded starting on dose day with observations recorded prior to exposure, during exposure, after exposure as the animals are returned to their home cages, and in the afternoon post-exposure. Observations will be recorded according to a standard lexicon (SOP TXP-1532-Pharmacologic and Toxicologic Observations of Experimental Animals). General observations include but are not limited to apnea, labored breathing, malaise, marked nasal discharge, etc. Special attention was paid to clinical signs associated with the respiratory tract. Animals showing severe signs of distress were euthanized immediately at the discretion of the Study Director in consultation with veterinary staff. Examinations were also oriented toward (1) identifying dead, weak, or moribund animals, and (2) documenting the onset and progression of any abnormal clinical signs. Moribund or dead animals were necropsied as soon as possible after being found but in no case later than 16 hours after being found.

Body Weights: All animals were weighed after release from quarantine and that weight will be the pre-study body weight used to randomize animals into dose groups. Body weights were collected in the morning prior to exposure and again at necropsy.

Blood Collection for Clinical Chemistry and Hematology: Blood samples for hematology and clinical chemistry were collected during necropsy. For Complete Blood Count (CBC) with absolute differentials, whole blood (target 0.5 mL) was collected and placed into tubes containing tripotassium ethylenediaminetetraacetate (K₃EDTA) as an anticoagulant. Hematology samples was analyzed by automated (ADVIA™ 120 Hematology System, Siemens Medical Solutions Diagnostics, Tarrytown, N.Y.) analyses. The parameters for hematology are: Red Blood Cell Count (RBC) 10⁶/μL; Hemoglobin (HGB) g/dL; Hematocrit (HCT) %; Mean Corpuscular Volume (MCV) fL; Concentration (MCHC) g/dL; Mean Corpuscular Hemoglobin (MCH) pg; Platelet Count (PLT) (10³/μL); Percent Reticulocytes (RETIC) % RBC; White Blood Cell Count (WBC) 10³/μL; Neutrophils (PMN) 10³/μL; Lymphocytes (LYM) 10³/μL; Monocytes (MONO) 10³/μL; Eosinophils (EOS)10³/μL; Basophils (BASO) 10³/μL; Large Unstained Cells (LUC)10³/μL.

For clinical chemistry analyses, whole blood (≥0.5 mL) was placed into serum separator or clot tube for centrifugation and separation into cellular and serum fractions. Serum samples were analyzed on a Hitachi Modular Analytics Clinical Chemistry System (Roche Diagnostics, Indianapolis, Ind.). The clinical chemistry parameters measured or calculated are: Alanine Aminotransferase (Alanine Transaminase)-Serum (ALT) IU/L; Albumin (ALB) g/dL; Aspartate Aminotransferase (Aspartate Transaminase)-Serum (AST) IU/L; Bilirubin (Total) (BILI-T) mg/dL; Blood Urea Nitrogen (BUN) mg/dL; Calcium (CA) mg/dL; Chloride (Serum) (CL-S) mmol/L; Cholesterol (Total) (CHOL) mg/dL; Creatinine (Serum)(CRE-S) mg/dL; Glucose (GLU) mg/dL; Gamma Glutamyltransferase (GGT) IU/L; Alkaline Phosphatase (ALP) IU/L; Phosphate (PHOS) mg/dL; Potassium (Serum) (K—S) mmol/L; Protein (Total) (TP) g/dL; Sodium (Serum) (NA-S) mmol/L; Triglycerides (TRIG) mg/dL; Albumin/Globulin (A/G) no units; Blood Urea Nitrogen/Creatinine (BUN/CRE) no units; Globulin (GLOBN) g/dL.

Hematology and clinical chemistry evaluations were performed on all study animals for which adequate sample volumes are obtained and for which no analytical problems are encountered. If target collection volumes are not obtained or if evaluations are not performed, a reason and notation will be included in the raw data. The remaining blood samples or serum will be discarded after the analyses.

Blood Collection for Bioanalytical Analysis: Following exposure, non-terminal blood collections were performed on 2 animals per sex at 0.5, 2, and 8 hours post exposure. Approximately 1 mL of systemic whole blood was collected by jugular vein into tubes containing K₃EDTA as an anticoagulant. The tubes were flash frozen with liquid nitrogen stored without processing at −70 to −90° C. until shipping for analysis using ICP-MS assay for quantitation of bismuth as a surrogate for BisEDT.

Euthanasia and necropsy: Animals were fasted overnight prior to scheduled necropsy (24 hr post exposure). At scheduled necropsy or in cases of morbidity, animals were euthanized by intraperitoneal injection of an overdose of a barbiturate-based sedative. Detailed gross necropsies were performed on all animals (found dead, moribund, or scheduled necropsy) and consisted of a complete external and internal examination including body orifices and cranial, thoracic, and abdominal organs and tissues. All gross findings will be recorded in descriptive terms. Whole blood was collected for bioanalytical analysis, hematology, and clinical chemistry at necropsy. Lungs collected and weighed. The left lobe were tied off and flash frozen for potential, future analysis. The right lobes were lavaged 3×using 4 mL/lavage of phosphate buffered saline (PBS). After lavage is complete the right lobes were flash frozen in liquid nitrogen for potential, future analysis.

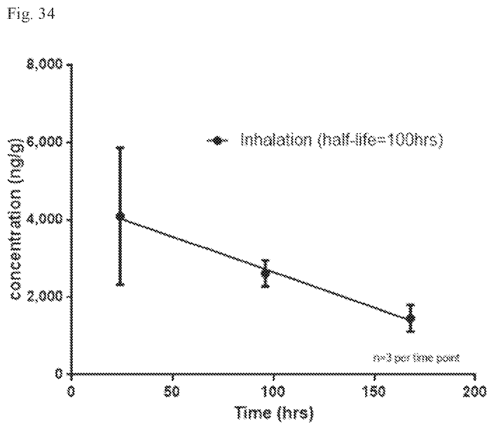

This Example describes the results of BisEDT single-dose rat PK studies comparing inhalation, IV, and oral dosing. The primary takeaways are that (1) BisEDT remains in lung tissue after inhalation dosing with a half-life of about 4 days (FIG. 34); (2) there are very low, but sustained and measurable blood concentrations after oral dosing; (3) IV dosing appears to follow a biphasic pattern with initial distribution into tissues for 18 hours, followed by a slow systemic elimination phase; (4) BisEDT does not appreciably partition into lung tissue after IV or oral dosing—no lung levels after oral and low lung levels detected after IV dosing (<5% vs inhalation group) and levels dropped rapidly with about 24 hour half-life. This indicates that systemic BisEDT does not partition into lung tissue and that lung levels measured after inhalation dosing are due to deposited drug particles on the apical surface; (5) after inhalation dosing, there is sustained, moderate, blood exposure with relatively stable concentrations across time, indicating the drug in the lungs is acting as a depot; (6) BisEDT was tolerated at 100 μg/kg inhalation and IV and 250 μg/kg oral. Based on these data, low doses given daily or every other day are likely to provide very stable drug levels in tissue and blood (small differences between min and max). If the safety margin is demonstrated to be large enough during GLP toxicology and/or clinical studies, it is possible to lengthen the dosing interval and the increase the dose accordingly. Increasing the dose and interval leads to larger fluctuations in tissue and blood levels between peak (after dosing) and trough levels (prior to dosing).

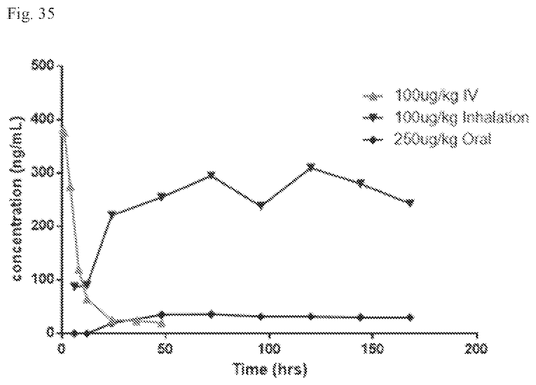

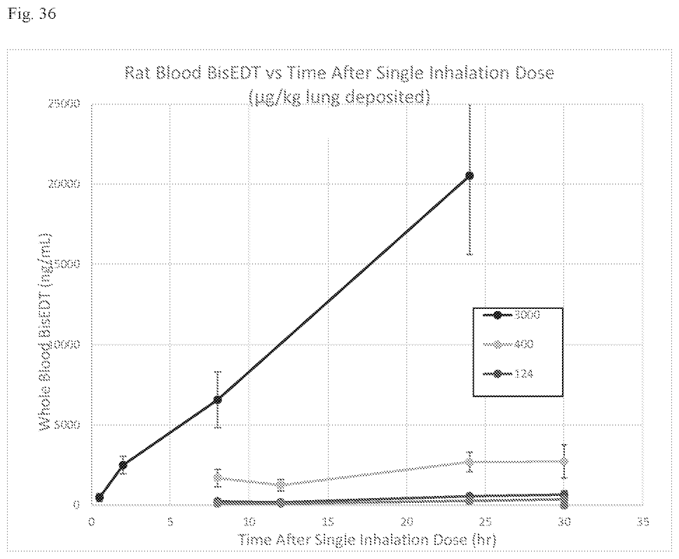

Based on existing in vivo and in vitro toxicology data as well as rat PK and in vitro MIC data, BisEDT suspension for inhalation can be reliably administered at doses providing efficacious lung levels that are tolerated, and it is therefore a viable clinical development candidate. As noted above, BisEDT remains in lung tissue long after inhalation dosing with a half-life of about 4 days (FIG. 35). 24 hours after single inhalation dose 4,093 ng/g was measured in the lung tissue (which equates to 123 μg/mL in lung fluid) and 4 days (96 hrs) after single dose 2,600 ng/g in lung tissue (which equates to 78 μg/mL in lung fluid). Without being bound by any particular theory, it is believed BisEDT's low solubility provides slow dissolution and long exposure on lung surface fluids with limited systemic exposure via diffusion through lung epithelium. The lung appears to act as a depot for slow systemic exposure with about 10× lower systemic levels than seen in the lung. The terminal phase of the IV data appears to show slow elimination after initial tissue distribution (FIG. 36).

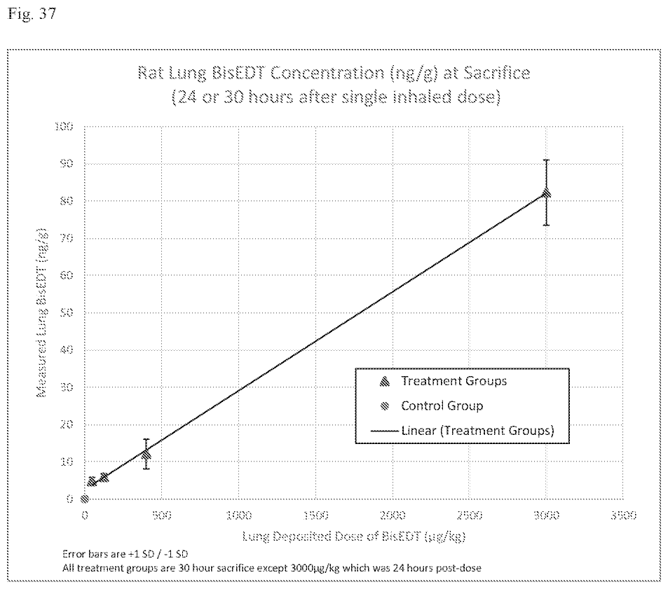

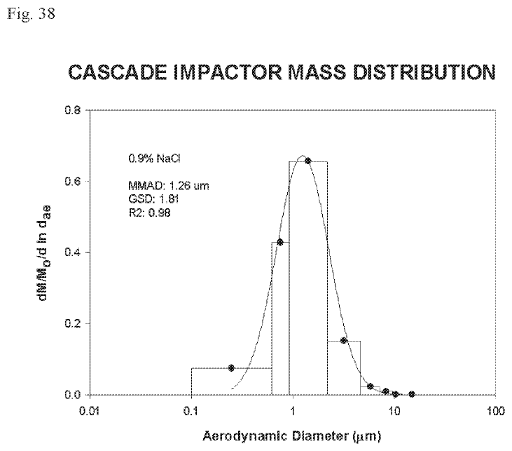

Tables 11 and 12 below show whole blood BisEDT versus time data and lung BisEDT concentration at sacrifice time data respectively. These results are also shown graphically in FIGS. 37 and 38.

TABLE 11
Whole Blood BisEDT versus Time Data

| | | Mean | |
|---|---|---|---|
| | | BisEDT | |
| Dose | Nominal | Conc | |
| (μg/kg) | Time (hr) | (ng/mL) | STDEV |
| 3000 | 1 | 485 | 244 |
| 3000 | 2 | 2504 | 547 |
| 3000 | 8 | 6570 | 1729 |
| 3000 | 24 | 20525 | 4907 |
| 47 | 8 | 114 | 64 |
| 47 | 12 | 98 | 35 |
| 47 | 24 | 282 | 84 |
| 47 | 30 | 373 | 201 |
| 400 | 8 | 1701 | 542 |
| 400 | 12 | 1252 | 368 |
| 400 | 24 | 2700 | 607 |
| 400 | 30 | 2738 | 1028 |
| 124 | 8 | 247 | 3 |
| 124 | 12 | 187 | 78 |
| 124 | 24 | 571 | 93 |
| 124 | 30 | 668 | 239 |
| 0 | 30 | 0 | N/A |
| 0 | 30 | 0 | N/A |
| 0 | 30 | 0 | N/A |

TABLE 12
Lung BisEDT Conc. at Sacrifice

| | | Mean | |
|---|---|---|---|
| | | BisEDT | |
| Dose | Sac time | Conc | |
| (μg/kg) | (hr) | (ng/g) | STDEV |
| 3000 | 24 | 82.3 | 8.7 |
| 400 | 30 | 12.1 | 4 |
| 124 | 30 | 5.9 | 0.9 |
| 47 | 30 | 4.8 | 1.1 |
| 0 | 30 | 0 | N/A |

The efficacy of BisEDT in reducing pulmonary bacterial burden associated with pulmonary infection in a rat model of pulmonary Pseudomonas aeruginosa infection was evaluated. The results follow.

Aerosol concentration: The results of aerosol concentration (gravimetric and chemistry) are presented in Table 13. No BisEDT was detected by chemical analysis of the vehicle, as expected. Gravimetric analysis is listed. The positive control (Tobramycin) concentration was determined gravimetrically and all exposure atmospheres varied by less than 10% through both cohorts and all exposure days. The test article concentration, determined by chemical analysis, varied by less than 5.1% for all exposure cohorts and days and was 11.5 μg/L.

TABLE 13
Average concentration of exposure atmospheres

| | Analysis | Concentration | | |
|---|---|---|---|---|
| Group | Method | Average | | RSD |
| Vehicle | Gravimetric | 0.11 ± 0.02 | mg/L | 14.8% |
| Tobramycin | Gravimetric | 1.26 ± 0.10 | mg/L | 8.3% |
| BisEDT | Chemical | 11.5 ± 0.6 | μg/L | 5.1% |

Dose Delivered: Tables 14 and 15 describe the calculated presented and theoretical deposited doses delivered to the animals in this study. The presented dose is defined as the total inhaled amount of material, comprising material deposited in the sinuses, throat, oropharyngeal region, lung, as well as exhaled material. The theoretical deposited dose, or amount of material that is actually deposited on the surface of the lung, is considered to be 10% of the presented dose in rats (Inhal Toxicol. 2008 October; 20(13):1179-89. doi: 10.1080/08958370802207318, which is hereby incorporated by reference in its entirey). Each animal's current body weight and exposure condition were factored into the equations above for dose determination. Group 3 and Group 4 exposures were 13 minutes and 30 minutes in duration, respectively. The vehicle filters were analyzed chemically for BisEDT and all filters were below detection limits. The positive control (tobramycin) filters were analyzed gravimetrically and the average presented dose for all cohorts and days was 29.0 mg/kg, higher than the 20 mg/kg value specified in the study protocol. The average presented doses for the low and high concentration groups of BisEDT were 0.114 mg/kg and 0.264 mg/kg, respectively. The study protocol called for doses of 0.1 mg/kg and 0.25 mg/kg for those groups. Details of doses received by exposure groups by day and cohort are listed in Tables 14 and 15 below.

TABLE 14
Presented Dose Summary

| | | Cohort 1 Dose | Cohort 2 Dose |
|---|---|---|---|
| Group | Day | (mg/kg) | (mg/kg) |
| Vehicle | 0 | BDL | BDL |
| Vehicle | 2 | BDL | BDL |
| Vehicle | 4 | BDL | BDL |
| Tobramycin | 1 | 31.0 ± 3.4 | 28.9 ± 1.8 |
| Tobramycin | 2 | 29.0 ± 1.9 | 27.9 ± 0.8 |
| Tobramycin | 3 | 27.9 ± 0.7 | 29.3 ± 1.8 |
| Tobramycin | 4 | 29.3 ± 1.7 | 28.5 ± 2.3 |
| BisEDT | 0 | 0.121 ± 0.002 | 0.110 ± 0.002 |
| (0.1 mg/kg) | | | |
| BisEDT | 2 | 0.120 ± 0.002 | 0.120 ± 0.002 |
| (0.1 mg/kg) | | | |
| BisEDT | 4 | 0.101 ± 0.002 | 0.114 ± 0.002 |
| (0.1 mg/kg) | | | |
| BisEDT | −1 | 0.263 ± 0.005 | 0.264 ± 0.003 |
| 0.25 mg/kg | | | |

TABLE 15
Theoretical Deposited Dose Summary

| Group | Day | Cohort 1 Dose | | Cohort 2 Dose | |
|---|---|---|---|---|---|
| Vehicle | 0 | BDL | | BDL | |
| Vehicle | 2 | BDL | | BDL | |
| Vehicle | 4 | BDL | | BDL | |
| Tobramycin | 1 | 3.1 | mg/kg | 2.9 | mg/kg |
| Tobramycin | 2 | 2.9 | mg/kg | 2.8 | mg/kg |
| Tobramycin | 3 | 2.8 | mg/kg | 2.9 | mg/kg |
| Tobramycin | 4 | 2.9 | mg/kg | 2.9 | mg/kg |
| BisEDT | 0 | 12.1 | μg/kg | 11.0 | μg/kg |
| (0.1 mg/kg) | | | | | |
| BisEDT | 2 | 12.0 | μg/kg | 12.0 | μg/kg |
| (0.1 mg/kg) | | | | | |
| BisEDT | 4 | 10.1 | μg/kg | 11.4 | μg/kg |
| (0.1 mg/kg) | | | | | |
| BisEDT | −1 | 26.5 | μg/kg | 26.4 | μg/kg |
| 0.25 mg/kg | | | | | |

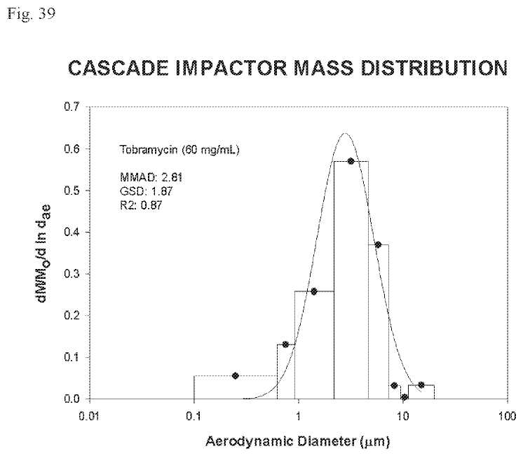

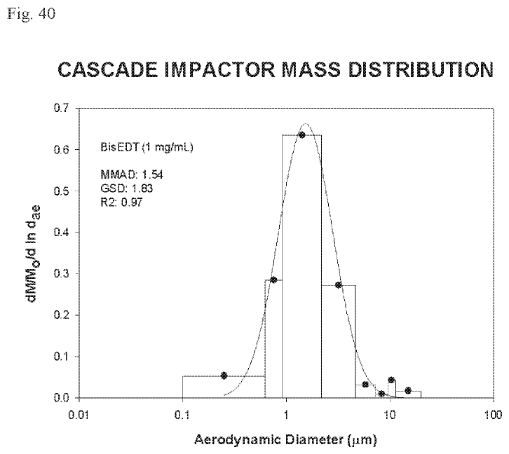

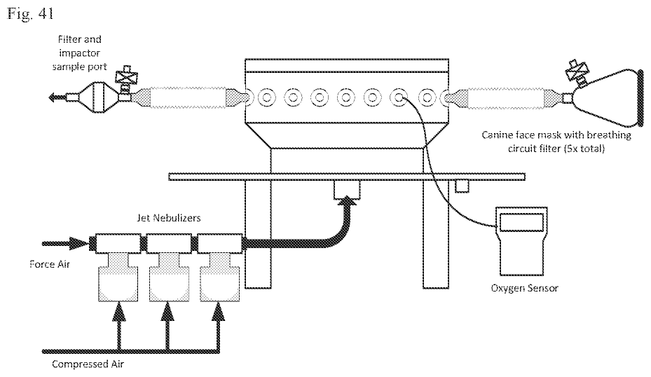

Particle Size Distribution: Particle size distributions (FIGS. 38-40) for each exposure condition was determined using an In-Tox Mercer Impactor. FIG. 39 shows the aerosol size distribution of BisEDT measured during the study. Table 16 provides summaries of particle size distributions (PSD) and geometric standard deviations (GSD) for each exposure type.

TABLE 16
Summary of size distributions for different exposure conditions

| Exposure Conditions | MMAD (μm) | GSD (μm) |
|---|---|---|
| Vehicle | 1.26 | 1.81 |
| Tobramycin | 2.81 | 1.87 |
| BisEDT | 1.54 | 1.83 |

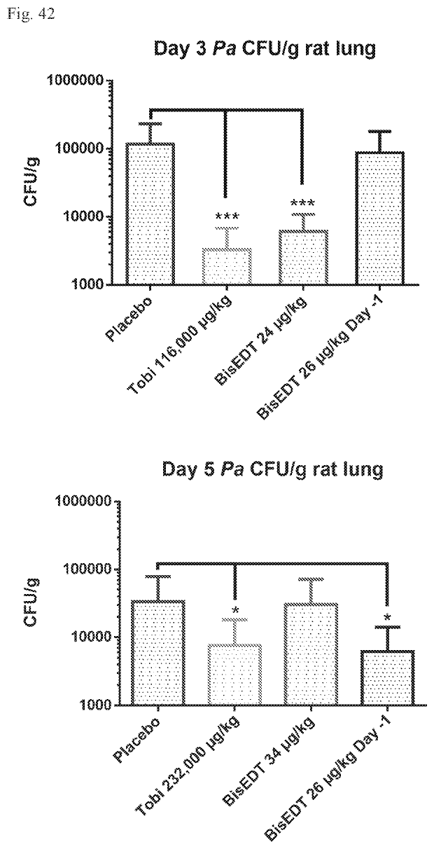

FIG. 42 shows rat efficacy figures showing cumulative (total) administered dose (lung deposited) at days 3 and 5. As can be seen from this figure, the mass ratio of delivered drug is staggering compared to Tobi.

Conclusion: Animals were divided into four experiment inhalational treatment groups: 1) Group 1, a negative control group treated with saline on Days 0, 2, and 4; 2) Group 2, a positive control group treated with tobramycin (target of 20 mg/kg, BID, Days 1-4); 3) Group 3, BisEDT (target of 0.1 mg/kg, QD, Days 0, 2, 4); and Group 4, BisEDT (target of 0.25 mg/kg, once, Day −1). Animals were further divided into two cohorts, separated by one day, to accommodate the challenge and treatment of the number of animals. All animals were exposed per protocol except Group 1 and 3, cohort 2. These animals were exposed on Days 0, 1, and 4 instead of on Days 0, 2, 4.

BisEDT was not detected in animals receiving saline (negative control). Animals treated twice daily (8 doses) with tobramycin received an average dose of 29.0 mg/kg. This dose represents 145% of the targeted dose. As tobramycin is used as a positive control to ensure the model function rather than as a direct comparison, or as a competitor, for the activity of the test article, the increased dose does not impact the study. Animals treated with three targeted doses of 0.1 mg/kg BisEDT received an average of 0.114 mg/kg which represents 114% of the targeted dose. For Group 3 (targeted dose of 0.1 mg/kg), cohort 1 and 2 average doses for each day were, 0.114 and 0.115, respectively. An unpaired t-test (GraphPad Prism 5.0) comparing average dose over the three treatment days demonstrated the differences between cohorts to not be significant (p-value=0.93). Animals which received the single prophylactic targeted dose of 0.25 mg/kg received nearly identical doses of 0.263 mg/kg (cohort 1) and 0.264 mg/kg (cohort 2) of BisEDT, or 105% and 106%, respectively of the targeted dose.

The particles generated for exposure were considered respirable. The positive control (tobramycin) average MMAD of 2.81 μm with a GSD 1.87 μm. Aerosol particle MMAD of negative control (saline) treatment and test article (BisEDT) treatment were 1.26 μm and 1.54 μm with a GSD of 1.81 and 1.83, respectively.

### Example 9: Study of BisEDT Following Face Mask Inhalation in Beagle Dogs

The objective of the study is to determine the maximum tolerated dose (MTD) or maximum feasible dose (MFD) of inhaled bismuth ethanedithiol (BisEDT) after face-mask inhalation exposure (up to 60 min of exposure) in male and female beagle dogs. Animals will be monitored for up to 14 days following the first day of exposure and blood will be collected at predetermined timepoints to analyze the pharmacokinetics of BisEDT. In addition, a coagulation panel will be conducted prior to necropsy. At the conclusion of the study animals will be euthanized and undergo necropsy during which the respiratory tract will be harvested for bioanalytical and histopathological analysis. Results obtained will inform a follow up repeated dose GLP study. This test article is currently being developed as a novel formulation of a broad spectrum antimicrobial/antibiofilm agent, indicated for the treatment of lung infections in patients with cystic fibrosis.

Materials: BisEDT was supplied by Dalton Pharma Services; Lot #ED268-I-II-O I and stored at room temperature. The vehicle used int his study was 0.5% Tween 80, 10 mM sodium phosphate, pH 7.4, in NaCl (adjusted to approximately 300 mOsm) in water which was stored at room temperature.

Canines: Male and female canine, beagle dogs, aged 5-7 months were used int his study. Males weighed 6-10 kg and females weighed 5-9 kg. 6 male and 6 female were used.

Housing: Animals are housed in indoor or outdoor dog kennels.

Conditioning: Animals are conditioned to restraint and face masks.

Feed: 2025C Harlan Global 25% Protein Diet (Harlan Teklad, Madison, Wis.) once daily (except during conditioning and inhalation exposures). All dogs are fasted the evening prior to all blood samples collected for hematology, clinical chemistry, and coagulation analyses. Each batch of feed is analyzed for contaminants by the manufacturer and used within the manufacturer's designated shelf-life. No contaminants are expected to be present at levels that would interfere with the outcome of the study. Municipal water with unlimited access was used, except during conditioning and inhalation exposures.

Environmental Conditions: The targeted conditions for kennel temperature and photoperiod will be as follows: Temperature: 18-29° C.; Light Cycle: 12-h (on each day of exposure the light cycle may be extended to accommodate blood sample collections).

Morbidity and Mortality: Animals are observed twice daily for morbidity and mortality. If found moribund, animals will be euthanized by an overdose of an approved euthanasia solution. Only healthy animals are studied.

Once adequately conditioned to the restraint device and face-masks, the animals are individually exposed to the test article aerosols. The experimental design is shown in Table 17. Animals receive a single face-mask inhalation exposure in a ramp study design to test article aerosols for up to 60 min. Animal observations during and post exposure determine the tolerance of inhaled BisEDT aerosols.

Group 1 (vehicle) are exposed to a vehicle atmosphere for 30 minutes. Group 2 (50 μg/kg) are exposed to BisEDT for 30 minutes. Target concentrations and exposure durations for Groups 3-5 are based on the presence of any toxicity via clinical observations or gross necropsy findings. In each of Groups 3-5, if no adverse clinical signs of toxicity are observed, the next groups' exposure will be conducted at a higher target dose. If adverse clinical signs of toxicity are observed, the next groups' exposure will be conducted at a lower target dose. This approach is repeated for all BisEDT doses. The final documented exposure duration is the maximum tolerated dose or the maximum feasible dose.

Blood is collected for pharmacokinetic (PK) analysis, hematology, clinical chemistry, and coagulation analysis.

At each scheduled timepoint (or in cases of morbidity) animals will be euthanized.

TABLE 17
Experimental Design

| | | | Gender/ | Blood |
|---|---|---|---|---|
| Group | Exposure^{a} | Target Dose | Animal IDs | Collection^{d,e} |
| 1 | Day 0 and Day 7: | n/a | Male: 1001 | NA |
| | TA Vehicle | | Female: 1002 | |
| 2 | Day 0 and Day 7: | Low | Male: 2001 | 4 hrs (±5 min), Day |
| | BisEDT | 50 μg/kg | Female: 2002 | 1, Day 2, Day 7 |
| 3 | Day 0 and Day 7: | Low-Mid: | Male: 3001 | (pre-exposure), |
| | BisEDT | TBD^{c} | Female: 3002 | Day 7 (post- |
| 4 | Day 0 and Day 7: | Mid-High: | Male: 4001, 4003 | exposure), Day 8, |
| | BisEDT | TBD^{c} | Female: 4002, 4004 | Day 9, Day 10, |
| 5^{b} | Day 0 and Day 7: | High: | Male: 5001 | Day 11, Day 12, |
| | BisEDT | TBD^{c} | Female: 5002 | Day 13, Day 14 |

^{a}All groups consist of 1 male and 1 female animal exposed on Day 0 and Day 7. All Groups are terminal on Day 14. Group 4 includes 2 additional animals (1 female and 1 male) with necropsy performed on Day 21.
^{b}Group 5 will be included if Group 4 does not show signs of toxicity.
^{c}TBD: Concentrations will be documented by memo and included in the final study report and will be based on the results of the previous exposure group.
^{d}Blood collections for the additional animals (4003 and 4004) in Group 4 are: Day 15, Day 16, Day 17, Day 18, Day 19, Day 20, Day 21.
^{e}Blood collection for PK analysis.

BisEDT formulation: The initial 5.0 mg/ml BisEDT formulation is prepared in 0.5% Tween 80, 10 mM sodium phosphate, pH 7.4, in NaCl (adjusted to approximately 300 mOsm). On each exposure day the formulation is analyzed.

Test Article Administration: A representative schematic is presented in FIG. 41. The aerosol generation system couples a commercially available compressed air jet nebulizer to a chamber that allows the aerosols to transition to the animals. Exposure is by face-mask inhalation per the study design above. Exposure durations do not exceed 60 min. Formulation concentration and exposure duration are modulated to achieve target doses if necessary.

Animals will be transported to the exposure room and placed onto exposure tables with restraint harnesses. Face-masks connected to the chamber will be placed on the animal immediately prior to the beginning of the exposure period. Temperature and exposure chamber oxygen (%) will be monitored throughout the exposure.

Concentration Monitoring: Aerosol concentration monitoring is conducted by collecting aerosols onto pre-weighed GF/A 47-mm filters. The filters are sampled from the exposure chamber throughout the exposure. The aerosol sampling flow rate through GF/A filters is maintained at 0.5 f 0.1 L/min. After sample collection, filters are weighed to determine the total aerosol concentration in the exposure system. The filters are extracted and analyzed. Based on BisEDT average exposure aerosol concentration the deposited dose is calculated.

Particle Size Determination: Particle size distribution of aerosols are measured from the breathing zone of the exposure chamber by a Mercer-style, seven-stage cascade impactor (Intox Products, Inc., Albuquerque, N. Mex.). The particle size distribution is determined in terms of mass median aerodynamic diameter (MMAD) and geometric standard deviation (GSD). Cascade impactor sample is collected at a flow rate of 2.0 f 0.1 L/min.

Determination of Pulmonary Dose: Deposited dose is calculated using the equation below. In this calculation, the average aerosol concentration measured from the exposure along with the individual animals' body weight for each specific exposure day will be used. In this manner, the estimated amount of BisEDT that is deposited in the lungs will be calculated using the measured BisEDT aerosol concentration.

$$DD ( μg / kg ) = \frac{AC ( μg / L ) \times RMV ( L / min . ) \times DF \times T ( min . )}{BW ( kg )}$$

### Where:

Deposited Dose=(DD) μg/kg
Respiratory minute volume (RMV)=0.608×BW^{0.852} (Alexander D J et al., 2008)
Aerosol exposure concentration (AC)=BisEDT aerosol concentration (μg/L)
Deposition Fraction (DF)=assumed deposition fraction of 25%
BW=body weight of the individual animal on study (kg)

### Observations and Measurements

Clinical Observations and Mortality Morbidity: Detailed clinical observations are recorded starting on dose day with observations recorded twice per day (morning and afternoon) from arrival to the day of exposure. General observations include but are not limited to apnea, labored breathing, malaise, marked nasal discharge, etc. Special attention are paid to clinical signs associated with the respiratory tract. Animals showing severe signs of distress are euthanized at the discretion of the Study Director and in consultation with veterinary staff. Examinations are oriented toward (1) identifying dead, weak, or moribund animals, and (2) documenting the onset and progression of any abnormal clinical signs. Moribund or dead animals are necropsied as soon as possible after being found but in no case later than 16 hrs after being found.

Body Weights: All animals are weighed after release from quarantine and that weight will be the pre-study body weight used to randomize animals into dose groups. Body weights are collected in the morning prior to exposure and at necropsy.

Blood Collections and Bioanalytical Analysis: Blood are collected for pharmacokinetic (PK) analysis prior to exposure, then at 4 hr (±5 min) post exposure, and again on Day 1, Day 2, Day 7 (pre- and 4 hr (±5 min)-post exposure), Day 8, Day 9, Day 10, Day 11, Day 12, Day 13, and Day 14. A total of 0.25 mL is collected at each timepoint. No PK blood is collected from the vehicle control (Group 1) animals; only animals exposed to BisEDT have blood collected for PK analysis. Collected samples are flash frozen without processing for shipment to Medpace for bioanalytical analysis. Additional blood is collected from all animals in all groups for hematology (1 mL) and clinical chemistry (1 mL) at baseline, Day 7 (pre-exposure), and again at euthanasia. Blood (2-2 mL) is collected from all animals at necropsy for coagulation analysis (d-Dimer, fibrinogen, PT, and PTT).

Blood samples are collected from either the jugular vein or another peripheral vein (cephalic or saphenous).

Blood for pharmacokinetic analysis (0.25 mL) is collected into tubes containing K₃EDTA as an anticoagulant. The tubes are flash frozen with liquid nitrogen and stored without processing at −70 to −90° C. until shipping for analysis using ICP-MS assay for quantitation of bismuth as a surrogate for BisEDT.

For hematology analyses, whole blood (1 ml) will be collected with vacutainers containing K₃EDTA as an anticoagulant.

For clinical chemistry analyses, whole blood (1 mL) is placed into serum separator or clot tube and processed to plasma by centrifugation at a minimum of 1300 g at 2 to 8° C. for 10 (±1) min. Plasma samples are stored at −70 to −90° C. until analysis.

For coagulation analyses, blood (2-2 mL samples) are collected into tubes containing sodium citrate anticoagulant, processed, and the citrated plasma frozen and held for analysis. Prothrombin Time (PT), activated Partial Thromboplastin Time (aPTT), d-Dimer, and fibrinogen are analyzed on each animal. Samples will be centrifuged (1500 rpm, 15 minutes), plasma aliquoted into cryogenic vials, and stored frozen (−70 to −90° C.) until analysis.

Hematology and Clinical Chemistry: All dogs are fasted the evening prior to all blood samples collected for hematology, clinical chemistry, and coagulation analyses.

Hematology samples are analyzed by automated (ADVIA™ 120 Hematology System, Siemens Medical Solutions Diagnostics, Tarrytown, N.Y.) analyses. Disposition will be recorded on the sample processing form. Parameters for hematology are shown in Table 18.

Clinical chemistry samples are analyzed on a Hitachi Modular Analytics Clinical Chemistry System (Roche Diagnostics, Indianapolis, Ind.). The clinical chemistry parameters are shown in Table 19.

If target collection volumes needed for hematology or clinical chemistry analysis are not obtained, no analyses will be performed for that animal. In addition, if evaluations cannot be performed, a reason and notation will be included in the study file.

TABLE 18
Hematology Parameters

| Parameter | Abbreviation^{a} | Units |
|---|---|---|
| Red Blood Cell Count | RBC | 10⁶/μL |
| Hemoglobin | HGB | g/dL |
| Hematocrit | HCT | % |
| Mean Corpuscular Volume | MCV | fL |
| Mean Corpuscular Hemoglobin | MCHC | g/dL |
| Concentration | | |
| Mean Corpuscular Hemoglobin | MCH | pg |
| Platelet Count | PLT | 10³/μL |
| Percent Reticulocytes | RETIC | % RBC |

White Blood Cell Count and Absolute Differential

| | | |
|---|---|---|
| White Blood Cell Count | WBC | 10³/μL |
| Neutrophils | PMN | 10³/μL |
| Lymphocytes | LYM | 10³/μL |
| Monocytes | MONO | 10³/μL |
| Eosinophils | EOS | 10³/μL |
| Basophils | BASO | 10³/μL |
| Large Unstained Cells | LUC | 10³/μL |

^{a}Abbreviations from the hematology system differ from those listed above, however the final results will be reported as described above

TABLE 19
Clinical Chemistry Parameters

| Analyte | Abbreviation^{a} | Units |
|---|---|---|
| Alanine Aminotransferase (Alanine | ALT | IU/L |
| Transaminase)-Serum | | |
| Albumin | ALB | g/dL |
| Aspartate Aminotransferase (Aspartate | AST | IU/L |
| Transaminase)-Serum | | |
| Bilirubin (Total) | BILI-T | mg/dL |
| Blood Urea Nitrogen | BUN | mg/dl |
| Calcium | CA | mg/dL |
| Chloride (Serum) | CL-S | mmol/L |
| Cholesterol (Total) | CHOL | mg/dL |
| Creatinine (Serum) | CRE-S | mg/dL |
| Glucose | GLU | mg/dL |
| Gamma Glutamyltransferase | GGT | IU/L |
| Alkaline Phosphatase | ALP | IU/L |
| Phosphate | PHOS | mg/dL |
| Potassium (Serum) | K-S | mmol/L |
| Protein (Total) | TP | g/dL |
| Sodium (Serum) | NA-S | mmol/L |
| Triglycerides | TRIG | mg/dL |

Calculated Parameters and Ratios

| | | |
|---|---|---|
| Albumin/Globulin | A/G | None |
| Blood Urea Nitrogen/Creatinine | BUN/CRE | None |
| Globulin | GLOBN | g/dL |

^{a}Abbreviations from the chemistry system differ from those listed above, however the final results will be reported as described above

### Euthanasia, Necropsy, and Histology

Euthanasia: At scheduled necropsies (Day 14 or Day 21, respectively) or in cases of moribundity, animals are tranquilized and euthanized by a veterinarian or their. Animals are tranquilized by administration of acepromazine (0.02-0.2 mg/kg, IM) and butorphanol (≥0.33 mg/kg, IM). After sedation, an intravenous catheter is placed to accommodate administration of a ketamine/diazepam cocktail and flushed with a saline solution. The cocktail is a proportional mixture of 1 mL of ketamine (100 mg/mL) and 1 mL of diazepam (5 mg/mL). The cocktail is then be administered at a dose of ≥1 mL/9 kg body weight. Note: if a dog is sufficiently sedated after use of acepromazine and butorphanol, administration of ketamine/diazepam cocktail may not be required or administered and will be documented. The animal is then euthanized by an overdose of a barbiturate-based sedative (Euthasol®, ≥1 mL/4.5 kg, IV) and flushed with a saline solution. If needed, exsanguination may be performed.

Necropsy: Detailed gross necropsies are performed on all animals and will consist of a complete external and internal examination including body orifices (ears, nostrils, mouth, anus, etc.) and cranial, thoracic, and abdominal organs and tissues. All gross findings are recorded in descriptive terms, typically including location(s), size (in mm), shape, color, consistency, and number. Animals found dead will be refrigerated until necropsy can be performed. A cause of death is determined if possible.

The left lobe of the lung is used for histopathology and will be fixed in 10% NBF. The right middle lobe has three approximately 1 gram sections collected and flash frozen with liquid nitrogen for bioanalytical analysis; samples will be stored at −70 to −90° C. until shipping for analysis using TCP-MS assay for quantitation of bismuth as a surrogate for BisEDT. Liver, spleen, kidney, brain, heart, and respiratory tract are collected and fixed for potential, future analysis.

Histology and Pathology: Lungs are, prepared, and embedded, cut and mounted on slides, stained with hematoxylin and eosin, and evaluated by a Pathologist. In addition, representative lesions observed and collected during necropsy may be evaluated. Histology is conducted on Group 1 and Group 4; additional analyses may be conducted by Amendment to the Approved Study Protocol.

### Results

Table 20 shows the actual deposited dose of BisEDT in the lung for dogs tested. Table 21 shows hematology results and Table 22 shows clinical chemistry results for the dogs tested.

TABLE 20
Actual lung deposited doses per group/animal #:

| | | Lung deposited |
|---|---|---|
| Animal # | Group | dose, ug/kg |
| 1001 | 1 | 0 |
| 1002 | 1 | 0 |
| 2001 | 2 | 55 |
| 2002 | 2 | 56 |
| 3001 | 3 | 112 |
| 3002 | 3 | 111 |
| 4001 | 4 | 176 |
| 4002 | 4 | 180 |
| 4003 | 4 | 195 |
| 4004 | 4 | 197 |

TABLE 21
Hematology Summary

| | Sex/ | | | | | | | |
|---|---|---|---|---|---|---|---|---|
| Subject/ | QC | WBC | RBC | HGB | HCT | MCV | MCH | MCHC |
| QC Lot | Identifier | \hat{ }3/uL | \hat{ }6/uL | g/dL | % | fL | pg | g/dL |
| 1001 | Male | 13.48 | 6.75 | 15.9 | 46.6 | 69.1 | 23.5 | 34.0 |
| 1001 | Male | 7.98 | 5.89 | 13.8 | 39.8 | 67.7 | 23.4 | 34.7 |
| 1001 | Male | 9.32 | 6.15 | 14.3 | 42.8 | 69.6 | 23.2 | 33.4 |
| 1002 | Female | 9.18 | 7.54 | 17.0 | 51.2 | 67.9 | 22.6 | 33.2 |
| 1002 | Female | 8.67 | 6.75 | 16.0 | 44.6 | 66.1 | 23.6 | 35.8 |
| 1002 | Female | 7.98 | 6.47 | 15.0 | 45.2 | 69.9 | 23.3 | 33.3 |
| Grp 1 | Avg | 11.33 | 7.15 | 16.5 | 48.9 | 68.5 | 23.1 | 33.6 |
| Grp 1 | Avg | 8.33 | 6.32 | 14.9 | 42.2 | 66.9 | 23.5 | 35.3 |
| Grp 1 | Avg | 8.65 | 6.31 | 14.7 | 44.0 | 69.8 | 23.3 | 33.4 |
| 2001 | Male | 9.18 | 6.83 | 15.6 | 45.8 | 67.0 | 22.8 | 34.1 |
| 2001 | Male | 9.13 | 6.49 | 15.0 | 43.1 | 66.4 | 23.2 | 34.9 |
| 2001 | Male | 5.99 | 6.28 | 14.2 | 41.8 | 66.5 | 22.7 | 34.1 |
| 2002 | Female | 6.99 | 7.05 | 16.4 | 49.0 | 69.5 | 23.2 | 33.4 |
| 2002 | Female | 7.77 | 6.74 | 15.0 | 43.7 | 64.8 | 22.3 | 34.4 |
| 2002 | Female | 8.10 | 6.50 | 14.3 | 42.9 | 66.0 | 21.9 | 33.2 |
| Grp 2 | Avg | 8.09 | 6.94 | 16.0 | 47.4 | 68.3 | 23.0 | 33.8 |
| Grp 2 | Avg | 8.45 | 6.62 | 15.0 | 43.4 | 65.6 | 22.8 | 34.7 |
| Grp 2 | Avg | 7.05 | 6.39 | 14.3 | 42.4 | 66.3 | 22.3 | 33.7 |
| 3001 | Male | 8.74 | 6.11 | 13.6 | 40.2 | 65.8 | 22.3 | 33.9 |
| 3001 | Male | 7.25 | 6.35 | 14.2 | 41.0 | 64.6 | 22.3 | 34.5 |
| 3001 | Male | 12.97 | 6.98 | 15.8 | 48.2 | 69.1 | 22.7 | 32.8 |
| 3002 | Female | 11.50 | 7.17 | 16.2 | 47.3 | 65.9 | 22.6 | 34.3 |
| 3002 | Female | 9.18 | 6.12 | 14.6 | 43.1 | 70.4 | 23.9 | 34.0 |
| 3002 | Female | 14.08 | 7.36 | 16.7 | 49.8 | 67.6 | 22.7 | 33.5 |
| Grp 3 | Avg | 10.12 | 6.64 | 14.9 | 43.8 | 65.9 | 22.5 | 34.1 |
| Grp 3 | Avg | 8.22 | 6.24 | 14.4 | 42.1 | 67.5 | 23.1 | 34.3 |
| Grp 3 | Avg | 13.53 | 7.17 | 16.3 | 49.0 | 68.4 | 22.7 | 33.2 |
| 4001 | Male | 8.23 | 6.44 | 14.8 | 42.7 | 66.3 | 23.0 | 34.7 |
| 4001 | Male | 10.09 | 6.81 | 16.0 | 47.5 | 69.7 | 23.4 | 33.6 |
| 4001 | Male | 9.86 | 6.37 | 14.6 | 45.4 | 71.3 | 22.9 | 32.2 |
| 4004 | Female | 10.41 | 6.46 | 14.7 | 41.9 | 64.8 | 22.8 | 35.2 |
| 4004 | Female | 10.37 | 7.19 | 16.6 | 50.6 | 70.4 | 23.1 | 32.8 |
| 4004 | Female | 10.50 | 6.71 | 15.4 | 47.6 | 70.9 | 22.9 | 32.4 |
| Grp 4 | Avg | 9.32 | 6.45 | 14.75 | 42.30 | 65.55 | 22.90 | 34.95 |
| Grp 4 | Avg | 10.23 | 7.00 | 16.30 | 49.05 | 70.05 | 23.25 | 33.20 |
| Grp 4 | Avg | 10.18 | 6.54 | 15.00 | 46.50 | 71.10 | 22.90 | 32.30 |
| 4002 | Female | 10.21 | 5.68 | 13.7 | 39.8 | 70.1 | 24.1 | 34.4 |
| 4002 | Female | 7.45 | 6.61 | 13.8 | 40.7 | 61.6 | 20.9 | 34.0 |
| 4003 | Male | 6.63 | 6.11 | 12.8 | 37.1 | 60.8 | 21.0 | 34.6 |
| 4003 | Male | 11.18 | 5.92 | 14.3 | 44.2 | 74.7 | 24.2 | 32.4 |
| Grp 4 | Avg | 8.42 | 5.90 | 13.25 | 38.45 | 65.45 | 22.55 | 34.50 |
| Grp 4 | Avg | 9.32 | 6.27 | 14.05 | 42.45 | 68.15 | 22.55 | 33.20 |
| | | | | | | | | RET |
| Subject/ | PLT | NEUT | LYMP | MONO | EOS | BASO | LUC | % |
| QC Lot | \hat{ }3/uL | \hat{ }3/uL | \hat{ }3/uL | \hat{ }3/uL | \hat{ }3/uL | \hat{ }3/uL | \hat{ }3/uL | % |
| 1001 | 250 | 8.56 | 3.73 | 0.82 | 0.15 | 0.20 | 0.03 | 0.72 |
| 1001 | 241 | 5.00 | 2.26 | 0.54 | 0.08 | 0.07 | 0.02 | 0.34 |
| 1001 | 106 | 6.37 | 2.22 | 0.57 | 0.05 | 0.08 | 0.01 | 0.39 |
| 1002 | 297 | 3.97 | 3.89 | 0.39 | 0.73 | 0.17 | 0.02 | 0.51 |
| 1002 | 168 | 5.92 | 1.83 | 0.68 | 0.10 | 0.13 | 0.01 | 0.23 |
| 1002 | 193 | 5.38 | 1.75 | 0.72 | 0.07 | 0.06 | 0.02 | 0.44 |
| Grp 1 | 273.5 | 6.27 | 3.81 | 0.61 | 0.44 | 0.19 | 0.03 | 0.62 |
| Grp 1 | 204.5 | 5.46 | 2.05 | 0.61 | 0.09 | 0.10 | 0.02 | 0.29 |
| Grp 1 | 149.5 | 5.88 | 1.99 | 0.65 | 0.06 | 0.07 | 0.02 | 0.42 |
| 2001 | 197 | 5.31 | 2.81 | 0.63 | 0.30 | 0.12 | 0.01 | 0.34 |
| 2001 | 166 | 5.11 | 2.73 | 0.67 | 0.45 | 0.15 | 0.01 | 0.55 |
| 2001 | 161 | 2.88 | 2.09 | 0.53 | 0.39 | 0.08 | 0.02 | 0.60 |
| 2002 | 206 | 3.96 | 2.27 | 0.50 | 0.14 | 0.11 | 0.02 | 0.65 |
| 2002 | 294 | 4.26 | 2.82 | 0.51 | 0.09 | 0.10 | 0.01 | 0.25 |
| 2002 | 304 | 4.83 | 2.61 | 0.51 | 0.06 | 0.07 | 0.01 | 0.31 |
| Grp 2 | 201.5 | 4.64 | 2.54 | 0.57 | 0.22 | 0.12 | 0.02 | 0.50 |
| Grp 2 | 230.0 | 4.69 | 2.78 | 0.59 | 0.27 | 0.13 | 0.01 | 0.40 |
| Grp 2 | 232.5 | 3.86 | 2.35 | 0.52 | 0.23 | 0.08 | 0.02 | 0.46 |
| 3001 | 310 | 4.36 | 3.29 | 0.44 | 0.57 | 0.07 | 0.02 | 0.61 |
| 3001 | 321 | 3.52 | 2.93 | 0.45 | 0.27 | 0.07 | 0.02 | 0.79 |
| 3001 | 255 | 7.28 | 4.35 | 0.83 | 0.38 | 0.11 | 0.02 | 1.02 |
| 3002 | 325 | 6.77 | 3.67 | 0.67 | 0.17 | 0.20 | 0.03 | 0.91 |
| 3002 | 247 | 5.29 | 2.86 | 0.79 | 0.12 | 0.10 | 0.02 | 0.94 |
| 3002 | 319 | 9.25 | 3.80 | 0.76 | 0.15 | 0.10 | 0.02 | 0.72 |
| Grp 3 | 317.5 | 5.57 | 3.48 | 0.56 | 0.37 | 0.14 | 0.03 | 0.76 |
| Grp 3 | 284.0 | 4.41 | 2.90 | 0.62 | 0.20 | 0.09 | 0.02 | 0.87 |
| Grp 3 | 287.0 | 8.27 | 4.08 | 0.80 | 0.27 | 0.11 | 0.02 | 0.87 |
| 4001 | 208 | 4.61 | 2.68 | 0.39 | 0.48 | 0.07 | 0.01 | 0.41 |
| 4001 | 214 | 5.96 | 3.18 | 0.37 | 0.50 | 0.07 | 0.02 | 0.33 |
| 4001 | 213 | 6.30 | 2.78 | 0.37 | 0.35 | 0.04 | 0.01 | 0.58 |
| 4004 | 246 | 5.98 | 2.88 | 0.49 | 0.95 | 0.09 | 0.02 | 0.27 |
| 4004 | 238 | 5.95 | 3.04 | 0.50 | 0.77 | 0.09 | 0.01 | 0.62 |
| 4004 | 251 | 6.74 | 2.82 | 0.39 | 0.47 | 0.06 | 0.01 | 0.43 |
| Grp 4 | 227.00 | 5.30 | 2.78 | 0.44 | 0.72 | 0.08 | 0.02 | 0.34 |
| Grp 4 | 226.00 | 5.96 | 3.11 | 0.44 | 0.64 | 0.08 | 0.02 | 0.48 |
| Grp 4 | 232.00 | 6.52 | 2.80 | 0.38 | 0.41 | 0.05 | 0.01 | 0.51 |
| 4002 | 257 | 6.73 | 2.46 | 0.73 | 0.18 | 0.09 | 0.01 | 1.07 |
| 4002 | 308 | 4.11 | 2.59 | 0.50 | 0.13 | 0.10 | 0.02 | 0.88 |
| 4003 | 313 | 3.60 | 2.32 | 0.45 | 0.19 | 0.04 | 0.02 | 0.51 |
| 4003 | 248 | 7.39 | 2.87 | 0.61 | 0.21 | 0.10 | 0.01 | 0.83 |
| Grp 4 | 285.00 | 5.17 | 2.39 | 0.59 | 0.19 | 0.07 | 0.02 | 0.79 |
| Grp 4 | 278.00 | 5.75 | 2.73 | 0.56 | 0.17 | 0.10 | 0.02 | 0.86 |

TABLE 22
Clinical Summary

| Subject/ | Sex/ | Na | K | Cl | GLUC | BUN | | P | TBIL | |
|---|---|---|---|---|---|---|---|---|---|---|
| QC | QC | mmo/ | mmo/ | mmo/ | mg/ | mg/ | CREA | mg/ | mg/ | ALPK |
| Lot | Identifier | L | L | L | dL | dL | mg/dL | dL | dL | IU/L |
| 1001 | Male | 149 | 4.7 | 109 | 90 | 14 | 0.7 | 7.6 | 0.15 | 248 |
| 1001 | Male | 146 | 4.7 | 111 | 78 | 15 | 0.5 | 6 4 | 0.15 | 243 |
| 1001 | Male | 150 | 4.2 | 113 | 92 | 17 | 0.5 | 6.6 | 0.15 | 235 |
| 1002 | Female | 148 | 5.1 | 109 | 92 | 13 | 0.7 | 6.4 | 0.15 | 91 |
| 1002 | Female | 147 | 4.9 | 113 | 88 | 15 | 0.7 | 6.4 | 0.15 | 125 |
| 1002 | Female | 148 | 4.5 | 113 | 89 | 13 | 0.7 | 6.2 | 0.15 | 120 |
| Grp 1 | Avg | 149 | 4.9 | 109 | 91 | 14 | 0.7 | 7.0 | 0.15 | 170 |
| Grp 1 | Avg | 147 | 4.8 | 112 | 83 | 15 | 0.6 | 6.4 | 0.15 | 184 |
| Grp 1 | Avg | 149 | 4.4 | 113 | 91 | 15 | 0.6 | 6.4 | 0.15 | 178 |
| 2001 | Male | 149 | 4.5 | 109 | 91 | 12 | 0.7 | 6.8 | 0.15 | 130 |
| 2001 | Male | 147 | 4.9 | 113 | 97 | 16 | 0.6 | 5.9 | 0.15 | 140 |
| 2001 | Male | 147 | 4.7 | 108 | 102 | 15 | 0.6 | 5.5 | 0.15 | 128 |
| 2002 | Female | 149 | 5.0 | 110 | 95 | 13 | 0.6 | 7.4 | 0.15 | 241 |
| 2002 | Female | 147 | 5.2 | 114 | 98 | 14 | 0.6 | 6.4 | 0.15 | 111 |
| 2002 | Female | 148 | 4.6 | 113 | 89 | 14 | 0.6 | 5.7 | 0.15 | 103 |
| Grp 2 | Avg | 149 | 4.8 | 110 | 93 | 13 | 0.7 | 7.1 | 0.15 | 186 |
| Grp 2 | Avg | 147 | 5.1 | 114 | 98 | 15 | 0.6 | 6.2 | 0.15 | 126 |
| Grp 2 | Avg | 148 | 4.7 | 111 | 96 | 15 | 0.6 | 5 6 | 0.15 | 116 |
| 3001 | Male | 146 | 4.7 | 111 | 100 | 13 | 0.6 | 6 5 | 0.15 | 142 |
| 3001 | Male | 146 | 5.1 | 110 | 88 | 17 | 0.7 | 6.8 | 0.15 | 135 |
| 3001 | Male | 148 | 5.8 | 109 | 51 | 33 | 1.0 | 7.2 | 0.15 | 121 |
| 3002 | Female | 149 | 5.5 | 112 | 112 | 11 | 0.6 | 6.6 | 0.15 | 164 |
| 3002 | Female | 145 | 5.3 | 109 | 90 | 12 | 0.5 | 6.5 | 0.15 | 90 |
| 3002 | Female | 149 | 5.9 | 110 | 65 | 25 | 0.9 | 7.5 | 0.15 | 176 |
| Grp 3 | Avg | 148 | 5.1 | 112 | 106 | 12 | 0.6 | 6.6 | 0.15 | 153 |
| Grp 3 | Avg | 146 | 5.2 | 110 | 89 | 15 | 0.6 | 6.7 | 0.15 | 113 |
| Grp 3 | Avg | 149 | 5.9 | 110 | 58 | 29 | 1.0 | 7.4 | 0.15 | 149 |
| 4001 | Male | 152 | 4.7 | 120 | 102 | 11 | 0.7 | 6.5 | 0.15 | 103 |
| 4001 | Male | 147 | 4.8 | 116 | 82 | 12 | 0.7 | 6.6 | 0.15 | 114 |
| 4001 | Male | 145 | 4.7 | 113 | 86 | 11 | 0.6 | 6 0 | 0.15 | 119 |
| 4004 | Female | 153 | 4.6 | 119 | 102 | 13 | 0.6 | 5.9 | 0.15 | 103 |
| 4004 | Female | 148 | 5.1 | 115 | 70 | 13 | 0.6 | 6.3 | 0.15 | 93 |
| 4004 | Female | 146 | 4.3 | 114 | 96 | 13 | 0.6 | 5.6 | 0.15 | 95 |
| Grp 4 | Avg | 153 | 4.7 | 120 | 102 | 12 | 0.7 | 6.2 | 0.15 | 103 |
| Grp 4 | Avg | 148 | 5.0 | 116 | 76 | 13 | 0.7 | 6.5 | 0.15 | 104 |
| Grp 4 | Avg | 146 | 4.5 | 114 | 91 | 12 | 0.6 | 5.8 | 0.15 | 107 |
| 4002 | Female | 153 | 5.3 | 116 | 106 | 13 | 0.6 | 6.4 | 0.15 | 147 |
| 4002 | Female | 149 | 5.3 | 112 | 79 | 12 | 0.5 | 6.9 | 0.15 | 103 |
| 4003 | Male | 149 | 4.9 | 117 | 109 | 16 | 0.6 | 7.2 | 0.15 | 146 |
| 4003 | Male | 147 | 4.9 | 112 | 81 | 15 | 0.7 | 7.1 | 0.15 | 129 |
| Subject/ | ALT | AST | | TP | ALB | CA2 | TRIG | CHOL | | |
| QC | IU/ | IU/ | GGT | g/d | g/d | mg/ | mg/ | mg/ | GLOB | |
| Lot | L | L | IU/L | L | L | dL | dL | dL | g/dL | AGR |
| 1001 | 25 | 29 | 3 | 5.0 | 3.3 | 11.0 | 53 | 132 | 1.7 | 1.9 |
| 1001 | 29 | 29 | 3 | 5.0 | 3.3 | 10.6 | 39 | 133 | 1.7 | 1.9 |
| 1001 | 21 | 33 | 3 | 5.5 | 3.7 | 10.9 | 38 | 130 | 1.8 | 2.1 |
| 1002 | 24 | 27 | 3 | 4.9 | 3.3 | 10.3 | 37 | 107 | 1.6 | 2.1 |
| 1002 | 26 | 33 | 3 | 5.0 | 3.6 | 10.7 | 32 | 129 | 1.4 | 2.6 |
| 1002 | 27 | 34 | 3 | 5.0 | 3.5 | 10.4 | 29 | 116 | 1.5 | 2.3 |
| Grp 1 | 25 | 28 | 3 | 5.0 | 3.3 | 10.7 | 45 | 120 | 1.7 | 2.0 |
| Grp 1 | 28 | 31 | 3 | 6.0 | 3.5 | 10.7 | 36 | 131 | 1.6 | 2.3 |
| Grp 1 | 24 | 34 | 3 | 5.3 | 3.6 | 10.7 | 34 | 123 | 1.7 | 2.2 |
| 2001 | 22 | 48 | 3 | 5.0 | 3.3 | 10.9 | 66 | 168 | 1.7 | 1.9 |
| 2001 | 22 | 36 | 3 | 5.1 | 3.4 | 10.8 | 80 | 167 | 1.7 | 2.0 |
| 2001 | 22 | 36 | 3 | 5.5 | 3.5 | 11.0 | 63 | 152 | 2.0 | 1.8 |
| 2002 | 18 | 35 | 3 | 5.2 | 3.3 | 10.9 | 38 | 138 | 1.9 | 1.7 |
| 2002 | 29 | 41 | 3 | 5.2 | 3.6 | 10.8 | 39 | 123 | 1.6 | 2.3 |
| 2002 | 27 | 38 | 3 | 5.3 | 3.7 | 10.6 | 31 | 114 | 1.6 | 2.3 |
| Grp 2 | 20 | 42 | 3 | 5.1 | 3.3 | 10.9 | 52 | 153 | 1.8 | 1.8 |
| Grp 2 | 26 | 39 | 3 | 5.2 | 3.5 | 10.8 | 60 | 145 | 1.7 | 2.2 |
| Grp 2 | 25 | 37 | 3 | 5.4 | 3.6 | 10.8 | 47 | 133 | 1.8 | 2.1 |
| 3001 | 26 | 30 | 3 | 4.8 | 3.2 | 10.7 | 26 | 100 | 1.6 | 2.0 |
| 3001 | 24 | 32 | 3 | 5.1 | 3.4 | 10.5 | 34 | 111 | 1.7 | 2.0 |
| 3001 | 22 | 33 | 3 | 5.0 | 3.4 | 10.0 | 52 | 111 | 1.6 | 2.1 |
| 3002 | 28 | 36 | 3 | 5.0 | 3.5 | 11.2 | 34 | 126 | 1.5 | 2.3 |
| 3002 | 20 | 28 | 3 | 5.5 | 3.5 | 10.8 | 43 | 175 | 2.0 | 1.8 |
| 3002 | 20 | 30 | 3 | 5.1 | 3.7 | 10.4 | 58 | 141 | 1.4 | 2.6 |
| Grp 3 | 27 | 30 | 3 | 4.9 | 3.4 | 11.0 | 30 | 113 | 1.6 | 2.2 |
| Grp 3 | 22 | 30 | 3 | 5.3 | 3.5 | 10.7 | 39 | 143 | 1.9 | 1.9 |
| Grp 3 | 21 | 32 | 3 | 5.1 | 3.6 | 10.2 | 55 | 126 | 1.5 | 2.4 |
| 4001 | 27 | 32 | 3 | 5.2 | 3.6 | 10.8 | 49 | 153 | 1.6 | 2.3 |
| 4001 | 27 | 39 | 3 | 5.3 | 3.4 | 10.6 | 46 | 147 | 1.9 | 1.8 |
| 4001 | 28 | 34 | 3 | 4.9 | 3.2 | 10.4 | 40 | 111 | 1.7 | 1.9 |
| 4004 | 28 | 33 | 3 | 5.1 | 3.4 | 10.8 | 53 | 125 | 1.7 | 2.0 |
| 4004 | 25 | 38 | 3 | 5.2 | 3.3 | 10.8 | 49 | 124 | 1.9 | 1.7 |
| 4004 | 24 | 31 | 3 | 5.0 | 3.3 | 10.6 | 44 | 112 | 1.7 | 1.9 |
| Grp 4 | 28 | 33 | 3 | 5.2 | 3.5 | 10.8 | 51 | 139 | 1.7 | 2.2 |
| Grp 4 | 26 | 39 | 3 | 5.3 | 3.4 | 10.7 | 48 | 136 | 1.9 | 1.8 |
| Grp 4 | 26 | 33 | 3 | 5.0 | 3.3 | 10.5 | 42 | 112 | 1.7 | 1.9 |
| 4002 | 20 | 32 | 3 | 5.6 | 3.5 | 11.1 | 34 | 182 | 2.1 | 1.7 |
| 4002 | 21 | 35 | 3 | 5.5 | 3.5 | 11.2 | 41 | 169 | 2.0 | 1.8 |
| 4003 | 40 | 30 | 3 | 5.4 | 3.5 | 11.2 | 55 | 152 | 1.9 | 1.8 |
| 4003 | 23 | 33 | 3 | 5.4 | 3.3 | 11.0 | 53 | 176 | 2.1 | 1.6 |

### REFERENCES

Alexander D J, Collins C J, Coombs D W, Gilkison, I S, Hardy, C J, Healey, G, Karantabias, G, Johnson, N, Karlsson, A, Kilgour, J D, and McDonald, P. Association of Inhalation Toxicologists (AIT) working party recommendation for standard delivered dose calculation and expression in non-clinical aerosol inhalation toxicology studies with pharmaceuticals. Inhalation Toxicology; 20(13): 1179-89, 2008.
National Research Council, 2011. Guide for the Care and Use of Laboratory Animals. National Academy Press, Washington, D.C.
Tepper, J S, Kuehl, P K, Cracknell, S, Nikula, K J, Pei, L, and Blanchard, J D. 2016. Symposium Summary: “Breath In, Breath Out, Its Easy: What You Need to Know About Developing Inhaled Drugs.” Int. J. Toxicol. 35(4) 376-392.

### Example 10: Antimicrobial Interaction of BisEDT with Agents Used to Treat Cystic

Fibrosis Infections Caused by Pseudomonas aeruginosa and Burkholderia cepacia Complex

Introduction: The interaction between BisEDT and a variety of agents used in the treatment of patients with cystic fibrosis (CF) was evaluated against P. aeruginosa and Burkholderia spp. The antimicrobial interaction was determined by measuring fractional inhibitory concentrations (FTC) in a checkerboard assay.

### Materials and Methods

Test articles: BisEDT was provided as a dry powder and was stored at room temperature in the dark prior to testing. The comparator compounds were handled in accordance with guidelines from the Clinical and Laboratory Standards Institute (CLSI; 1, 2). Specific information on the individual drug lots and concentration ranges tested is shown in the Table 23 below:

TABLE 23
drug lots and concentration ranges tested

| | | | | Concentration | Concentration |
|---|---|---|---|---|---|
| | | | | Ranges tested | Ranges tested |
| Test Articles | Supplier | Lot Number | Solvent | (μg/ml) (MIC) | (μg/ml) (FIC) |
| BisEDT | Microbion | ED268-1-11-01 | DMSO | 64-0.06 | 16-0.25; 4-0.06 |
| Tobramycin | Sigma | 109K1184 | Sterile dH₂O | 256-0.25; 2-0.002 | 256-0.25 |
| Amikacin | Sigma | 058K0803 | Sterile dH₂O | 256-0.25; 2-0.002 | 256-0.25 |
| Aztreonam | USP | R041F0 | Saturated | 256-0.25; 2-0.002 | 256-0.25 |
| | | | NaHCO₃ | | |
| Meropenem | USP | J0K434 | Sterile dH₂O | 256-0.25; 2-0.002 | 64-0.06 |
| Ciprofloxacin | USP | R05170 | Sterile dH₂O | 256-0.25; 2-0.002 | 256-0.25 |
| Colistin | Sigma | SLBV1747 | Sterile dH₂O | 256-0.25, 2-0.002 | 256-0.25 |

Appropriate solvents were added to the drugs which were prepared at 40-fold the top testing concentration. The stock solutions were allowed to stand for approximately 1 hr at room temperature in the dark to auto-sterilize before being used for testing. Drug stocks of the comparators were frozen and stored at −80° C.

Organisms: The test organisms were clinical isolates previously acquired by Micromyx or from the American Type Culture Collection (ATCC). Upon receipt at Micromyx, the isolates were streaked under suitable conditions onto agar medium appropriate to each organism. The organisms were incubated for 18-24 hr at 35° C. Colonies harvested from these growth plates were resuspended in the appropriate medium containing a cryoprotectant. Aliquots of each suspension were then frozen at −80° C. Prior to the assay, the organisms were streaked onto trypticase soy agar plus 5% sheep blood (BD; Sparks, Md.; Lot No. 8179506) and were incubated as described above.

Test Media: The medium employed for the assay was cation-adjusted Mueller Hinton broth (MHB II; Becton-Dickinson, Sparks, Md.; Lot No 8096574). The medium was prepared according to CLSI guidelines (2).

MIC Assay Methodology: MIC assay plates were prepared using the CLSI broth microdilution procedure (1, 2). Automated liquid handlers (Multidrop 384, Biomek 2000 and Biomek FX) were used to conduct serial dilutions and liquid transfers. All wells in columns 2 through 12 of a standard 96-well microdilution plate (Costar 3795) were filled with 150 μL of the proper diluent. Three hundred μL of each test drug (at 40X) were added to each well in Column 1 of the plates. This plate was used to prepare the drug “mother plate” which provided the serial drug dilutions for the replicate “daughter plates”. The Biomek 2000 was used to complete the serial transfers through Column 11 in the mother plates. The wells of Column 12 contained no drug and were the organism growth control wells.

The daughter plates were loaded with 185 μL per well of the CAMHB described using the Multidrop 384. The daughter plates were completed on the Biomek FX instrument which transferred 5 μL of drug solution from each well of a mother plate to each corresponding well of each daughter plate in a single step.

A standardized inoculum of each organism was prepared per CLSI methods (2). Suspensions were prepared to equal a 0.5 McFarland standard, followed by dilution in test media 1:20. The inocula were dispensed into sterile reservoirs divided by length (Beckman Coulter) and the Biomek 2000 was used to inoculate the plates. Daughter plates were placed on the Biomek 2000 work surface in reverse orientation so that inoculation took place from low to high drug concentration. The Biomek 2000 delivered 10 μL of the diluted suspension into each well resulting in a final concentration of approximately 5×10⁵ CFU/mL. Plates were stacked 3-4 high, covered with a sterile lid on the top plate, placed in plastic bags, and incubated at 35° C. for approximately 18 hr.

The microplates were viewed from the bottom using a plate viewer and the MIC was read. The MIC was recorded as the lowest concentration of drug that inhibited visible growth of the organism. Uninoculated solubility control plates were also observed for evidence of drug precipitation.

FIC Assay Procedure: FIC test ranges were set based on broth microdilution MIC test data. FIC assay plates were prepared using the CLSI broth microdilution procedure (1, 2) and automated liquid handlers (Multidrop 384, Biomek 2000 and Biomek FX) to conduct serial dilutions and liquid transfers.

The wells of a standard 96-well microdilution plate (Costar) were filled with 150 μL of the appropriate diluent in columns 2 through 12. A 300 μL aliquot at 40X the highest final concentration to be tested was added to each well in Column 1 of the plate. The Biomek 2000 was used to make eleven 2-fold serial dilutions in the “combination agent mother” plate from columns 2 through 11.

The wells of the “test agent mother” plate were filled with 150 μL of diluent in rows B-H. Row A of this plate was filled with 300 μL of the test agent stock solutions at 40X the highest final concentration to be tested. Serial 2-fold dilutions were then prepared from row B-G by hand using a multichannel pipette.

The “daughter plates” were loaded with 180 μL of CAMHB using the Multidrop 384. The Biomek FX was used to transfer 5 μL of drug solution from each well of the combination agent mother plate to the corresponding well in all of the daughter plates in a single step. Then a 5 μL aliquot from each well of the test agent mother plate was transferred with the Biomek FX into the corresponding well of the daughter plate. Row H and Column 12 each contained serial dilutions of combination agent and the test agent alone, respectively, for determination of the MIC. This procedure was repeated for test agents and combination agents evaluated.

A standardized inoculum of each organism was prepared per CLSI methods (2). Colonies were picked from the primary plate and a suspension was prepared to equal a 0.5 McFarland turbidity standard. The suspensions were additionally diluted 1:20. A 10 μL standardized inoculum was delivered into each well using the Biomek 2000 from low to high concentration. These inoculations yielded a final cell concentration in the daughter plates of approximately 5×10⁵ CFU/mL in each well.

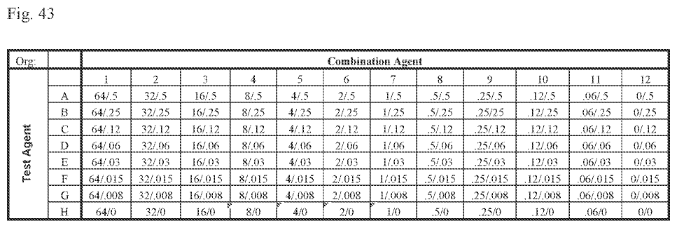

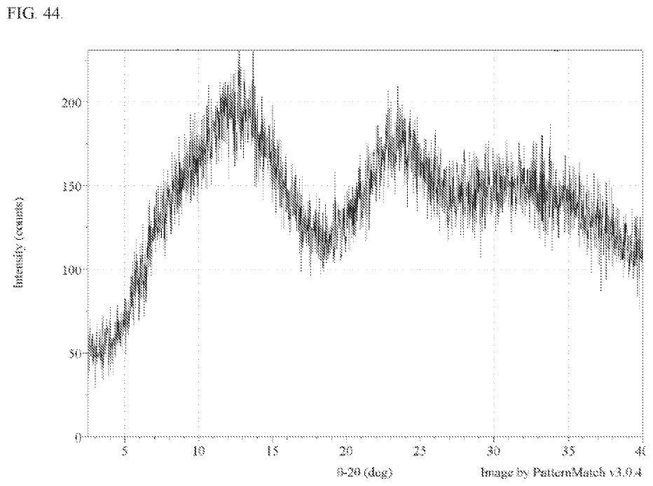

The test format resulted in the creation of an 8×12 checkerboard where each compound was tested alone (Column 12 and Row H) and in combination at varying ratios of drug concentration (see FIG. 43).

Plates were stacked 3-4 high, covered with a sterile lid on the top plate, placed in plastic bags, and incubated at 35° C. for approximately 18 hr (with the exception of P. aeruginosa isolate 8798 which was incubated for 42 hr due to poor growth at 18 hr). Plates were viewed from the bottom using a plate viewer. Prepared reading sheets were marked for the MIC of the combination agent (row H), the MIC of test agent (column 12), and the wells of the growth-no growth interface for wells containing test agent and combination agent at varying ratios. The FIC was read and recorded as the lowest concentration of drug that exhibited no growth of the organism by row where agents were tested in combination (rows B through G). Pinpoint trailing was not interpreted as growth.

FIC/FICI Calculations: FTCs were calculated essentially as described by Eliopoulos, et al. (3), as applicable.

For each relevant row of the panel, the FIC index (FICI) was calculated as below:

FIC_{drug A}/MIC_{drug A}+FIC_{drug B}/MIC_{drug B}=FIC index(FICI)

Mean FICI were determined for the combination.

In the instance where an MIC for one of the test agents was off-scale (greater than the highest test concentration evaluated, e.g. >32 μg/mL), the MIC was set to the next highest 2-fold concentration for determination of the FIC (e.g. if the MIC was >32 μg/mL, the FIC was calculated based on an MC of 64 μg/mL).

Using the criteria described by Odds (4), the mean FICI for the combination was interpreted as follows: ≤0.5=synergy, >0.5-4=additive/indifferent, and >4=antagonism.

An interpretation of “synergy” is consistent with inhibition of organism growth by combinations at concentrations significantly below (>4-fold) the MIC of either compound alone, resulting in a low FICI value (≤0.50). An interpretation of “indifference” is consistent with growth inhibition at concentrations at or slightly below/above the MIC values of the individual compounds alone, resulting in an FICI value of >0.50 but less than or equal to 4.0. An interpretation of “antagonism” results when the concentrations of the compounds in combination that are required to inhibit organism growth are substantially greater (>4-fold) than those for the compounds individually, resulting in an FICI value of >4.0.

### Results and Discussion

Broth microdilution MIC values for the evaluated agents against the test organisms as observed during initial MIC testing are summarized in Table 24. MIC values for BisEDT and the other test agents against P. aeruginosa ATCC 27853 were within CLSI QC ranges (1). BisEDT maintained activity across the evaluated isolates despite the high degree of resistance to other agents. Based on the resulting phenotypes, isolates shaded in grey were selected for subsequent evaluation in checkerboard assays with BisEDT in combination with the other test agents.

The MIC values observed during FIC testing are summarized in Table 25. As expected, these results were consistent (typically identical or within 2-fold) with those observed during initial MIC testing (Table 24). The rare instances where MIC values differed 4-fold with those observed during initial MIC testing are shaded in grey. As during initial MIC testing, BisEDT and the other test agents had MIC values within the QC ranges for P. aeruginosa ATCC 27853. The median MIC value of BisEDT as observed across 6 checkerboard panels is reported in Table 25 alongside the MIC range. The MIC values observed with BisEDT were consistent across checkerboard panels during FIC testing as expected.

All test data from FIC panels are shown by organism in Tables 28-87. The mean FICI values observed for BisEDT in combination with all evaluated agents across the selected isolates are summarized in Table 26. Instances where individual FICI values on checkerboard panels exhibited synergy/antagonism are also noted.

Excluding colistin and ciprofloxacin, the majority of the interactions observed between BisEDT and other agents by FICI were additive/indifferent with mean FICI values and individual FICI values across checkerboard panels generally between 0.5 to 4. For a subset of isolates, synergy between BisEDT and colistin was observed based on mean FICI values ≤0.50; for the lone colistin-R P. aeruginosa (isolate 8798), for both isolates of B. cepacia, and for one isolate of B. cenocepacia (isolate 0548). For one of the P. aeruginosa (isolate 9108), antagonism between BisEDT and ciprofloxacin was observed based on a mean FICI value >4. For BisEDT in combination with ciprofloxacin, there were 3 additional isolates where there was at least one row on the FiC panel that had an FICI value indicative of antagonism. There was also one isolate of B. cepacia (isolate 1793) where there was one row with an FICI value indicative of antagonism for BisEDT in combination with meropenem.

In summary, the overall interaction between BisEDT and other agents used to treat CF was additive/indifferent against the CF pathogens P. aeruginosa and B. cepacia complex with the exception of select instances where synergy was apparent for BisEDT in combination with colistin and select instances where antagonism was apparent for BisEDT in combination with ciprofloxacin. Whether these instances indicate true synergy or antagonism for these combinations requires further investigation by time-kill kinetic analysis.

TABLE 24
Summary of activity as observed during initial MIC testing

| | | | MIC (μg/mL) | | | | | | |
|---|---|---|---|---|---|---|---|---|---|
| Organism | Isolate ID | Phenotype¹ | BisEDT | MEM | TOB | AMK | CIP | AZT | COL |
| P. aeruginosa | 103 | — | 1 | 1 | 1 | 2 | 0.5 | 8 | 0.5 |
| | (ATCC 25922) | | (0.5-4)² | (0.12-1) | (0.25-1) | (1-4) | (0.12-1) | (2-8) | (0.5-4) |
| | 1497 | TOB-R AMK-R, CIP-I | 0.5 | 0.06 | 16 | 64 | 2 | 32 | 4 |
| | | AZT-R, COL-R | | | | | | | |
| | 1530 | TOB-R, AMK-R, CIP-R | 1 | 0.5 | 32 | 64 | 8 | 4 | 0.5 |
| | 1553 | TOB-R, AMK-R, CIP-R | 7 | 0.5 | >256 | 64 | 4 | 8 | 0.5 |
| | 6322 | MEM-R, TOB-R, AMK-R, | 7 | 16 | 128 | 64 | 64 | 64 | 0.5 |
| | | CIP-R, AZT-R | | | | | | | |
| | 6977 | MEM-I, TOB-R, CIP-R, | 1 | 4 | 128 | 16 | 128 | 16 | 1 |
| | | AZT-I | | | | | | | |
| | 7745 | MEM-R, CIP-R, AZT-R | 2 | 16 | 0.5 | 4 | 4 | 32 | 0.5 |
| | 7754 | MEM-R, TOB-R, CIP-R, | 1 | 32 | 32 | 8 | 8 | 16 | 0.5 |
| | | AZT-I | | | | | | | |
| | 7762 | MEM-I, CIP-I | 7 | 4 | 1 | 8 | 7 | 8 | 1 |
| | 7871 | MEM-R, AZT-I | 1 | 8 | 0.5 | 2 | 0.25 | 16 | 0.5 |
| | 7886 | MEM-R, CIP-R, AZT-R | 2 | 32 | 1 | 8 | 8 | 64 | 0.5 |
| | 7874 | — | 1 | 1 | 0.5 | 2 | 0.12 | 4 | 1 |
| | 8797 | MEM-R, TOB-R, AMK-R, | 0.25 | 32 | >256 | >256 | 8 | >256 | >256 |
| | | CIP-R, AZT-R, COL-R | | | | | | | |
| | 8798 | MEM-R, TOB-R, AMK-R, | 2 | 64 | 32 | 128 | 4 | 256 | >256 |
| | | CIP-R, AZT-R, COL-R | | | | | | | |
| | 9108 | CIP-R, AZT-I | 2 | 0.5 | 0.5 | 4 | 32 | 16 | 0.5 |

TABLE 25
Summary of activity as observed during initial MIC testing

| | Isolate | | MIC (μg/mL) | | | | | | |
|---|---|---|---|---|---|---|---|---|---|
| Organism | ID | Phenotype¹ | BisEDT | MEM | TOB | AMK | CIP | AZT | COL |
| B. cepacia | 0546 | — | 2 | 4 | 64 | 64 | 2 | 16 | >256 |
| | 0547 | — | 1 | 2 | 128 | 128 | 1 | 16 | >256 |
| | 9040 | — | 1 | 4 | 8 | 8 | 1 | 32 | >256 |
| | 1793 | — | 0.5 | 4 | 64 | 64 | 0.5 | 128 | >256 |
| | 1794 | — | 2 | 4 | 64 | 128 | 1 | 32 | >256 |
| B. | 0548 | CIP-R | 8 | 4 | 64 | 256 | 8 | 64 | >256 |
| cenocepacia | 0813 | CPI-I | 2 | 2 | 256 | >256 | 4 | 8 | >256 |
| | 1631 | — | 2 | 4 | 128 | 256 | 2 | 32 | >256 |
| | 1783 | — | 2 | 4 | 128 | 256 | 2 | 32 | >256 |
| | 8555 | — | 2 | 4 | 128 | 256 | 2 | 256 | >256 |
| B. | 1580 | CIP-R | 9 | 2 | 128 | 128 | 8 | 64 | >256 |
| multivorans | 1791 | — | 1 | 4 | 64 | 128 | 1 | 4 | >256 |
| | 1795 | MEM-I, CIP-R | 2 | 8 | >256 | >256 | 32 | 4 | >256 |
| | 5665 | CIP-I | 1 | 2 | 64 | 256 | 4 | 4 | >256 |
| | 8952 | CIP-I | 4 | 2 | 16 | 64 | 4 | 8 | >256 |

MEM = meropenem,
TOB = tobramycin,
AMK = amikacin,
CIP = ciprofloxacin,
AZT = aztreonam,
COL = colistin,
-R = resistant,
-I = intermediate
¹Phenotype is based off of MIC interpretation in accordance with CLSI breakpoints (1) or in the case of colistin and P. aeruginosa EUCAST breakpoints (v.8.1)
Note
that B. cepacia complex are intrinsically resistant to aminoglycosides, aztreonam, and colistin (1)
²CLSI QC range shown in parenthesis where applicable
Cells shaded grey were selected for FIC testing

TABLE 26
Summary of activity as observed during FJC testing

| | | | MIC (μg/mL) | | | | | | |
|---|---|---|---|---|---|---|---|---|---|
| Organism | Isolate ID | Phenotype¹ | BisEDT³ | MEM | TOB | AMK | CIP | AZT | COL |
| P. aeruginosa | 103 | — | 1 | 1 | 1 | 4 | 1 | 8 | 0.5 |
| | (ATCC 25922)² | | (0.5-4) | (0.12-1) | (0.25-1) | (1-4) | (0.12-1) | (2-8) | (0.5-4) |
| | 6322 | MEM-R, TOB-R, AMK-R, | 1-2 (2) | 16 | 128 | 64 | 128 | 32 | 0.5 |
| | | CIP-R, AZT-R | | | | | | | |
| | 6977 | MEM-T, TOB-R, CIP-R, | 1 (1) | 8 | 128 | 16 | 128 | 64 | 1 |
| | | AZT-I | | | | | | | |
| | 7745 | MEM-R, CIP-R, AZT-R | 1-2 (2) | 16 | 0.5 | 4 | 2 | 16 | 0.5 |
| | 8798 | MEM-R, TOB-R, AMK-R, | 2 (2) | 32 | 16 | 128 | 8 | >256 | >256 |
| | | CIP-R, AZT-R, COL-R | | | | | | | |
| | 9108 | CIP-R, AZT-I | 2-4 (2) | I | 0.5 | 4 | 16 | 16 | 0.5 |
| B. cepacia | 0546 | | 2-4 (2, 4) | 4 | 32 | 64 | 2 | 32 | >256 |
| | 1793 | — | 1-2 (1) | 4 | 16 | 32 | 0.5 | 128 | >256 |
| B. cenocepacia | 0548 | CIP-R | 4 (4) | 4 | 32 | 128 | 2 | 64 | >256 |
| | 0813 | CIP-I | 2-4 (2, 4) | 9 | 256 | >256 | 2 | 32 | >256 |
| B. multivorans | 1795 | MEM-I, CIP-R | 2 (2) | 4 | >256 | >256 | 32 | 16 | >256 |

MEM = meropenem,
TOB = tobramycin,
AMK = amikacin,
CIP = ciprofloxacin,
AZT = aztreonam,
COL = colistin,
-R = resistant,
-I = intermediate
¹Phenotype determined based on initial MIC testing as shown in Table 1
Note
that B. cepacia complex are intrinsically resistant to aminoglycosides, aztreonam, and colistin (1)
²CLSI QC range shown in parenthesis for ATCC 25922; ATCC 25922 was not tested on checkerboard panels with agents in combination, each agent was tested alone solely for the purpose of QC
³With the exception of ATCC 25922 where only one replicate was tested for the purpose of quality control (QC range shown in parenthesis), the MIC result for BisEDT represents the MIC range and mode as observed across six different FIC panels.

TABLE 27
Summary of mean FICI data for BisEDT in combination with evaluated agents

| | | Mean FICI | | | | | |
|---|---|---|---|---|---|---|---|
| Isolate ID | Phenotype¹ | MEM | TOB | AMK | CIP | AZT | COL |
| 6322 | MEM-R, TOB-R, AMK-R, | 1.19 | 1.19 | 2.23 | 1.19 | 1.19 | 1.23 |
| | CIP-R, AZT-R | | | | | | |
| 6977 | MEM-I, TOB-R, CIP-R, | 1.73 | 2.23 | 2.23 | 2.23 | 1.23 | 1.23 |
| | AZT-I | | | | | | |
| 7745 | MEM-R, CIP-R, AZT-R | 0.64 | 1.19 | 1.23 | 2.59* | 0.99 | 1.19 |
| 8798 | MEM-R, TOB-R, AMK-R, | 1.19 | 0.91 | 1.09 | 0.99 | 0.89 | 0.22 |
| | CIP-R, AZT-R, COL-R | | | | | | |
| 9108 | CIP-R, AZT-I | 1.19 | 1.19 | 1.29 | 4.50 | 0.79 | 1.08 |
| 0546 | — | 1.99 | 0.69 | 0.64 | 1.35 | 1.16 | 0.25 |
| 1793 | — | 2.59* | 1.11 | 1.29 | 1.98 | 1.23 | 0.33 |
| 0548 | CIP-R | 1.11 | 0.67 | 0.67 | 3.23* | 1.23 | 0.41 |
| 0813 | CIP-I | 1.08 | 0.91 | 0.99 | 2.85* | 1.19 | 0.72 |
| 1795 | MEM-I, CIP-R | 1.33 | 1.33 | 1.33 | 1.19 | 1.39 | 1.33 |

MEM = meropenem,
TOB = tobramycin,
AMK = amikacin,
CIP = ciprofloxacin,
AZT = aztreonam,
COL = colistin,
-R = resistant,
-I = intermediate
¹Phenotype determined based on initial MIC testing as shown in Table 1
**indicates that at least one row on the test panel had an individual FICI value ≤ 0.5 (indicative of synergy)
*indicates that at least one row on the test panel had an individual FICI value > 4 (indicative of antagonism)

TABLE 28
MIC (μg/mL), FIC, FICI, for BisEDT in combination with Meropenem

| Organism: P. aeruginosa | 6322 | | | FICI (N): | 5 |
|---|---|---|---|---|---|
| Drug A: BisEDT | Drag A MIC: | | 2 | SUM FICI: | 5.97 |
| DrugB: Meropenem | Drag B MIC: | | 16 | MEAN FICI: | 1.19 |
| Row | MIC_{A} | FIC_{A} | MIC_{B} | FIC_{B} | FICI |
| A | | | | | |
| B | | | | | |
| C | 1 | 0.5 | 16 | 1 | 1.5 |
| D | 0.5 | 0.25 | 16 | 1 | 1.25 |
| E | 0.25 | 0.125 | 16 | 1 | 1.125 |
| F | 0.12 | 0.06 | 16 | 1 | 1.06 |
| G | 0.06 | 0.03 | 16 | 1 | 1.03 |
| H | | | | | |

TABLE 29
MIC (μg/mL), FIC, FICI, for BisEDT in combination with Tobramycin

| Organism: P. aeruginosa | 6322 | | | FICI (N): | 5 |
|---|---|---|---|---|---|
| Drug A: BisEDT | Drug A MIC: | | 2 | SUM FICI: | 5.97 |
| DrugB: Tobramycin | Drag B MIC: | | 128 | MEAN FICI: | 1.19 |
| Row | MIC_{A} | FIC_{A} | MIC_{B} | FIC_{B} | FICI |
| A | | | | | |
| B | | | | | |
| C | 1 | 0.5 | 128 | 1 | 1.5 |
| D | 0.5 | 0.25 | 128 | 1 | 1.25 |
| E | 0.25 | 0.125 | 128 | 1 | 1.125 |
| F | 0.12 | 0.06 | 128 | 1 | 1.06 |
| G | 0.06 | 0.03 | 128 | 1 | 1.03 |
| H | | | | | |

TABLE 30
MIC (μg/mL), FIC, FICI, for BisEDT in combination with Amikacin

| Organism: P. aeruginosa | 6322 | | | FICI (N): | 4 |
|---|---|---|---|---|---|
| Drug A: BisEDT | Drug A MIC: | | 1 | SUM FICI: | 8.93 |
| DrugB: Amikacin | Drug B MIC: | | 64 | MEAN FICI: | 2.23 |
| Row | MIC_{A} | FIC_{A} | MIC_{B} | FIC_{B} | FICI |
| A | | | | | |
| B | | | | | |
| C | | | | | |
| D | 0.5 | 0.5 | 128 | 2 | 2.5 |
| E | 0.25 | 0.25 | 128 | 2 | 2.25 |
| F | 0.12 | 0.12 | 128 | 2 | 2.12 |
| G | 0.06 | 0.06 | 128 | 2 | 2.06 |
| H | | | | | |

TABLE 31
MIC (μg/mL), FIC, FICI, for BisEDT in combination with Ciprofloxacin

| Organism: P. aeruginosa | 6322 | | | FICI (N): | 5 |
|---|---|---|---|---|---|
| Drag A: BisEDT | Drag A MIC: | | 2 | SUM FICI: | 5.97 |
| DragB: Ciprofloxacin | Drag B MIC: | | 128 | MEAN FICI: | 1.19 |
| Row | MIC_{A} | FIC_{A} | MIC_{B} | FIC_{B} | FICI |
| A | | | | | |
| B | | | | | |
| C | 1 | 0.5 | 128 | 1 | 1.5 |
| D | 0.5 | 0.25 | 128 | 1 | 1.25 |
| E | 0.25 | 0.125 | 128 | 1 | 1.125 |
| F | 0.12 | 0.06 | 128 | 1 | 1.06 |
| G | 0.06 | 0.03 | 128 | 1 | 1.03 |
| H | | | | | |

TABLE 32
MIC (μg/mL), FIC, FICI, for BisEDT in combination with Aztreonam

| | | | | |
|---|---|---|---|---|
| Organism: | P. aeruginosa | 6322 | FICI (N): | 5 |

| | | | | | |
|---|---|---|---|---|---|
| Drug A: | BisEDT | Drug A MIC: | 2 | SUM FICI: | 5.97 |
| Drug B: | Aztreonam | Drug B MIC: | 32 | MEAN FICI: | 1.19 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 36 | 0.5 | 1 |
| D | 0.5 | 0.25 | 16 | 0.5 | 0.75 |
| E | 0.25 | 0.125 | 30 | 1 | 1.125 |
| F | 0.12 | 0.06 | 32 | 1 | 1.06 |
| G | 0.06 | 0.03 | 64 | 2 | 2.03 |
| H | | | | | |

TABLE 33
MIC (μg/mL), FIC, FICI, for BisEDT in combination with Colistin

| | | | | |
|---|---|---|---|---|
| Organism: | P. aeruginosa | 6322 | FICI (N): | 4 |

| | | | | | |
|---|---|---|---|---|---|
| Drug A: | BisEDT | Drug A MIC: | 1 | SUM FICI: | 4.93 |
| Drug B: | Colistin | Drug B MIC: | 0.5 | MEAN FICI: | 1.23 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 0.5 | 1 | 1.5 |
| E | 0.25 | 0.25 | 0.5 | 1 | 1.25 |
| F | 0.12 | 0.12 | 0.5 | 1 | 1.12 |
| G | 0.06 | 0.06 | 0.5 | 1 | 1.06 |
| H | | | | | |

TABLE 34
MIC (μg/mL), FIC, FICI, for BisEDT in combination with Meropenem

| | | | | |
|---|---|---|---|---|
| Organism: | P. aeruginosa | 6977 | FICI (N): | 4 |

| | | | | | |
|---|---|---|---|---|---|
| Drug A: | BisEDT | Drug A MIC: | 1 | SUM FICI: | 6.93 |
| Drug B: | Meropenem | Drug B MIC: | 8 | MEAN FICI: | 1.73 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 16 | 2 | 2.5 |
| E | 0.25 | 0.25 | 16 | 2 | 2.25 |
| F | 0.12 | 0.12 | 8 | 1 | 1.12 |
| G | 0.06 | 0.06 | 8 | 1 | 1.06 |
| H | | | | | |

TABLE 35
MIC (μg/mL), FIC, FICI, for BisEDT in combination with Tobramycin

| | | | | |
|---|---|---|---|---|
| Organism: | P. aeruginosa | 6977 | FICI (N): | 4 |

| | | | | | |
|---|---|---|---|---|---|
| Drug A: | BisEDT | Drug A MIC: | 1 | SUM FICI: | 8.93 |
| Drug B: | Tobramycin | Drug B MIC: | 128 | MEAN FICI: | 2.23 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 256 | 2 | 2.5 |
| E | 0.25 | 0.25 | 256 | 2 | 2.25 |
| F | 0.12 | 0.12 | 256 | 2 | 2.12 |
| G | 0.06 | 0.06 | 256 | 2 | 2.06 |
| H | | | | | |

TABLE 36
MIC (μg/mL), FIC, FICI, for BisEDT in combination with Amikacin

| | | | | |
|---|---|---|---|---|
| Organism: | P. aeruginosa | 6977 | FICI (N): | 4 |

| | | | | | |
|---|---|---|---|---|---|
| Drug A: | BisEDT | Drug A MIC: | 1 | SUM FICI: | 8.93 |
| Drug B: | Amikacin | Drug B MIC: | 16 | MEAN FICI: | 2.23 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 32 | 2 | 2.5 |
| E | 0.25 | 0.25 | 32 | 2 | 2.25 |
| F | 0.12 | 0.12 | 32 | 2 | 2.12 |
| G | 0.06 | 0.06 | 32 | 2 | 2.06 |
| H | | | | | |

TABLE 37
MIC (μg/mL), FIC, FICI, for BisEDT in combination with Ciprofloxacin

| | | | | |
|---|---|---|---|---|
| Organism: | P. aeruginosa | 6977 | FICI (N): | 4 |

| | | | | | |
|---|---|---|---|---|---|
| Drug A: | BisEDT | Drug A MIC: | 1 | SUM FICI: | 8.93 |
| Drug B: | Ciprofloxacin | Drug B MIC: | 128 | MEAN FICI: | 2.23 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 256 | 2 | 2.5 |
| E | 0.25 | 0.25 | 256 | 2 | 2.25 |
| F | 0.12 | 0.12 | 256 | 2 | 2.12 |
| G | 0.06 | 0.06 | 256 | 2 | 2.06 |
| H | | | | | |

TABLE 38
MIC (μg/mL), FIC, FICI, for BisEDT in combination with Aztreonam

| | | | | |
|---|---|---|---|---|
| Organism: | P. aeruginosa | 6977 | FICI (N): | 4 |

| | | | | | |
|---|---|---|---|---|---|
| Drug A: | BisEDT | Drug A MIC: | 1 | SUM FICI: | 4.93 |
| Drug B: | Aztreonam | Drug B MIC: | 64 | MEAN FICI: | 1.23 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 64 | 1 | 1.5 |
| E | 0.25 | 0.25 | 64 | 1 | 1.25 |
| F | 0.12 | 0.12 | 64 | 1 | 1.12 |
| G | 0.06 | 0.06 | 64 | 1 | 1.06 |
| H | | | | | |

TABLE 39
MIC (μg/mL), FIC, FICI, for BisEDT in combination with Colistin

| | | | | |
|---|---|---|---|---|
| Organism: | P. aeruginosa | 6977 | FICI (N): | 4 |

| | | | | | |
|---|---|---|---|---|---|
| Drug A: | BisEDT | Drug A MIC: | 1 | SUM FICI: | 4.93 |
| Drug B: | Colistin | Drug B MIC: | 1 | MEAN FICI: | 1.23 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 1 | 1 | 1.5 |
| E | 0.25 | 0.25 | 1 | 1 | 1.25 |
| F | 0.12 | 0.12 | 1 | 1 | 1.12 |
| G | 0.06 | 0.06 | 1 | 1 | 1.06 |
| H | | | | | |

TABLE 40
MIC (μg/mL), FIC, FICI, for BisEDT in combination with Meropenem

| | | | | |
|---|---|---|---|---|
| Organism: | P. aeruginosa | 7745 | FICI (N): | 5 |

| | | | | | |
|---|---|---|---|---|---|
| Drug A: | BisEDT | Drug A MIC: | 2 | SUM FICI: | 3.22 |
| Drug B: | Meropenem | Drug B MIC: | 16 | MEAN FICI: | 0.64 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 4 | 0.25 | 0.75 |
| D | 0.5 | 0.25 | 8 | 0.5 | 0.75 |
| E | 0.25 | 0.125 | 8 | 0.5 | 0.625 |
| F | 0.12 | 0.06 | 8 | 0.5 | 0.56 |
| G | 0.06 | 0.03 | 8 | 0.5 | 0.53 |
| H | | | | | |

TABLE 41
MIC (μg/mL), FIC, FICI, for BisEDT in combination with Tobramycin

| | | | | |
|---|---|---|---|---|
| Organism: | P. aeruginosa | 7745 | FICI (N): | 5 |

| | | | | | |
|---|---|---|---|---|---|
| Drug A: | BisEDT | Drug A MIC: | 2 | SUM FICI: | 5.97 |
| Drug B: | Tobramycin | Drug B MIC: | 0.5 | MEAN FICI: | 1.19 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 0.5 | 1 | 1.5 |
| D | 0.5 | 0.25 | 0.5 | 1 | 1.25 |
| E | 0.25 | 0.125 | 0.5 | 1 | 1.125 |
| F | 0.12 | 0.06 | 0.5 | 1 | 1.06 |
| G | 0.06 | 0.03 | 0.5 | 1 | 1.03 |
| H | | | | | |

TABLE 42
MIC (μg/mL), FIC, FICI, for BisEDT in combination with Amikacin

| | | | | |
|---|---|---|---|---|
| Organism: | P. aeruginosa | 7745 | FICI (N): | 4 |

| | | | | | |
|---|---|---|---|---|---|
| Drug A: | BisEDT | Drug A MIC: | 1 | SUM FICI: | 4.93 |
| Drug B: | Amikacin | Drug B MIC: | 4 | MEAN FICI: | 1.23 |

| Row | MIC_{A} | FIC_{A} | MIC_{B} | FIC_{B} | FICI |
|---|---|---|---|---|---|
| A | | | | | |
| B | | | | | |
| C | | | | | |
| D | 0.5 | 0.5 | 4 | 1 | 1.5 |
| E | 0.25 | 0.25 | 4 | 1 | 1.25 |
| F | 0.12 | 0.12 | 4 | 1 | 1.12 |
| G | 0.06 | 0.06 | 4 | 1 | 1.06 |
| H | | | | | |

TABLE 43
MIC (μg/mL), FIC, FICI, for BisEDT in combination with Ciprofloxacin

| | | | | |
|---|---|---|---|---|
| Organism: | P. aeruginosa | 7745 | FICI (N): | 5 |

| | | | | | |
|---|---|---|---|---|---|
| Drug A: | BisEDT | Drug A MIC: | 2 | SUM FICI: | 12.97 |
| Drug B: | Ciprofloxacin | Drug B MIC: | 2 | MEAN FICI: | 2.59 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 4 | 2 | 2.5 |
| D | 0.5 | 0.25 | 8 | 4 | 4.25 |
| E | 0.25 | 0.125 | 4 | 2 | 2.125 |
| F | 0.12 | 0.06 | 4 | 2 | 2.06 |
| G | 0.06 | 0.03 | 4 | 2 | 2.03 |
| H | | | | | |

TABLE 44
MIC (μg/mL), FIC, FICI, for BisEDT in combination with Aztreonam

| | | | | |
|---|---|---|---|---|
| Organism: | P. aeruginosa | 7745 | FICI (N): | 5 |

| | | | | | |
|---|---|---|---|---|---|
| Drug A: | BisEDT | Drug A MIC: | 2 | SUM FICI: | 4.97 |
| Drug B: | Aztreonam | Drug B MIC: | 16 | MEAN FICI: | 0.99 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 8 | 0.5 | 1 |
| D | 0.5 | 0.25 | 8 | 0.5 | 0.75 |
| E | 0.25 | 0.125 | 16 | 1 | 1.125 |
| F | 0.12 | 0.06 | 16 | 1 | 1.06 |
| G | 0.06 | 0.03 | 16 | 1 | 1.03 |
| H | | | | | |

TABLE 45
MIC (μg/mL), FIC, FICI, for BisEDT in combination with Colistin

| | | | | |
|---|---|---|---|---|
| Organism: | P. aeruginosa | 7745 | FICI (N): | 5 |

| | | | | | |
|---|---|---|---|---|---|
| Drug A: | BisEDT | Drug A MIC: | 2 | SUM FICI: | 5.97 |
| Drug B: | Colistin | Drug B MIC: | 0.5 | MEAN FICI: | 1.19 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 0.5 | 1 | 1.5 |
| D | 0.5 | 0.25 | 0.5 | 1 | 1.25 |
| E | 0.25 | 0.125 | 0.5 | 1 | 1.125 |
| F | 0.12 | 0.06 | 0.5 | 1 | 1.06 |
| G | 0.06 | 0.03 | 0.5 | 1 | 1.03 |
| H | | | | | |

TABLE 46
MIC (μg/mL), FIC, FICI, for BisEDT in combination with Meropenem

| | | | | |
|---|---|---|---|---|
| Organism: | P. aeruginosa | 8798 | FICI (N): | 5 |

| | | | | | |
|---|---|---|---|---|---|
| Drug A: | BisEDT | Drug A MIC: | 2 | SUM FICI: | 5.97 |
| Drug B: | Meropenem | Drug B MIC: | 32 | MEAN FICI: | 1.19 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 32 | 1 | 1.5 |
| D | 0.5 | 0.25 | 32 | 1 | 1.25 |
| E | 0.25 | 0.125 | 32 | 1 | 1.125 |
| F | 0.12 | 0.06 | 32 | 1 | 1.06 |
| G | 0.06 | 0.03 | 32 | 1 | 1.03 |
| H | | | | | |

TABLE 47
MIC (μg/L), FIC, FICI, for BisEDT in combination with Tobramycin

| Organism: P. aeruginosa | 8798 | | | FICI (N): | | 6 |
|---|---|---|---|---|---|---|
| Drug A: BisEDT | Drug A MIC: | | 2 | SUM FICI: | | 5.47 |
| DrugB: Tobramycin | Drug B MIC: | | 16 | MEAN FICI: | | 0.9 |
| Row | MIC_{A} | FIC_{A} | MIC_{B} | FIC_{B} | FICI | |
| A | | | | | | |
| B | | | | | | |
| C | 1 | 0.5 | 8 | 0.5 | 1 | |
| D | 0.5 | 0.25 | 16 | 1 | 1.25 | |
| E | 0.25 | 0.125 | 16 | 1 | 1.125 | |
| F | 0.12 | 0.06 | 16 | 1 | 1.06 | |
| G | 0.06 | 0.03 | 16 | 1 | 1.03 | |
| H | | | | | | |

TABLE 48
MIC (μg/L), FIC, FICI, for BisEDT in combination with Amikacin

| Organism: P. aeruginosa | 8798 | | | FICI (N): | | 5 |
|---|---|---|---|---|---|---|
| Drug A: BisEDT | Drug A MIC: | | 2 | SUM FICI: | | 5.47 |
| DrugB: Amikacin | Drug B MIC: | | 128 | MEAN FICI: | | 1.09 |
| Row | MIC_{A} | FIC_{A} | MIC_{B} | FIC_{B} | FICI | |
| A | | | | | | |
| B | | | | | | |
| C | 1 | 0.5 | 64 | 0.5 | 1 | |
| D | 0.5 | 0.25 | 128 | 1 | 1.25 | |
| E | 0.25 | 0.125 | 128 | 1 | 1.125 | |
| F | 0.12 | 0.06 | 128 | 1 | 1.06 | |
| G | 0.06 | 0.03 | 128 | 1 | 1.03 | |
| H | | | | | | |

TABLE 49
MIC (μg/L), FIC, FICI, for BisEDT in combination with Ciprofloxacin

| Organism: P. aeruginosa | 8798 | | | FICI (N): | | 5 |
|---|---|---|---|---|---|---|
| Drug A: BisEDT | Drug A MIC: | | 2 | SUM FICI: | | 4.97 |
| DrugB: Ciproofloxacin | Drug B MIC: | | 8 | MEAN FICI: | | 0.99 |
| Row | MIC_{A} | FIC_{A} | MIC_{B} | FIC_{B} | FICI | |
| A | | | | | | |
| B | | | | | | |
| C | 1 | 0.5 | 4 | 0.5 | 1 | |
| D | 0.5 | 0.25 | 4 | 0.5 | 0.75 | |
| E | 0.25 | 0.125 | 8 | 1 | 1.125 | |
| F | 0.12 | 0.06 | 8 | 1 | 1.06 | |
| G | 0.06 | 0.03 | 8 | 1 | 1.03 | |
| H | | | | | | |

TABLE 50
MIC (μg/L), FIC, FICI, for BisEDT in combination with Aztreonam

| Organism: P. aeruginosa | 8798 | | | FICI (N): | | 5 |
|---|---|---|---|---|---|---|
| Drug A: BisEDT | Drug A MIC: | | 2 | SUM FICI: | | 4.47 |
| DrugB: Aztreonam | Drug B MIC: | | >256 | MEAN FICI: | | 0.89 |
| Row | MIC_{A} | FIC_{A} | MIC_{B} | FIC_{B} | FICI | |
| A | | | | | | |
| B | | | | | | |
| C | 1 | 0.5 | 256 | 0.5 | 1 | |
| D | 0.5 | 0.25 | 256 | 0.5 | 0.75 | |
| E | 0.25 | 0.125 | 256 | 0.5 | 1.625 | |
| F | 0.12 | 0.06 | >256 | 1 | 1.06 | |
| G | 0.06 | 0.03 | >256 | 1 | 1.03 | |
| H | | | | | | |

TABLE 51
MIC (μg/L), FIC, FICI, for BisEDT in combination with Colistin

| Organism: | 8798 | | | FICI (N): | | 5 |
|---|---|---|---|---|---|---|
| Drug A: BisEDT | Drug A MIC: | | 2 | SUM FICI: | | 1.12 |
| DrugB: Colistin | Drug B MIC: | | >256 | MEAN FICI: | | 0.22 |
| Row | MIC_{A} | FIC_{A} | MIC_{B} | FIC_{B} | FICI | |
| A | | | | | | |
| B | | | | | | |
| C | 1 | 0.5 | 8 | 0.16 | 0.516 | |
| D | 0.5 | 0.25 | 8 | 0.016 | 0.266 | |
| E | 0.25 | 0.125 | 16 | 0.031 | 0.156 | |
| F | 0.12 | 0.06 | 16 | 0.031 | 0.091 | |
| G | 0.06 | 0.03 | 32 | 0.063 | 0.093 | |
| H | | | | | | |

TABLE 52
MIC (μg/L), FIC, FICI, for BisEDT in combination with Meropenem

| Organism: | P. aeruginosa 9108 | | | FICI (N): | | 5 |
|---|---|---|---|---|---|---|
| Drug A: | BisEDT | Drug A MIC: | 1 | SUM FICI: | | 5.97 |
| Drug B: | Meropenem | Drug B MIC: | 1 | MEAN FICI: | | 1.19 |
| Row | MIC_{A} | FIC_{A} | MIC_{B} | FIC_{B} | FICI | |
| A | | | | | | |
| B | | | | | | |
| C | 1 | 0.5 | 1 | 1 | 1.5 | |
| D | 0.5 | 0.25 | 1 | 1 | 1.25 | |
| E | 0.25 | 0.125 | 1 | 1 | 1.125 | |
| F | 0.12 | 0.06 | 1 | 1 | 1.06 | |
| G | 0.06 | 0.03 | 1 | 1 | 1.03 | |
| H | | | | | | |

TABLE 53
MIC (μg/L), FIC, FICI, for BisEDT in combination with Tobramycin

| Organism: | P. aeruginosa 9108 | | | FICI (N): | | 5 |
|---|---|---|---|---|---|---|
| Drug A: | BisEDT | Drug A MIC: | 2 | SUM FICI: | | 5.97 |
| Drug B: | Tobramycin | Drug B MIC: | 0.5 | MEAN FICI: | | 1.19 |
| Row | MIC_{A} | FIC_{A} | MIC_{B} | FIC_{B} | FICI | |
| A | | | | | | |
| B | | | | | | |
| C | 1 | 0.5 | 0.5 | 1 | 1.5 | |
| D | 0.5 | 0.25 | 0.5 | 1 | 1.25 | |
| E | 0.25 | 0.125 | 0.5 | 1 | 1.125 | |
| F | 0.12 | 0.06 | 0.5 | 1 | 1.06 | |
| G | 0.06 | 0.03 | 0.5 | 1 | 1.03 | |
| H | | | | | | |

TABLE 54
MIC (μg/L), FIC, FICI, for BisEDT in combination with Amikacin

| Organism: | P. aeruginosa 9108 | | | FICI (N): | | 5 |
|---|---|---|---|---|---|---|
| Drug A: | BisEDT | Drug A MIC: | 2 | SUM FICI: | | 4.47 |
| Drug B: | Amikacin | Drug B MIC: | 4 | MEAN FICI: | | 1.29 |
| Row | MIC_{A} | FIC_{A} | MIC_{B} | FIC_{B} | FICI | |
| A | | | | | | |
| B | | | | | | |
| C | 1 | 0.5 | 2 | 0.5 | 1 | |
| D | 0.5 | 0.25 | 8 | 2 | 2.25 | |
| E | 0.25 | 0.125 | 4 | 1 | 1.125 | |
| F | 0.12 | 0.06 | 4 | 1 | 1.06 | |
| G | 0.06 | 0.03 | 4 | 1 | 1.03 | |
| H | | | | | | |

TABLE 55
MIC (μg/L), FIC, FICI, for BisEDT in combination with Ciprofloxacin

| Organism: | P. aeruginosa 9108 | | | FICI (N): | | 6 |
|---|---|---|---|---|---|---|
| Drug A: | BisEDT | Drug A MIC: | 4 | SUM FICI: | | 27.00 |
| Drug B: | Ciprofloxacin | Drug B MIC: | 16 | MEAN FICI: | | 4.50 |
| Row | MIC_{A} | FIC_{A} | MIC_{B} | FIC_{B} | FICI | |
| A | | | | | | |
| B | 2 | 0.5 | 0.25 | 0.01563 | 0.51563 | |
| C | 1 | 0.25 | 128 | 8 | 8.25 | |
| D | 0.5 | 0.25 | 128 | 8 | 8.125 | |
| E | 0.25 | 0.063 | 64 | 4 | 4.063 | |
| F | 0.12 | 0.03 | 64 | 4 | 4.03 | |
| G | 0.06 | 0.015 | 32 | 2 | 2.015 | |
| H | | | | | | |

TABLE 56
MIC (μg/L), FIC, FICI, for BisEDT in combination with Aztreonam

| Organism: | P. aeruginosa 9108 | | | FICI (N): | | 5 |
|---|---|---|---|---|---|---|
| Drug A: | BisEDT | Drug A MIC: | 2 | SUM FICI: | | 3.97 |
| Drug B: | Aztreonam | Drug B MIC: | 16 | MEAN FICI: | | 0.79 |
| Row | MIC_{A} | FIC_{A} | MIC_{B} | FIC_{B} | FICI | |
| A | | | | | | |
| B | | | | | | |
| C | 1 | 0.5 | 8 | 0.5 | 1 | |
| D | 0.5 | 0.25 | 8 | 0.5 | 0.75 | |
| E | 0.25 | 0.125 | 8 | 0.5 | 0.625 | |
| F | 0.12 | 0.06 | 8 | 0.5 | 0.56 | |
| G | 0.06 | 0.03 | 16 | 1 | 1.03 | |
| H | | | | | | |

TABLE 57
MIC (μg/L), FIC, FICI, for BisEDT in combination with Colistin

| Organism: | P. aeruginosa 9108 | | | FICI (N): | | 5 |
|---|---|---|---|---|---|---|
| Drug A: | BisEDT | Drug A MIC: | 4 | SUM FICI: | | 6.48 |
| Drug B: | Colistin | Drug B MIC: | 0.5 | MEAN FICI: | | 1.08 |
| Row | MIC_{A} | FIC_{A} | MIC_{B} | FIC_{B} | FICI | |
| A | | | | | | |
| B | 2 | 0.5 | 0.25 | 0.5 | 1 | |
| C | 1 | 0.25 | 0.5 | 1 | 1.25 | |
| D | 0.5 | 0.125 | 0.5 | 1 | 1.125 | |
| E | 0.25 | 0.0625 | 0.5 | 1 | 1.0625 | |
| F | 0.12 | 0.03 | 0.5 | 1 | 1.03 | |
| G | 0.06 | 0.015 | 0.5 | 1 | 1.015 | |
| H | | | | | | |

TABLE 58
MIC (μg/L), FIC, FICI, for BisEDT in combination with Meropenem

| Organism: | B. cepacia 546 | | | FICI (N): | | 5 |
|---|---|---|---|---|---|---|
| Drug A: | BisEDT | Drug A MIC: | 2 | SUM FICI: | | 9.97 |
| Drug B: | Meropenem | Drug B MIC: | 4 | MEAN FICI: | | 1.99 |
| Row | MIC_{A} | FIC_{A} | MIC_{B} | FIC_{B} | FICI | |
| A | | | | | | |
| B | | | | | | |
| C | 1 | 0.5 | 4 | 1 | 1.5 | |
| D | 0.5 | 0.25 | 8 | 2 | 2.25 | |
| E | 0.25 | 0.125 | 8 | 2 | 2.125 | |
| F | 0.12 | 0.06 | 8 | 2 | 2.06 | |
| G | 0.06 | 0.03 | 8 | 2 | 2.03 | |
| H | | | | | | |

TABLE 59
MIC (μg/L), FIC, FICI, for BisEDT in combination with Topramycin

| Organism: | B. cepacia 546 | | | FICI (N): | | 5 |
|---|---|---|---|---|---|---|
| Drug A: | BisEDT | Drug A MIC: | 2 | SUM FICI: | | 3.47 |
| Drug B: | Topramycin | Drug B MIC: | 32 | MEAN FICI: | | 0.69 |
| Row | MIC_{A} | FIC_{A} | MIC_{B} | FIC_{B} | FICI | |
| A | | | | | | |
| B | | | | | | |
| C | 1 | 0.5 | 16 | 0.5 | 1 | |
| D | 0.5 | 0.25 | 16 | 0.5 | 0.75 | |
| E | 0.25 | 0.125 | 16 | 0.5 | 0.625 | |
| F | 0.12 | 0.06 | 16 | 0.5 | 0.56 | |
| G | 0.06 | 0.03 | 16 | 0.5 | 0.53 | |
| H | | | | | | |

TABLE 60
MIC (μg/L), FIC, FICI, for BisEDT in combination with Amikacin

| Organism: | B. cepacia 546 | | | FICI (N): | | 5 |
|---|---|---|---|---|---|---|
| Drug A: | BisEDT | Drug A MIC: | 2 | SUM FICI: | | 3.22 |
| Drug B: | Amikacin | Drug B MIC: | 64 | MEAN FICI: | | 0.64 |
| Row | MIC_{A} | FIC_{A} | MIC_{B} | FIC_{B} | FICI | |
| A | | | | | | |
| B | | | | | | |
| C | 1 | 0.5 | 16 | 0.25 | 0.75 | |
| D | 0.5 | 0.25 | 32 | 0.5 | 0.75 | |
| E | 0.25 | 0.125 | 32 | 0.5 | 0.625 | |
| F | 0.12 | 0.06 | 32 | 0.5 | 0.56 | |
| G | 0.06 | 0.03 | 32 | 0.5 | 0.53 | |
| H | | | | | | |

TABLE 61
MIC (μg/L), FIC, FICI, for BisEDT in combination with Amikacin

| Organism: | B. cepacia 546 | | | FICI (N): | | 5 |
|---|---|---|---|---|---|---|
| Drug A: | BisEDT | Drug A MIC: | 4 | SUM FICI: | | 8.11 |
| Drug B: | Amikacin | Drug B MIC: | 2 | MEAN FICI: | | 1.35 |
| Row | MIC_{A} | FIC_{A} | MIC_{B} | FIC_{B} | FICI | |
| A | | | | | | |
| B | 2 | 0.5 | 0.25 | 0.125 | 0.625 | |
| C | 1 | 0.25 | 4 | 2 | 2.25 | |
| D | 0.5 | 0.125 | 4 | 2 | 2.125 | |
| E | 0.25 | 0.063 | 2 | 1 | 1.063 | |
| F | 0.12 | 0.03 | 2 | 1 | 1.03 | |
| G | 0.06 | 0.015 | 2 | 1 | 1.015 | |
| H | | | | | | |

TABLE 62
MIC (μg/mL), FIC, FICI, for BisEDT in combination with Aztreonam

| Organism: | B. cepacia 546 | | | FICI (N): | | 6 |
|---|---|---|---|---|---|---|
| Drug A: | BisEDT | Drag A MIC: | 4 | SUM FICI: | | 6.98 |
| Drug B: | Aztreonam | Drug B MIC: | 32 | MEAN FICI: | | 1.16 |
| Row | MIC_{A} | FIC_{A} | MIC_{B} | FIC_{B} | FICI | |
| A | | | | | | |
| B | 2 | 0.5 | 32 | 1 | 1.5 | |
| C | 1 | 0.25 | 32 | 1 | 1.25 | |
| D | 0.5 | 0.125 | 32 | 1 | 1.125 | |
| E | 0.25 | 0.063 | 32 | 1 | 1.063 | |
| F | 0.12 | 0.03 | 32 | 1 | 1.03 | |
| G | 0.06 | 0.015 | 32 | 1 | 1.015 | |
| H | | | | | | |

TABLE 63
MIC (μg/mL), FTC, FICI, for BisEDT in combination with Colistin

| Organism: | B. cepacia 546 | | | FICI (N): | | 6 |
|---|---|---|---|---|---|---|
| Drug A: | BisEDT | Drag A MIC: | 4 | SUM FICI: | | 1.44 |
| Drug B: | Colistin | Drug B MIC: | >256 | MEAN FICI: | | 0.24 |
| Row | MIC_{A} | FIC_{A} | MIC_{B} | FIC_{B} | FICI | |
| A | | | | | | |
| B | 2 | 0.5 | 0.25 | 0.0005 | 0.500 | |
| C | 1 | 0.25 | 8 | 0.016 | 0.266 | |
| D | 0.5 | 0.125 | 32 | 0.063 | 0.188 | |
| E | 0.25 | 0.063 | 64 | 0.125 | 0.188 | |
| F | 0.12 | 0.03 | 64 | 0.125 | 0.155 | |
| G | 0.06 | 0.01.5 | 64 | 0.125 | 0.14 | |
| H | | | | | | |

TABLE 64
MIC (μg/mL), FIC, FICI, for BisEDT in combination with Meropenem

| Organism: | B. cepacia 1793 | | | FICI (N): | | 5 |
|---|---|---|---|---|---|---|
| Drug A: | BisEDT | Drug A MIC: | 2 | SUM FICI: | | 12.97 |
| Drug B: | Meropenem | Drug B MIC: | 4 | MEAN FICI: | | 2.59 |
| Row | MIC_{A} | FIC_{A} | MIC_{B} | FIC_{B} | FICI | |
| A | | | | | | |
| B | | | | | | |
| C | 1 | 0.5 | 8 | 2 | 2.5 | |
| D | 0.5 | 0.25 | 8 | 2 | 2.25 | |
| E | 0.25 | 0.125 | 16 | 4 | 4.125 | |
| F | 0.12 | 0.06 | 8 | 2 | 2.06 | |
| G | 0.06 | 0.03 | 8 | 2 | 2.03 | |
| H | | | | | | |

TABLE 65
MIC (μg/mL), FIC, FICI, for BisEDT in combination with Tobramycin

| Organism: | B. cepacia 1793 | | | FICI (N): | | 4 |
|---|---|---|---|---|---|---|
| Drug A: | BisEDT | Drag A MIC: | 1 | SUM FICI: | | 4.43 |
| Drug B: | Tobramycin | Drug B MIC: | 16 | MEAN FICI: | | 1.11 |
| Row | MIC_{A} | FIC_{A} | MIC_{B} | FIC_{B} | FICI | |
| A | | | | | | |
| B | | | | | | |
| C | | | | | | |
| D | 0.5 | 0.5 | 8 | 0.5 | 1 | |
| E | 0.25 | 0.25 | 16 | 1 | 1.25 | |
| F | 0.12 | 0.12 | 16 | 1 | 1.12 | |
| G | 0.06 | 0.06 | 16 | 1 | 1.06 | |
| H | | | | | | |

TABLE 66
MIC (μg/mL), FIC, FICI, for BisEDT in combination with Amikacin

| Organism: | B. cepacia 1793 | | | FICI (N): | | 5 |
|---|---|---|---|---|---|---|
| Drug A: | BisEDT | Drug A MIC: | 1 | SUM FICI: | | 6.43 |
| Drug B: | Amikacin | Drug B MIC: | 32 | MEAN FICI: | | 1.29 |
| Row | MIC_{A} | FIC_{A} | MIC_{B} | FIC_{B} | FICI | |
| A | | | | | | |
| B | | | | | | |
| C | 1 | 1 | 16 | 0.5 | 1.5 | |
| D | 0.5 | 0.5 | 32 | 1 | 1.5 | |
| E | 0.25 | 0.25 | 32 | 1 | 1.25 | |
| F | 0.12 | 0.12 | 32 | 1 | 1.12 | |
| G | 0.06 | 0.06 | 32 | 1 | 1.06 | |
| H | | | | | | |

TABLE 67
MIC (μg/mL), FIC, FICI, for BisEDT in combination with Ciprofloxacin

| Organism: | B. cepacia 1793 | | | FICI (N): | | 4 |
|---|---|---|---|---|---|---|
| Drug A: | BisEDT | Drag A MIC: | 1 | SUM FICI: | | 7.93 |
| Drug B: | Ciprofloxacin | Drug B MIC: | 0.5 | MEAN FICI: | | 1.98 |
| Row | MIC_{A} | FIC_{A} | MIC_{B} | FIC_{B} | FICI | |
| A | | | | | | |
| B | | | | | | |
| C | | | | | | |
| D | 0.5 | 0.5 | 1 | 2 | 2.5 | |
| E | 0.25 | 0.25 | 1 | 2 | 2.25 | |
| F | 0.12 | 0.12 | 1 | 2 | 2.12 | |
| G | 0.06 | 0.06 | 0.5 | 1 | 1.06 | |
| H | | | | | | |

TABLE 68
MIC (μg/mL), FIC, FICI, for BisEDT in combination with Aztreonam

| Organism: | B. cepacia 1793 | | | FICI (N): | | 4 |
|---|---|---|---|---|---|---|
| Drag A: | BisEDT | Drug A MIC: | 1 | SUM FICI: | | 4.93 |
| Drug B: | Aztreonam | Drug B MIC: | 128 | MEAN FICI: | | 1.23 |
| Row | MIC_{A} | FIC_{A} | MIC_{B} | FIC_{B} | FICI | |
| A | | | | | | |
| B | | | | | | |
| C | | | | | | |
| D | 0.5 | 0.5 | 128 | 1 | 1.5 | |
| E | 0.25 | 0.25 | 128 | 1 | 1.25 | |
| F | 0.12 | 0.12 | 128 | 1 | 1.12 | |
| G | 0.06 | 0.06 | 128 | 1 | 1.06 | |
| H | | | | | | |

TABLE 69
MIC (μg/mL), FIC, FICI, for BisEDT in combination with Colistin

| Organism: | B. cepacia 1793 | | | FICI (N): | | 4 |
|---|---|---|---|---|---|---|
| Drug A: | BisEDT | Drag A MIC: | 1 | SUM FICI: | | 1.32 |
| Drug B: | Colistin | Drug B MIC: | >256 | MEAN FICI: | | 0.33 |
| Row | MIC_{A} | FIC_{A} | MIC_{B} | FIC_{B} | FICI | |
| A | | | | | | |
| B | | | | | | |
| C | | | | | | |
| D | 0.5 | 0.5 | 8 | 0.016 | 0.516 | |
| E | 0.25 | 0.25 | 64 | 0.125 | 0.375 | |
| F | 0.12 | 0.12 | 64 | 0.125 | 0.245 | |
| G | 0.06 | 0.06 | 128 | 0.125 | 0.185 | |
| H | | | | | | |

TABLE 70
MIC (μg/mL), FIC, FICI, for BisEDT in combination with Meropenem

| Organism: | B. cenocepacia 548 | | | FICI (N): | | 4 |
|---|---|---|---|---|---|---|
| Drug A: | BisEDT | Drug A MIC: | 4 | SUM FICI: | | 4.44 |
| Drug B: | Meropenem | Drug B MIC: | 4 | MEAN FICI: | | 1.11 |
| Row | MIC_{A} | FIC_{A} | MIC_{B} | FIC_{B} | FICI | |
| A | | | | | | |
| B | | | | | | |
| C | | | | | | |
| D | 2 | 0.5 | 2 | 0.5 | 1 | |
| E | 1 | 0.25 | 4 | 1 | 1.25 | |
| F | 0.5 | 0.125 | 4 | 1 | 1.125 | |
| G | 0.25 | 0.063 | 4 | 1 | 1.063 | |
| H | | | | | | |

TABLE 71
MIC (μg/mL), FIC, FICI, for BisEDT in combination with Tobramycin

| Organism: | B. cenocepacia 548 | | | FICI (N): | | 4 |
|---|---|---|---|---|---|---|
| Drug A: | BisEDT | Drug A MIC: | 4 | SUM FICI: | | 2.69 |
| Drug B: | Tobramycin | Drug B MIC: | 32 | MEAN FICI: | | 0.67 |
| Row | MIC_{A} | FIC_{A} | MIC_{B} | FIC_{B} | FICI | |
| A | | | | | | |
| B | | | | | | |
| C | | | | | | |
| D | 2 | 0.5 | 8 | 0.25 | 0.75 | |
| E | 1 | 0.25 | 16 | 0.5 | 0.75 | |
| F | 0.5 | 0.125 | 16 | 0.5 | 0.62.5 | |
| G | 0.25 | 0.063 | 16 | 0.5 | 0.563 | |
| H | | | | | | |

TABLE 72
MIC (μg/mL), FIC, FICI, for BisEDT in combination with Amikacin

| Organism: | B. cenocepacia 548 | | | FICI (N): | | 4 |
|---|---|---|---|---|---|---|
| Drag A: | BisEDT | Drug A MIC: | 4 | SUM FICI: | | 2.69 |
| Drug B: | Amikacin | Drug B MIC: | 128 | MEAN FICI: | | 0.67 |
| Row | MIC_{A} | FIC_{A} | MIC_{B} | FIC_{B} | FICI | |
| A | | | | | | |
| B | | | | | | |
| C | | | | | | |
| D | 2 | 0.5 | 32 | 0.25 | 0.75 | |
| E | 1 | 0.25 | 64 | 0.5 | 0.75 | |
| F | 0.5 | 0.125 | 64 | 0.5 | 0.625 | |
| G | 0.25 | 0.063 | 64 | 0.5 | 0.563 | |
| H | | | | | | |

TABLE 73
MIC (μg/mL), FIC, FICI, for BisEDT in combination with Ciprofloxacin

| Organism: | B. cenocepacia 548 | | | FICI (N): | | 4 |
|---|---|---|---|---|---|---|
| Drag A: | BisEDT | Drug A MIC: | 4 | SUM FICI: | | 12.94 |
| Drug B: | Ciprofloxacin | Drug B MIC: | 2 | MEAN FICI: | | 3.23 |
| Row | MIC_{A} | FIC_{A} | MIC_{B} | FIC_{B} | FICI | |
| A | | | | | | |
| B | | | | | | |
| C | | | | | | |
| D | 2 | 0.5 | 8 | 4 | 4.5 | |
| E | 1 | 0.25 | 8 | 4 | 4.25 | |
| F | 0.5 | 0.125 | 4 | 2 | 2.125 | |
| G | 0.25 | 0.063 | 4 | 2 | 2.063 | |
| H | | | | | | |

TABLE 4
MIC (μg/mL), FIC, FICI, for BisEDT in combination with Aztreonam

| Organism: | B. cenocepacia 548 | | | FICI (N): | | 4 |
|---|---|---|---|---|---|---|
| Drug A: | BisEDT | Drug A MIC: | 4 | SUM FICI: | | 4.94 |
| Drug B: | Aztreonam | Drug B MIC: | 64 | MEAN FICI: | | 1.23 |
| Row | MIC_{A} | FIC_{A} | MIC_{B} | FIC_{B} | FICI | |
| A | | | | | | |
| B | | | | | | |
| C | | | | | | |
| D | 2 | 0.5 | 64 | 1 | 1.5 | |
| E | 1 | 0.25 | 64 | 1 | 1.25 | |
| F | 0.5 | 0.125 | 64 | 1 | 1.125 | |
| G | 0.25 | 0.063 | 64 | 1 | 1.063 | |
| H | | | | | | |

TABLE 75
MIC (μg/mL), FIC, FICI, for BisEDT in combination with Colistin

| Organism: | B. cenocepacia 548 | | | FICI (N): | | 4 |
|---|---|---|---|---|---|---|
| Drug A: | BisEDT | Drag A MIC: | 4 | SLIM FICI: | | 1.63 |
| Drug B: | Colistin | Drug B MIC: | >256 | MEAN FICI: | | 0.41 |
| Row | MIC_{A} | FIC_{A} | MIC_{B} | FIC_{B} | FICI | |
| A | | | | | | |
| B | | | | | | |
| C | | | | | | |
| D | 2 | 0.5 | 32 | 0.063 | 0.563 | |
| E | 1 | 0.25 | 64 | 0.125 | 0.375 | |
| F | 0.5 | 0.125 | 128 | 0.25 | 0.375 | |
| G | 0.25 | 0.063 | 128 | 0.25 | 0.313 | |
| H | | | | | | |

TABLE 76
MIC (μg/mL), FIC, FICI, for BisEDT in combination with Meropenem

| Organism: | B. cenocepacia 813 | | | FICI (N): | | 6 |
|---|---|---|---|---|---|---|
| Drug A: | BisEDT | Drag A MIC: | 4 | SUM FICI: | | 6.48 |
| Drug B: | Meropenem | Drug B MIC: | 2 | MEAN FICI: | | 1.08 |
| Row | MIC_{A} | FIC_{A} | MIC_{B} | FIC_{B} | FICI | |
| A | | | | | | |
| B | 2 | 0.5 | 1 | 0.5 | 1 | |
| C | 1 | 0.25 | 2 | 1 | 1.25 | |
| D | 0.5 | 0.125 | 2 | 1 | 1.125 | |
| E | 0.25 | 0.063 | 2 | 1 | 1.063 | |
| F | 0.12 | 0.03 | 2 | 1 | 1.03 | |
| G | 0.06 | 0.015 | 2 | 1 | 1.015 | |
| H | | | | | | |

TABLE 77
MIC (μg/mL), FIC, FICI, for BisEDT in combination with Tobramycin

| Organism: | B. cenocepacia 813 | | | FICI (N): | 6 |
|---|---|---|---|---|---|
| Drug A: | BisEDT | Drug A MIC: | 4 | SUM FICI: | 5.48 |
| Drug B: | Tobramycin | Drug B MIC: | 256 | MEAN FICI: | 0.91 |
| Row | MIC_{A} | FIC_{A} | MIC_{B} | FIC_{B} | FICI |
| A | | | | | |
| B | 2 | 0.5 | 128 | 0.5 | 1 |
| C | 1 | 0.25 | 128 | 0.5 | 1.75 |
| D | 0.5 | 0.125 | 128 | 0.5 | 1.625 |
| E | 0.25 | 0.063 | 256 | 1 | 1.063 |
| F | 0.12 | 0.03 | 256 | 1 | 1.03 |
| G | 0.06 | 0.015 | 256 | 1 | 1.015 |
| H | | | | | |

TABLE 78
MIC (μg/mL), FIC, FICI, for BisEDT in combination with Amikacin

| Organism: | B. cenocepacia 813 | | | FICI (N): | 5 |
|---|---|---|---|---|---|
| Drug A: | BisEDT | Drug A MIC: | 2 | SUM FICI: | 4.97 |
| Drug B: | Amikacin | Drug B MIC: | >256 | MEAN FICI: | 0.91 |
| Row | MIC_{A} | FIC_{A} | MIC_{B} | FIC_{B} | FICI |
| A | | | | | |
| B | | | | | |
| C | 1 | 0.5 | 256 | 0.5 | 1 |
| D | 0.5 | 0.25 | 256 | 0.5 | 0.75 |
| E | 0.25 | 0.125 | >256 | 1 | 1.125 |
| F | 0.12 | 0.06 | >256 | 1 | 1.06 |
| G | 0.06 | 0.03 | >256 | 1 | 1.03 |
| H | | | | | |

TABLE 79
MIC (μg/mL), FIC, FICI, for BisEDT in combination with Ciprofloxacin

| Organism: | B. cenocepacia 813 | | | FICI (N): | 6 |
|---|---|---|---|---|---|
| Drug A: | BisEDT | Drug A MIC: | 4 | SUM FICI: | 17.11 |
| Drug B: | Ciprofloxacin | Drug B MIC: | 2 | MEAN FICI: | 2.85 |
| Row | MIC_{A} | FIC_{A} | MIC_{B} | FIC_{B} | FICI |
| A | | | | | |
| B | 2 | 0.5 | 0.25 | 0.125 | 0.625 |
| C | 1 | 0.25 | 8 | 4 | 4.25 |
| D | 0.5 | 0.125 | 8 | 4 | 4.125 |
| E | 0.25 | 0.063 | 8 | 4 | 4.063 |
| F | 0.12 | 0.03 | 4 | 2 | 2.03 |
| G | 0.06 | 0.015 | 4 | 2 | 2.015 |
| H | | | | | |

TABLE 80
MIC (μg/mL), FIC, FICI, for BisEDT in combination with Aztreonam

| Organism: | B. cenocepacia 813 | | | FICI (N): | 5 |
|---|---|---|---|---|---|
| Drug A: | BisEDT | Drug A MIC: | 2 | SUM FICI: | 5.97 |
| Drug B: | Aztreonam | Drug B MIC: | 32 | MEAN FICI: | 1.19 |
| Row | MIC_{A} | FIC_{A} | MIC_{B} | FIC_{B} | FICI |
| A | | | | | |
| B | | | | | |
| C | 1 | 0.5 | 32 | 1 | 1.5 |
| D | 0.5 | 0.25 | 32 | 1 | 1.25 |
| E | 0.25 | 0.125 | 32 | 1 | 1.125 |
| F | 0.12 | 0.06 | 32 | 1 | 1.06 |
| G | 0.06 | 0.03 | 32 | 1 | 1.03 |
| H | | | | | |

TABLE 81
MIC (μg/mL), FIC, FICI, for BisEDT in combination with Colistin

| Organism: | B. cenocepacia 813 | | | FICI (N): | 5 |
|---|---|---|---|---|---|
| Drug A: | BisEDT | Drug A MIC: | 2 | SUM FICI: | 3.59 |
| Drug B: | Colistin | Drug B MIC: | >256 | MEAN FICI: | 0.72 |
| Row | MIC_{A} | FIC_{A} | MIC_{B} | FIC_{B} | FICI |
| A | | | | | |
| B | | | | | |
| C | 1 | 0.5 | 64 | 0.125 | 0.625 |
| D | 0.5 | 0.25 | 256 | 0.5 | 0.75 |
| E | 0.25 | 0.125 | 256 | 0.5 | 0.625 |
| F | 0.12 | 0.06 | 256 | 0.5 | 0.56 |
| G | 0.06 | 0.03 | >256 | 1 | 1.03 |
| H | | | | | |

TABLE 82
MIC (μg/mL), FIC, FICI, for BisEDT
in combination with Meropenem

| Organism: | B. multivorans 1795 | | | FICI (N): | 6 |
|---|---|---|---|---|---|
| Drug A: | BisEDT | Drug A MIC: | 2 | SUM FICI: | 7.97 |
| Drug B: | Meropenem | Drug B MIC: | 4 | MEAN FICI: | 1.33 |

| Row | MIC_{A} | FIC_{A} | MIC_{B} | FIC_{B} | FICI |
|---|---|---|---|---|---|
| A | | | | | |
| B | 2 | 1 | 4 | 1 | 2 |
| C | 1 | 0.5 | 4 | 1 | 1.5 |
| D | 0.5 | 0.25 | 4 | 1 | 1.25 |
| E | 0.25 | 0.125 | 4 | 1 | 1.125 |
| F | 0.12 | 0.06 | 4 | 1 | 1.06 |
| G | 0.06 | 0.03 | 4 | 1 | 1.03 |
| H | | | | | |

TABLE 83
MIC (μg/mL), FIC, FICI, for BisEDT
in combination with Tobramycin

| Organism: | B. multivorans 1795 | | | FICI (N): | 6 |
|---|---|---|---|---|---|
| Drug A: | BisEDT | Drug A MIC: | 2 | SUM FICI: | 7.97 |
| Drug B: | Tobramycin | Drug B MIC: | >256 | MEAN FICI: | 1.33 |

| Row | MIC_{A} | FIC_{A} | MIC_{B} | FIC_{B} | FICI |
|---|---|---|---|---|---|
| A | | | | | |
| B | 2 | 1 | >256 | 1 | 2 |
| C | 1 | 0.5 | >256 | 1 | 1.5 |
| D | 0.5 | 0.25 | >256 | 1 | 1.25 |
| E | 0.25 | 0.125 | >256 | 1 | 1.125 |
| F | 0.12 | 0.06 | >256 | 1 | 1.06 |
| G | 0.06 | 0.03 | >256 | 1 | 1.03 |
| H | | | | | |

TABLE 84
MIC (μg/mL), FIC, FICI, for
BisEDT in combination with Amikacin

| Organism: | B. multivorans 1795 | | | FICI (N): | 6 |
|---|---|---|---|---|---|
| Drug A: | BisEDT | Drug A MIC: | 2 | SUM FICI: | 7.97 |
| Drug B: | Amikacin | Drug B MIC: | >256 | MEAN FICI: | 1.33 |

| Row | MIC_{A} | FIC_{A} | MIC_{B} | FIC_{B} | FICI |
|---|---|---|---|---|---|
| A | | | | | |
| B | 2 | 1 | >256 | 1 | 2 |
| C | 1 | 0.5 | >256 | 1 | 1.5 |
| D | 0.5 | 0.25 | >256 | 1 | 1.25 |
| E | 0.25 | 0.125 | >256 | 1 | 1.125 |
| F | 0.12 | 0.06 | >256 | 1 | 1.06 |
| G | 0.06 | 0.03 | >256 | 1 | 1.03 |
| H | | | | | |

TABLE 85
MIC (μg/mL), FIC, FICI, for BisEDT
in combination with Ciprofloxacin

| Organism: | B. multivorans 1795 | | | FICI (N): | 5 |
|---|---|---|---|---|---|
| Drug A: | BisEDT | Drug A MIC: | 2 | SUM FICI: | 5.97 |
| Drug B: | Ciprofloxacin | Drug B MIC: | 32 | MEAN FICI: | 1.19 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 32 | 1 | 1.5 |
| D | 0.5 | 0.25 | 32 | 1 | 1.25 |
| E | 0.25 | 0.125 | 32 | 1 | 1.125 |
| F | 0.12 | 0.06 | 32 | 1 | 1.06 |
| G | 0.06 | 0.03 | 32 | 1 | 1.03 |
| H | | | | | |

TABLE 86
MIC (μg/mL), FIC, FICI, for BisEDT
in combination with Aztreonam

| Organism: | B. multivorans 1795 | | | FICI (N): | 5 |
|---|---|---|---|---|---|
| Drug A: | BisEDT | Drug A MIC: | 2 | SUM FICI: | 6.97 |
| Drug B: | Aztreonam | Drug B MIC: | 16 | MEAN FICI: | 1.39 |

| Row | MIC_{A} | FIC_{A} | MIC_{B} | FIC_{B} | FICI |
|---|---|---|---|---|---|
| A | | | | | |
| B | | | | | |
| C | 1 | 0.5 | 16 | 1 | 1.5 |
| D | 0.5 | 0.25 | 16 | 1 | 1.25 |
| E | 0.25 | 0.125 | 16 | 1 | 1.125 |
| F | 0.12 | 0.06 | 16 | 1 | 1.06 |
| G | 0.06 | 0.03 | 32 | 2 | 1.03 |
| H | | | | | |

TABLE 87
MIC (μg/mL), FIC, FICI, for BisEDT in combination with Colistin

| Organism: | B. multivorans 1795 | | | FICI (N): | 6 |
|---|---|---|---|---|---|
| Drug A: | BisEDT | Drug A MIC: | 2 | SUM FICI: | 7.97 |
| Drug B: | Colistin | Drug B MIC: | >256 | MEAN FICI: | 1.33 |

| Row | MIC_{A} | FIC_{A} | MIC_{B} | FIC_{B} | FICI |
|---|---|---|---|---|---|
| A | | | | | |
| B | 2 | | >256 | 1 | 2 |
| C | 1 | 0.5 | >256 | 1 | 1.5 |
| D | 0.5 | 0.25 | >256 | 1 | 1.25 |
| E | 0.25 | 0.125 | >256 | 1 | 1.125 |
| F | 0.12 | 0.06 | >256 | 1 | 1.06 |
| G | 0.06 | 0.03 | >256 | 1 | 1.03 |
| H | | | | | |

### REFERENCES

1.) Clinical and Laboratory Standards Institute (CLSI). Performance Standards for Antimicrobial Susceptibility Testing. 28th ed. CLSI supplement M100. CLSI, 950 West Valley Road, Suite 2500, Wayne, Pa. 19087 USA, 2018.
2.) CLSI. Methods for Dilution Antimicrobial Susceptibility Tests for Bacteria That Grow Aerobically; Approved Standard-11^{th} Edition. CLSI standard M07. CLSI, 950 West Valley Road, Suite 2500, Wayne, Pa. 19087 USA, 2018.
3.) Eliopoulos G and R Moellering. 1991. Antimicrobial combinations. In Antibiotics in Laboratory Medicine, Third Edition, edited by V Lorian. Williams and Wilkins, Baltimore, Md., pp. 432-492.
4.) Odds FC. 2003. Synergy, antagonism, and what the chequerboard puts between them. J Antimicrob Chemother 52(1):1.

### Example 11: In Vitro Activity of Bismuth Thiols and Comparators Against Drug Resistant Gram-Positive and -Negative Bacteria and Yeast

Introduction: The in vitro activity of three Bismuth Thiol compounds were evaluated against organisms currently identified by the Centers for Disease Control (CDC; 1) as top drug-resistant threats in the United States, including ESKAPE pathogens (2, 3), C. difficile, resistant gonococci, and azole-resistant Candida spp. The susceptibility of test isolates to the Bismuth Thiol compounds MB-1-B3, MB-2B, and MB-6 and relevant comparators was evaluated in accordance with guidelines from the Clinical and Laboratory Standards Institute (CLSI; 4-8).

### Materials and Methods

Test and Comparator Agents: The test agents were stored at room temperature until assayed. All test agents were suspended and diluted in 100% dimethylsulfoxide (DMSO), and were ultimately tested at a final concentration of 0.06-64 μg/mL. The stock solutions were allowed to stand for at least 1 hr prior to use to auto-sterilize.

Comparator drugs were tested over a concentration range spanning established quality control ranges and breakpoints. Information on comparator compounds used during testing are described in Table 88 below.

TABLE 88
Comparator Comounds

| Drug | Manufacturer | Lot No. | Solvent | Diluent |
|---|---|---|---|---|
| Levofloxacin | Sigma | BCBC2112V | 0.1M NaOH | Water |
| Meropenem | USP | I0J244 | Water | Water |
| Ceftazidime | USP | L1K237 | Water | Water |
| Gentamicin | Sigma | SLBM9736V | Water | Water |
| Vancomycin | | | Water | Water |
| Penicillin | Sigma | 071M0740V | Water | Water |
| Oxacillin | Sigma | BCBF5635V | Water | Water |
| Clindamycin | Sigma | 021M1533V | Water | Water |
| Erythromycin | Sigma | 011M1510V | 95% ethanol | Water |
| Ciprofloxacin | USP | 1134335 | Water | Water |
| Metronidazole | Sigma | 095K0693 | DMSO | Water |
| Fidaxomicin | Merck | SE-B13-01-001885 | DMSO | Water |
| Fluconazole | USP | H1L308 | DMSO | DMSO |
| Amphotericin B | Sigma | 063M4043V | DMSO | DMSO |
| Trimethoprim | Sigma | 080M4044 | Water | Water |
| Sulfamethoxazole | Fluka | BCBC7096V | Water and 2.5M | Water |
| | | | NaOH dropwise | |
| Ceftriaxone | USP | J1L040 | Water | Water |

Test Organisms: The test organisms consisted of reference strains from the American Type Culture Collection (ATCC; Manassas, Va.) or clinical isolates from the MMX repository. The spectrum of organisms evaluated and their corresponding phenotypic information is shown in Tables 89-95. Relevant quality control organisms were included on each day of testing as specified by CLSI (4-8). The isolates were sub-cultured onto an appropriate agar medium prior to testing.

TestMedia: Test media were prepared and stored in accordance with guidelines from CLSI (4, 6, 7). Broth microdilution susceptibility testing of aerobic bacteria was performed using cation adjusted Mueller-Hinton Broth (CAMHB; Becton Dickinson [BD], Sparks, Md.; Lot No. 6117994) with the exception of streptococci where CAMHB was supplemented with 5% (v/v) lysed horse blood (Cleveland Scientific, Bath, Ohio; Lot No. 322799). Neisseria gonorrhoeae were evaluated by agar dilution using agar consisting of GC medium base (BD; Lot No. 4274618) supplemented with 1% IsoVitaleX (BD; Lot No. 5246843).

The susceptibility of anaerobic bacteria was determined by agar dilution using Brucella Agar (BD/BBL; Lot No. 5237692) supplemented with 5 μg/mL hemin (Sigma, St. Louis, Mo.; Lot No. 108K₁₀₈₈), 1 μg/mL Vitamin K₁ and 5% (v/v) laked sheep blood (Cleveland Scientific, Bath, Ohio; Lot No. 322799).

The susceptibility of yeast isolates was determined by broth microdilution in RPMI medium (HyClone Laboratories, Logan, Utah; Lot No. AZC184041B) buffered with 0.165 M MOPS (Calbiochem, Billerica, Mass.; Lot No. 2694962). The pH of the medium was adjusted to 7.0 with 1 N NaOH, sterile filtered using a 0.2 μm PES filter, and stored at 4° C. until used.

Broth Microdilution MIC Testing (Aerobic Bacteria and Yeast): The broth microdilution assay method employed for the susceptibility testing of aerobic bacteria (excluding N. gonorrhoeae which was evaluated by agar dilution) and yeast essentially followed the procedures described by CLSI (3, 4, 7, 8) and employed automated liquid handlers to conduct serial dilutions and liquid transfers. Automated liquid handlers included the Multidrop 384 (Labsystems, Helsinki, Finland) and Biomek 2000 (Beckman Coulter, Fullerton Calif.). The wells in columns 2-12 in standard 96-well microdilution plates (Costar 3795) were filled with 150 μl of the correct diluent. These would become the ‘mother plates’ from which ‘daughter’ or test plates would be prepared. The drugs (300 μL at 40X the desired top concentration in the test plates) were dispensed into the appropriate well in Column 1 of the mother plates. The Biomek 2000 was used to make serial two-fold dilutions through Column 11 in the “mother plate”. The wells of Column 12 contained no drug and ultimately served as the organism growth control wells.

The daughter plates were loaded with 185 μL per well of the appropriate test media using the Multidrop 384. The daughter plates were prepared using the Biomek FX which transferred 5 μL of drug solution from each well of a mother plate to the corresponding well of the correct daughter plate in a single step.

A standardized inoculum of each organism was prepared per CLSI methods (3, 7). Isolated colonies of each test isolate were picked from the primary plate and a suspension was prepared to equal a 0.5 McFarland turbidity standard. Standardized suspensions were then diluted 1:100 in test media (1:100 for yeast, 1:20 for bacteria). After dilution, the inoculum suspensions were then transferred to compartments of sterile reservoirs divided by length (Beckman Coulter), and the Biomek 2000 was used to inoculate all plates. Daughter plates were placed on the Biomek 2000 in reverse orientation so that plates were inoculated from low to high drug concentration.

The Biomek 2000 delivered 10 μL of standardized inoculum into each well of the appropriate daughter plate for an additional 1:20 dilution. The wells of the daughter plates ultimately contained 185 μL of the appropriate media, 5 μL of drug solution, and 10 μL of inoculum which corresponded to a final inoculum concentration of 0.5-2.5×103 CFU/mL of yeast and approximately 5×10⁵ CFU/mL of bacteria per test well. The final concentration of DMSO (if used as a solvent) in the test well was 2.5%.

Plates were stacked 4 high, covered with a lid on the top plate, placed into plastic bags, and incubated at 35° C. for approximately 24-48 hr for all yeast isolates and 16-24 hr for aerobic bacteria. Plates were viewed from the bottom using a plate viewer. An un-inoculated solubility control plate was observed for evidence of drug precipitation. MIC endpoints for the test agents and control compounds were read per CLSI criteria (3, 7).

Agar Dilution MIC Testing (Anaerobic Bacteria and Gonococci): MIC values for anaerobic bacteria were determined using a reference agar dilution method as described by CLSI (6). Organisms were grown at 35° C. in the Bactron II Anaerobic Chamber (Shel Lab, Cornelius, Oreg.) for approximately 48 hr prior to the assay. Drug dilutions and drug-supplemented agar plates were prepared manually per CLSI (6). The plates were allowed to stand at room temperature for 1 hr to allow the agar surface to dry and pre-reduced for approximately 1 hr in the anaerobe chamber prior to inoculation. Each isolate was suspended to the equivalent of a 0.5 McFarland standard in Brucella broth using a turbidity meter (Dade Behring MicroScan, West Sacramento, Calif.). Each bacterial cell suspension was then diluted 1:10 in Brucella broth and transferred to wells in a stainless steel replicator block which was used to inoculate the test plates. The prongs on the replicator deliver approximately 1-2 μl of inoculum to an agar surface. The resulting inoculum spots contained approximately 1×10⁵ CFU/spot. After the inoculum dried, the inoculated drug-supplemented agar plates and no drug growth control plates were incubated at 35° C. for 42-48 hr in the anaerobe chamber. The MIC was read per CLSI guidelines (6).

MIC values for N. gonorrhoeae were determined using the reference agar dilution method as described by CLSI (4). This method followed the same agar dilution method described above for anaerobes with the exception that agar plates contained GC medium base supplemented with 1% IsoVitaleX and, after inoculation, plates were incubated aerobically at 35° C. in 5% C02 for 20-24 hr.

### Results and Discussion

The activity of the Bismuth Thiol test agents MB-1-B3, MB-2B, and MB-6 and comparators are shown below for Enterobacteriaceae (Table 89), Pseudomonas aeruginosa and Acinetobacter baumannii (Table 90), Staphylococcus aureus and Enterococcus spp. (Table 91), streptococci (Table 92), N. gonorrhoeae (Table 93), anaerobes (Table 94), and Candida spp. (Table 95). Across all evaluated organisms, MIC results for comparator agents were within the established CLSI QC ranges for the relevant ATCC QC isolate (5, 8).

The evaluated Enterobacteriaceae (Table 1) consisted of the Escherichia coli ATCC QC isolate, ESBL-positive E. coli and Klebsiella pneumoniae, KPC-positive K. pneumoniae, and NDM-1 positive E. coli, K. pneumoniae, and Enterobacter cloacae. Excluding the ATCC QC isolate of E. coli which was susceptible, the activity of the comparators illustrates the drug-resistant nature of these isolates. Regardless of the high degree of drug resistance, the evaluated Bismuth Thiol test agents had consistent activity across all isolates. MB-1-B3 (MIC values of 0.5-4 μg/mL) and MB-6 (MIC values of 0.5-2 μg/mL) had similar activity and this activity was typically 2- to 16-fold lower than that observed with MB-2B (MIC values of 2-32 μg/mL).

The evaluated P. aeruginosa and A. baumannii (Table 90) consisted of the susceptible P. aeruginosa ATCC QC isolate, and various isolates with either metallo-beta-lactamases or multi-drug resistance. Excluding the QC isolate, the activity of comparators illustrates the drug-resistant nature of these isolates. Regardless of the high degree of drug resistance, the evaluated Bismuth Thiol test agents had consistent activity across all isolates. MB-1-B3 and MB-6 (MIC values of 0.5-2 μg/mL) had similar activity and this activity was typically 2- to 32-fold lower than that observed with MB-2B (MIC values of 2-16 μg/mL).

Against S. aureus and Enterococcus spp. (Table 91), all 3 Bismuth Thiol test compounds had potent activity regardless of resistance phenotype (MRSA for S. aureus and VRE for E. faecalis and E. faecium). The evaluated MRSA were largely susceptible to vancomycin and gentamicin but resistant to the remaining comparators. Regardless of resistance, MB-1-B3 and MB-6 had MIC values of <0.06 μg/mL against MRSA and MB-2B also had MIC values of <0.06 μg/mL with the exception of the QC isolate and MRSA MMX 9203 (MIC values of 0.5 and 0.25 μg/mL, respectively). Against enterococci, there was little activity observed with the evaluated comparators. The Bismuth Thiol test agents were active though with slightly higher MIC values for vancomycin-resistant E. faecium relative to vancomycin-resistant E. faecalis. As with the Gram-negative aerobic isolates, for enterococci MB-1-B3 (MIC values of 0.25-2 μg/mL) and MB-6 (MIC values of 0.25-1 μg/mL) had similar activity and this activity was typically 4- to 8-fold lower than that observed with MB-2B (MIC values of 2-4 μg/mL).

The evaluated streptococci (Table 92) consisted of the susceptible S. pneumoniae QC isolate, multi-drug resistant pneumococci, macrolide-resistant S. pyogenes, and clindamycin-resistant S. agalactiae. Regardless of drug-resistance phenotype, the bismuth-thiol test agents maintained activity against streptococci. Among the 3 Bismuth Thiol test agents, there was trend towards slightly higher MIC values against pneumococci relative to beta-hemolytic streptococci for MB-1-B3 and MB-6. Against pneumococci, MB-1-B3 (MIC values of 0.5-1 μg/mL) and MB-6 (MIC values of 0.5-8 μg/mL) had similar activity and this activity was typically 8- to 16-fold lower than that observed with MB-2B (MIC values of 1-8 μg/mL). Against beta-hemolytic streptococci, MB-1-B3 (MIC values of 0.03-1 μg/mL) and MB-6(MIC values of 0.03-2 μg/mL) had similar activity and this activity was typically 4- to 16-fold lower than that observed with MB-2B (MIC values of 0.25-8 μg/mL).

As shown in Table 93, the Bismuth Thiol test agents had potent activity against the susceptible QC isolate of N. gonorrhoeae, the 3 ciprofloxacin-resistant isolates, and the single ceftriaxone non-susceptible isolate. Similar activity was observed with MB-1-B3 (MIC values of 0.06-0.12 μg/mL) and MB-6 (MIC values of 0.06-0.25 μg/mL) and this activity was slightly greater than that observed for MB-2B (MIC values of 0.12-0.5 μg/mL).

Against the evaluated anaerobes (Table 94) which consisted of the susceptible Bacteroides fragilis and Clostridium difficile QC isolates and C. difficile with various clinically important ribotypes including 027 (hypervirulent strain), MB-1-B3 (MIC values of 0.25-2 μg/mL) and MB-6 (MIC values of 1-4 μg/mL) had similar activity and this activity was typically slightly greater than that observed with MB-2B (MIC values of 2-16 μg/mL). Resistance to comparators clindamycin, metronidazole, and fidaxomicin appeared to have no impact of the activity of the Bismuth Thiol test agents.

Finally, against azole-resistant isolates of clinically prevalent Candida spp. (Table 95) including C. parapsilosis, C. albicans, C. glabrata, and C. tropicalis, the Bismuth Thiol test agents were active. A trend towards higher MIC values for the test agents was observed with C. albicans and C. tropicalis relative to C. parapsilosis and C. glabrata. All 3 Bismuth Thiol test agents had similar activity against yeast, with MIC values of 0.25-0.5 μg/mL at 24 hr against C. parapsilosis and C. glabrata, 1-4 μg/mL against C. albicans, and 1-16 μg/mL against C. tropicalis.

In summary, the broad spectrum activity of the Bismuth Thiol test agents evaluated in this study was clear and the activity observed against susceptible QC isolates was maintained against drug resistant isolates regardless of the organism or resistance phenotype evaluated. The Bismuth Thiol test agents were the most active against MRSA, N. gonorrhoeae, and beta-hemolytic streptococci based on MIC values but were also highly active against Gram-negative aerobes, S. pneumoniae, C. difficile, and yeast. In general, test agents MB-1-B3 and MB-6 had similar activity by MIC and both were more potent than MB-2B, with the exception of yeast and to a lesser extent N. gonorrhoeae where all 3 compounds had similar activity profiles.

TABLE 89
Minimal Inhibitory Concentration (MIC) Values for Microbion Bismuth Thiol Test Agents and Comparators Against
Enterobacteriaceae

| | | MIC (μg/mL) | | | | | | | |
|---|---|---|---|---|---|---|---|---|---|
| Isolate | Type | MB-1-B3 | MB-2B | MB-6 | Levofloxacin | Ciprofloxacin | Meropenem | Ceftazidime | Gentamicin |
| E. coli | QC | 0.5 | 2 | 0.5 | 0.015 | 0.008 | 0.03 | 0.12 | 1 |
| MMX 102 | | | | | (0.008-0.06)¹ | (0.004-0.015) | (0.008-0.06) | (0.06-0.5) | (0.25-1) |
| (ATCC 25922) | | | | | | | | | |
| E. coli | ESBL | 1 | 2 | 1 | 16 | 32 | 0.03 | 32 | 64 |
| MMX 8423 | Lev^{R} CAZ^{R} | | | | | | | | |
| | Gm^{R} | | | | | | | | |
| E. coli | ESBL | 0.5 | 4 | 0.5 | 0.06 | 0.015 | 0.03 | 16 | 0.25 |
| MMX 8424 | CAZ^{R} | | | | | | | | |
| E. coli | ESBL | 1 | 2 | 1 | 16 | >64 | 0.015 | 16 | >64 |
| MMX 8425 | Lev^{R}CAZ^{R} | | | | | | | | |
| | Gm^{R} | | | | | | | | |
| E. coli | NDM-1 | 1 | 2 | 1 | 16 | >64 | 32 | >32 | >64 |
| MMX 5980 (ATCC | Lev^{R} | | | | | | | | |
| BAA-2469) | MEM^{R} | | | | | | | | |
| | CAZ^{R} | | | | | | | | |
| | Gm^{R} | | | | | | | | |
| K. pneumoniae | KPC-2 | 1 | 8 | 1 | >64 | >64 | 32 | >32 | 1 |
| MMX 4683 | Lev^{R} | | | | | | | | |
| | MEM^{R} | | | | | | | | |
| | CAZ^{R} | | | | | | | | |
| K. pneumoniae | KPC-2 | 2 | 16 | 1 | 1 | 0.03 | >64 | >32 | 0.25 |
| MMX 4622 | MEM^{R} | | | | | | | | |
| | CAZ^{R} | | | | | | | | |
| K. pneumoniae | KPC-2 | 2 | 32 | 0.5 | 1 | 2 | >64 | >32 | 64 |
| MMX 4623 | MEM^{R} | | | | | | | | |
| | CAZ^{R} | | | | | | | | |
| | Gm^{R} | | | | | | | | |
| K. pneumoniae | KPC-3 | 2 | 8 | 1 | 64 | >64 | 32 | >32 | 8 |
| MMX 4694 | Lev^{R} | | | | | | | | |
| | MEM^{R} | | | | | | | | |
| | CAZ^{R} | | | | | | | | |
| K. pneumoniae | KPC-3 | 4 | 16 | 2 | 64 | >64 | >64 | >32 | 1 |
| MMX 4653 | Lev^{R} | | | | | | | | |
| | MEM^{R} | | | | | | | | |
| | CAZ^{R} | | | | | | | | |
| K. pneumoniae | ESBL | 4 | 32 | 2 | 0.03 | 0.5 | 8 | 8 | 0.25 |
| MMX 4684 | MEM^{R} | | | | | | | | |
| K. pneumoniae | ESBL | 2 | 8 | 1 | 32 | 64 | 4 | >32 | 1 |
| MMX 4685 | Lev^{R} | | | | | | | | |
| | MEM^{R} | | | | | | | | |
| | CAZ^{R} | | | | | | | | |
| K. pneumoniae | NDM-1 | 2 | 16 | 1 | >64 | >64 | >64 | >32 | >64 |
| MMX 5979 | Lev^{R} | | | | | | | | |
| | MEM^{R} | | | | | | | | |
| | CAZ^{R} | | | | | | | | |
| | Gm^{R} | | | | | | | | |
| E. cloacae | NDM-1 | 4 | 32 | 2 | 64 | >64 | >64 | >32 | >64 |

| | |
|---|---|
| MMX 5981 | Lev^{R} |
| (ATCC BAA-2468) | MEM^{R} |
| | CAZ^{R} |
| | Gm^{R} |

QC = quality control;
ESBL = extended-spectrum beta-lactamase;
KPC = K. pneumoniae carbapenemase;
NDM = New Delhi Metallo-beta-lactamase;
Lev^{R} = Levofloxacin-resistant;
MEM^{R} = Meropenem-resistant;
CAZ^{R} = ceftazidime-resistant;
Gm^{R} = Gentamicin-resistant
¹CLSI QC ranges shown in parenthesis where applicable

TABLE 90
Minimal Inhibitory Concentration (MIC) Values for Microbion Bismuth Thiol Test Agents and Comparators Against P.
aeruginosa and A. baumannii

| | | MI | | | | | | | |
|---|---|---|---|---|---|---|---|---|---|
| Isolate | Type | MB-1-B3 | MB-2B | MB-6 | Levofloxacin | Ciprofloxacin | Meropenem | Ceftazidime | Gentamicin |
| P. aeruginosa | QC | 1 | 8 | 1 | 1 | 0.5 | 0.5 | 2 | 1 |
| MMX 103 | | | | | (0.5-4) | (0.25-1) | (0.25-1) | (1-4) | (0.5-2) |
| (ATCC 27853) | | | | | | | | | |
| P. aeruginosa | VIM-2 | 1 | 16 | 2 | 32 | 32 | 8 | 32 | 4 |
| MMX 4697 | Lev^{R} | | | | | | | | |
| | Cip^{R} | | | | | | | | |
| | MEM^{R} | | | | | | | | |
| | CAZ^{R} | | | | | | | | |
| P. aeruginosa | IMP-7 | 2 | 8 | 2 | 64 | 32 | >64 | >32 | >64 |
| MMX 4654 | Lev^{R} | | | | | | | | |
| | Cip^{R} | | | | | | | | |
| | MEM^{R} | | | | | | | | |
| | CAZ^{R} | | | | | | | | |
| P. aeruginosa | MDR | 1 | 4 | 2 | 64 | 64 | 32 | >32 | 8 |
| MMX 2562 | Lev^{R} | | | | | | | | |
| | Cip^{R} | | | | | | | | |
| | MEM^{R} | | | | | | | | |
| | CAZ^{R} | | | | | | | | |
| P. aeruginosa | MDR | 1 | 2 | 1 | 64 | 32 | 16 | 32 | 8 |
| MMX 1381 | Lev^{R} | | | | | | | | |
| | Cip^{R} | | | | | | | | |
| | MEM^{R} | | | | | | | | |
| | CAZ^{R} | | | | | | | | |
| P. aeruginosa | MDR | 1 | 8 | 2 | 64 | 32 | 16 | 16 | 4 |
| MMX 3991 | Lev^{R} | | | | | | | | |
| | Cip^{R} | | | | | | | | |
| | MEM^{R} | | | | | | | | |
| | CAZ^{R} | | | | | | | | |
| A. baumanmi | MDR: | 0.5 | 16 | 0.5 | 8 | 32 | 64 | >32 | 8 |
| MMX 4651 | OXA-27 | | | | | | | | |
| (NCTC 13304) | Lev^{R} | | | | | | | | |
| | Cip^{R} | | | | | | | | |
| | MEM^{R} | | | | | | | | |
| | CAZ^{R} | | | | | | | | |
| | Gm¹ | | | | | | | | |
| A. baumanmi | MDR | 0.5 | 16 | 0.5 | 64 | >64 | 64 | 32 | >64 |
| MMX 2592 | Lev^{R} | | | | | | | | |
| | Cip^{R} | | | | | | | | |
| | MEM^{R} | | | | | | | | |
| | CAZ^{R} | | | | | | | | |
| | Gm^{R} | | | | | | | | |
| A., baumanmi | MDR | 1 | 16 | 0.5 | 32 | >64 | 64 | 32 | >64 |
| MMX 2593 | Lev^{R} | | | | | | | | |
| | Lip^{R} | | | | | | | | |
| | MEM^{R} | | | | | | | | |
| | CAZ^{R} | | | | | | | | |
| | Gm^{R} | | | | | | | | |
| A. baumannii | MDR | 1 | 16 | 1 | 1 | 4 | 4 | 16 | 0.5 |
| MMX 3372 | Cip^{R} | | | | | | | | |
| | MEM¹ | | | | | | | | |
| | CAZ¹ | | | | | | | | |
| A. baumannii | Sensitive | 1 | 16 | 0.5 | 0.12 | 0.5 | 0.5 | 4 | 0.12 |
| MMX 3373 | | | | | | | | | |

QC = quality control;
VIM/IMP = metallo-beta lactamase type;
OXA = type D extended-spectrum beta-lactamase;
MDR = multi-drug resistant (based on resistance to at least different classes of antibiotic);
Lev^{R} = levofloxacin-resistant;
CIP^{R} = Ciprofloxacin-resistant;
MEM¹ = meropenem intermediate resistance;
MEM^{R} = meropenem-resistant;
CAZ^{R} = ceftazidime-resistant;
CAZ¹ = ceftazidime intermediate resistance;
Gm^{R} = gentamicin-resistant;
Gm¹ = gentamicin intermediate resistance.
¹ CLSI QC ranges shown in parenthesis where applicable

TABLE 91
Minimal Inhibitory Concentration (MIC) Values for Microbion Bismuth Thiol Test
Agents and Comparators Against S. aureus and Enterococcus spp.

| | | MIC (μg/mL) | | | | | |
|---|---|---|---|---|---|---|---|
| Isolate | Type | MB-1-B3 | MB-2B | MB-6 | Levofloxacin | Ciprofloxacin | Gentamicin |
| S. aureus | QC; | ≤0.06 | 0.5 | ≤0.06 | 0.12 | 0.5 | 0.12 |
| MMX 101 | MSSA | | | | (0.06-0.5)¹ | (0.12-0.5) | (0.12-1) |
| (ATCC 29213) | | | | | | | |
| S. aureus | MRSA² | ≤0.06 | 0.25 | ≤0.06 | 4 | 16 | 0.12 |
| MMX 9203 | Lev^{R} | | | | | | |
| | Cip^{R} | | | | | | |
| | Ery^{R} | | | | | | |
| | Ox^{R} | | | | | | |
| S. aureus | MRSA | ≤0.06 | ≤0.06 | ≤0.06 | A | 16 | 0.25 |
| MMX 9204 | Lev^{R} | | | | | | |
| | Cip^{R} | | | | | | |
| | Ery^{R} | | | | | | |
| | Ox^{R} | | | | | | |
| S. aureus | MRSA | ≤0.06 | ≤0.06 | ≤0.06 | >64 | >64 | 0.25 |
| MMX 9205 | Lev^{R} | | | | | | |
| | Cip^{R} | | | | | | |
| | CC^{R} | | | | | | |
| | Ery^{R} | | | | | | |
| | Ox^{R} | | | | | | |
| S. aureus | MRSA | ≤0.06 | ≤0.06 | ≤0.06 | 32 | >64 | 0.25 |
| MMX 5373 | Lev^{R} | | | | | | |
| | Cip^{R} | | | | | | |
| | CC^{R} | | | | | | |
| | Ery^{R} | | | | | | |
| | Ox^{R} | | | | | | |
| S. aureus | MRSA | ≤0.06 | ≤0.06 | ≤0.06 | 32 | >64 | 0.25 |
| MMX 6311 | Lev^{R} | | | | | | |
| | Cip^{R} | | | | | | |
| | CC^{R} | | | | | | |
| | Ery^{R} | | | | | | |
| | Ox^{R} | | | | | | |
| E. faecalis | VRE | 0.5 | | 0.5 | 32 | 64 | >64 |
| MMX 8960 | Lev^{R} | | | | | | |
| | Cip^{R} | | | | | | |
| | Ery^{R} | | | | | | |
| E. faecalis | VRE | 0.25 | 2 | 0.25 | 64 | 64 | 8 |
| MMX 8961 | Lev^{R} | | | | | | |
| | Cip^{R} | | | | | | |
| | Ery^{R} | | | | | | |
| E. faecalis | VRE | 0.5 | 2 | 0.25 | 64 | 64 | >64 |
| MMX 8962 | Lev^{R} | | | | | | |
| | Cip^{R} | | | | | | |
| | Ery^{R} | | | | | | |
| E. faecium | VRE | 2 | 4 | 1 | >64 | >64 | >64 |
| MMX 752 | vanA | | | | | | |
| | Lev^{R} | | | | | | |
| | Cip^{R} | | | | | | |
| | Ery^{R} | | | | | | |
| E. faecium | VRE | 1 | 2 | 0.5 | >64 | >64 | >64 |
| MMX 485 | vanA | | | | | | |
| | Lev^{R} | | | | | | |
| | Cip^{R} | | | | | | |
| | Ery^{R} | | | | | | |
| | | MIC (μg/mL) | | | | | |
| Isolate | Type | Vancomycin | Meropenem | Ceftazidime | Clindamycin | Erythromycin | Oxacillin |
| S. aureus | QC; | 1 | 0.06 | 8 | 0.12 | 0.5 | 0.25 |
| MMX 101 | MSSA | (0.5-2) | (0.03-0.12) | (4-16) | (0.06-0.25) | (0.25-1) | (0.12-0.25) |
| (ATCC 29213) | | | | | | | |
| S. aureus | MRSA² | 2 | 4 | >32 | 0.12 | >64 | 64 |
| MMX 9203 | Lev^{R} | | | | | | |
| | Cip^{R} | | | | | | |
| | Ery^{R} | | | | | | |
| | Ox^{R} | | | | | | |
| S. aureus | MRSA | 1 | 4 | >32 | 0.12 | >64 | 64 |
| MMX 9204 | Lev^{R} | | | | | | |
| | Cip^{R} | | | | | | |
| | Ery^{R} | | | | | | |
| | Ox^{R} | | | | | | |
| S. aureus | MRSA | 2 | 64 | >32 | >32 | >64 | >64 |
| MMX 9205 | Lev^{R} | | | | | | |
| | Cip^{R} | | | | | | |
| | CC^{R} | | | | | | |
| | Ery^{R} | | | | | | |
| | Ox^{R} | | | | | | |
| S. aureus | MRSA | 2 | 32 | >32 | >32 | >64 | >64 |
| MMX 5373 | Lev^{R} | | | | | | |
| | Cip^{R} | | | | | | |
| | CC^{R} | | | | | | |
| | Ery^{R} | | | | | | |
| | Ox^{R} | | | | | | |
| S. aureus | MRSA | 0.25 | 8 | >32 | >32 | >64 | 32 |
| MMX 6311 | Lev^{R} | | | | | | |
| | Cip^{R} | | | | | | |
| | CC^{R} | | | | | | |
| | Ery^{R} | | | | | | |
| | Ox^{R} | | | | | | |
| E. faecalis | VRE | >64 | 8 | >32 | >32 | >64 | >64 |
| MMX 8960 | Lev^{R} | | | | | | |
| | Cip^{R} | | | | | | |
| | Ery^{R} | | | | | | |
| E. faecalis | VRE | >64 | 8 | >32 | >32 | >64 | 64 |
| MMX 8961 | Lev^{R} | | | | | | |
| | Cip^{R} | | | | | | |
| | Ery^{R} | | | | | | |
| E. faecalis | VRE | >64 | 2 | >32 | >32 | >64 | 16 |
| MMX 8962 | Lev^{R} | | | | | | |
| | Cip^{R} | | | | | | |
| | Ery^{R} | | | | | | |
| E. faeciurn | VRE | >64 | >64 | >32 | >32 | >64 | >64 |
| MMX 752 | vanA | | | | | | |
| | Lev^{R} | | | | | | |
| | Cip^{R} | | | | | | |
| | Ery^{R} | | | | | | |
| E. faecium | VRE | >64 | >64 | >32 | >32 | >64 | >64 |
| MMX 485 | vanA | | | | | | |
| | Lev^{R} | | | | | | |
| | Cip^{R} | | | | | | |
| | Ery^{R} | | | | | | |

QC = quality control;
MSSA = methicillin-susceptible S. aureus;
MRSA = methicillin-resistant S. aureus;
VRE = vancomycin-resistant enterococci;
vanA = vanA-type VRE (based on vancomycin- and teicoplanin-resistant phenotype);
Lev^{R} = levofloxacin-resistant;
CIP^{R} = Ciprofloxacin-resistant;
CC^{R} = Clindamycin-resistant;
Ery^{R} = Erythromycin-resistant;
Ox^{R} = oxacillin-resistant
¹ CLSI QC ranges shown in parenthesis where applicable
² MRSA do not have breakpoints for ceftazidime and meropenem-resistance is presumed.

TABLE 92
Minimal Inhibitory Concentration (MIC) Values for Microbion Bismuth Thiol Test Agents and Comparators Against
Streptococcus spp.

| | | MIC (μg/mL) | | | | | |
|---|---|---|---|---|---|---|---|
| Isolate | Type | MB-1-B3 | MB-2B | MB-6 | Levofloxacin | Ciprofloxacin | Meropenem |
| S. pneumoniae | QC | 0.5 | 8 | 0.5 | 0.5 | 0.5 | 0.06 |
| MMX 6837 | | | | | (0.5-2) | | (0.06-0.25) |
| (ATCC 49619) | | | | | | | |
| S. pneumoniae | Lev^{R} | 1 | 8 | 0.5 | 8 | 32 | 0.5 |
| MMX 880 | SXT^{R} | | | | | | |
| | CC^{R} | | | | | | |
| | Ery^{R}Pen^{R} | | | | | | |
| S. pneumoniae | Mem^{R} | 1 | 1 | 8 | 1 | 1 | 1 |
| MMX 937 | SXT^{R} | | | | | | |
| | CC^{R} | | | | | | |
| | Ery^{R} Per^{R} | | | | | | |
| S. pneumoniae | CC^{R} | 1 | 8 | 1 | 1 | 4 | 0.03 |
| MMX 3959 | Ery^{R} | | | | | | |
| S. pneumoniae | Mem^{R} | 1 | 8 | 0.5 | 1 | 2 | 1 |
| MMX 5440 | SXT^{R} | | | | | | |
| | CC^{R} | | | | | | |
| | Ery^{R} Pen^{R} | | | | | | |
| S. pneumoniae | Mem^{R} | 0.5 | 8 | 0.5 | 1 | 2 | 1 |
| MMX 5445 | SXT^{R} | | | | | | |
| | CC^{R} | | | | | | |
| | Ery^{R} Pen^{R} | | | | | | |
| S. pneumoniae | Mem^{R} | 0.25 | 8 | 0.5 | 1 | 1 | 1 |
| MMX 8133 | SXT^{R} | | | | | | |
| | CC^{R} | | | | | | |
| | Ery^{R} Pen^{R} | | | | | | |
| S. pyogenes | Ery^{R} | 0.12 | 2 | 0.12 | 0.5 | 0.5 | ≤0.008 |
| MMX 3068 | | | | | | | |
| S. pyogenes | ERY^{R} | 0.25 | 1 | 0.5 | 0.5 | 0.12 | ≤0.008 |
| MMX 3820 | CLI^{R} | | | | | | |
| S. pyogenes | ERY^{R} | 0.03 | 0.25 | 0.03 | 0.25 | 0.25 | ≤0.008 |
| MMX 3919 | | | | | | | |
| S. pyogenes | ERY^{R} | 0.25 | 1 | 0.5 | 0.12 | 0.5 | ≤0.008 |
| MMX 3929 | CC^{R} | | | | | | |
| S. pyogenes | ERY^{R} | 0.5 | 1 | 1 | 0.5 | 0.25 | ≤0.008 |
| MMX 5091 | CC^{R} | | | | | | |
| S. agalactiae | ERY^{R} | 0.5 | 4 | 0.25 | 0.25 | 0.5 | 0.03 |
| MMX 3741 | CC^{R} | | | | | | |
| S. agalactiae | ERY^{R} | 0.25 | 4 | 0.25 | 1 | 1 | 0.06 |
| MMX 3743 | CC^{R} | | | | | | |
| S. agalactiae | ERY^{R} | 0.5 | 4 | 0.25 | 0.5 | 1 | 0.06 |
| MMX 4077 | CC^{R} | | | | | | |
| S. agalactiae | ERY^{R} | 1 | 8 | 0.5 | 1 | 1 | 8 |
| MMX 4079 | CC^{R} Pen^{R} | | | | | | |
| | MEM^{R} | | | | | | |
| S. agalactiae | ERY^{R} | 0.25 | 2 | 0.25 | 0.5 | 0.5 | 0.06 |
| MMX 4086 | CC^{R} | | | | | | |

| | | MIC (μg/mL) | | | | |
|---|---|---|---|---|---|---|
| Isolate | Type | Vancomycin | Trimeth/Sulfa | Clindamycin | Erythromycin | Penicillin |
| S. pneumoniae | QC | 0.25 | 0.25/4.8 | 0.06 | 0.03 | 0.25 |
| MMX 6837 | | (0.12-0.5) | (0.12/2.4-1/19) | (0.03-0.12) | (0.03-0.12) | (0.25-1) |
| (ATCC 49619) | | | | | | |
| S. pneumoniae | Lev^{R} | 0.5 | 4/76 | >8 | >8 | 2 |
| MMX 880 | SXT^{R} | | | | | |
| | CC^{R} | | | | | |
| | Ery^{R}Pen^{R} | | | | | |
| S. pneumoniae | Mem^{R} | 0.5 | 16/304 | >8 | >8 | 4 |
| MMX 937 | SXT^{R} | | | | | |
| | CC^{R} | | | | | |
| | Ery^{R} Per^{R} | | | | | |
| S. pneumoniae | CC^{R} | 0.5 | 2/38 | >8 | >8 | 0.12 |
| MMX 3959 | Ery^{R} | | | | | |
| S. pneumoniae | Mem^{R} | 0.25 | 16/304 | >8 | >8 | 4 |
| MMX 5440 | SXT^{R} | | | | | |
| | CC^{R} | | | | | |
| | Ery^{R} Pen^{R} | | | | | |
| S. pneumoniae | Mem^{R} | 0.25 | 8/152 | >8 | >8 | 4 |
| MMX 5445 | SXT^{R} | | | | | |
| | CC^{R} | | | | | |
| | Ery^{R} Pen^{R} | | | | | |
| S. pneumoniae | Mem^{R} | 0.25 | 8/152 | >8 | >8 | 4 |
| MMX 8133 | SXT^{R} | | | | | |
| | CC^{R} | | | | | |
| | Ery^{R} Pen^{R} | | | | | |
| S. pyogenes | Ery^{R} | 0.5 | 0.12/2.4 | 0.06 | >8 | ≤0.008 |
| MMX 3068 | | | | | | |
| S. pyogenes | ERY^{R} | 0.5 | 0.12/2.4 | 1 | >8 | ≤0.008 |
| MMX 3820 | CLI^{R} | | | | | |
| S. pyogenes | ERY^{R} | 0.5 | 0.06/1.2 | 0.12 | >8 | ≤0.008 |
| MMX 3919 | | | | | | |
| S. pyogenes | ERY^{R} | 0.5 | 0.25/4.8 | >8 | >8 | ≤0.008 |
| MMX 3929 | CC^{R} | | | | | |
| S. pyogenes | ERY^{R} | 0.5 | 0.06/1.2 | >8 | >8 | ≤0.008 |
| MMX 5091 | CC^{R} | | | | | |
| S. agalactiae | ERY^{R} | 0.5 | 0.12/2.4 | >8 | 8 | 0.03 |
| MMX 3741 | CC^{R} | | | | | |
| S. agalactiae | ERY^{R} | 0.5 | 0.12/2.4 | >8 | >8 | 0.06 |
| MMX 3743 | CC^{R} | | | | | |
| S. agalactiae | ERY^{R} | 0.5 | 0.12/2.4 | >8 | >8 | 0.06 |
| MMX 4077 | CC^{R} | | | | | |
| S. agalactiae | ERY^{R} | 2 | 0.12/2.4 | >8 | >8 | 2 |
| MMX 4079 | CC^{R} Pen^{R} | | | | | |
| | MEM^{R} | | | | | |
| S. agalactiae | ERY^{R} | 1 | 0.12/2.4 | >8 | >8 | 0.06 |
| MMX 4086 | CC^{R} | | | | | |

QC = quality control;
Trimeth = trimethoprim;
Sulfa = sulfamethoxazole;
MDR = mult-drug resistant (based on resistance to at least 3 different classes of antibiotic);
Ery^{R} = erythromycin-resistant;
CC^{R} = clindamycin-resistant;
SXT^{R} = Trimethoprim/Sulfamethoxazole-resistant;
MEM^{R} = Meropenem-resistant;
Pen^{R} = Penicillin-resistant
¹CLSI QC ranges shown in parenthesis where applicable

TABLE 93
Minimal Inhibitory Concentration (MIC) Values for Microbion Bismuth Thiol Test
Agents and Comparators Against N. gonorrhoeae

| | | MIC (μg/mL) | | | | |
|---|---|---|---|---|---|---|
| Isolate | Type | MB-1-B3 | MB-2B | MB-6 | Ciprofloxacin | Ceftriaxone |
| N. gonorrhoeae | QC | 0.06 | 0.12 | 0.06 | 0.008 | 0.015 |
| MMX 683 | | | | | (0.001-0.008)¹ | (0.004-0.015) |
| (ATCC 49226) | | | | | | |
| N. gonorrhoeae | CIP^{R} | 0.12 | 0.12 | 0.12 | >8 | 0.06 |
| MMX 6791 | | | | | | |
| N. gonorrhoeae | CIP^{R} | 0.12 | 0.12 | 0.12 | >8 | 0.06 |
| MMX 6792 | | | | | | |
| N. gonorrhoeae | CIP^{R} | 0.12 | 0.5 | 0.25 | >8 | 0.03 |
| MMX 6793 | | | | | | |
| N. gonorrhoeae | CTX NS | 0.06 | 0.5 | 0.06 | 0.03 | 1 |
| MMX 6757 | | | | | | |

QC = quality control;
CIP^{R} = ciprofloxacin-resistant;
CTX NS = ceftriaxone non-susceptible
¹CLSI QC ranges shown in paresthesis where applicable

TABLE 94
Minimal Inhibitory Concentration (MIC) Values for Microbion Bismuth
Thiol Test Agents and Comparators Against Anaerobes

| | | MIC (μg/mL) | | | | | |
|---|---|---|---|---|---|---|---|
| Isolate | Type | MB-1-B3 | MB-2B | MB-6 | Clindamycin | Metronidazole | Fidaxomicin |
| B. fragilis | QC | 2 | 8 | 1 | 1 | 0.5 | >64 |
| MMX 123 | | | | | (0.5-2)¹ | (0.25-1) | |
| (ATCC 25285) | | | | | | | |
| C. difficile | QC | 4 | 8 | 4 | 4 | 0.5 | 0.25 |
| MMX 4381 | CC¹ | | | | (2-8) | (0.12-0.5) | (0.06-0.25) |
| (ATCC 700057) | | | | | | | |
| C. difficile | ribotype 012 | 4 | 8 | 2 | >64 | 0.5 | 0.5 |
| MMX 5681 | CC^{R} | | | | | | |
| (NCTC 13307) | | | | | | | |
| C. difficile | ribotype 027 | 1 | 2 | 2 | 4 | >64 | 4 |
| MMX 5680 | CC¹ | | | | | | |
| (NCTC 13336) | MET^{R} | | | | | | |
| C. difficile | ribotype 255 | 4 | 8 | 2 | 4 | 0.5 | 0.5 |
| MMX 8272 | CC¹ | | | | | | |
| C. difficile | ribotype 005 | 4 | 16 | 2 | >64 | 0.5 | 0.5 |
| MMX 8279 | CC^{R} | | | | | | |
| C. difficile | ribotype 010 | 2 | 8 | 2 | >64 | 8 | 0.5 |
| MMX 8281 | CC^{R} | | | | | | |

QC = quality control;
CC = Clindamycin intermediate resistance;
CC^{R} = Clindamycin-resistant;
MET^{R} = Metronidazole-resistant
¹CLSI QC ranges shown in paresthesis where applicable

TABLE 95
Minimal Inhibitory Concentration (MIC) Values for Microbion Bismuth Thiol Test
Agents and Comparators Against Candida species

| | | MIC¹ (μg/mL) | | | | |
|---|---|---|---|---|---|---|
| Isolate | Type | MB-1-B3 | MB-2B | MB-6 | Fluconazole | Amphotericin B |
| C. parapsilosis | QC | 0.5, 1 | 0.5, 1 | 0.5, 1 | 2, 2 | 0.5, 1 |
| MMX 2323 | | | | | (0.5-4, 1-4)² | (0.25-2, 0.5-4) |
| (ATCC 22019) | | | | | | |
| C. parapsilosis | FLU^{R} | 0.5, 0.5 | 0.5, 0.5 | 0.25, 0.5 | 32, 32 | 0.5, 1 |
| MMX 7370 | | | | | | |
| C. parapsilosis | FLU^{R} | 0.5, 1 | 0.25, 1 | 0.25, 0.5 | 32, 64 | 0.5, 0.5 |
| MMX 7555 | | | | | | |
| C. albicans | Sensitive | 4, 16 | 2, 2 | 2, 2 | 0.5, 0.5 | 0.5, 0.5 |
| MMX 7039 | | | | | | |
| C. albicans | Sensitive | 2, 16 | 1, 4 | 2, 2 | 0.25,0.5 | 0.25, 0.5 |
| MMX 7055 | | | | | | |
| C. glabrata | FLU^{R} | 0.5, 1 | 0.5, 1 | 0.5, 1 | 32, 64 | 0.5, 1 |
| miX 7086 | | | | | | |
| C. glabrata | FLU^{R} | 0.5, 1 | 0.25, 1 | 0.25, 0.5 | >64, >64 | 0.5, 1 |
| MMX 7318 | | | | | | |
| C. tropicalis | FLU^{R} | 4, 16 | 2, 4 | 4, 4 | 64, >64 | 0.5, 1 |
| MMX 7247 | | | | | | |
| C. tropicalis | FLU^{R} | 16, 32 | 4, 4 | 4, 8 | 32, >64 | 0.5, 1 |
| MMX 7248 | | | | | | |
| C. tropicalis | FLU^{R} | 2, 16 | 1, 2 | 2, 2 | 64, >64 | 0.5, 0.5 |
| MMX 7360 | | | | | | |

QC = quality control;
FLU^{R} = fluconazole-resistant
¹MIC reported after incubation at 24 and 48 hr
²CLSI QC ranges shown in parenthesis where applicable

### REFERENCES

1.) Centers for Disease Control and Prevention. Antibiotic resistance threats in the United States, 2013. Available from http://www.cdc.gov/drugresistance/pdf/ar-threats-2013-508.pdf. Accessed on Jun. 13, 2016.
2.) Boucher H W, Talbot G H, Bradley J S, Edwards J E, Gilbert D, Rice L B, Scheld M, Spellberg B, Bartlett J. Bad bugs, no drugs: no ESKAPE! An update from the Infectious Diseases Society of America. Clin Infect Dis 2009; 48: 1-12.
3.) Rice L B. Federal funding for the study of antimicrobial resistance in nosocomial pathogens: no ESKAPE. J Infect Dis 2008; 197: 1079-1081.
4.) Clinical and Laboratory Standards Institute (CLSI). Methods for Dilution Antimicrobial Susceptibility Tests for Bacteria That Grow Aerobically; Approved Standard-Tenth Edition. Clinical and Laboratory Standards Institute document M07-A10. CLSI, 940 West Valley Road, Suite 1400, Wayne, Pa. 19087-1898 USA, 2015.
5.) CLSI. Performance Standards for Antimicrobial Susceptibility Testing; Twenty-Sixth Informational Supplement. CLSI document M100-S26. CLSI, 940 West Valley Road, Suite 1400, Wayne, Pa. 19087 USA, 2016.
6.) CLSI. Methods for Antimicrobial Susceptibility Testing of Anaerobic Bacteria; Approved Standard-Eighth Edition. CLSI document M11-A8. CLSI, 940 West Valley Road, Suite 1400, Wayne, Pa. 19087-1898 USA, 2012.
7.) CLSI. Reference Method for Broth Dilution Antifungal Susceptibility Testing of Yeasts; Approved Standard-Third Edition. CLSI document M27-A3. CLSI, 940 West Valley Road, Suite 1400, Wayne, Pa. 19087-1898 USA, 2008.
8.) CLSI. Reference Method for Broth Dilution Antifungal Susceptibility Testing of Yeasts; Fourth Informational Supplement. CLSI document M27-S4. CLSI, 940 West Valley Road, Suite 1400, Wayne, Pa. 19087-1898 USA, 2012.

### Example 12: Susceptibility Testing of Three Bismuth Thiols Against Vancomycin-Intermediate S. Aureus and β-Lactamase Producing Gram-Negative Bacteria

### Introduction

The in vitro activity of BisEDT and two additional bismuth-thiol investigational agents (MB-2B and MB-6) was determined for isolates of Escherichia coli, Klebsiella pneumoniae, Pseudomonas aeruginosa and Acinetobacter baumannii characterized for extended-spectrum pi-lactamases (ESBL) and/or carbapenem resistance. In addition, vancomycin-intermediate Staphylococcus aureus (VISA) were evaluated. The majority of the isolates tested in the current study were multidrug-resistant (MDR) as defined by resistance to at least three different antibiotic classes. Susceptibility to the investigational compounds and relevant comparators was determined by broth microdilution conducted in accordance with guidelines from the Clinical and Laboratory Standards Institute (CLSI; 2,3).

### Materials and Methods

Test Compounds. The test agents BisEDT (MB-1-B3; Lot No. ED268-1-11-01), MB-2B, and MB-6 were stored at room temperature, in the dark, until assayed. The solvent and diluent for the test agents was DMSO (Sigma; St. Louis, Mo.; Lot No. SHBB9319V) and the prepared stock concentration was 6,464 μg/mL (101X the final test concentration).

Comparator drugs were supplied and shown in Table 96 below:

TABLE 96
Comparator drugs

| Test Agents | Supplier | Catalog Number | Lot Number | Solvent/Diluent |
|---|---|---|---|---|
| BisEDT | Microbion | — | ED268-1-11-01 | DMSO/DMSO |
| MB-2B | Microbion | — | TA-8-167-01 | DMSO/DMSO |
| MB-6 | Microbion | — | 5-21-14 | DMSO/DMSO |
| Amikacin | Sigma | A2324-5G | 058K0803 | H₂O/H₂O |
| Ceftazidime | Sigma | C3809-1G | 076M4770V | H₂O/H₂O |
| Clavulanate | Sigma | 33454-100MG | STBH5214 | Phos. buff. pH 6.0 |
| Clindamycin | Sigma | C5269-100MG | 021M1533 | H₂O/H₂O |
| Daptomycin | Cubist | — | MCB2009 | H₂O/H₂O |
| Levofloxacin | Sigma | 28266-1G-F | BCBF7004V | H₂O + NaOH/H₂O |
| Linezolid | Selleck Chemicals | S1408 | S140802 | H₂O/H₂O |
| Meropenem | USP | 1392454 | J0K434 | H₂O/H₂O |
| Vancomycin | Sigma | V2002-1G | 080M1341V | H₂O/H₂O |

Test compounds were evaluated at a concentration range of 0.06-64 μg/mL. For Gram-negative test isolates, amikacin and ceftazidime (alone and with clavulanate at a fixed concentration of 4 μg/mL) were evaluated over a concentration range of 0.06-64 μg/mL; meropenem and levofloxacin were evaluated over a concentration range of 0.008-8 μg/mL. For the testing of S. aureus, clindamycin, daptomycin, levofloxacin and vancomycin were evaluated at a concentration range of 0.008-8 μg/mL; linezolid was tested from 0.03-32 μg/mL.

Organisms: The test organisms as shown in Tables 97-101 consisted of clinical isolates from the Micromyx (MMX) repository and reference strains from the American Type Culture Collection (ATCC; Manassas, Va.), National Collection of Type Cultures (NCTC; Public Health England, Salisbury, UK), the Network on Antimicrobial Resistance in Staphylococcus aureus (NARSA; BEI Resources, Manassas, Va.), and the Centers for Disease Control and Prevention (CDC; Atlanta, Ga.). The test organisms were maintained frozen at −80° C. Prior to testing, the isolates were cultured on Tryptic Soy Agar with 5% sheep blood (BAP; Becton Dickson [BD]/BBL; Sparks, Md.; Lot Nos. 9080650 and 9108563) at 35° C. Relevant ATCC quality control (QC) organisms (Table 102) were included during testing in accordance with CLSI guidelines (3). Further details on the genetic characterization of the isolates where available can be found in Table 103.

Media: Cation-adjusted Mueller Hinton broth (CAMHB; BD: Lot No. 8190586) was used as the medium for testing (2, 3). For testing daptomycin, calcium was supplemented with 25 mg/mL Ca²⁺, resulting in a final concentration of 50 mg/mL Ca²⁺ (2, 3).

MIC Assay Procedure: MIC values were determined using a broth microdilution procedure described by CLSI (2, 3). Automated liquid handlers (Multidrop 384, Labsystems, Helsinki, Finland; Biomek 2000 and Biomek FX, Beckman Coulter, Fullerton Calif.) were used to conduct serial dilutions and liquid transfers.

To prepare the drug mother plates, which would provide the serial drug dilutions for the replicate daughter plates, the wells of columns 2 through 12 of standard 96-well microdilution plates (Costar 3795) were filled with 150 μl of the appropriate diluent for each row of drug. The test articles and comparator compounds (300 μl at 101X the highest concentration to be tested) were dispensed into the appropriate wells in column 1. The Biomek 2000 was then used to make 2-fold serial dilutions in the mother plates from column 1 through column 11. The wells of column 12 contained no drug and served as the organism growth control wells for the assay.

The daughter plates were loaded with 190 μL per well of RPMI using the Multidrop 384. The test panels were prepared on the Biomek FX instrument which transferred 2 μL of drug solution from each well of a mother plate to the corresponding well of each daughter plate in a single step.

A standardized inoculum of each test organism was prepared per CLSI methods to equal a 0.5 McFarland standard, followed by a dilution of 1:20. The plates were then inoculated with 10 μL of the diluted inoculum using the Biomek 2000 from low to high drug concentration resulting in a final concentration of approximately 5×10⁵ CFU/mL.

Plates were stacked 3-4 high, covered with a lid on the top plate, placed into plastic bags, and incubated at 35° C. for 16 to 20 hr (vancomycin was read for S. aureus after 24 hr incubation time). The MIC was recorded as the lowest concentration of drug that inhibited visible growth of the organism.

### Results and Discussion

As shown in Table 102, results for BisEDT and comparators were within CLSI established QC ranges against the relevant ATCC QC isolates, thus validating the susceptibility testing conducted during the study.

The activity of BisEDT, MB-2B, and MB-6, against the resistant Gram-negative bacilli are shown by species in Tables 97-100. BisEDT maintained potent activity with MIC values of 0.5-2 μg/mL across isolates with the exception of one isolate of P. aeruginosa (CDC 241) which had an MIC of 4 μg/mL (Table 99) and several isolates of K. pneumoniae with MIC values of 4-8 μg/mL (Table 98). The activity of BisEDT was not impacted by D3-lactamase production or resistance to aminoglycosides (amikacin MIC ≥64 μg/mL), fluoroquinolones (levofloxacin MIC ≥2, 4, and 8≥64 for Enterobacteriaceae, P. aeruginosa, and A. baumannii, respectively).

Overall, MB-6 had MIC values that were either identical or within 2-fold of those observed with BisEDT; exceptions included select K. pneumoniae where MB-6 MIC values were lower than those of BisEDT. The activity of BisEDT and MB-6 was greater than that of MB-2B, particularly for P. aeruginosa and A. baumannii. The MIC values observed with BisEDT, MB-2B, and MB-6 against Gram-negative bacilli were comparable to those observed in prior studies (1, 4).

The activity of BisEDT, MB-2B, and MB-6 against VISA is shown in Table 101. BisEDT had potent MIC values of ≤0.06-0.25 μg/mL against these isolates. As with Gram-negative bacilli, the activity of BisEDT was comparable to that observed with MB-6 and was greater than that observed with MB-2B. Of note, two of the VISA isolates (NRS 13 and 27) from NARSA had vancomycin MIC values of 2 μg/mL, which indicated that during testing in this study they tested as vancomycin-susceptible. The other two isolates with vancomycin MIC values in the susceptible range (NRS 2 and 24) are heterogenous VISA (hVISA) for which vancomycin MIC values are known to vary. Resistance to levofloxacin and clindamycin (MIC values >4 μg/mL) was observed with all isolates except NRS 13 and did not impact BisEDT activity. Two of the isolates were also non-susceptible to daptomycin (NRS 13 and 22); all were susceptible to linezolid (MIC values <4 μg/mL). The activity observed with BisEDT in this study was comparable to that observed previously (1, 4).

In summary, BisEDT showed potent activity against genetically characterized p-lactam-resistant Gram-negative bacilli, the majority of which were MDR, and reference isolates of VISA. The activity of BisEDT was not impacted by resistance to R-lactams or any other class evaluated in this study. Finally, the activity of BisEDT and MB-6 was comparable against the evaluated bacteria and exceeded that observed with MB-2B.

TABLE 97
Activity of BisEDT, MB-2B, MB-6 and comparators against Escherichia coli

| | | MIC (μg/mL) | | | | | | | |
|---|---|---|---|---|---|---|---|---|---|
| | β-lactamase | | | | | CAZ/ | | | |
| Isolate | Type | BisEDT | MB-2B | MB-6 | CAZ | CLAV | MEM | LVX | AMK |
| ATCC 35218 | ESBL | 1 | 2 | 1 | 0.12 | ≤0.06/4 | ≤0.008 | ≤0.008 | 1 |
| MMX 5755 | ESBL | I | 4 | 2 | >64 | 1/4 | 0.015 | 8 | 16 |
| MMX 5756 | ESBL | 1 | 2 | 1 | >64 | 0.5/4 | ≤0.008 | 4 | 64 |
| MMX 5758 | ESBL | 1 | 4 | 1 | 16 | 0.25/4 | ≤0.008 | 8 | 1 |
| CDC 451 | KPC | 1 | 2 | 1 | 64 | 16/4 | 1 | >8 | 4 |
| MMX 5745 | KPC | 1 | 4 | 2 | 32 | 16/4 | 2 | 8 | 0.5 |
| CDC 114 | ESBL/KPC | 2 | 2 | 2 | >64 | 64/4 | 2 | 4 | 1 |
| ATCC BAA-2471 | NDM | 1 | 2 | 2 | >64 | >64/4 | >8 | >8 | 64 |
| CDC 435 | NDM | 1 | 2 | 1 | >64 | >64/4 | >8 | >8 | >64 |
| CDC 503 | ESBL/NDM | 1 | 2 | 2 | >64 | >64/4 | 8 | >8 | >64 |
| CDC 118 | ESBL/NDM | 1 | 4 | 1 | >64 | >64/4 | 0.06 | 8 | >64 |

ATCC = American Type Culture Collection,
MMX = Micromyx,
CDC = Centers for Disease Control and Prevention,
ESBL = extended-spectrum β-lactamase,
KPC = K. pneumoniae carbapenemase,
NDM = New Delhi metallo-β-lactamase,
CAZ = ceftazidime,
CLAV = clavulanate,
MEM = meropenem,
LVX = levofloxacin,
AMK = amikacin

TABLE 98
Activity of BisEDT, MB-2B, MB-6 and comparators against Klebsiella pneumoniae

| | | MIC (μg/mL) | | | | | | | |
|---|---|---|---|---|---|---|---|---|---|
| | β-lactamase | | | | | CAZ/ | | | |
| Isolate | Type | BisEDT | MB-2B | MB-6 | CAZ | CLAY | MEM | LVX | AMK |
| MMX 9029 | ESBL | 4 | 2 | 2 | 4 | 0.12/4 | 0.015 | 0.12 | 0.5 |
| CDC 112 | KPC | 2 | 4 | 2 | >64 | >64/4 | >8 | 4 | 16 |
| ATCC BAA-1705 | ESBL/KPC | 2 | 4 | 1 | >64 | >64/4 | 8 | >8 | 16 |
| CDC 113 | ESBL/KPC | 8 | 32 | 1 | >64 | >64/4 | >8 | 4 | 16 |
| CDC 115 | ESBL/KPC | 8 | 16 | 2 | >64 | >64/4 | >8 | >8 | 0.5 |
| CDC 120 | ESBL/KPC | 2 | 4 | 2 | >64 | 64/4 | >8 | >8 | 16 |
| CDC 126 | ESBL/KPC | 8 | 32 | 2 | 4 | 8 | 4 | 0.015 | 2 |
| CDC 129 | ESBL/KPC | 2 | 4 | 4 | >64 | >64/4 | 8 | >8 | 32 |
| CDC 135 | ESBL/VIM | 4 | 8 | 4 | >64 | >64/4 | 1 | >8 | 16 |
| CDC 138 | ESBL/NDM | 4 | 16 | 2 | >64 | >64/4 | >8 | >8 | >64 |
| CDC 158 | ESBL/NDM/OXA | 2 | 4 | 2 | >64 | >64/4 | >8 | 4 | 2 |

ATCC = American Type Culture Collection,
MMX = Micromyx,
CDC = Centers for Disease Control and Prevention,
ESBL = extended-spectrum β-lactamase,
KPC = K. pneumoniae carbapenemase,
NDM = New Delhi metallo-β-lactamase,
VIM = metallo-β-lactamase,
OXA = class D carbapenemases,
CAZ = ceftazidime,
CLAV = clavulanate,
MEM = meropenem,
LVX = levofloxacin.
AMK = amikacin

TABLE 99
Activity of BisEDT, MB-2B, MB-6 and comparators against Pseudomonas aeruginosa

| | | MIC (μg/mL) | | | | | | | |
|---|---|---|---|---|---|---|---|---|---|
| | β-lactamase | | | | | CAZ/ | | | |
| Isolate | Type | BisEDT | MB-2B | MB-6 | CAZ | CLAY | MEM | LVX | AMK |
| CDC 356 | KPC | 1 | 4 | 1 | 64 | 64/4 | >8 | 0.12 | 2 |
| CDC 439 | IMP | 2 | 4 | 1 | >64 | >64/4 | >8 | 8 | >64 |
| CDC 444 | VIM | 1 | 4 | 1 | 32 | 32/4 | >8 | 8 | >64 |
| CDC 457 | VIM | 1 | 32 | 2 | >64 | 64/4 | >8 | >8 | 2 |
| CDC 231 | KPC/OXA | 2 | 16 | 2 | >64 | >64/4 | >8 | >8 | 8 |
| CDC 230 | VIM/OXA | 0.5 | 4 | 2 | 64 | 32/4 | >8 | 8 | >64 |
| CDC 241 | IMP/OXA | 4 | 32 | 8 | >64 | >64/4 | >8 | 8 | 32 |
| CDC 246 | NDM/OXA | 2 | 8 | 2 | >64 | >64/4 | >8 | >8 | >64 |
| CDC 250 | NDM/OXA | 2 | 8 | 2 | >64 | >64/4 | >8 | >8 | >64 |
| CDC 516 | KPC/AmpC | 1 | 4 | 1 | 64 | 64/4 | >8 | 0.25 | 2 |
| CDC 518 | KPC/AmpC | 1 | 4 | 1 | 32 | 32/4 | >8 | >8 | 16 |

CDC = Centers for Disease Control and Prevention,
KPC = K. pneumoniae carbapenemase,
NDM = New Delhi metallo-β-lactamase,
IMP = metallo-β-lactamase,
VIM = metallo-β-lactamase,
OXA = class D carbapenemases,
AmpC = class C cephalosporinase,
CAZ = ceftazidime,
CLAV = clavulanate,
MEM = meropenem,
LVX = levofloxacin,
AMK = amikacin

TABLE 100
Activity of BisEDT, MB-2B, MB-6 and comparators against Acinetobacter baumannii

| | | MIC (μg/mL) | | | | | | | |
|---|---|---|---|---|---|---|---|---|---|
| | β-lactamase | | | | | CAZ/ | | | |
| Isolate | Type | BisEDT | MB-2B | MB-6 | CAZ | CLAV | MEM | LVX | AMK |
| NCTC 13304 | OXA | 0.5 | >16 | 0.5 | >64 | >64/4 | >8 | 1 | 0.5 |
| CDC 307 | OXA | 1 | 32 | 0.5 | 64 | 64/4 | 8 | 8 | >64 |
| CDC 311 | OXA | 1 | 32 | 0.5 | >64 | >64/4 | >8 | 2 | >64 |
| CDC 312 | OXA | 1 | 32 | 0.5 | >64 | >64/4 | 4 | 2 | 1 |
| CDC 273 | ESBL/OXA | 1 | 32 | 0.5 | 64 | 64/4 | >8 | >8 | >64 |
| CDC 274 | ESBL/OXA | 1 | 32 | 0.5 | >64 | >64/4 | >8 | 8 | 16 |
| CDC 275 | ESBL/OXA | 0.5 | 32 | 0.5 | >64 | >64/4 | >8 | 4 | >64 |
| CDC 277 | ESBL/OXA | 1 | >16 | 1 | >64 | >64/4 | >8 | 8 | 16 |
| CDC 284 | ESBL/OXA | 1 | >16 | 1 | 64 | 16/4 | >8 | 8 | 32 |
| CDC 308 | ESBL/OXA | 1 | 32 | 0.5 | 64 | 64/4 | 8 | 4 | >64 |
| CDC 313 | ESBL/OXA | 1 | 32 | 0.5 | >64 | >64/4 | >8 | 2 | 4 |

NCTC = National Collection of Type Cultures, CDC = Centers for Disease Control and Prevention, ESBL = extended-spectrum β-lactamase, OXA = class D carbapenemases, CAZ = ceftazidime, CLAV = clavulanate, MEM = meropenem, LVX = levofloxacin, AMK = amikacin

TABLE 101
Activity of BisEDT, MB-2B, MB-6 and comparators against
vancomycin-intermediate Staphylococcus aureus

| | MIC (μg/mL) | | | | | | | |
|---|---|---|---|---|---|---|---|---|
| Isolate | BisEDT | MB-2B | MB-6 | VAN | DAP | CLI | LVX | LZD |
| NRS 1 (hVISA) | 0.12 | 1 | 0.12 | 4 | 1 | >8 | 4 | 1 |
| NRS 2 | ≤0.06 | 0.5 | ≤0.06 | 0.5 | 0.25 | >8 | 4 | 4 |
| NRS 3 | 0.25 | 2 | 0.25 | 8 | 1 | >8 | >8 | 1 |
| NRS 22 | 0.25 | 2 | 0.25 | 4 | 2 | >8 | 8 | 1 |
| NRS 4 | 0.25 | 1 | 0.25 | 4 | 0.5 | >8 | 4 | 1 |
| NRS 13 | 0.12 | 1 | 0.12 | 2 | 2 | 0.06 | 0.12 | 2 |
| NRS 18 | 0.25 | 1 | 0.25 | 4 | 0.5 | >8 | 4 | 1 |
| NRS 24 (hVISA) | 0.25 | 1 | 1 | 2 | 0.5 | >8 | >8 | 2 |
| NRS 27 | 0.25 | 1 | 0.25 | 2 | 0.25 | >8 | 8 | 2 |

NRS = Network on Antimicrobial Resistance in Staphylococcus aureus, MMX = Micromyx, VISA = vancomycin-intermediate S. aureus, hVISA = heterogenous vancomycin-intermediate S. Aureus, VAN = vancomycin, DAP = daptomycin, CLI = clindamycin, LVX = levofloxacin, LZD = linezolid

TABLE 102
Activity of Bis-EDT, MB-2B, MB-6 and comparators against relevant ATCC QC organisms

| | | MIC (μg/mL) | | | | | | | |
|---|---|---|---|---|---|---|---|---|---|
| | | | | | | CAZ + | | | |
| Organism | Isolate | BisEDT | MB-2B | MB-6 | CAZ | CLAV | MEM | LVX | AMK |
| E. coli | ATCC 25922 | 0.5 | 2 | 1 | 0.25 | 0.25/4 | ≤0.008 | ≤0.008 | 1 |
| | | (0.5-4)¹ | | | (0.06-0.5) | | (0.008-0.06) | (0.008-0.06) | |
| | ATCC 35218 | 1 | 2 | 1 | 0.12 | ≤0.06/4 | ≤0.008 | ≤0.008 | 1 |
| | | | | | | | (0.008-0.06) | | |
| K. pneumoniae | ATCC BAA-1705 | 2 | 4 | 1 | >64 | >64/4 | 8 | >8 | 16 |
| | | | | | | | (8-64) | | |
| P. aeruginosa | ATCC 27853 | 1 | 2 | 1 | 2 | 2/4 | 0.25 | 0.5 | 2 |
| | | (0.5-4) | | | (1-4) | | (0.12-1) | (0.5-4) | (1-4) |
| | | MIC (μg/mL) | | | | | | | |
| Organism | Isolate | BisEDT | MB-2B | MB-6 | VAN | DAP | CLI | LVX | LZD |
| S. aureus | ATCC 29213 | 0.12 | 1 | 0.12 | 0.5 | 0.5 | 0.25 | 0.06 | 4 |
| | | (0.12-1) | | | (0.5-2) | (0.12-1) | (0.06-0.25) | (0.06-0.5) | (1-4) |

ATCC = American Type Culture Collection, CAZ = ceftazidime, CLAV = clavalanate, MEM = meropenem, LVX = levofloxacin, AMK = amikacin, VAN = vancomycin, DAP = daptomycin, CLI = clindamycin, LVX = levofloxacin, LZD = linezolid QC ranges in parentheses

TABLE 103
Available genetic characterization data on test isolates

| Organism | Isolate | Genetic Characterization Information |
|---|---|---|
| E. coli | ATCC 35218 | TEM-1 |
| | BAA-2471 | NDM-1 |
| | CDC 435 | NDM |
| | CDC 451 | KPC |
| | MMX 5745 | KPC, TEM, DFR |
| | MMX 5755 | SHV, TEM, OXA-1, AAD, ANT, SUL1, SUL2, GYR |
| | MMX 5756 | SHV, TEM, OXA-9, AAD, SUL2, GYR |
| | MMX 5758 | SHV, TEM, CTX-M-1, GYR |
| | CDC 503 | CTX-M-15, NDM-1, OXA-181 |
| | CDC 114 | aadB, cmlA1, dfrA5, KPC-3, strA, strB, sul1, sul2, TEM-1B |
| | CDC 118 | aac(3)-IIa, catA1, CMY-6, dfrA29, NDM-1, OmpF, OXA-2, rmtC, |
| | | strA, strB, sul1, TEM-1A |
| K. pneumoniae | BAA-1705 | KPC-2, TEM, SHV |
| | MMX 9029 | CTX-M1, SHV, TEM, AAC, SUL2 |
| | CDC 112 | aac(6′), aph(3′), aph(4), catA1, cmlA1, dfrA12, KPC-3, mph(A), |
| | | oqxA, oqxA, oqxB, sul1, sul3 |
| | CDC 113 | aac(6′)-Ib, aph(3′)-Ia, aph(4)-Ia, catA1, cmlA1, dfrA12, KPC-3, |
| | | mph(A), OmpK35, OmpK36, oqxA, oqxA, oqxB, SHV-11, sul1, |
| | | sul3 |
| | CDC 115 | aph(3′)-Ia, aph(4)-Ia, catA1, cmlA1, dfrA12, KPC-3, mph(A), |
| | | OmpK35, oqxA, oqxA, oqxB, sul1, sul3, TEM-1A |
| | CDC 120 | aac(6′)-33, aac(6′)-Ib, aadA2, aadB, aph(3′)-Ia, dfrA12, KPC-2, |
| | | mph(A), OmpK35, oqxA, oqxA, oqxB, sul1, sul2, TEM-1D |
| | CDC 126 | aac(6′ )Ib-cr, catB3, dfrA1, fosA, KPC-2, OmpK36, oqxA, oqxA, |
| | | OXA-1, sul1, TEM-1B |
| | CDC 129 | aac(6′)-Ib, aadA2, aph(3′)-Ia, catA1, dfrA12, KPC-3, mph(A), |
| | | OmpK35, oqxA, oqxA, oqxB, sul1, TEM-1A |
| | CDC 135 | aac(3)-IIa, aac(6′)-Ib, aph(3′)-XV, catB2, dfrA14, OmpK35, oqxA, |
| | | oqxA, OXA-9, SHV-12, sul1, TEM-1A, tet(D), VIM-1 |
| | CDC 138 | aadA2, ARR-3, CTX-M-15, dfrA12, dfrA14, mph(A), NDM-7, |
| | | oqxA, oqxA, SHV-11, strA, strB, sul1, sul2, TEM-1B |
| | CDC 158 | aac(3)-IId, aac(6′)Ib-cr, CTX-M-15, dfrA14, dfrA30, fosA, NDM-1, |
| | | oqxA, oqxA, oqxB, OXA-1, strA, strB, sul2, TEM-1B, tet(B) |
| P. aeruginosa | CDC 439 | IMP |
| | CDC 444 | VIM |
| | CDC 457 | VIM |
| | CDC 356 | KPC |
| | CDC 230 | aac(3)-Id, aadA2, cmlA1, dfrB5, OXA-4, OXA-50, PAO, |
| | | tet(G), VIM-2 |
| | CDC 231 | aac(6′)-IIc, KPC-5, OXA-2, OXA-50, PAO |
| | CDC 241 | aac(6′)-IIc, aadA7, catB7, IMP-1, OXA-101, OXA-50, OXA-9, |
| | | PAO, sul1 |
| | CDC 246 | aadB, NDM-1, OXA-10, OXA-50, PAO, rmtD2, tet(G), VEB-1 |
| | CDC 250 | aadB, NDM-1, OXA-10, OXA-50, PAO, rmtD2, tet(G), VEB-1 |
| | CDC 516 | PDC-101; KPC-2 |
| | CDC 518 | PDC-103; KPC-2 |
| A. baumannii | NCTC 13304 | OXA-27 |
| | CDC 273 | aac(3)-IIa, ADC-25, aph(3′)-Ic, aph(3′)-VIa, OXA-23, OXA-66, |
| | | strA, strB, sul2 |
| | CDC 274 | aac(3)-Ia, ADC-25, aph(3′)-Ic, OXA-66, OXA-72, strA, strB, sul1, |
| | | sul2, TEM-1D |
| | CDC 275 | ADC-25, aph(3′)-Ic, armA, mph(E), msr(E), OXA-23, OXA-66, |
| | | strA, strB, sul2, TEM-1D |
| | CDC 277 | aac(3)-IIa, OXA-24, OXA-65, strA, strB, sul2, TEM-1B |
| | CDC 284 | aac(3)-IIa, OXA-24, OXA-65, strA, strB, sul2, TEM-1B |
| | CDC 307 | ADC-25, aph(3′)-Ic, armA, catB8, mph(E), msr(E), OXA-23, OXA- |
| | | 66, strA, strB, sul1, sul2 |
| | CDC 308 | ADC-25, armA, catB8, mph(E), msr(E), OXA-71, strA, strB, |
| | | sul1, TEM-1D |
| | CDC 311 | ADC-25, aph(3′)-Ic, armA, catB8, mph(E), msr(E), OXA-23, OXA- |
| | | 82, strA, strB, sul1 |
| | CDC 312 | aph(3′)-Ic, catA1, OXA-69, sul2, tet(B) |
| | CDC 313 | aac(3)-Ia, aph(3′)-Ic, catA1, OXA-23, OXA-69, TEM-1D, tet(A) |

### REFERENCES

1.) Beckman E, Wolfe C, Pillar C. In vitro Activity of Bismuth Thiols and Comparators Against Drug Resistant Gram-positive and -negative Bacteria and Yeast. Final Report Aug. 24, 2016-Microbion 22. Micromyx, Kalamazoo, Mich. 2016.
2.) Clinical and Laboratory Standards Institute (CLSI). Methods for Dilution Antimicrobial Susceptibility Tests for Bacteria That Grow Aerobically; 11^{th} ed. CLSI standard M07. CLSI, 950 West Valley Road, Suite 2500, Wayne, Pa. 19087 USA, 2018.
3.) CLSI. Performance Standards for Antimicrobial Susceptibility Testing; 29^{th} ed. CLSI supplement M100. CLSI, 950 West Valley Road, Suite 2500, Wayne, Pa. 19087 USA, 2019.
4.) Schaadt R D, Peterson M, Sweeney D. In vitro Activity of Bismuth-1,2-ethanedithiol (BisEDT) Against Multiple Clinical Isolates of Gram-positive and -negative Bacteria. Final Report Aug. 8, 2008-Microbion 2. Micromyx, Kalamazoo, Mich. 2008.

### Example 13: Susceptibility Testing of Three Test Compounds Against Yeast (Candida Spp.) and Mold (Aspergillus Spp.)

### Introduction

The in vitro activity of BisEDT and two additional bismuth-thiol investigational agents (MB-2B and MB-6) was determined for Candida spp. (C. albicans, C. glabrata, C. krusei, and C. auris) and Aspergillus spp. (A. niger, A. terreus, A. fumigatus and A. flavus). Comparators included amphotericin B, fluconazole, voriconazole, caspofungin and micafungin. Susceptibility was determined by broth microdilution conducted in accordance with guidelines from the Clinical and Laboratory Standards Institute (CLSI; 2,3).

### Material and Methods

Test Compounds: The test agents BisEDT (MB-1-B3; Lot No. ED268-1-11-01), MB-2B, and MB-6 were shipped and stored at room temperature, in the dark, until assayed. The solvent and diluent for the test agents was DMSO (Sigma; St. Louis, Mo.; Lot No. SHBB9319V) and the prepared stock concentration was 6,464 μg/mL (101X the final test concentration for yeast and fungi). Comparator drugs are shown in Table 104 below:

TABLE 104
Comparator drugs

| | | | | Working Stock |
|---|---|---|---|---|
| Comparator | | | Solvent/ | Concentration |
| Drug | Supplier | Lot No. | Diluent | (μg/mL) |
| Amphotericin B | Sigma | 086M4012V | DMSO | 6464 |
| Fluconazole | USP | H11308 | DMSO | 6464 |
| Caspofungin | Sigma | 086M4750V | DMSO | 808 |
| Micafungin | Astellas | 023070 | DMSO | 808 |
| Voriconazole | USP | R032E0 | DMSO | 808 |

Microbion test compounds, amphotericin B, and fluconazole were evaluated over a concentration range of 0.06-64 μg/mL for yeast and mold. Caspofungin, micafungin, and voriconazole were tested from 0.008-8 μg/mL.

Organisms: The test organisms consisted of clinical isolates from the Micromyx (MMX) repository and reference strains from the American Type Culture Collection (ATCC, Manassas, Va.). The test organisms were maintained frozen at −80° C. Prior to testing, yeast were sub-cultured on Sabouraud Dextrose Agar (Becton, Dickson and Company; Sparks, Md.; Lot Nos. 9032625, 9074672) at 35° C. The molds were obtained from enumerated fungal stocks previously prepared at Micromyx and stored in a 0.1% Tween saline solution, at 4° C., until use. C. krusei ATCC 6258, C. parapsilosis ATCC 22019, A. fumigatus ATCC MYA-3626, and A. flavus ATCC 204304 were included for purposes of quality control (4, 5).

Media: The medium employed for the testing of the yeast and mold isolates was RPMI 1640 from Hyclone Laboratories (Logan, Utah; Lot No. AC10257966A) buffered with MOPS from EMD Millipore (Burlington, Mass.; Lot No. 3173588) (2, 3).

MIC Assay Procedures: MIC values were determined using a broth microdilution procedure described by CLSI (2, 3). Automated liquid handlers (Multidrop 384, Labsystems, Helsinki, Finland; Biomek 2000 and Biomek FX, Beckman Coulter, Fullerton Calif.) were used to conduct serial dilutions and liquid transfers.

To prepare the drug mother plates, which would provide the serial drug dilutions for the replicate daughter plates, the wells of columns 2 through 12 of standard 96-well microdilution plates (Costar 3795) were filled with 150 μl of DMSO for each row of drug. The test articles and comparator compounds (300 μl at 101X the highest concentration to be tested) were dispensed into the appropriate wells in column 1. The Biomek 2000 was then used to make 2-fold serial dilutions in the mother plates from column 1 through column 11. The wells of column 12 contained no drug and served as the organism growth control wells for the assay.

The daughter plates were loaded with 190 μL per well of RPMI using the Multidrop 384. The daughter plates were prepared on the Biomek FX instrument which transferred 2 μL of drug solution from each well of a mother plate to the corresponding well of each daughter plate in a single step.

A standardized inoculum of each organism was prepared per CLSI methods (2, 3). For the yeast, colonies were picked from the streak plate and a suspension was prepared in saline to equal a 0.5 McFarland standard. This suspension was diluted 1:100 in RPMI resulting in a final concentration of approximately 0.5-2.5×10³ CFU/mL in the assay. For the molds, based on the previously determined spore count (CFU/mL) from a spore suspension of each Aspergillus spp. isolate, the suspension was diluted in RPMI such that a final concentration of approximately 0.2-2.5×10⁴ CFU/mL was achieved in the assay. The inoculum for each organism was dispensed into sterile reservoirs divided by length (Beckman Coulter), and the Biomek 2000 was used to inoculate the plates. Daughter plates were placed on the Biomek 2000 work surface reversed so that inoculation took place from low to high drug concentration. The Biomek 2000 delivered 10 μL of standardized inoculum into each well. DMSO was present at a final concentration of 1% in the test wells.

Plates were stacked four high, covered with a lid on the top plate, placed into plastic bags, and incubated at 35° C. The Candida spp. and Aspergillus isolates were read after a 24 hr incubation and again at 48 hr.

The microplates were viewed from the bottom using a plate viewer. For each mother plate, an un-inoculated solubility control plate was observed for evidence of drug precipitation. For yeast, the MIC₀ was reported as the lowest concentration of drug that completely inhibited visible growth of the organism for Microbion test articles, amphotericin B, and voriconazole; the lowest concentration that showed 50% inhibition relative to the growth control was reported as the MIC₂ for all Microbion test articles, micafungin, caspofungin, and fluconazole. For molds, MIC values were reported as the lowest concentration at which visible growth was inhibited, and minimum effective concentrations (MECs) were reported for caspofungin, micafungin and the Microbion test agents as the lowest concentration where the growth shifted to a small, rounded, compact hyphal form at the bottom of the well as compared to the hyphal growth seen throughout the medium in the growth control well. MECs were only reported where observed.

### Results and Discussion

As shown in Table 110, results for BisEDT and comparators were within CLSI established QC ranges against the relevant ATCC QC isolates with the exception of amphotericin B at 48 hr against C. krusei ATCC 6258. In this instance, amphotericin B was within the QC range at 24 hr for the same isolate and was within the QC range at 48 hr for C. parapsilosis ATCC 22019, A. flavus ATCC 204304, and A. fumigatus MYA-3626. These results validate the susceptibility testing conducted during the study.

The activity of BisEDT, MB-2B, and MB-6, against yeast are shown by species in Tables 105-108. BisEDT maintained potent activity with 50% inhibition (MIC₂ values) observed from 0.25-1 μg/mL across species and 100% inhibition (MIC₀ values) observed from 0.5-2 μg/mL for all species except C. albicans, where MIC₀ values were 1-8 μg/mL. The activity of BisEDT was not impacted by resistance to echinocandins or azole anti-fungal agents and was notably maintained against C. auris for which multi-drug resistance and azole resistance is particularly an issue (6). The activity of MB-2B and MB-6 was similar to that of BisEDT, with MIC values identical or within 2-fold those of BisEDT. The MIC values observed with BisEDT, MB-2B, and MB-6 against yeast were comparable to those observed in a prior study (1).

The activity of BisEDT, MB-2B, and MB-6 against Aspergillus spp. is shown in Table 109. Overall, BisEDT resulted in complete inhibition at 24 hr with MIC₀ values of 2-8 μg/mL for most isolates. However, no complete inhibition was observed with A. flavus MMX 7935 (MIC₀ of >64 μg/mL), and an MIC₀ of 0.5 μg/mL was observed against A. terreus MMX 8229. After 48 hr of incubation, complete inhibition by BisEDT was less commonly observed across the tested isolates but in instances where MIC₀ values were not evident, MEC values were apparent and these values were typically consistent with the MIC₀ values reported at 24 hr. As with yeast, the activity of BisEDT was comparable to that observed with MB-2B and MB-6. The activity observed among the comparators against the Aspergillus spp. was typically more potent than BisEDT, MB-2B, and MB-6 with the exception of fluconazole which, as expected, was inactive.

In summary, BisEDT showed potent activity against both Candida spp., including the notoriously difficult to treat C. auris, and Aspergillus spp. The activity of BisEDT against yeast was not impacted by azole or echinocandin resistance. Finally, the activity of BisEDT, MB-2B, and MB-6 were comparable against the evaluated yeast and mold.

TABLE 105
Activity of BisEDT, MB-2B, MB-6 and comparators against Candida albicans

| | | Test agent and activity (μg/mL) at 24 hr | | | | | | | | | | |
|---|---|---|---|---|---|---|---|---|---|---|---|---|
| | | BisEDT | | MB-2B | | MB-6 | | AMP B | FLU | VORI | CAST | MICA |
| Isolate No. | Type | MIC₂ | MIC₀ | MIC₂ | MIC₀ | MIC₂ | MIC₀ | MIC₀ | MIC₂ | MIC₂ | MIC₂ | MIC₂ |
| ATCC 90028 | Susceptible | 0.5 | 4 | 0.5 | 2 | 0.5 | 2 | 0.25 | 0.25 | ≤0.008 | 0.06 | 0.03 |
| ATCC 204276 | MICA-R | 0.5 | 1 | 0.5 | 1 | 1^{a} | 1 | 0.25 | 0.25 | ≤0.008 | 0.25 | 4 |
| ATCC MYA-2732 | FLU-R, VORI-I | 1 | 2 | 1 | 2 | 1 | 2 | 0.25 | 16 | 0.5 | 0.06 | 0.03 |
| MMX 7067 | FLU-R, VORI-R | 1 | 2 | 0.5 | 1 | 0.5 | 1 | 0.25 | >64 | >8 | 0.06 | 0.03 |
| MMX 7424 | CASP-R, MICA-R | 1 | 4 | 1 | 2 | 1 | 2 | 0.25 | 0.5 | ≤0.008 | 8 | 4 |
| MMX 7053 | Susceptible | 0.5 | 1 | 0.5 | 1 | 0.5 | 1 | 0.12 | 0.12 | ≤0.008 | 0.06 | 0.06 |
| MMX 7445 | FLU-R, VORI-R | 1 | 8 | 1 | 2 | 1 | 2 | 0.12 | >64 | >8 | 0.06^{s} | 0.03 |
| MMX 7430 | Susceptible | 1 | 8 | 0.5 | 1 | 0.25 | 0.5 | 0.25 | 0.5 | ≤0.008 | 0.06 | 0.03 |
| MMX 7437 | FLU-I | 1 | 8 | 1 | 2 | 1 | 2 | 0.25 | 4 | 0.12 | 0.06 | 0.03 |
| MMX 7403 | Susceptible | 0.5 | 2 | 0.5 | 2 | 0.25 | 2 | 0.25 | 1 | ≤0.008 | 0.06 | 0.03 |

MIC₂ = 50% inhibition, MIC₀ = complete inhibition, AMP B = amphotericin B, FLU = fluconazole, VORI = voriconazole, CASP = caspofungin, MICA = micafungin, I = intermediate, R = resistant
^{a}insufficient growth at 24 hr to determine 50% inhibitory endpoint, restsit after 48 hr incubation reported
*there are no AMP B CLSI breakpoints for yeast (4)

TABLE 106
Activity of BisEDT, MB-2B, MB-6 and comparators against Candida glabrata

| | | Test agent and activity (μg/mL) | | | | | | | | | | |
|---|---|---|---|---|---|---|---|---|---|---|---|---|
| | | BisEDT | | MB-2B | | MB-6 | | AMP B | FLU | VORI | CASP | MICA |
| Isolate No. | Type | MIC₂ | MIC₀ | MIC₂ | MIC₀ | MIC₂ | MIC₀ | MIC₀ | MIC₂ | MIC₂ | MIC₂ | MIC₂ |
| ATCC 90030 | Susceptible | 0.5 | 1 | 0.5 | 1 | 0.5 | 1 | 0.25 | 8 | 0.12 | 0.12 | 0.06 |
| ATCC MYA-2950 | Susceptible | 0.5 | 1 | 0.5 | 1 | 0.5 | 1 | 0.12 | 8 | 0.12 | 0.12 | 0.03 |
| MMX 7103 | CASP-R, MICA-R | 0.5 | 1 | 0.5 | 1 | 0.5 | 1 | 0.25 | 4 | 0.12 | >8 | 8 |
| MMX 7307 | CASP-R, MICA-R, | 0.5 | 1 | 0.5 | 1 | 0.5 | 1 | 0.25 | >64 | 4 | >8 | 8 |
| | FLU-R | | | | | | | | | | | |
| MMX 7285 | CASP-R, MICA-R | 0.25 | 0.5 | 0.25 | 0.5 | 0.25 | 0.5 | 0.25 | 32 | 1 | 0.5 | 0.5 |
| MMX 7093 | FLU-R | 0.5 | 1 | 0.5 | 1 | 1 | 2 | 0.25 | 64 | 2 | 0.12 | 0.06 |
| MMX 7101 | FLU-R | 0.5 | 1 | 0.5 | 1 | 0.5 | 1 | 0.25 | 64 | 2 | 0.12 | 0.06 |
| MMX 7087 | Susceptible | 0.5 | 1 | 0.5 | 1 | 0.5 | 1 | 0.12 | 1 | 0.03 | 0.12 | 0.03 |
| MMX 7549 | Susceptible | 0.5^{a} | 0.5 | 0.5 | 1 | 0.5 | 1 | 0.25 | 4 | 0.06 | 0.06 | 0.06 |
| MMX 7111 | Susceptible | 0.5 | 1 | 0.5 | 1 | 0.5 | 1 | 0.25 | 2 | 0.06 | 0.12 | 0.06 |

MIC₂ = 50% inhibition, MIC₀ = complete inhibition, AMP B = amphotericin B, FLU = fluconazole, VORI = voriconazole, CASP = caspofungin, MICA = micafungin, I = intermediate, R = resistant
*there are no AMP B CLSI breakpoints for yeast and there are no VORI breakpoints for C. glabrata (4)

TABLE 107
Activity of BisEDT, MB-2B, MB-6 and comparators against Candida krusei

| | | Test agent and activity (μg/mL) | | | | | | | | | | |
|---|---|---|---|---|---|---|---|---|---|---|---|---|
| | | BisEBT | | MB-28 | | MB-6 | | AMP B | FLU | VORI | CASP | MICA |
| Isolate No. | Type | MIC₂ | MIC₀ | MIC₂ | MIC₀ | MIC₂ | MIC₀ | MIC₀ | MIC₂ | MIC₂ | MIC₂ | MIC₂ |
| ATCC 14243 | Susceptible | 0.5 | 1 | 1 | 2 | 0.5 | 1 | 0.25 | 16 | 0.06 | 0.012 | 0.25 |
| ATCC 6258 | MICA-I | 0.5 | 1 | 1 | 2 | 0.5 | 1 | 0.5 | 16 | 0.12 | 0.25 | 0.5 |
| MMX 7125 | Susceptible | 0.5 | 1 | 1 | 2 | 0.5 | 1 | 0.5 | 32 | 0.25 | 0.25 | 0.25 |
| MMX 7128 | Susceptible | 0.5 | 1 | 1 | 2 | 1 | 2 | 0.5 | 16 | 0.12 | 0.25 | 0.25 |
| MMX 7141 | Susceptible | 0.5 | 1 | 1 | 2 | 1 | 2 | 0.25 | 16 | 0.12 | 0.25 | 0.25 |
| MMX 7153 | MICA-R | 0.5 | 1 | 1 | 2 | 0.5 | 1 | 0.5 | 32 | 0.25 | 0.25 | 1 |
| MMX 7155 | Susceptible | 0.5 | 1 | 1 | 2 | 0.5 | 1 | 0.25 | 32 | 0.25 | 0.25 | 0.25 |
| MMX 7563 | Susceptible | 0.5 | 1 | 1 | 2 | 0.5 | 1 | 0.5 | 32 | 0.25 | 0.12 | 0.25 |
| ATCC 96685 | Susceptible | 0.25 | 0.5 | 0.25 | 0.5 | 0.25 | 0.5 | 0.5 | 32 | 0.5 | 0.12 | 0.25 |
| MMX 9878 | MICA-I, VORI-I | 0.5 | 1 | 0.5 | 1 | 0.5 | 1 | 0.5 | 64 | 1 | 0.25 | 0.5 |

MIC₂ = 50% inhibition, MIC₀ = complete inhibition, AMP B = amphotericin B, FLU = fluconazole, VORI = voriconazole, CASP = caspofungin, MICA = micafungin, I = intermediate, R = resistant
*there are no AMP B CLSI breakpoints for yeast and there are no FLU breakpoints for C. krusei (4)

TABLE 108
Activity of BisEDT, MB-2B, MB-6 and comparators against Candida auris

| | | Test agent and activity (μg/mL) | | | | | | | | | | |
|---|---|---|---|---|---|---|---|---|---|---|---|---|
| | | BisEDT | | MB-2B | | MB-6 | | AMP B | FLU | VORI | CASP | MICA |
| Isolate No. | Type | MIC₂ | MIC₀ | MIC₂ | MIC₀ | MIC₂ | MIC₀ | MIC₀ | MIC₂ | MIC₂ | MIC₂ | MIC₂ |
| MMX 9862 | Susceptible | 0.5 | 2 | 0.5 | 1 | 0.5 | 1 | 0.25 | 2 | 0.03 | 0.12 | 0.12 |
| MMX 9863 | FLU-R, VORI-R^{c} | 1^{a} | 1 | 1^{a} | 1 | 1 | 2 | 0.12 | >64 | >8 | 0.12^{b} | 0.5 |
| MMX 9864 | FLU-R, VORI-R | 0.5 | 1 | 1^{a} | 1 | 1^{a} | 1 | 0.25 | >64 | 7 | 0.25^{b} | 0.5 |
| MMX 9865 | FLU-R, VORI-R | 1^{a} | 1 | 1^{a} | 1 | 1 | 2 | 0.25 | >64 | 8 | 0.25^{b} | 0.25 |
| MMX 9866 | FLU-R, VORI-R | 1 | 2 | 1^{a} | 1 | 1 | 2 | 0.5 | >64 | 8 | 0.12^{b} | 0.5 |
| MMX 9867 | FLU-R, VORI-R | 1 | 2 | 1 | 2 | 1 | 2 | 0.5 | >64 | 8 | 0.12 | 0.5 |
| MMX 9868 | FLU-R, VORI-R | 1^{b} | 1^{a} | ND | 1 | 1 | 4 | 0.25 | >64 | >8 | 0.25 | 0.25 |
| MMX 9869 | FLU-R, VORI-R | 1^{b} | 2 | 1 | 2 | 1 | 4 | 1 | >64 | 2 | 0.25 | 0.25 |
| MMX 9870 | FLU-R, VORI-R | 1 | 2 | 1 | 2 | 1 | 2 | 1 | >64 | 4 | 0.25 | 0.5 |
| MMX 9871 | Susceptible | 1^{a} | 1 | 1^{a} | 1 | 1 | 2 | 1 | >64 | 1 | 0.25^{b} | 0.5 |

MIC₂ = 50% inhibition, MIC₀ = complete inhibition, AMP B = amphotericin B, FLU = fluconazole, VORI = voriconazole, CASP = caspofungin, MICA = micafungin, Azole-R = azole-resistant, ND = not determined (no MIC₂ was apparent)
^{a}insufficient growth at 24 hr to determine 50% inhibitory endpoint, result after 48 hr incubation reported
^{b}eagle effect observed (clear MIC₂ endpoint but regrowth at higher test concentrations potentially due to inducible resistance or compound precipitation in vitro)
^{c} based on breakpoints published by Lockhart et al. 2017 (6); CLSE breakpoints have not been established for C. auris

TABLE 109
Activity of BisEDT, MB-2B, MB-6 and comparators against Aspergillus spp.

| | | Test agent and activity (μg/mL) | | | | | | | |
|---|---|---|---|---|---|---|---|---|---|
| | | BisEDT | MB-2B | MB-6 | | | | | |
| | | MIC₀ | MIC₀ | MIC₀ | AMP B | FLU | VORI | CASP | MICA |
| Organism | Isolate No. | 24/48 hr | 24/48 hr | 24/48 hr | MIC₀ | MIC₂ | MIC₀ | MEC | MEC |
| A. fumigatus | ATCC MYA-3626 | 4/8 | 4/8 | 2/4 | 1 | >64 | 0.5 | 0.06 | 0.03 |
| | ATCC 204305 | 8/8^{a} | 4/8^{a} | 8/4^{a} | 1 | >64 | 0.5 | 0.25 | 0.25 |
| | MMX 5934 | 4/8^{a} | 8/8^{a} | 2/4^{a} | 1 | >64 | 0.25 | 0.12 | 0.06 |
| | MMX 5938 | —/2^{b} | —/2^{b} | —/1^{b} | 1 | >64 | 0.25 | 0.12^{b} | ≤0.008^{b} |
| | MMX 5939 | 4/8^{a} | 4/8^{a} | 2/4^{b} | 1 | >64 | 7 | 0.12 | 0.06 |
| A. flavus | ATCC 204304 | 4/8^{a} | 4/4^{a} | 4/4^{a} | 1 | >64 | 2 | 0.06 | 0.12 |
| | ATCC 22546 | 2/4 | 1/2 | 1/2 | 1 | >64 | 1 | 0.06 | ≤0.008 |
| | MMX 7935 | >64/8^{a} | 32/4^{a} | 8/8 | 1 | >64 | 0.5 | 0.03 | ≤0.008 |
| A. niger | ATCC 29508 | —/2^{b} | —/2^{b} | —/2^{b} | 0.25 | >64 | 0.5 | 0.06^{b} | ≤0.008^{b} |
| A. terreus | MMX 8229 | 0.5/1 | 0.5/1 | 0.25/1 | 0.25 | >64 | 0.25 | 0.12^{b} | ≤0.008^{b} |

MIC₀ = complete inhibition, MEC = minimum effective concentration, AMP B = amphotericin B, FLU = fluconazole, VORI = voriconazole, CASP = caspofungin, MICA = micafungin
^{a}result shown at 48 hr is the MEC, complete inhibition (MIC₀) not observed at this timepoint
^{b}result reported for 48 hr only, insufficient growth for 24 hr read
^{c}there are no CLSI breakpoints for Aspergillus spp. (5)

TABLE 110
Activity of Bis-EDT, MB-2B, MB-6 and comparators
against relevant ATCC QC organisms

| | | QC | QC | | |
|---|---|---|---|---|---|
| | | Range | Range | MIC | MIC |
| Organism | Test Agent | 24 hr | 48 hr | 24 hr | 48 hr |
| E. coli | BisEDT | 0.5-4 | — | 0.5^{a} | — |
| ATCC 25922 | MB-2B | — | — | 2^{a} | — |
| | MB-6 | — | — | 0.5^{a} | — |
| | Amphotericin B | — | — | >64 | — |
| | Fluconazole | — | — | >64 | — |
| | Voriconazole | — | — | >64 | — |
| | Caspofungin | — | — | >64 | — |
| | Micafungin | — | — | >64 | — |
| S. aureus | BisEDT | 0.12-1 | — | 0.5^{a} | — |
| ATCC 29213 | MB-2B | — | — | 1^{a} | — |
| | MB-6 | — | — | 1^{a} | — |
| | Amphotericin B | — | — | >64 | — |
| | Fluconazole | — | — | >64 | — |
| | Voriconazole | — | — | >64 | — |
| | Caspofungin | — | — | >64 | — |
| | Micafungin | — | — | >64 | — |
| C. parapsilosis | BisEDT | — | — | 0.5/1^{b} | 1/2^{b} |
| ATCC 22019 | MB-2B | — | — | 1/2^{b} | 1/2^{b} |
| | MB-6 | — | — | 1/2^{b} | 1/4^{b} |
| | Amphotericin B | 0.25-2 | 0.5-4 | 0.5 | 0.5 |
| | Fluconazole | 0.5-4 | 1-4 | 1 | 2 |
| | Voriconazole | 0.015-0.12 | 0.03-0.25 | 0.12 | 0.12 |
| | Caspofungin | 0.25-1 | 0.5-4 | 0.25 | 0.5 |
| | Micafungin | 0.5-2 | 0.5-4 | 0.5 | 2 |
| C. krusei | BisEDT | — | — | 0.5/1^{b} | 1/2^{b} |
| ATCC 6258 | MB-2B | — | — | 1/2^{b} | 1/4^{b} |
| | MB-6 | — | — | 0.5/1^{b} | 1/4^{b} |
| | Amphotericin B | 0.5-2 | 1-4 | 0.5 | 0.5 |
| | Fluconazole | 8-64 | 16-128 | 16 | 32 |
| | Voriconazole | 0.06-0.5 | 0.12-1 | 0.12 | 0.5 |
| | Caspofungin | 0.12-1 | 0.25-2 | 0.25 | 0.25 |
| | Micafungin | 0.12-0.5 | 0.12-0.5 | 0.5 | 0.5 |
| | BisEDT | — | — | 4 | 8^{c} |
| | MB-2B | — | — | 4 | 8^{c} |
| A. flavus | MB-6 | — | — | 4 | 4^{c} |
| ATCC 204304 | Amphotericin B | — | 0.5-4 | 0.5 | 1 |
| | Fluconazole | — | — | >64 | >64 |
| | Voriconazole | — | 0.5-4 | 0.12 | 2 |
| | Caspofungin | — | — | 0.12 (MEC) | 0.06 (MEC) |
| | Micafungin | — | — | 0.06 (MEC) | 0.008 (MEC) |
| A. fumigatus | BisEDT | — | — | 4 | 8 |
| ATCC | MB-2B | — | — | 4 | 8 |
| MYA-3626 | MB-6 | — | — | 2 | 4 |
| | Amphotericin B | — | 0.5-4 | 0.5 | 1 |
| | Fluconazole | — | — | >64 | >64 |
| | Voriconazole | — | 0.25-1 | 0.25 | 0.5 |
| | Caspofungin | — | — | 0.06 (MEC) | 0.06 (MEC) |
| | Micafungin | — | — | 0.03 (MEC) | 0.008 (MEC) |

### REFERENCES

1.) Beckman E, Wolfe C, Pillar C. In vitro Activity of Bismuth Thiols and Comparators Against Drug Resistant Gram-positive and -negative Bacteria and Yeast. Final Report Aug. 24, 2016-Microbion 22. Micromyx, Kalamazoo, Mich. 2016.
2.) Clinical and Laboratory Standards Institute (CLSI). Reference Method for Broth Dilution Antifungal Susceptibility Testing of Yeasts. 4^{th} ed. CLSI standard M27. CLSI, 940 West Valley Road, Suite 1400, Wayne, Pa. 19087-1898 USA, 2017.
3.) CLSI. Reference Method for Broth Dilution Antifungal Susceptibility Testing of Filamentous Fungi; Approved Standard. 3^{rd} ed. CLSI standard M38. CLSI, 940 West Valley Road, Suite 1400, Wayne, Pa. 19087-1898 USA, 2017.
4.) CLSI. Performance Standards for Antifungal Susceptibility Testing of Yeasts. 1^{st} ed. CLSI supplement M60. CLSI, 940 West Valley Road, Suite 1400, Wayne, Pa. 19087-1898 USA, 2017.
5.) CLSI. Performance Standards for Antifungal Susceptibility Testing of Filamentous Fungi; Approved Standard. 1^{st} ed. CLSI supplement M61. CLSI, 940 West Valley Road, Suite 1400, Wayne, Pa. 19087-1898 USA, 2017.
6.) Lockhart S R, Etienne K A, Vallabhaneni S, Farooqi J, Chowdhary A, Govender N, et al. Simultaneous Emergence of Multidrug-Resistant Candida auris on 3 Continents Confirmed by Whole-Genome Sequencing and Epidemiological Analyses. Clin Infect Dis 2017; 64:134-140.

### Example 14: Studies on Processing Conditions on BisEDT Particle Size Distribution

It was observed that careful control of the reaction temperature and the rate of 1,2 ethanedithiol addition had pronounced impact on the BisEDT particle size distribution. Representative syntheses are shown below for BisEDT synthesized at 20° C. with a 1.25 hour addition of 1,2-ethane via syringe pump and BisEDt synthesized at 15° C. with a 1 hour addition of 1,2-ethane via syringe pump. Table 111 below shows that temperature conditions play a critical role in particle size distribution, with processing temperatures in the range of 20-30° C. providing BisEDT particle size distribution that are both small and uniform in particle size (such as a D90 below 2 microns).

Representative synthesis of BisEDT at 20° C. with 1.25 hour addition of thiol via syringe pump, and polypropylene cloth for filtration BisEDT synthesis was performed on 10-g scale. To a 1-L jacketed reactor was charged USP water (480 mL, 48 vol), followed by 70% HNO3 (34 mL, 3.4 vol). A solution of bismuth subnitrate (10 g, 6.84 mmols) in water (43 mL, 4.3 vol) and 70% HNO3 (14 mL, 1.4 vol) was added at 20° C. The reaction mixture was cooled to 15° C. for addition of 95% Ethanol. The 95% ethanol (180 mL, 18 vol) was then added slowly. (Ethanol addition is exothermic, temperature reached 22° C.). The temperature was then adjusted back to 20° C. This was followed by dropwise addition of 1,2 ethanedithiol (4.3 mL, 7.5 mmols in 95% ethanol in 94 mL, 9.4 vol) over a period of 1.25 hour with the batch temperature at 20° C. during which time it turned into a yellow suspension. The reaction was stirred at 20° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.81 g 91.0%).

Representative synthesis of BisEDT at 15° C. with 1 hour addition of thiol via syringe pump, and polypropylene cloth for filtration: The synthesis BisEDT was performed on 10-g scale, temperature profile was studied with data logger. Ethane dithiol was added at 15° C. over 1 hour via syringe pump and the filtration was performed using PP filter cloth. To a 1-L jacketed reactor was charged USP water (480 mL, 48 vol) and cooled to 15° C., followed by 70% HNO3 (34 mL, 3.4 vol). A solution of bismuth subnitrate (10 g, 6.84 mmols) in water (43 mL, 4.3 vol) and 70% HNO3 (14 mL, 1.4 vol) was added at the same temperature. The 95% ethanol (180 mL, 18 vol) was then added slowly. (Ethanol addition is exothermic, temperature reached 22.5° C.). It was then allowed to cool to 15° C. This was followed by dropwise addition of 1,2 ethanedithiol (4.3 mL, 7.5 mmols in 95% ethanol in 94 mL, 9.4 vol) over an hour with the batch temperature at 15° C. The reaction was allowed to stir at 15° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.43 g 87.8%).

TABLE 111
Particle Size Distribution of BisEDT samples

| Sample | D(10) μm | D(50) μm | D(90) μm | D[4, 3] μm | Conditions |
|---|---|---|---|---|---|
| 1 | 0.80 | 2.4 | 5.9 | 2.9 | Dalton Synthesis Conditions |
| 2 | 0.58 | 1.7 | 3.9 | 2.0 | Dalton Synthesis Conditions |
| 3 | 0.59 | 1.9 | 4.5 | 2.3 | 30° C., 5 h addition of 1,2-ethane dithiol |
| | | | | | via addition funnel |
| 4 | 0.44 | 1.48 | 3.1 | 1.7 | 30° C., 4 hour addition of 1,2-ethane |
| | | | | | dithiol via syringe pump |
| 5 | 0.33 | 0.72 | 1.6 | 0.86 | 20° C., 1 h addition of 1,2-ethane dithiol |
| | | | | | via addition funnel |
| 6 | 0.34 | 0.87 | 1.8 | 0.98 | 20° C., 4 h addition of 1,2-ethane dithiol |
| | | | | | via addition funnel |
| 7 | 0.39 | 1.3 | 1.6 | 1.4 | 20° C., 1 hour addition of 1,2-ethane |
| | | | | | dithiol via syringe pump. Sample |
| | | | | | slurried in EtOH. Cloth filtration |
| 8 | 0.36 | 1.0 | 1.8 | 1.0 | 20° C., 1 hour addition of 1,2-ethane |
| | | | | | dithiol via syringe pump. Sample |
| | | | | | slurried in MeOH. Cloth filtration |
| 9 | 0.67 | 1.0 | 1.9 | 1.1 | 20° C., 1 hour addition of 1,2-ethane |
| | | | | | dithiol via syringe pump. Sample |
| | | | | | slurried in Abs, MeOH. Cloth filtration |
| 10 | 0.36 | 0.88 | 1.7 | 0.97 | 20° C., 1 hour addition of 1,2-ethane |
| | | | | | dithiol via syringe pump. Sample |
| | | | | | slurried in IPA. Cloth filtration |
| 11 | 0.38 | 1.2 | 2.4 | 1.4 | 15° C. 1.5 hour addition of 1,2-ethane |
| | | | | | dithiol via syringe pump. Cloth filtration |
| 12 | 0.37 | 1.2 | 2.4 | 1.3 | 20° C., 1.25 hour addition of 1,2-ethane |
| | | | | | via syringe pump |
| 13 | 0.36 | 0.98 | 2.1 | 1.2 | 10° C., 1 h addition of 1,2-ethane dithiol |
| | | | | | via syringe pump |
| 14 | 0.36 | 1.0 | 2.1 | 1.2 | 10° C. 1 hour addition of 1,2-ethane dithiol |
| | | | | | via syringe pump. Cloth filtration |
| 15 | 0.32 | 0.72 | 1.6 | 0.86 | 10° C., 4 hours addition of 1,2-ethane |
| | | | | | dithiol via syringe pump. Cloth filtration. |

### Example 15: Use of BisEDT to Treat Respiratory Viral Infections and Secondary Infections

There is currently an urgent, ongoing, substantial unmet need for broad-spectrum antibacterial and antifungal drugs for the treatment of pulmonary infections. These infections can occur as primary infections, or as infections secondary to viral pulmonary infections which are known to occur in relatively frequent outbreaks, and devastating periodic pandemics. Such secondary bacterial and fungal infections have been responsible for up to 30% of deaths, and in some cases for the vast majority of deaths, in past viral pandemics. Because BisEDT has extremely broad-spectrum activity, it is believed to reduce morbidity and mortality in secondary bacterial and fungal infections associated with viral pandemics by overcoming deadly secondary infections (superinfections) caused by bacterial and/or fungal pathogens.

This Example examines secondary bacterial pulmonary infections, or superinfections, that opportunistically complicate primary respiratory viral infections in viral pandemics, including COVID-19, and highlights the promising potential of BisEDT to reduce associated mortality and morbidity. The species of bacteria causing secondary infections may vary, depending on the regional microbial differences, and on the species of virus that is causing the primary viral infection. Due to difficulty in testing for presence of specific bacterial species, empiric use of broad-spectrum antibiotics is critical.

Treatment of patients affected by respiratory viral infections in pandemics, such as the COVID-19 pandemic, may derive substantial benefit from the targeted, inhaled administration of BisEDT.^{19,20} BisEDT has been demonstrated in clinical and non-clinical studies, to provide a strategic constellation of beneficial attributes.

BisEDT has demonstrated an excellent safety profile in human clinical studies in moderate-to-severe diabetic foot ulcer infections and orthopedic infections. In addition, preclinical non-GLP toxicology studies with inhaled BisEDT drug product in two animal species provide strong feasibility support for easily achieving therapeutic dose levels for treatment of respiratory infections.

BisEDT is an extremely broad-spectrum anti-infective drug, with potent activity against the key MDR bacterial and fungal pulmonary pathogens that complicate viral pulmonary infections, and are an important cause of death. BisEDT has been demonstrated effective in both in vitro and in vivo studies, overcoming highly resistant bacterial and fungal pathogens. BisEDT has demonstrated excellent efficacy in an animal model of lung infection caused by Pseudomonas aeruginosa. BisEDT is believed to have species-specific antiviral activity.

BisEDT is the first drug candidate from a new class of anti-infective drugs (bismanes); its mechanism of action is unique. In vitro studies conducted to date have failed to demonstrate even a single example of cross-resistance to clinically utilized antibiotics by bacteria or fungi resistant to those clinically utilized antibiotics.

Furthermore, BisEDT has an extremely favorable spontaneous mutation frequency for a broad-spectrum of bacteria (10¹⁰). It has been demonstrated experimentally that bacteria subjected to extended exposure to BisEDT are not able to accumulate mutations that could conceivably be expected to result in stable resistance to BisEDT. BisEDT's resistance profile is equal to, or superior, to that of vancomycin. In short, BisEDT will likely be effective for many years because it is extremely difficult, if not impossible, for bacteria to develop resistance to it.

Broad-spectrum Activity of BisEDT Against Highly Resistant Bacterial and Fungal Pathogens: The key bacterial and fungal pathogens demonstrated to be involved in coronavirus secondary infections comprise at least the following bacterial and fungal species. Bacterial: Acinetobacter baumannii, Klebsiella pneumoniae, Pseudomonas aeruginosa, Staphylococcus aureus and other Staphylococcal spp., Stenotrophomonas maltophilia, Haemophilus influenzae, and Escherichia coli. Fungal: Aspergillus flavus, Candida glabrata, and Candida albicans. Data is provided that are relevant to the antimicrobial potency of BisEDT against the above described bacterial and fungal pathogens that complicate viral pandemic infections, including multidrug-resistant (MDR) strains of these pathogens. Susceptibility to BisEDT and relevant comparators was determined by broth microdilution conducted in accordance with guidelines from the Clinical and Laboratory Standards Institute (CLSI) and is expressed as minimum inhibitory concentration (MIC).

Worthy of special recognition is the activity of BisEDT against carbapenem-resistant bacterial pathogens. The former Director of the CDC, Dr. Tom Frieden, referred to carbapenem-resistant bacterial pathogens as the “nightmare bacteria”. Infections by carbapenem-resistant bacteria such as Acinetobacter baumannii are associated with up to a 60% rate of mortality.²¹⁻²³ “The use of carbapenems has led to the surge of carbapenem-resistant Enterobacteriaceae (CRE), which represents a threat to global public health.”^{22,24,25} “Recent data suggests the prevalence of CRE is increasing across the world.^{22,26,27}” Such carbapenem-resistant strains of these coronavirus infection-relevant bacterial pathogens are frequently found in hospital settings in which nosocomial (hospital-caused) transmission of viral, bacterial and fungal pathogens is common. Nosocomial or community-acquired infection is caused by these highly antibiotic-resistant bacteria are far more deadly than their antibiotic-susceptible counterparts. Nosocomial pathogen transmission is a significant concern relevant to all microbial pathogens. Even with respect to SARS-CoV-2, nosocomial transmission is responsible for over 3000 cases of COVID-19 in health care workers in China, representing a very substantial risk to patients and to health care workers worldwide.²⁸

Activity of BisEDT Against Gram-Negative Species, Including Antibiotic-Resistant Strains: The data in Table 112 and Table 113 demonstrates the potential for BisEDT to treat this problematic, influenza- and coronavirus-relevant group of Gram-negative bacterial pathogens, including carbapenem-resistant strains of Acinetobacter baumannii, Klebsiella pneumonia, Pseudomonas aeruginosa, and E. coli. Remarkably, BisEDT is more potent in almost every strain than any of the comparator agents: levofloxacin, ciprofloxacin, meropenem, ceftazidime, and gentamicin. BisEDT has demonstrated potent activity against the Gram-negative pathogen Haemophilus influenzae, including Beta lactamase-negative ampicillin-resistant isolates, as shown in Table. BisEDT was also active against Stenotrophomonas maltophilia with an MIC of 0.25 μg/mL in comparison to amikacin which had an MIC of 64 μg/mL.

TABLE 112
MIC values for BisEDT and comparators against MDR P. aeruginosa and MDR A. baumannii

| Isolate | | | | | | | |
|---|---|---|---|---|---|---|---|
| (MMX strain ID #) | Type | BisEDT | LVX | CIP | MEM | CAZ | GEN |
| P. aeruginosa (103) | QC | 1 | 1 | 0.5 | 8.5 | 2 | 1 |
| | | | (0.5-4)^{a} | (0.25-1) | (0.25-1) | (1.4) | (0.5-2) |
| P. aeruginosa (4697) | VIM-2; LVX^{R}, CIP^{R}, MEM^{R}, CAZ^{R} | 1 | 32 | 32 | 8 | 32 | 4 |
| P. aeruginosa (4654) | IMP-7; LVX^{R}, CIP^{R}, MEM^{R}, CAZ^{R} | 2 | 64 | 32 | >64 | >32 | >64 |
| P. aeruginosa (2562) | MDR; LVX^{R}, CIP^{R}, MEM^{R}, CAZ^{R} | 1 | 64 | 64 | 32 | >32 | 8 |
| P. aeruginosa (1381) | MDR; LVX^{R}, CIP^{R}, MEM^{R}, CAZ^{R} | 1 | 64 | 32 | 16 | 32 | 8 |
| P. aeruginosa (3991) | MDR; LVX^{R}, CIP^{R}, MEM^{R}, CAZ^{R} | 1 | 64 | 32 | 16 | 16 | 4 |
| A. baumonnii (4651) | MDR; OXA-27, LVX^{R}, CIP^{R}, MEM^{R}, | 0.5 | 8 | 32 | 64 | >32 | 8 |
| | CAZ^{R}, GEN^{I} | | | | | | |
| A. baumonnii (2592) | MDR; LVX^{R}, CIP^{R}, MEM^{R}, CAZ^{R}, GEN^{R} | 0.5 | 64 | >64 | 64 | 32 | >64 |
| A. baumonnii (2593) | MDR; LVX^{R}, CIP^{R}, MEM^{R}, CAZ^{R}, GEN^{R} | 5 | 32 | >64 | 64 | 32 | >64 |
| A. baumannii (3372) | MDR; CIP^{R}, MEM^{I}, CAZ^{I} | 1 | 1 | 4 | 4 | 16 | 0.5 |
| A. baumonnii (3373) | Sensitive | 1 | 0.12 | 0.5 | 0.5 | 4 | 0.12 |

MMX = Micromyx; QC = quality control: VIM/IMP = metallo-beta lactamase type; OXA = type D extended-spectrum beta-lactamase; MDR = multi-drug resistant (based on resistance to at least 3 different classes of antibiotic); LVX = levofloxacin; CIP = ciprofloxacin; MEM = meropenem; CAZ = ceftazidime; GEN = gentamicin; ^{I} = intermediate resistance; ^{R} = resistant.
^{a}Clinical and Laboratory Standards Institute (CLSI) Quality Control Range

TABLE 113
MIC values for BisEDT and comparator agents against antibiotic-resistant (including carbapenem-resistant) Enterobacteriaceae spp.

| Isolate | | MIC (μg/mL) | | | | | |
|---|---|---|---|---|---|---|---|
| (MMX strain ID#) | Type | BisEDT | LVX | CIP | MEM | CAZ | GEN |
| E. coli (102) | QC | 0.5 | 0.015 | 0.008 | 0.03 | 0.12 | 1 |
| | | | (0.008-0.06)^{a} | (0.004-0.015) | (0.008-0.06) | (0.06-0.5) | (0.25-1) |
| E. coli (8423) | ESBL; LVX^{R}, CAZ^{R}, GEN^{R} | 1 | 16 | 32 | 0.03 | 32 | 64 |
| E. coli (8424) | ESBL; CAZ^{R} | 0.5 | 0.06 | 0.015 | 0.03 | 16 | 0.25 |
| E. coli (8425) | ESBL; LVX^{R}, CAZ^{R}, GEN^{R} | 1 | 16 | >64 | 0.015 | 16 | >64 |
| E. coli (5980) | NDM-1; LVX^{R}, MEM^{R}, CAZ^{R}, GEN^{R} | 1 | 16 | >64 | 32 | >32 | >64 |
| K. pneumoniae (4683) | KPC-2; LVX^{R}, MEM^{R}, CAZ^{R} | 1 | >64 | >64 | 32 | >32 | 1 |
| K. pneumoniae (4622) | KPC-2; MEM^{R}, CAZ^{R} | 2 | 1 | 0.03 | >64 | >32 | 0.25 |
| K. pneumoniae (4623) | KPC-2; MEM^{R}, CAZ^{R}, GEN^{R} | 2 | 1 | 2 | >64 | >32 | 64 |
| K. pneumoniae (4694) | KPC-3; LVX^{R}, MEM^{R}, CAZ^{R} | 2 | 64 | >64 | 32 | >32 | 8 |
| K. pneumoniae (4653) | KPC-3; LVX^{R}, MEM^{R}, CAZ^{R} | 4 | 64 | >64 | >64 | >32 | 1 |
| K. pneumoniae (4684) | ESBL; MEM^{R} | 4 | 0.03 | 0.5 | 8 | 8 | 0.25 |
| K. pneumoniae (4685) | ESBL; LVX^{R}, MEM^{R}, CAZ^{R} | 2 | 32 | 64 | 4 | >32 | 1 |
| K. pneumoniae (5979) | NDM-1; LVX^{R}, MEM^{R}, CAZ^{R}, GEN^{R} | 2 | >64 | >64 | >64 | >32 | >64 |
| E. cloacae (5981) | NDM-1; LVX^{R}, MEM^{R}, CAZ^{R}, GEN^{R} | 4 | 64 | >64 | >64 | >32 | >64 |

MMX = Micromyx; QC = quality control; ESBL = extended-spectrum beta-lactamase; KPC = K. pneumoniae; NDM = New Delhi Metallo-beta-lactamase; LVX= levofloxacin; MEM = meropenem; CAZ = ceftazidime; GEN = gentamicin; ^{R} = resistant.
^{a}Clinical and Laboratory Standards Institute (CLSI) Quality Control Range

TABLE 114
MIC values for BisEDT and comparators against isolates of H. influenzae

| Organism | MMX No. | Type | BisEDT | AZM | AMP | CFA | LEV |
|---|---|---|---|---|---|---|---|
| H. influenzae | 1224 | — | ≤0.06 | 2 (1-4)^{a} | 2 (2-8) | 4 | 0.015 |
| | (ATCC 49247) | | | | | | (0.008-0.03) |
| H. influenzae | 1302 | — | ≤0.06 | 2 | 0.25 | 2 | 0.015 |
| H. influenzae | 1304 | — | ≤0.06 | 2 | 0.25 | 64 | 0.015 |
| H. influenzae | 1740 | — | ≤0.06 | 4 | 4 | 8 | 0.015 |
| H. influenzae | 1741 | — | ≤0.06 | 1 | 4 | 4 | 0.015 |
| H. influenzae | 5458 | — | ≤0.06 | 1 | 0.12 | 16 | 0.015 |
| H. influenzae | 5461 | — | ≤0.06 | 2 | 0.5 | 8 | 0.03 |
| H. influenzae | 5469 | — | ≤0.06 | 2 | 4 | 4 | 0.015 |
| H. influenzae | 5494 | — | ≤0.06 | 1 | 0.12 | 0.5 | 0.015 |
| H. influenzae | 5495 | — | ≤0.06 | 0.5 | 0.25 | 2 | 0.015 |
| H. influenzae | 5499 | — | ≤0.06 | 2 | 0.25 | 1 | 0.015 |
| H. influenzae | 2795 | BLNAR | ≤0.06 | 2 | 4 | 1 | 0.015 |
| H. influenzae | 2796 | BLNAR | ≤0.06 | 2 | 2 | 4 | 0.015 |
| H. influenzae | 0797 | BLNAR | ≤0.06 | 2 | 4 | 4 | 0.015 |
| H. influenzae | 2798 | BLNAR | ≤0.06 | 0.5 | 4 | 4 | 0.015 |
| H. influenzae | 2799 | BLNAR | ≤0.06 | 0.5 | 4 | 8 | 0.015 |

MMX = Micromyx; ATCC = American Type Culture Collection; AZM = Azithromycin; AMP = Ampicillin; CFA = Cefuroxime; LEV = Levofloxacin; BLNAR = Beta lactamase-negative ampicillin-resistant.
^{a}Clinical and Laboratory Standards Institute (CLSI) Quality Control Range

Additional studies were conducted to assess the antimicrobial activity of BisEDT and comparators against isolates of Gram-negative pathogens including K. pneumoniae (Table 98), P. aeruginosa (Table 99), A. baumannii (Table 100) and E. coli (Table 97) characterized for extended-spectrum β-lactamases (ESBL) and/or carbapenem resistance. BisEDT maintained potent activity with MBC values of 0.5-2 μg/mL across isolates with the exception of one isolate of P. aeruginosa (CDC 241) which had an MIC of 4 μg/mL (Table 99) and several isolates of K. pneumoniae with MIC values of 4-8 μg/mL (Table 98). The activity of BisEDT was not impacted by β-lactamase production or resistance to aminoglycosides (amikacin MIC ≥64 μg/mL), or fluoroquinolones (levofloxacin).

Activity of BisEDT Against Gram-Positive Species, Including Antibiotic-Resistant Strains: BisEDT has also demonstrated potent activity against Gram-positive bacteria that are important pathogens involved in secondary bacterial lung infections associated with viral respiratory infections including S. aureus, MRSA, and CA-MRSA (Table 115), antibiotic-resistant S. aureus (Table 91) and Streptococcus pneumoniae (Table 92). BisEDT achieved MIC₉₀ values of 0.12-2.0 μg/mL and was 4 to 8-fold more active than linezolid against MRSA and CA-MRSA. Overall, BisEDT was more active than linezolid and the other relevant marketed antibiotics, against most Gram-positive bacteria tested.

TABLE 115
MIC⁹⁰ values for BisEDT and comparator agents against 150 clinical isolates
of Gram-positive aerobic bacteria

| Organisms | | MIC₉₀ (μg/mL) | | | | | | | |
|---|---|---|---|---|---|---|---|---|---|
| (# of isolates) | Type | BisEDT | LZD | DAP | VAN | CAZ | IPM | CIP | GEN |
| S. aureus (50) | MSSA | 0.5 | 4 | 0.5 | 1 | 16 | 0.03 | 2 | 1 |
| S. aureus (50) | MRSA | 0.5 | 4 | 0.5 | 2 | >64 | >8 | >32 | 2 |
| S. aureus (50) | CA-MRSA | 0.5 | 4 | 0.5 | 1 | >64 | 2 | >32 | 1 |

MSSA = methicillin-susceptible Staphylococcus aureus; MRSA = methicillin-resistant Staphylococcus aureus; CA-MRSA = community-acquired methicillin-resistant Staphylococcus aureus;; LZD = linezolid; DAP = daptomycin; VAN = vancomycin; CAZ = ceftazidime; IPM = imipenem; CIP = ciprofloxacin; GEN = gentamicin

Of note, as shown in Table 101, BisEDT had potent MIC values of ≤0.06-0.25 μg/mL against vancomycin-intermediate S. aureus (VISA) (VRSA is not readily available for testing). Of note, two of the VISA isolates (NRS 13 and 27) had vancomycin MIC values of 2 μg/mL, which indicated that during testing in this study they tested as vancomycin-susceptible. The other two isolates with vancomycin MIC values in the susceptible range (NRS 2 and 24) are heterogenous VISA (hVISA) for which vancomycin MIC values are known to vary. Resistance to levofloxacin and clindamycin (MIC values ≥4 μg/mL) was observed with all isolates except NRS 13 and did not impact BisEDT activity. Two of the isolates were also non-susceptible to daptomycin (NRS 13 and 22); all were susceptible to linezolid (MIC values ≤4 μg/mL).

Antifungal Activity of BisEDT: The potent in vitro antifungal activity of BisEDT was determined for antifungal-resistant and susceptible Aspergillus spp. (Table 109) and Candida spp. (Tables 95 and 105-108). Comparators included amphotericin B, fluconazole, voriconazole, caspofungin and micafungin. Notable among these fungal pathogens are highly resistant strains of Candida auris which have recently become notorious, highly resistant or pan-resistant pathogens in clinical settings, against which BisEDT demonstrated potent activity. Susceptibility was determined by broth microdilution conducted in accordance with guidelines from the Clinical and Laboratory Standards Institute. An additional BisEDT anti-fungal efficacy was generated in an NIH/NIAID sponsored study conducted at The University of Texas Health Science Center at San Antonio. Data from that study is presented in Table 116.

TABLE 116
Antifungal activity of BisEDT in an NIH/NIAID sponsored study

| | | BisEQT | | Fluconazole | Voriconazole | Posaconazole |
|---|---|---|---|---|---|---|
| | | (μg/mL) | | (μg/mL) | (μg/mL) | (μg/mL) |
| Species | Isolate No. | 50% | 100% | 50% | 100% | 100% |
| C. parapsilosis | ATCC22019 | 1 | 4 | 1 | — | — |
| C. krusei | ATCC6258 | 1 | 4 | 16 | — | — |
| P. variotii | CLSI QC | 4 | 16 | — | 0.125 | ≤0.03 |
| C. albicans | CA1 | 4 | 8 | ≤0.125 | — | — |
| | CA2 | 2 | 8 | ≤0.125 | — | — |
| | CA3 | 4 | 16 | >64 | — | — |
| C. auris | Cau1 | 4 | 16 | >64 | — | — |
| | Cau2 | 2 | 8 | 2 | — | — |
| | Cau3 | 4 | 8 | >64 | — | — |
| C. neoformans | CN1 | 2 | 8 | 64 | — | — |
| | CN2 | 4 | 8 | 64 | — | — |
| | CN3 | 4 | 16 | 2 | — | — |
| A. fumigatus | AF1 | 8 | 16 | — | 0.5 | — |
| | AF2 | 4 | 16 | — | 4 | — |
| | AF3 | 4 | 16 | — | 8 | — |
| R. arrhizus | RA1 | 8 | 16 | — | — | 4 |
| | RA2 | 4 | 8 | — | — | 0.06 |
| | RA3 | 4 | 8 | — | — | 0.125 |

| | | BisEDT | | Voriconazole |
|---|---|---|---|---|
| | | (μg/mL) | | (μg/mL) |
| Species | Isolate No. | 50% | 100% | 100% |
| B. dermatitidis | BD1 | 0.5 | 1 | 0.125 |
| | BD2 | 1 | 1 | ≤0.03 |
| | BD3 | 0.5 | 1 | 0.06 |

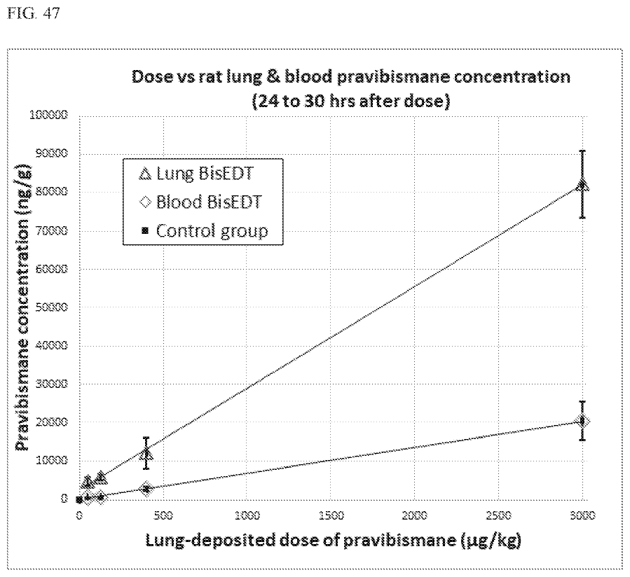

Formulation, Toxicology and PK: A formulation of BisEDT that is suitable for inhalation by nebulization has been developed and characterized with respect to aerodynamic properties and in vivo deposition in rats and dogs. Using common, commercially available jet nebulizers (Pari LC SPRINT® and Pari LC PLUS®), the formulation has provided reproducible aerosolization and lung deposition of BisEDT with an MMAD below 4 μm across a 100-fold concentration range (FIG. 2; Table 1). For reference, aerosols with MMAD ≤5 μm are considered to be respirable, which means they will deposit in the lungs instead of the upper airways and oropharyngeal region. Impressively, the in vitro data translated into in vivo lung deposition in rats that was nearly linear as measured 24 to 30 hours after a single inhalation dose in four dose groups (FIG. 47).

Based on rat tox and PK data to date, an acute no observed adverse effect level (NOAEL) in rats is approximately 100 μg/kg lung-deposited dose. The NOAEL was established through clinical observation during and after treatment, gross necropsy, lung lavage analysis, clinical chemistry, and hematology results. Encouragingly, data in rats show that 24 hours after dosing, lung tissue drug levels are 4- to 5-fold higher than blood levels (FIG. 47) and PK analysis from repeat dose TK show lung tissue exposure (AUC) is 26-fold greater than blood exposure. Furthermore, lung surface fluid concentrations are approximately 30-fold higher than the corresponding total lung tissue concentrations for BisEDT which appears to persist primarily on the lumen. This implies lung fluid exposure (AUC) over 700-fold greater than the systemic exposure.

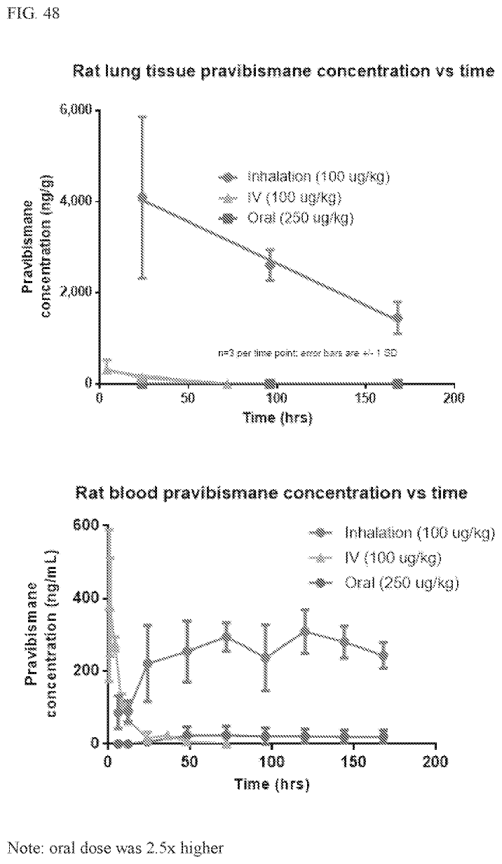

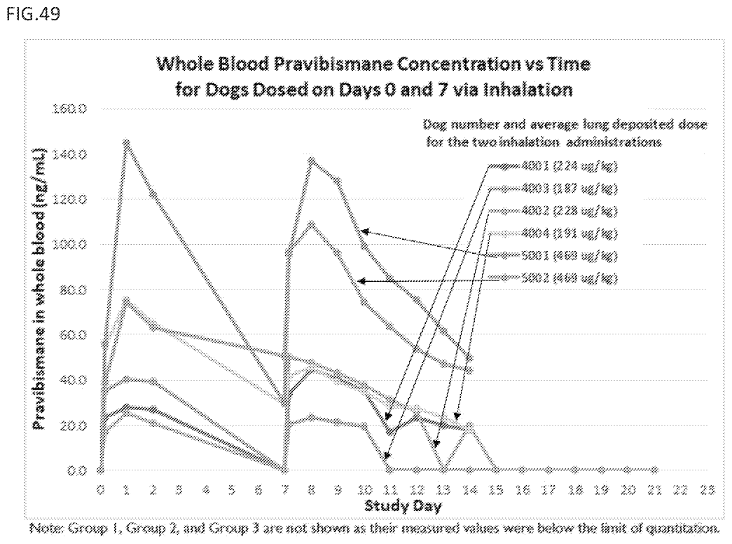

Not only is exposure to lung tissue high specifically at the site of infection, but data from longer-term single-dose and repeat-dose rat studies have demonstrated that BisEDT has a 4 to 7-day half-life in lung tissue (FIG. 48). Oral dosing data show low systemic bioavailability, and IV data show the drug does not partition into lung tissue (FIG. 48), again supporting that pulmonary delivery deposits drug on the luminal (lung) surface where it slowly dissolves over an extended period of time.

A two-dose inhalation PK study in dogs was performed covering four doses from 50 μg/kg to 450 μg/kg lung deposited dose. FIG. 49 shows the whole blood levels of BisEDT present throughout the course of the study. Group 1, 2, and 3 dogs had blood BisEDT levels below the lower limit of quantitation and are thus not displayed. The results from whole blood analysis demonstrated a direct correlation between lung deposited dose and both Cₘₐₓ and AUC with no accumulation trend observed (except for some slight accumulation seen for Animal 5002 following the second inhalation exposure). The systemic half-life is approximately 4 days, and the lung tissue half-life is approximately 7 days. Tₘₐₓ values were near the 24-hour post-dose time points after both doses, with no change in Tₘₐₓ based on dose level or on whether it was the first or second dose. These data corroborate liver microsome (CYP) and hepatocyte inhibition and induction data that show BisEDT has no significant effect on liver cell metabolism (no inhibition of or induction of standard substrates). If there was an appreciable effect on metabolism in vivo, the second dose PK curve would be expected to vary more significantly from that of the first dose.

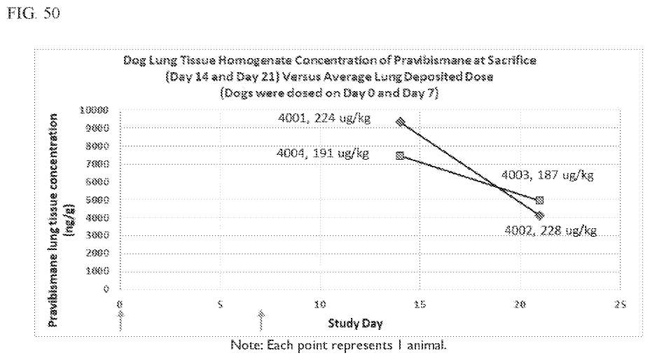

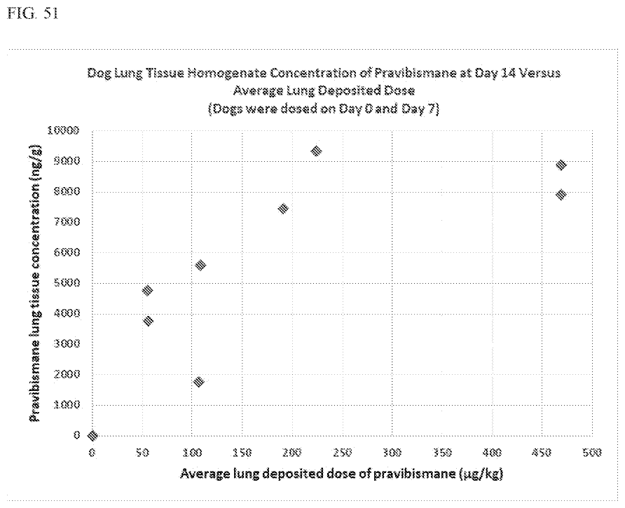

Lung tissue samples demonstrated significantly higher concentrations of BisEDT than whole blood samples. For example, at the sacrifice on Day 14 (7 days after their last dose) Animals 4001 and 4004 had an average blood level of less than 20 ng/mL whereas their lung tissue level was over 7,000 ng/g (FIG. 50). The lung tissue BisEDT also has a long half-life of greater than 4 days. Lung tissue levels correlate with lung deposited dose, however at the highest dose group the lung levels were not as high as would be expected from the trend seen at lower doses (FIG. 51).

Given BisEDT's low aqueous solubility (<5 μg/mL), pharmacokinetics (absorption rate limited clearance) is both hypothesized and observed. To optimally treat a topical lung infection, this is the preferred physicochemical profile of a model drug. In fact, Novartis and Bayer clinically tested an inhaled low solubility zwitterionic form of ciprofloxacin (ciprofloxacin betaine) that was intended to increase the residence time on the lung lumen. Of note, Cayston® (aztreonam for inhalation solution), a standard of care treatment for CF lung infections, is an unstable (refrigerated storage), highly soluble, and rapidly cleared (serum t_{1/2}=1.7 h) drug that requires patients to reconstitute the lyophilized powder and nebulize the resulting solution three times a day. Although efficacious, there is clearly room to improve patient compliance and convenience with a new and less-frequently administered anti-infective treatment option.

It is believed that BisEDT's extended residence time in the lung, nebulized treatment frequency may allow a wide variety of treatment regimens, such as once per day administration or once per week administration, representing a very strong advantage over existing inhaled drugs, both in terms of continual extended activity, and in terms of convenience. Apart from BisEDT's favorable long persistence in the lungs, lung tissue levels at doses near the NOAEL in rats have been measured to be approximately 5 μg/g of total wet lung tissue 24 hours after dosing. In an average rat, this correlates to 150-175 μg/mL in lung epithelial lining fluid, suggesting efficacious concentrations are exceeded by over an order of magnitude and maintained for at least 24 h with aerosolized BisEDT dosed at the NOAEL.

An in vivo efficacy study was designed to assess the efficacy of inhaled BisEDT in a chronic model of P. aeruginosa pulmonary infection. Sprague Dawley rats were divided into four groups: Group 1, negative control (saline), Group 2, positive control (tobramycin; 20 mg/kg (presented dose), twice daily (BID)), Group 3, BisEDT (concentration 0.1 mg/kg (presented dose), once daily on Day 0, 2, 4), and Group 4, BisEDT (concentration 0.25 mg/kg (presented dose), once on Day −1). Animals were challenged intratracheally (IT) on Day 0 with a bolus of bacteria enmeshed in agar beads. Animals were euthanized at Study Day 3 and 5 and assessed for level of pulmonary bacterial burden. The doses of BisEDT were much lower than the NOAEL and no adverse effects were noted due to test article administration. The positive control group was dosed twice a day (BID) for 4 days with high doses of tobramycin. The positive control group was not intended to provide a relative comparison to the treatment groups but rather a confirmation that the model is valid under the study conditions.

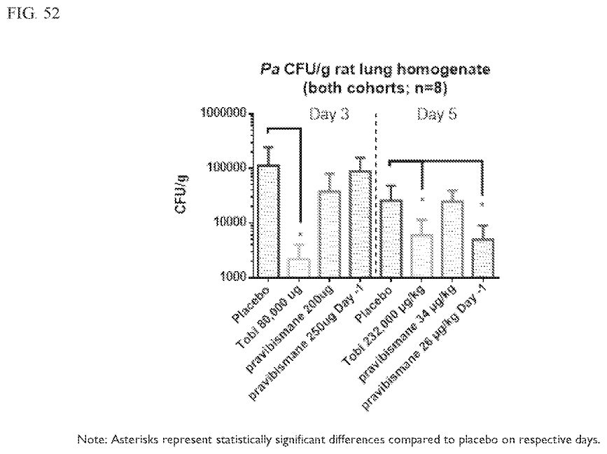

A statistically significant effect was observed on Day 5 for rats treated with a single 26 μg/kg dose of BisEDT. Despite the vastly higher inhaled tobramycin dose (which was spread over 8 doses), the efficacy was not significantly different between the tobramycin positive control group and single dose of BisEDT group by Day 5 (FIG. 52). This advanced level of activity was demonstrated, despite the fact that inhaled BisEDT was administered at a dose several thousand-fold lower than the dose of inhaled tobramycin (notated in the FIG. 52, as Tobi).

Several key advantages to inhaled BisEDT are apparent, based on the studies and development program carried out to date. The long half-life of BisEDT in the lungs, as administered by inhalation of nebulized BisEDT, is anticipated to provide persistent, continual antibacterial, antifungal activity in the lungs of pulmonary infection patients. Inhaled administration to the lungs has been demonstrated to be flexible, predictable, efficacious, and very well-tolerated at doses substantially in excess of anticipated therapeutic doses. GLP studies are anticipated to confirm non-GLP studies performed to date, enabling initiation of human clinical studies.

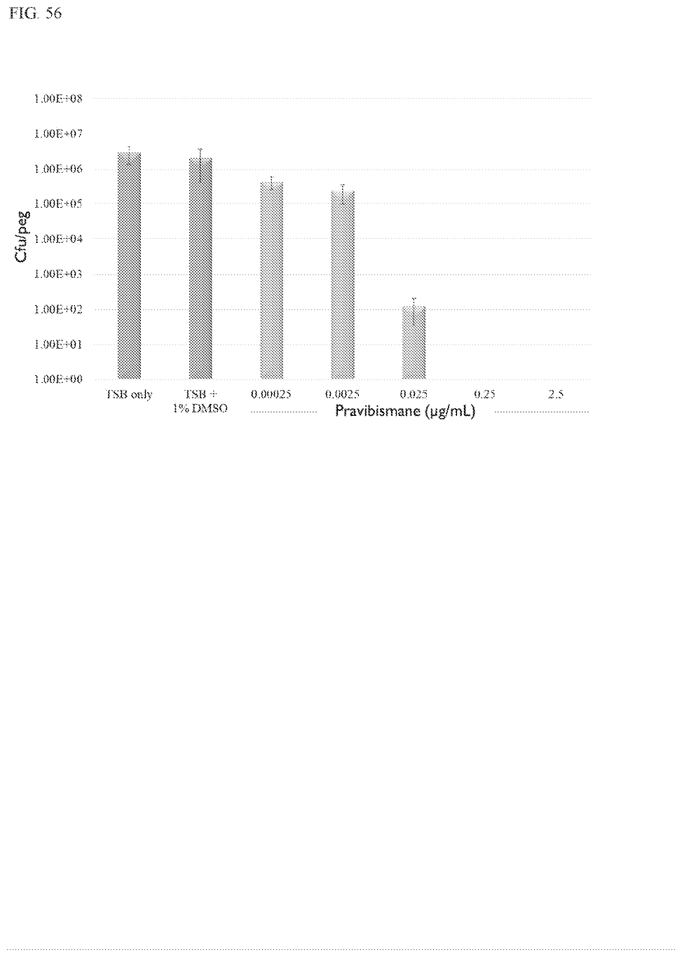

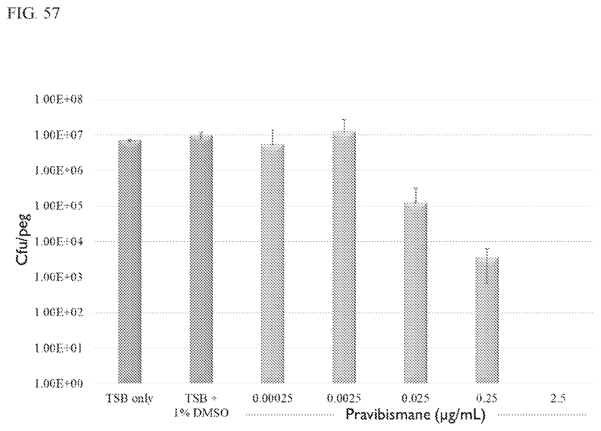

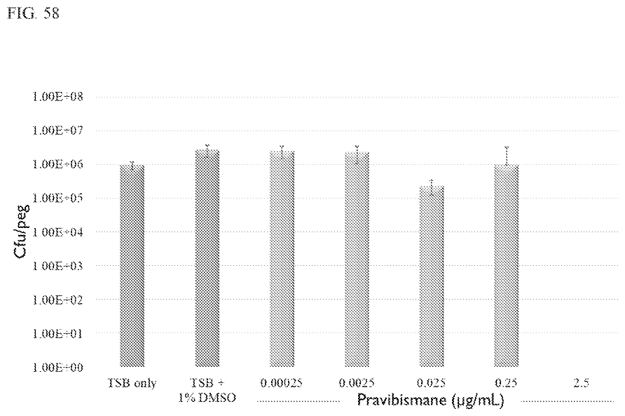

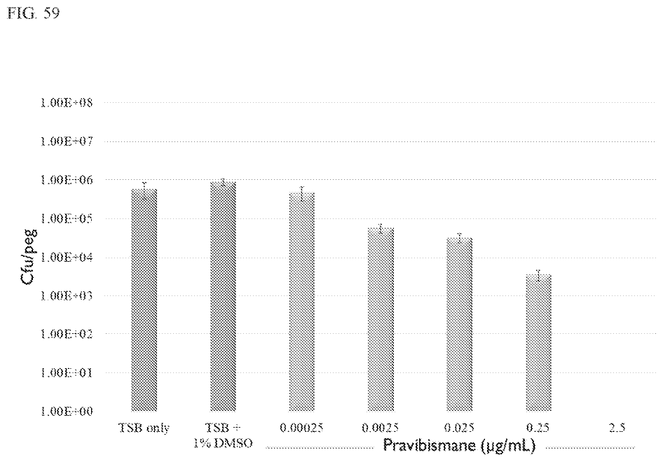

Further feasibility studies related to the potential value of BisEDT as a drug to treat CF pulmonary infections were conducted the results of which are seen in Table 117 and FIGS. 56-59. These studies have established that treatment with 0.25-2.5 μg/mL BisEDT results in complete (6-7 log) reduction of colony-forming units (CFUs) from biofilms formed by multiple species of multidrug-resistant Pseudomonas aeruginosa, Burkholderia cenocepacia, and non-tuberculous mycobacteria (NTM) isolates from CF patients. These studies indicate a powerful biofilm eradication effect of BisEDT on a range of antibiotic-resistant clinical CF isolates. The data presented demonstrate a clear concentration-dependent response against CF isolates.

TABLE 117
MIC values for BisEDT and comparator agents in CF pathogen isolates

| | | MIC (μg/mL) | | |
|---|---|---|---|---|
| Species (Strain ID#) | Type | Pravibismane | AMK | CLR |
| P. aeruginosa (AGR1) | — | 1 | 16 | na |
| P. aeruginosa (AGR14) | MDR | 0.5 | >64 | na |
| P. aeruginosa (MR14) | MDR | 1 | >64 | na |
| M. abscessus/massiliense complex | Macrolide^{R} | 0.06² | 8² | >32¹ |
| (ATCL119977) | (inducible) | | | |
| M. abscessus/massiliense complex | — | 0.06² | 16² | 1 |
| (AMT01130-8) | | | | |
| M. abscessus/massiliense complex | — | 0.13² | 32² | 2 |
| (AMT1539) | | | | |
| M. abscessus/massiliense complex | — | 0.25² | 32² | 1 |
| (AMT0068-40) | | | | |
| M. abscessus/massiliense complex | — | 0.06² | 32² | 1 |
| (AMT0119-7) | | | | |
| M. abscessus/massiliense complex | AMK^{R} | 0.5² | >64² | 2 |
| (AMT0493-2) | | | | |
| S. maltophilia (SM21) | — | 0.25 | 64 | na |
| Achromobacter spp. (AX1) | — | 1 | 64 | na |
| A. xylosoxidans (AX4) | — | 0.25 | >64 | na |
| B. multivorans (B. cepacia complex) | — | 0.25 | >64 | na |
| (BC5b) | | | | |
| B. cenocepacia (B. cepacia complex) | — | 2 | >64 | na |
| (BC15) | | | | |
| B. cepacia (B. cepacia complex) | — | 8 | >64 | na |
| (BC17) | | | | |
| B. cenocepacia (B. cepacia complex) | — | 0.5 | >64 | na |
| (AU197) | | | | |

Conclusion: Treatment of patients affected by respiratory viral infections in pandemics, the COVID-19 pandemic included, may derive substantial benefit from the targeted, inhaled administration of BisEDT. BisEDT is the first drug candidate from a new class of anti-infective drugs (bismanes), with a unique mechanism of action. In clinical and non-clinical studies it has shown to be very safe, extremely effective as a broad-spectrum anti-infective drug, potent against key MDR bacterial and fungal pulmonary pathogens, and comparatively impervious to resistance development. Importantly, with respect to bacterial and fungal pathogens that can complicate coronavirus infections (and other vial respiratory infections), BisEDT is more potent against almost every Gram-negative species than any of the comparator antibiotic agents, and is more active against most Gram-positive bacteria tested than linezolid and the other relevant marketed antibiotics. BisEDT also demonstrated in vitro activity for antifungal-resistant and susceptible Aspergillus spp, Candida spp., and highly resistant strains of Candida auris which have recently become notorious, pan-resistant pathogens in clinical settings. BisEDT has been demonstrated in non-clinical and clinical studies to date that it can be successfully administered directly to the site of the infection, including the lungs. Inhaled BisEDT will become a very important tool in the treatment armamentarium, both for the treatment of primary bacterial and fungal infections of the lungs, and also for the treatment of the secondary bacterial and/or fungal pulmonary infections that complicate viral pulmonary infections during viral epidemics and pandemics. BisEDT should be prioritized and expedited, in order to reduce mortality associated with primary bacterial or fungal infections, and to reduce related mortality associated with viral pandemics.

### Example 16: Evaluation of the Mechanism of BisEDT in the Escherichia coli Macromolecular Synthesis Assay

The macromolecular synthesis (MMS) assay can be a useful tool for determining the mechanism of action of BisEDT against microbes. The goal of the current study was to evaluate potential inhibition of all MMS pathways by BisEDT using the Escherichia coli ATCC 25922 MMS model. Initial minimum inhibitory concentration (MIC) values were determined using methodology recommended by the Clinical and Laboratory Standards Institute (CLSI. Methods for Dilution Antimicrobial Susceptibility Tests for Bacteria That Grow Aerobically; 11 th Ed. CLSI standard M07. CLSI, 950 West Valley Road, Suite 2500, Wayne, Pa. 19087 USA, 2018; CLSI. Performance Standards for Antimicrobial Susceptibility Testing. 30^{th} Ed. CLSI supplement M100. CLSI, 950 West Valley Road, Suite 2500, Wayne, Pa. 19087 USA, 2020). Inhibition of DNA, RNA, protein, cell wall, and lipid synthesis was evaluated by measuring incorporation of radiolabeled precursors for these pathways after incubation with BisEDT and control agents.

Materials and Methods: BisEDT was stored at room temperature until testing. For broth microdilution, BisEDT and comparators were prepared at 101X the highest final concentration and used on the same day. For macromolecular synthesis (MMS), these stock solutions were aliquoted and stored at −20° C. Ciprofloxacin, imipenem, and tetracycline were prepared fresh on the assay day to avoid freezing.

The control agents, imipenem, ciprofloxacin, irgasan, rifampicin and tetracycline were supplied by Micromyx. Information regarding the manufacturer, lot number, solvent, and pathway inhibited for each comparator is listed in Table 118 below:

TABLE 118
Comparator drugs

| | | | | Concentration | |
|---|---|---|---|---|---|
| | | | | Ranges Tested | |
| | | | | (μg/mL) in | Pathway |
| Drug | Supplier | Lot No. | Solvent | MIC assay | Inhibited |
| Rifampicin | Sigma | 080M1506V | DMSO | 64-0.06 | RNA |
| Ciprofloxacin | USP | J1L040 | Water | 1-0.001 | DNA |
| Irgasan | Sigma | 0001412854 | DMSO | 1-0.001 | Lipid |
| Imipenem | USP | R038R0 | Phosphate | 1-0.001 | Cell Wall |
| | | | buffer pH 7.2 | | |
| Tetracycline | Sigma | 110M1693V | Water | 64-0.06 | Protein |
| BisEDT | Microbion | ED268-1-11-01 | DMSO | 64-0.06 | n/a |

The test organism for the assay was the E. coli reference strain, ATCC 25922, acquired from the American Type Culture Collection (ATCC, Manassas, Va.). In addition, E. coli MMX 121, an E. coli K12 tolC::TN10 efflux pump mutant, was evaluated for RNA inhibition only.

Test Organism: Upon receipt, the isolates were streaked onto the appropriate agar plate and incubated at optimal conditions for growth. Colonies were harvested from this plate and a cell suspension was prepared in the appropriate medium containing cryoprotectant. Aliquots were then frozen at −80° C. Prior to testing, the isolate was streaked from the frozen vial onto Trypticase Soy agar plates with sheep blood (Becton Dickinson [BD]; Lot No. 0254268; Sparks, Md.) and incubated overnight at 35° C.

Test Medium: The medium employed for the MIC assay and the macromolecular synthesis assays was cation-adjusted Mueller Hinton II Broth (CAMHB; BD; Lot No. 9324795), except for cell wall evaluation which was performed RPMI-1640 medium (Hyclone; Lot No. AC10257966A: Logan, Utah) with MOPS (EMD Millipore; Lot No. 3462216; Billerica, Mass.). Both media were prepared according to CLSI guidelines (CLSI. Methods for Dilution Antimicrobial Susceptibility Tests for Bacteria That Grow Aerobically; 11th Ed. CLSI standard M07. CLSI, 950 West Valley Road, Suite 2500, Wayne, Pa. 19087 USA, 2018; CLSI. Performance Standards for Antimicrobial Susceptibility Testing. 30th Ed. CLSI supplement M100. CLSI, 950 West Valley Road, Suite 2500, Wayne, Pa. 19087 USA, 2020).

Susceptibility Testing: The concentration of each individual compound utilized in the macromolecular synthesis assay is highly dependent upon the MIC value for the organism being tested (E. coli ATCC 25922). The MIC for the test agent and the positive control antibiotics listed above were determined with triplicate independent inocula using the broth microdilution method described by CLSI. The assay employed automated liquid handlers to conduct serial dilutions and liquid transfers. Automated liquid handlers included the Biomek 2000 and Biomek F/X (Beckman Coulter, Fullerton Calif.).

The wells of columns 2 through 12 of a standard 96-well microdilution plate (Costar 3795) were filled with 150 μL of appropriate diluent by hand. The drugs (300 μL at 101X the highest final concentration) were dispensed into Column 1 of the appropriate row in these plates. These would become the mother plates from which the test plates (daughter plates) were prepared. The Biomek 2000 completed serial transfers through Column 11 in the mother plates. The wells of Column 12 contained no drug and were the organism growth control wells in the daughter plates. The daughter plates were loaded with 190 μL of the appropriate test media by hand. The daughter plates were prepared on the Biomek FX instrument which transferred 2 □L of drug solution from each well of a mother plate to each corresponding well of each daughter plate in a single step.

A standardized inoculum of E. coli ATCC 25922 was prepared per CLSI methods (CLSI. Methods for Dilution Antimicrobial Susceptibility Tests for Bacteria That Grow Aerobically; 11^{th} Ed. CLSI standard M07. CLSI, 950 West Valley Road, Suite 2500, Wayne, Pa. 19087 USA, 2018). A suspension was prepared in CAMHB to equal the turbidity of a 0.5 McFarland standard. The suspension was diluted 1:20 in the appropriate medium. The inoculum was dispensed into sterile reservoirs divided by length (Beckman Coulter), and the Biomek 2000 was used to inoculate the plates. Daughter plates were placed on the Biomek 2000 work surface reversed so that inoculation took place from low to high drug concentration. The Biomek 2000 delivered 10 μL of standardized inoculum into each well. This yielded a final cell concentration in the daughter plates of approximately 5×10⁵ colony-forming-units/mL. The wells of the daughter plates ultimately contained 190 μL of broth, 2 μL of drug solution, and 10 μL of bacterial inoculum.

Plates were stacked 3 high, covered with a lid on the top plate, placed in plastic bags, and incubated at 35° C. for approximately 18 hours. The microplates were viewed from the bottom using a plate viewer. An un-inoculated solubility control plate was observed for evidence of drug precipitation. The MIC was read and recorded as the lowest concentration of drug that inhibited visible growth of the organism.

MMS Assays: Bacteria and growth conditions: Macromolecular synthesis inhibition of the test agent was investigated using E. coli ATCC 25922. Cells were grown at 35° C. overnight on Trypticase Soy Agar plus 5% sheep blood (Becton Dickinson; Cat No. 221261; Lot No. 0254268). Isolated colonies were used to inoculate 30 mL of media. The culture was grown to early exponential growth phase (OD600=0.2 to 0.3) while incubating in a shaker at 35° C. and 150 rpm.

DNA and RNA synthesis: When cells reached early exponential phase, 97.5 μl of culture was added to triplicate 1.5 ml microfuge tubes containing various concentrations of test compound or control antibiotics (2.5 μl at 40X the required final concentration). A “no drug” control was included for all experiments. Following a 30 min pre-incubation at room temperature to allow for drug inhibition of a pathway, either 5 μl of [3H] thymidine at 2 μCi per reaction (DNA synthesis) (Perkin Elmer; Lot No. 202003L; Boston, Mass.) or 5 μl of [3H] uridine at 0.5 μCi per reaction (RNA synthesis) (Perkin Elmer; Lot No. 2541586) was added. Reactions were allowed to proceed at room temperature for 30 min and then stopped by adding 6 μl of cold 100% trichloroacetic acid (TCA). Reactions were incubated on ice for 30 minutes. The TCA precipitated material (103 μl) was spotted onto pre-wet Whatman Glass microfiber filters (GF/A) (GE Healthcare; Buckinghamshire, UK). After washing three times with 5 mL of cold 5% TCA and two times with 5 mL of ethanol, the filters were allowed to dry, and then counted using a Perkin Elmer Tri-Carb 4810TR Liquid Scintillation Analyzer.

Following the initial RNA macromolecular synthesis study using E. coli ATCC 25922, a second assay was done with both E. coli ATCC 25922 and E. coli MMX 121 (K12 tolC mutant) using a higher concentration range for BisEDT. In this study, a previously determined MIC value was used for E. coli MMX 121. The reactions were carried out in triplicate in the wells of a 96 well plate, and 5 μl of [3H] uridine at 0.5 μCi per well (Perkin Elmer; Lot No. 2773912) was added.

Protein Synthesis; When cells reached early exponential phase in MHB II, they were resuspended in M9 minimal medium (1X M9 salts, 0.4% glucose, 2 mM MgSO4, 0.1 mM CaCl₂)) (MEM) and 97.5 μl of culture was added to triplicate 1.5 ml microfuge tubes containing various concentrations of test compound or control antibiotics (2.5 μl at 40X the required final concentration). Following a 30 min pre-incubation at room temperature to allow for drug inhibition of protein synthesis, 5 μl of [3H] leucine (Perkin Elmer; Lot No. 2748912) was added at 2.0 μCi per well. Reactions were allowed to proceed at room temperature for 30 min and then stopped by adding 12 μl of cold 50% trichloroacetic acid (TCA)/20% casamino acids. Reactions were incubated on ice for 30 minutes. The TCA precipitated material (103 μl) was spotted onto pre-wet Whatman Glass microfiber filters (GF/A). After washing three times with 5 mL of cold 5% TCA and two times with 5 mL of ethanol, the filters were allowed to dry, and then counted using a Perkin Elmer Tri-Carb 4810TR Liquid Scintillation Analyzer.

Cell Wall Synthesis: When cells reached early exponential phase in RPMI 1640 Medium, they were added to 1.5 ml microfuge tubes (100 μL/tube in triplicate) containing various concentrations of test compound or control antibiotics (2.5 μl) at 40X the required final concentration. Following a 60-min pre-incubation at 37° C. to allow for drug inhibition of cell wall synthesis, [14C] N-acetylglucosamine (0.4 μCi/reaction) was added to each tube and incubated for 30 min in a 37° C. heating block. Reactions were stopped through the addition of 100 μl of 8% SDS to each tube. Reactions were then heated at 95° C. for 30 min in a heating block, cooled and briefly centrifuged. A one hundred and eighty microliter aliquot of each reaction was spotted onto pre-wet nitrocellulose membrane filters (0.8 μM). After washing three times with 5 ml of 0.1% SDS, the filters were rinsed two times with 5 ml of deionized water and allowed to dry. After drying, the filters were placed in scintillation vials, liquid scintillation fluid was added, and counts were determined using a Perkin Elmer Tri-Carb 4810TR Liquid Scintillation Analyzer.

Lipid Synthesis: Bacterial cells were grown to early exponential growth phase in CAMHB and 100 μL was added to 1.5 ml microfuge tubes (in triplicate) containing various concentrations of test compound or control antibiotics as described above. Following a 30-min pre-incubation at room temperature to allow for inhibition of lipid synthesis, [3H] glycerol was added at 0.5 μCi per reaction. Reactions were allowed to proceed at room temperature for 50 min and then stopped through the addition of 375 μl chloroform/methanol (1:2), followed by vortexing for 20 seconds after each addition. Chloroform (125 μL) was then added to each reaction and vortexed, followed by the addition of 125 μl dH2O and vortexing. Reactions were centrifuged at 13,000 rpm in a microfuge for 10 min, and then 150 μl of the organic phase was transferred to a scintillation vial and allowed to dry in a fume hood for at least 1 hr. Liquid scintillation fluid was added and samples were counted using a Perkin Elmer Tri-Carb 4810TR Liquid Scintillation Analyzer.

Results and Discussion: The in vitro activity of BisEDT and the positive control agents utilized in the MMS assay is shown in Table 119. BisEDT demonstrated a median MIC of 0.5 μg/mL against E. coli ATCC 25922 (assay conducted in triplicate). In addition, for the positive control agents ciprofloxacin, rifampicin, and tetracycline, median MIC values against E. coli ATCC 25922 were within published CLSI QC ranges, thus validating this assay (3).

TABLE 119
Susceptibility testing of Escherichia coli ATCC 25922 to BisEDT and comparators

| | | MIC (μg/mL) | | | | | |
|---|---|---|---|---|---|---|---|
| Organism | Inoculum | Pravibismane | Imipenem | Ciprofloxacin | Irgasan | Rifampicin | Tetracycline |
| E. coli | 1 | 0.5 | 0.12 | 0.008 | 0.12 | 8 | 1 |
| ATCC | | | (0.06-0.25)¹ | (0.004-0.016) | | (4-16) | (0.5-2) |
| 25922 | 2 | 0.5 | 0.12 | 0.008 | 0.12 | 8 | 1 |
| | | | (0.06-0.25)¹ | (0.004-0.016) | | (4-16) | (0.5-2) |
| | 3 | 0.5 | 0.12 | 0.015 | 0.12 | 8 | 1 |
| | | | (0.06-0.25)¹ | (0.004-0.016) | | (4-16) | (0.5-2) |

¹CLSI QC range shown in parentheses

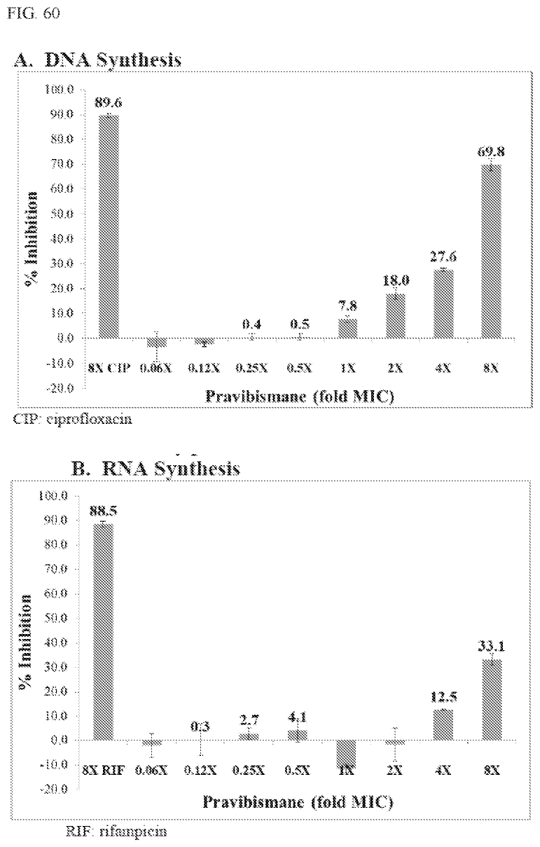

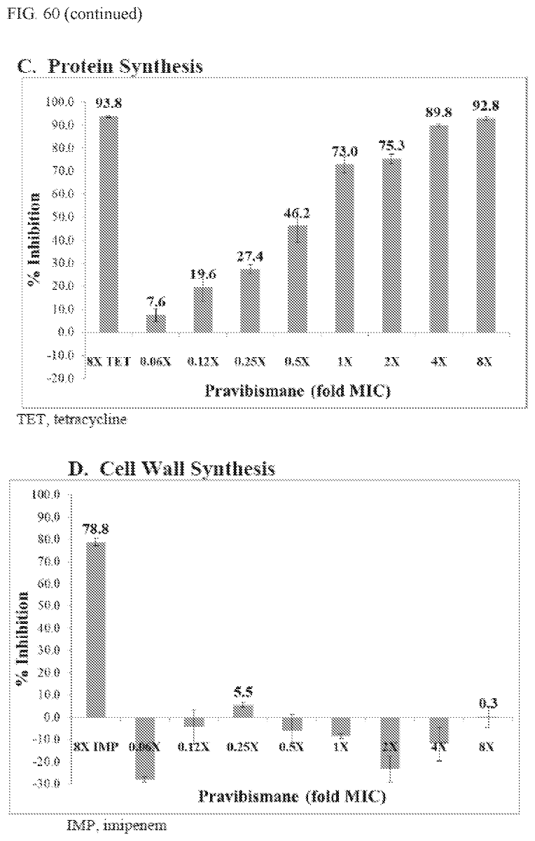

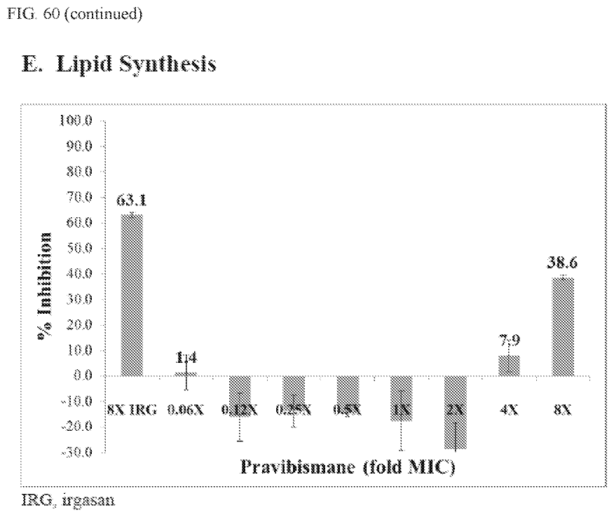

The results of performing macromolecular synthesis inhibition assays in E. coli ATCC 25922 with BisEDT (indicated as Pravibismane in Table 119) at multiples of the MIC are shown in FIG. 60 for each of the five reactions. For DNA synthesis (FIG. 60A), BisEDT inhibited the reaction in a dose-dependent fashion from 7.8% to 69.8% between 1X and 8X the MIC. This compares with 89.6% inhibition observed for ciprofloxacin at 16X the MIC.

In the RNA synthesis reaction (FIG. 60B), BisEDT exhibited inhibition starting at 4X the MIC (12.5% inhibition) to 33.1% inhibition by 8X the MIC. Rifampicin demonstrated 88.5% inhibition at 8X the MIC in this assay. As this result demonstrated less inhibition with BisEDT than expected based on prior results with a TolC mutant of E. coli (1), the inhibition of RNA synthesis by BisEDT was re-evaluated as described below.

In FIG. 60C, dose-dependent inhibition of protein synthesis was observed with BisEDT from 7.6% to 92.8% between 0.06X and 8X the MIC. The control drug tetracycline demonstrated 93.8% inhibition at 8X the MIC in this assay. There appeared to be no effect of BisEDT on cell wall synthesis (FIG. 60D), as substantial inhibition was not seen. The control drug imipenem demonstrated 78.8% inhibition in this assay at 8X the MIC.

As shown in FIG. 60E, BisEDT did not inhibit lipid synthesis until 8X the MIC (38.6% inhibition), and no dose-dependent response was observed. The control drug irgasan showed 63.1% inhibition in this assay.

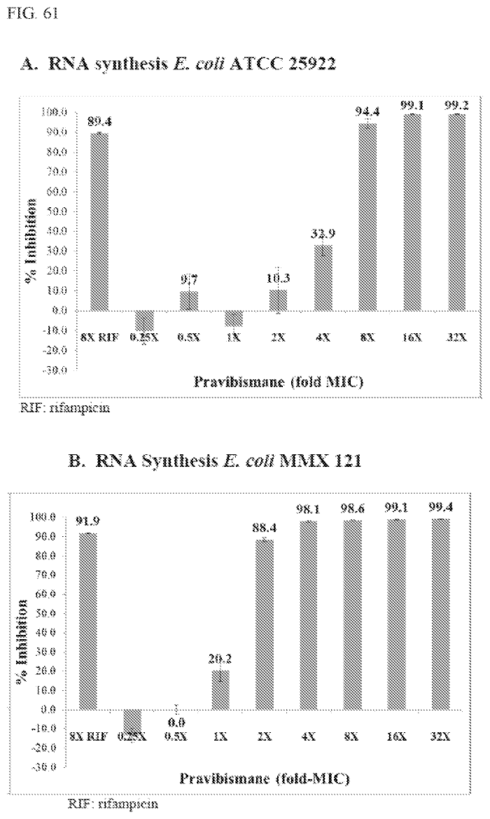

Since RNA inhibition by BisEDT was not observed until 4X the MIC and was less robust than that observed previously with a TolC mutant of E. coli (1), inhibition of this pathway was repeated for E. coli ATCC 25922 using a higher concentration range alongside E. coli MMX 121, the E. coli TolC efflux mutant (FIGS. 61A and 61B). In this experiment, BisEDT displayed dose-dependent inhibition of E. coli ATCC 25922 from 2X the MIC (10.3% inhibition) to 16X the MIC (99.1% inhibition) (FIG. 61A). The control drug, rifampicin showed 89.4% inhibition at 8X the MIC (FIG. 61A). The E. coli K12 tolC mutant (MMX 121) displayed dose-dependent RNA inhibition at 1X the MIC (20.2% inhibition) to 16X the MIC (99.1% inhibition) (FIG. 2B). At 8X the MIC, BisEDT displayed 98.6% inhibition of RNA synthesis, compared to 91.9% observed with rifampicin at 8X the MIC (FIG. 61B).

In summary, BisEDT inhibited DNA, RNA and protein syntheses in a dose-dependent manner. During initial testing, RNA was inhibited by this drug but only at the higher concentrations tested starting at around 4X the MIC (FIG. 60B). Repeat testing of RNA synthesis inhibition with both E. coli ATCC 25922 and the TolC efflux mutant MMX 121 demonstrated robust dose-dependent inhibition of RNA synthesis (FIGS. 61A and 61B).

### Example 17: Amorphous BisEDT Characterization

Amorphous BisEDT was characterized by XRPD and DSC. Modulated and temperature cycling DSC analyses were also conducted to determine the glass transition temperature and investigate any potential form change/recrystallization events upon heating.

By XRPD, the pattern contains diffuse scattering without any sharp peaks (FIG. 44).

The conventional DSC for the amorphous BisEDT shows multiple events and displays broad, small endotherms at 64° C. and 112° C. (FIG. 45). These are potentially associated with volatile release and relaxation enthalpy due to a glass transition, respectively. The thermogram also shows a small exotherm at 145° C. (peak max) and a very sharp exotherm at 168° C. (peak max.).

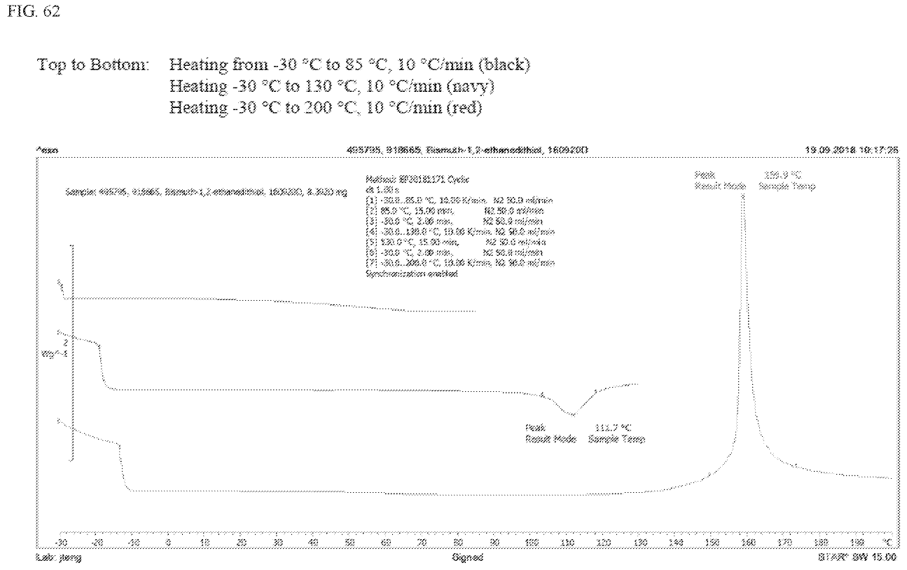

For temperature cycling DSC the material was heated to the temperature past the first broad endotherm to remove any associated volatiles (85° C.) and was quenched to sub-ambient temperature. The sample was then heated to 130° C., above the temperature of the second small endotherm, quenched to sub-ambient temperature, and finally re-heated to 200° C. Based on the data, no apparent glass transition is observed which could be due to crystallization of the amorphous solids upon quenching. The strong exotherm at 160° C. (peak max) is possibly due to degradation, based on hotstage microscopy of the crystalline lot (FIG. 62).

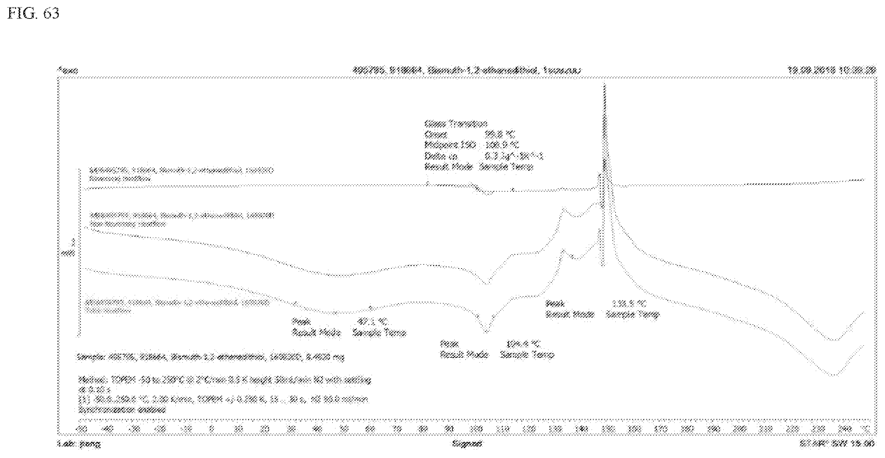

In an attempt to further investigate the glass transition temperature, modulated DSC was conducted with lot 160920D (FIG. 63). A step change on the reversing heat flow curve is observed with a midpoint at 101° C. and is typical of the glass transition. An endotherm seen at 104° C. (peak max) on the total heat flow thermogram is likely associated with the relaxation enthalpy of the glass transition event. The small exotherm at 134° C. (peak max) could be due to crystallization; however, this is immediately followed by heat flow fluctuations which are attributable to decomposition. Thus, the amorphous material exhibited a glass transition at about 101° C.

TABLE 120
Physical Characterization of Amorphous BisEDT

| | Analytical | |
|---|---|---|
| Compound | Technique | Results |
| X-ray | XRPD | Diffuse scatter |
| amorphous | DSC | Broad endotherm at 64° C. (peak max) |
| BisEDT | | Small endotherm at 112° C. (peak max) |
| | | Small exotherm at 145° C. (peak max) |
| | | Sharp strong exotherm at 168° C. |
| | | (peak max) |
| | Temperature | Cycle 1: Heat to 85° C./min |
| | cycling DSC | Broad, weak endotherm at 75° C. |
| | | (approximate peak max) |
| | | Cycle 2: Hold, 15 min |
| | | Cycle 3: Quench cool to −30° C., hold, |
| | | 2 min |
| | | Cycle 4: Heat to 130° C., 10° C./min |
| | | Small endotherm at 112° C. (peak max) |
| | | Cycle 5: Hold, 15 min |
| | | Cycle 6: Quench cool to −30° C., hold, |
| | | 2 min |
| | | Cycle 7: Heat to 200° C., 10° C./min |
| | | Sharp exotherm at 160° C. (peak max) |
| | Modulated | Reversing heat flow |
| | DSC | T_{g} at 101° C. (midpoint of step change) |
| | | (ΔCₚ 0.3 J/(g*K) |
| | | Non-reversing and total heat flow |
| | | Broad weak endotherm at 47° C. |
| | | (peak max). |
| | | Small endotherm at 104° C. (peak max) |
| | | Small exotherm at 134° C. (peak max) |
| | | Heat flow fluctuations above 140° C. |

### Example 18: Synthesis of Amorphous BisEDT

General Procedure using Bismuth (III) Sub-Nitrate: Solids of Bi (III) sub-nitrate were suspended in H₂O (initial solid loading was ˜210-230 mg/mL). 70% HNO₃ was then added until a clear solution was obtained. This solution was added to a 5% aqueous solution of HNO₃, followed by addition of the specified solvent. In a separate vessel, a solution of 1,2-ethanedithiol was prepared in the specified solvent (at a concentration of ˜5% or 13%) and added in portions to the main reactor while stirring. The resulting solids were slurried at ambient temperature for a given duration. The suspension was then allowed to settle and the majority of solvent decanted and replaced with the same solvent. After the suspension was slurried at ambient temperature for a given amount of time, solids were isolated via vacuum filtration and either collected/analyzed or the cycle was repeated.

General Procedure using Bismuth (III) Nitrate Pentahydrate: Solids of Bi (III) nitrate pentahydrate were suspended in H₂O (initial solid loading was ˜230 mg/mL). 70% HNO₃ was then added until a clear solution was obtained. This solution was added to a 5% aqueous solution of HNO₃ followed by addition of the specified solvent. In a separate vessel, a solution of 1,2-ethanedithiol was prepared in the specified solvent (at a concentration of 5%) and added in portions to the main reactor while stirring. The resulting solids were slurried at ambient temperature for a given duration. The suspension was then allowed to settle and the majority of solvent decanted and replaced with the same solvent. After the suspension was slurried at ambient temperature for a given amount of time, solids were isolated via vacuum filtration and either collected/analyzed or the cycle was repeated.

Solvent Studies: International Patent Application Nos. PCT/US2010/023108, PCT/US2011/023549, PCT/US2011/047490 described a synthesis of BisEDT under ethanolic solvent conditions, the product of which was believed to be amorphous; however, unexpectedly this form of BisEDT was discovered to be crystalline. The inventors of the present invention discovered that solvent selection played a critical role in the formation of crystalline and amorphous forms of BisEDT. While ethanol and a variety of other common solvents produced crystalline forms of BisEDT, acetone, acetonitrile, 1,2-dichloroethane, dimethyl sulfoxide, ethyl acetate, isopropanol, and methyl tert-butyl ether were discovered to produce amorphous BisEDT.

TABLE 121
Effect of solvent on the synthesis of amorphous BisEDT From Bi (III) Sub-Nitrate

| | | Initial Solid | 1,2-Ethanedithiol | | |
|---|---|---|---|---|---|
| Scale | Solvent (b) | Loading | molar equiv. | Work-up Conditions | XRPD Results |
| 256 mg | Acetone | 213 mg/mL | 4.5 eq. total (1.5 eq. | Slurry, RT, 1 day. Re-suspended | X-ray |
| | (4 mL) | | initially added as | solids in Acetone; slurry, RT, 1 | amorphous + |
| | | | a ~5% solution in | day. Isolated solids via VF, | peaks of |
| | | | Acetone, 3 eq. | washed filtrate with Acetone. | Form A |
| | | | added neat after | Re-suspended solids its Acetone; | |
| | | | 1 day stirring) | slurry, RT, 4 days, isolated via | |
| | | | | VF. | |
| 509 mg | Acetone | 232 mg/mL | 3 eq. total (1.5 eq. | Slurry, RT, 1 day. Re-suspended | X-ray |
| | (9 mL) | | initially added as | solids in Acetone; slurry, RT, 4 | amorphous + |
| | | | a ~5% solution in | days. Isolated solids via VF. | peaks of |
| | | | Acetone, 1.5 eq. | | Form A |
| | | | added neat after | | |
| | | | 1 day stirring) | | |
| 253 mg | CAN | 211 mg/mL | 4.5 eq. total (1.5 eq. | Slurry, RT, 1 day. Re-suspended | X-ray |
| | (4 mL) | | initially added as | solids in CAN; slurry, RT, 1 day. | amorphous |
| | | | a ~5% solution in | Isolated solids via VF, washed | |
| | | | ACN, 3 eq. | filtrate with CAN. Re-suspended | |
| | | | added neat after | solids in CAN; slurry, RT, 4 | |
| | | | 1 day stirring) | days. Isolated via VF. | |
| 499 mg | CAN | 227 mg/mL | 3 eq. total (1.5 eq. | Slurry, RT, 1 day. Re-suspended | Insufficient |
| | (9 mL) | | initially added as | solids in CAN; slurry, RT, 4 | amount of |
| | | | a ~5% solution in | days. Isolated solids via VF. | solids for |
| | | | ACN, 1.5 eq. | | analysis |
| | | | added after | | |
| | | | 1 day stirring) | | |
| 503 mg | DCE | 229 mg/mL | 4.5 eq. (added as | Slurry, RT, 5 days. | X-ray |
| | (9 mL) | | a ~5% solution in | Re-suspended solids in DCE; | amorphous |
| | | | DCE) | slurry, RT, 1 day. Isolated solids | |
| | | | | via VF, washed DCE. Re- | |
| | | | | suspended solids in DCE; slurry, | |
| | | | | RT, 1 day. Isolated via VF. | |
| 505 | Dioxane | 230 mg/mL | 4.5 eq.(added as | Slurry, RT, 1 day. Re-suspended | Material B |
| | (9 mL) | | a ~13% solution in | solids in Dioxane; slurry, RT, 4 | |
| | | | Dioxane) | days (pale yellow solids). | |
| | | | | Isolated solids via VF. | |
| 508 mg | DMF | 231 mg/mL | 4.5 eq. (added as | Slurry, RT, 1 day. Re-suspended | Form A |
| | (9 mL) | | a ~13% solution in | solids in DMF; slurry, RT, 4 | |
| | | | DMF) | days. Isolated solids via VF. | |
| 499 mg | DMSO | 227 mg/mL | 4.5 eq.(added as | Slurry, RT, 5 days. Attempted to | X-ray |
| | (9 mL) | | a ~5% solution in | re-suspend solids in DMSO; | amorphous |
| | | | DMSO) | obtained clear solution. Added | |
| | | | | H₂O (yellow solids precipitated); | |
| | | | | slurry, RT, 2 days. Isolated | |
| | | | | solids via VF, washed with H₂O. | |
| 502 mg | EtOAc | 228 mg/mL | 4.5 eq.(added as | Slurry, RT, 1 day | X-ray |
| | (9 mL) | | a ~13% solution in | (flocculent solids dumped | amorphous |
| | | | EtOAc) | together). Re-suspended solids in | |
| | | | | EtOAc; slurry, RT, 4 days. | |
| | | | | Isolated solids via VF. | |
| 260 mg | EtOH | 217 mg/mL | 4.5 eq. total (1.5 eq. | Slurry, RT, 1 day. Re-suspended | Form A, |
| | (4 mL) | | initially added as | solids in EtOH; slurry, RT, 1 | disordered |
| | | | a ~5% solution in | day. Isolated solids via VF, | |
| | | | EtOH, 3 eq. | washed filtrate with EtOH. Re- | |
| | | | added neat after | suspended solids in EtOH; | |
| | | | 1 day stirring) | slurry, RT, 4 days. Isolated, via | |
| | | | | VF. | |
| 505 mg | EtOH | 230 mg/mL | 3 eq. total (1.5 eq. | Slurry, RT, 1 day. Re-suspended | Form A, |
| | (9 mL) | | initially added as | solids in EtOH; slurry, RT, 4 | halos |
| | | | a ~5% solution in | days. Isolated solids via VF. | present |
| | | | EtOH, 1.5 eq. | | |
| | | | added neat after | | |
| | | | 1 day stirring) | | |
| 502 mg | EtOH/H₂O | 228 mg/mL | 4.5 eq. (added as | Slurry, RT, 4 days. | Form A + |
| | 50/50 (c) | | a ~5% solution in | Re-suspended solids in 50/50 | Material F |
| | | | EtOH) | EtOH/H₂O; slurry, RT, 3 days. | |
| | | | | Isolated solids via VF. | |
| 508 mg | H₂O | 231 mg/mL | 3 eq. total (1.5 eq. | Slurry, RT, 1 day. Re-suspend | Form A |
| | | | initially added | solids in H₂O; slurry, RT, 4 days. | |
| | | | neat, 1.5 eq. | Isolated solids via VF. | |
| | | | added neat after | | |
| | | | 1 day stirring) | | |
| 254 mg | IPA | 212 mg/mL | 4.5 eq. total (1.5 eq. | Slurry, RT, 1 day. Re-suspend | X-ray |
| | (4 mL) | | initially added as | solids in IPA; slurry, RT, 1 day. | amorphous |
| | | | a ~5% solution in | Isolated solids via VF, washed | |
| | | | IPA, 3 eq. | solids with IPA. Re-suspended | |
| | | | added neat after | solids in IPA; slurry, RT, 4 days. | |
| | | | 1 day stirring) | Isolated solids via VF. | |
| 506 mg | IPA | 230 mg/mL | 3 eq. total (1.5 eq. | Slurry, RT, 1 day. Re-suspend | X-ray |
| | (9 mL) | | initially added | solids in IPA; slurry, RT, 4 days. | amorphous |
| | | | neat, ~5% solution in | Isolated solids via VF. | |
| | | | IPA, 1.5 eq. | | |
| | | | added neat after | | |
| | | | 1 day stirring) | | |
| 509 mg | MeOH | 231 mg/mL | 4.5 eq. added as | Slurry, RT, 1 day. Re-suspended | Form A |
| | (9 mL) | | a ~13% solution in | solids in MeOH; slurry, RT, 4 | |
| | | | MeOH) | days, isolated via VF. | |
| 505 mg | MTBE | 230 mg/mL | 4.5 eq. added as | Slurry, RT, 4 days. | X-ray |
| | | | a ~5% solution in | Re-suspended solids in MTBE; | amorphous |
| | | | MTBE) | slurry, RT, 3 days. Isolated | |
| | | | | solids via VF. | |
| 495 mg | THF | 225 mg/mL | 4.5 eq. added as | Slurry, RT, 5 days. | Material B |
| | (9 mL) (d) | | a ~5% solution in | Re-suspended solids in THF; | |
| | | | THF) | slurry, RT, 1 day. Isolated solids | |
| | | | | via VF, washed with THF (solids | |
| | | | | had a pale yellow, almost white, | |
| | | | | color). Re-suspended white | |
| | | | | solids in THF; slurry, RT, 1 day. | |
| | | | | Isolated via VF. | |

(a) Amounts, temperature, and duration of experiments are approximate.
(b) Specified amount does not include the solvent used to prepare the 1,2-ethanedithiol solution.
(c) 23.5 mL EtOH added, does not include EtOH used to prepare the 1,2-ethanedithiol solution; 50/50 ratio includes total amount of solvents used throughout experiment.
(d) Non-stabilized THF.

TABLE 121
Effect of solvent on the synthesis of amorphous BisEDT From Bi (III) nitrate pentahydrate

| | | Initial Solid | 1,2-Ethanedithiol | | XRPD |
|---|---|---|---|---|---|
| Scale | Solvent (b) | Loading | molar equiv. | Work-up Conditions | Results |
| 503 mg | Acetone | 229 mg/mL | 1.5 eq. (added as | Slurry, RT, 2 days. Re-suspended | Insufficient |
| | (9 mL) | | a ~5% solution in | solids in Acetone; slurry, RT, 4 | amount for |
| | | | Acetone) | days. Isolated solids via VF, washed | analysis |
| | | | | several times with Acetone. | |
| 508 mg | | 231 mg/mL | 1.5 eq (added as | Slurry, RT, 2 days. Re-suspended | Form A, |
| | | | a ~5% solution in | solids in Acetone; shiny, RT, 4 | slight |
| | | | Acetone) | days. Isolated solids via VF and re- | increase in |
| | | | | suspended in Acetone; slurry, RT, 1 | crystallinity |
| | | | | day. Isolated solids via VF. | |
| 497 mg | ACN | 226 mg/mL | 1.5 eq. (added as | Slurry, RT, 2 days. Re-suspended | Form A |
| | (9 mL) | | a ~5% solution in | solids in ACN; slurry, RT, 4 days. | |
| | | | ACN) | Isolated solids via VF; washed | |
| | | | | several times with ACN. | |
| 502 mg | DCE | 228 mg/mL | 1.5 eq. (added as | Slurry, RT, 5 days. Re-suspended | X-ray |
| | (9 mL) | | a ~5% solution in | solids in DCE; slurry, RT, 7 days. | amorphous |
| | | | DCE) | Isolated solids via VF and re- | |
| | | | | suspended in DCE; slurry, RT, 2 | |
| | | | | days. Isolated solids via VF. | |
| 508 mg | Dioxane | 231 mg/mL | 1.5 eq. (added as | Slurry, RT, 5 days. Re-suspended | Form A |
| | (9 mL) | | a ~5% solution in | solids in dioxane; slurry, RT, 7 | |
| | | | Dioxane) | days. Isolated solids via VF and re- | |
| | | | | suspended in dioxane; slurry, RT, 2 | |
| | | | | days. Isolated solids via VF. | |
| 499 mg | DMF | 227 mg/mL | 1.5 eq. (added as | Slurry, RT, 2 days. Re-suspended | Form A |
| | (9 mL) | | a ~5% solution in | solids in DMF; slurry, RT, 4 days. | |
| | | | DMF) | Isolated solids via VF and re- | |
| | | | | suspended in DMF; slurry, RT, 1 | |
| | | | | day. Isolated solids via VF. | |
| 504 mg | DMSO | 229 mg/mL | 1.5 eq (added as | Slurry, RT, 5 days. Re-suspended | Form A |
| | (9 mL) | | a ~5% solution in | solids in DMSO/H₂O; slurry, RT, 7 | |
| | | | DMSO) | days. Isolated solids via VF and re- | |
| | | | | suspended in H₂O and DMSO (~2:1 | |
| | | | | H₂O:DMSO). Slurry, RT, 2 days. | |
| 504 mg | EtOAc | 229 mg/mL | 1.5 eq. (added as | Slurry, RT, 2 days. Re-suspended | Form A |
| | (9 mL) | | a ~5% solution in | solids in EtOAc; slurry, RT, 4 days. | |
| | | | EtOAc) | Isolated solids via VF; washed | |
| | | | | several times with EtOAc. | |
| 509 mg | EtOH | 231 mg/mL | 1.5 eq. (added as | Slurry, RT, 2 days. Re-suspended | Form A |
| | (9 mL) | | a ~5% solution in | solids in EtOH; slurry, RT, 4 days. | |
| | | | EtOH) | Isolated solids via VF; washed | |
| | | | | several times with EtOH, | |
| 503 mg | EtOH/H₂O | 229 mg/mL | 1.5 eq. (added as | Slurry, RT, 4 days. Re-suspended | Form A |
| | 50/50 (c) | | a ~5% solution in | solids in 50/50 EtOH/H₂O; slurry, | |
| | | | EtOH) | RT, 3 days. Isolated solids via VF. | |
| 494 mg | H₂O | 225 mg/mL | 1.5 eq. | Slurry, RT, 2 days. Re-suspended | Form A |
| | | | (added neat) | solids in H₂O; slurry, RT, 4 days. | |
| | | | | Isolated solids via VF and re- | |
| | | | | suspended in H₂O; slurry, RT, 1 | |
| | | | | day. Isolated solids via VF. | |
| 512 mg | IPA | 233 mg/mL | 1.5 eq. (added as | Slurry, RT, 2 days. Re-suspended | Form A |
| | (9 mL) | | a ~5% solution in | solids in IPA; slurry, RT, 4 days. | disordered |
| | | | IPA) | Isolated solids via VF and re- | |
| | | | | suspended in IPA; slurry, RT, 1 day. | |
| | | | | Isolated solids via AF. | |
| 512 mg | MeOH | 233 mg/mL | 1.5 eq. (added as | Shiny, RT, 2 days. Re-suspended | Form A |
| | (9 mL) | | a ~5% solution in | solids in MeOH; slurry, RT, 4 days. | |
| | | | MeOH) | Isolated solids via VF; washed | |
| | | | | several times with MeOH. | |
| 498 mg | MTBE | 226 mg/mL | 1.5 eq. (added as | Slurry, RT, 4 days. Re-suspended | Material G |
| | (9 mL) | | a ~5% solution in | solids in MTBE; slurry, RT, 3 days. | |
| | | | MTBE) | Isolated solids via VF. | |
| 509 mg | THF | 231 mg/mL | 1.5 eq. (added as | Slurry, RT, 5 days (white/pale | Material B |
| | (9 mL) (d) | | a ~5% solution in | yellow solids). Re-suspended solids | |
| | | | THF) | in THF; shiny, RT, 7 days. Isolated | |
| | | | | solids via VF and re-suspended in | |
| | | | | THF; slurry, RT, 2 days. Isolated | |
| | | | | solids via VF. | |

(a) Amounts, temperature, and duration of experiments are approximate.
(b) Specified amount does not include the solvent used to prepare the 1,2-ethanedithiol solution.
(c) 23.5 mL EtOH added, does not include EtOH used to prepare the 1,2-ethanedithiol solution; 50/50 ratio includes total amount of solvents used throughout experiment.
(d) Non-stabilized THF.

### Example 19: Instrumental Techniques

X-ray Powder Diffraction (XRPD): Transmission Geometry

XRPD patterns were collected with a PANalytical X′Pert PRO MPD diffractometer Using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and an antiscatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 2.2b.

XRPD patterns were collected with a PANalytical Empyrean diffractometer using an incident beam of CU radiation produced using a long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X′Celerator) located 240 mm from the specimen and Data Collector software v. 5.5.

XRPD: Reflection Geometry. XRPD patterns were collected with a PANalytical X′Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a /thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X′Celerator) located 240 mm from the sample and Data Collector software v. 2.2b.

Differential Scanning Calorimetry (DSC). DSC was performed using a Mettler-Toledo DSC3+ differential scanning calorimeter. A tau lag adjustment was performed with indium, tin, and zinc. The temperature and enthalpy were adjusted with octane, phenyl salicylate, indium, tin and zinc. The adjustment was then verified with octane, phenyl salicylate, indium, tin, and zinc. The sample was placed into a hermetically sealed aluminum DSC pan, and the weight was accurately recorded. The pan lid was pierced and then inserted into the DSC cell. A weighed aluminum pan configured as the sample pan was placed on the reference side of the cell. The pan lid was pierced prior to sample analysis. The data acquisition parameters for the thermogram are displayed in the image in the Physical Characterization Data section of this report.

Temperature Modulated Differential Scanning Calorimetry (modulated DSC): Temperature Modulated DSC (TMDSC) was performed using Mettler-Toledo DSC3+ differential scanning calorimeter. TOPEM® overlays the isothermal or ramped temperature with a time series of random temperature pulses of different durations. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan lid was pierced and the pan was then inserted into the DSC cell. A weighed aluminum pan configured as the sample pan was placed on the reference side of the cell. The pan lid was pierced prior to sample analysis. The data was collected from −50° C. to 250° C. at 2° C./min with a modulation amplitude of =0.25 K and a 15 to 30 second period with an underlying heating rate of 2.0 K/minute. The reported glass transition temperature is obtained from the inflection point of the step change in the reversing heat flow versus temperature curve.

### Example 20: Studies on Processing Conditions on BisEDT Particle Size Distribution

It was observed that careful control of the reaction temperature and the rate of 1,2 ethanedithiol addition had pronounced impact on crystalline BisEDT particle size distribution. Representative syntheses are shown below for BisEDT synthesized at 20° C. with a 1.25 hour addition of 1,2-ethane via syringe pump and BisEDt synthesized at 15° C. with a 1 hour addition of 1,2-ethane via syringe pump. Table 122 below shows that temperature conditions play a critical role in particle size distribution, with processing temperatures in the range of 20-30° C. providing BisEDT particles that are both small and uniform in particle size (such as a D90 below 2 microns).

Representative synthesis of BisEDT at 20° C. with 1.25 hour addition of thiol via syringe pump, and polypropylene cloth for filtration: BisEDT synthesis was performed on 10-g scale. To a 1-L jacketed reactor was charged USP water (480 mL, 48 vol), followed by 70% HNO₃ (34 mL, 3.4 vol). A solution of bismuth subnitrate (10 g, 6.84 mmols) in water (43 mL, 4.3 vol) and 70% HNO₃ (14 ML, 1.4 VOL) WAS ADDED AT 20° C. THE REACTION MIXTURE WAS COOLED TO 15° C. FOR ADDITION of 95% Ethanol. The 95% ethanol (180 mL, 18 vol) was then added slowly. (Ethanol addition is exothermic, temperature reached 22° C.). The temperature was then adjusted back to 20° C. This was followed by dropwise addition of 1,2 ethanedithiol (4.3 mL, 7.5 mmols in 95% ethanol in 94 mL, 9.4 vol) over a period of 1.25 hour with the batch temperature at 20° C. during which time it turned into a yellow suspension. The reaction was stirred at 20° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.81 g 91.0%).

Representative synthesis of BisEDT at 15° C. with 1 hour addition of thiol via syringe pump, and polypropylene cloth for filtration: The synthesis BisEDT was performed on 10-g scale, temperature profile was studied with data logger. Ethane dithiol was added at 15° C. over 1 hour via syringe pump and the filtration was performed using PP filter cloth. To a 1-L jacketed reactor was charged USP water (480 mL, 48 vol) and cooled to 15° C., followed by 70% HNO₃ (34 mL, 3.4 vol). A solution of bismuth subnitrate (10 g, 6.84 mmols) in water (43 mL, 4.3 vol) and 70% HNO₃ (14 mL, 1.4 vol) was added at the same temperature. The 95% ethanol (180 mL, 18 vol) was then added slowly. (Ethanol addition is exothermic, temperature reached 22.5° C.). It was then allowed to cool to 15° C. This was followed by dropwise addition of 1,2 ethanedithiol (4.3 mL, 7.5 mmols in 95% ethanol in 94 mL, 9.4 vol) over an hour with the batch temperature at 15° C. The reaction was allowed to stir at 15° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.43 g 87.8%).

TABLE 122
Particle Size Distribution of crystalline BisEDT samples

| Sample | D(10) μm | D(50) μm | D(90) μm | D[4, 3] μm | Conditions |
|---|---|---|---|---|---|
| 1 | 0.80 | 2.4 | 5.9 | 2.9 | Dalton Synthesis |
| | | | | | Conditions |
| 2 | 0.58 | 1.7 | 3.9 | 2.0 | Dalton Synthesis |
| | | | | | Conditions |
| 3 | 0.59 | 1.9 | 4.5 | 2.3 | 30° C., 5 h addition of 1,2- |
| | | | | | ethane dithiol via addition |
| | | | | | funnel |
| 4 | 0.44 | 1.48 | 3.1 | 1.7 | 30° C., 4 hour addition of |
| | | | | | 1,2-ethane dithiol via |
| | | | | | syringe pump |
| 5 | 0.33 | 0.72 | 1.6 | 0.86 | 20° C., 1 h addition of 1,2- |
| | | | | | ethane dithiol via addition |
| | | | | | funnel |
| 6 | 0.34 | 0.87 | 1.8 | 0.98 | 20° C., 4 h addition of 1,2- |
| | | | | | ethane dithiol via addition |
| | | | | | funnel |
| 7 | 0.39 | 1.3 | 1.6 | 1.4 | 20° C., 1 hour addition of |
| | | | | | 1,2-ethane dithiol via |
| | | | | | syringe pump. Sample |
| | | | | | slurried in EtOH. Cloth |
| | | | | | filtration |
| 8 | 0.36 | 1.0 | 1.8 | 1.0 | 20° C., 1 hour addition of |
| | | | | | 1,2-ethane dithiol via |
| | | | | | syringe pump. Sample |
| | | | | | slurried in MeOH. Cloth |
| | | | | | filtration |
| 9 | 0.67 | 1.0 | 1.9 | 1.1 | 20° C., 1 hour addition of |
| | | | | | 1,2-ethane dithiol via |
| | | | | | syringe pump. Sample |
| | | | | | slurried in Abs. MeOH. |
| | | | | | Cloth filtration |
| 10 | 0.36 | 0.88 | 1.7 | 0.97 | 20° C., 1 hour addition of |
| | | | | | 1,2-ethane dithiol via |
| | | | | | syringe pump. Sample |
| | | | | | slurried in IPA. Cloth |
| | | | | | filtration |
| 11 | 0.38 | 1.2 | 2.4 | 1.4 | 15° C. 1.5 hour addition of |
| | | | | | 1,2-ethane dithiol via |
| | | | | | syringe pump. Cloth |
| | | | | | filtration |
| 12 | 0.37 | 1.2 | 2.4 | 1.3 | 20° C., 1.25 hour addition |
| | | | | | of 1,2-ethane via syringe |
| | | | | | pump |
| 13 | 0.36 | 0.98 | 2.1 | 1.2 | 10° C., 1 h addition of 1,2- |
| | | | | | ethane dithiol via syringe |
| | | | | | pump |
| 14 | 0.36 | 1.0 | 2.1 | 1.2 | 10° C. 1 hour addition of |
| | | | | | 1,2-ethane dithiol via |
| | | | | | syringe pump. Cloth |
| | | | | | filtration |
| 15 | 0.32 | 0.72 | 1.6 | 0.86 | 10° C., 4 hours addition of |
| | | | | | 1,2-ethane dithiol via |
| | | | | | syringe pump. Cloth |
| | | | | | filtration. |

### Example 21. A Phase 1b/2a Randomized, Double-Blind, Placebo-Controlled, Multi-Center Study to Assess the Efficacy of Topical BisEDT in Patients with Moderate to Severe Diabetic Foot Infection

Adjunctive local administration of BisEDT was tested for its efficacy in resolving both infection and critical colonization, thereby removing these barriers to wound repair and closure. This represents a fundamental, clinically meaningful benefit.

Topical BisEDT suspension and placebo were used in combination with SOC systemic antibiotics (such as levofloxacin) for a duration of 2-3 weeks. This resulted in a similar proportion of patients in the BisEDT treated groups as compared to placebo who were clinically cured at the test of cure visit, which occurred two weeks following the end of treatment. The 3-D photographic data of wound size suggested that BisEDT may be beneficial in facilitating wound repair and closure, as compared to placebo. A larger proportion of BisEDT treated subjects had a >50% reduction in wound surface area from baseline at the end of treatment (Week 2-3), test of cure (Week 4-5), and at the end of study (Week 8-9) (Table 123). BisEDT, when used in conjunction with systemic antibiotics, for a duration of four weeks (3× per week) may be beneficial in resolving both infection and critical colonization, therefore enabling wound repair and closure, a fundamental, clinically meaningful benefit.

TABLE 123
Proportion of patients with a >50% reduction in wound surface
area in Study topical BisEDT suspension, using 3D Digital Photographs

| | Treatment Group | | | | |
|---|---|---|---|---|---|
| | BisEDT | BisEDT | BisEDT | BisEDT | |
| | 3 μg/cm² | 7.5 μg/cm² | 15 μg/cm² | Pooled | Placebo |
| Time Point | (N = 12) | (N = 12) | (N = 15) | (N = 39) | (N = 13) |
| End of Treatment | 42% | 55% | 33% | 42% | 23% |
| (Week 2-3) | | | | | |
| Test of Cure | 55% | 67% | 57% | 59% | 39% |
| (Week 4-5) | | | | | |
| End of Study | 55% | 63% | 64% | 61% | 42% |
| (Week 8-9) | | | | | |

Example 22. Evaluating the Effect of Pulmonary Surfactant on BisEDT Antimicrobial Activity

In this study, the impact of varying concentrations of pulmonary surfactant (Survanta) on pravibismane (BisEDT) activity against clinical isolates of Staphylococcus aureus, Streptococcus, pneumoniae, Pseudomonas aeruginosa, Escherichia coli, and Acinetobacter baumannii were evaluated by determining minimal inhibitory concentrations (MIC) via broth microdilution in accordance with guidelines established by the Clinical and Laboratory Standards Institute.

Test Compounds and Comparators: The test agent was provided by Microbion and stored at 240° C. prior to testing. The comparator compounds imipenem and daptomycin were supplied by Micromyx. Stock solutions of all compounds were prepared on the day of testing using solvents recommended by CLSI. Stock solutions of all compounds were made at 50.5X the final testing concentration. Information regarding compound source, lot number, testing concentrations and drug diluent for the comparator and test agent is detailed below:

TABLE 124
Test Compounds

| | | | | Testing |
|---|---|---|---|---|
| Test | | Catalog/ | Solvent/ | Range |
| agent | Supplier | Lot No. | Diluent | (μg/mL) |
| BisEDT | Microbion | ED268-1-11-01 | DMSO/ | 32-0.002 |
| | | | DMSO | |
| Imipenem | USP | 1337809/ | Phosphate | 16-0.015 |
| | | R038R0 | buffer 7.2/ | |
| | | | Phosphate | |
| | | | buffer 7.2 | |
| Daptomycin | Merck | NHU04502015 | 32-0.03 | 32-0.03 |

Test Organisms: The test organisms evaluated in this study consisted of clinical isolates from the Micromyx Repository (MMX; Kalamazoo, Mich.) and reference isolates from the American Type Culture Collection (ATCC; Manassas, Va.). Upon initial receipt at Micromyx, the organisms were sub-cultured onto an appropriate agar medium. Following incubation, colonies were harvested from these plates, and cell suspensions were prepared and frozen at −80° C. with a cryoprotectant.

Prior to testing, the isolates were streaked from frozen vials onto trypticase soy agar with 5% sheep blood (Becton Dickenson BD/BBL; Sparks, Md.; Lot No. 0267570) and incubated at 37° C. for 24 hr in ambient atmosphere with the following exceptions: H. influenzae was streaked onto Chocolate Agar (BD; Lot No. 0205420), and S. pneumoniae and H. influenzae were incubated in 5% carbon dioxide.

Test Media: Cation-adjusted Mueller Hinton broth (CAMHB, BD; Lot No. 0224810) was used for MIC testing of aerobic organisms. For Streptococci, this medium was supplemented with 3% lysed horse blood (LHB; Hemostat, Dixon, Calif.; Lot No. 474990). MIC testing of H. influenzae was performed in Haemophilus Test Medium Broth (HTM) which was made by supplementing CAMHB with 15 μg/mL nicotinamide adenine dinucleotide (NAD; Sigma; St. Louis, Mo.; Lot. No. SLBX4629), 15 μg/mL porcine hematin (Sigma; Lot. No. SLBD4979V), 5 g/L yeast extract (Sigma; Lot. No. SLBV4386). Broth used for testing daptomycin was supplemented with 50 ug/mL Ca2+ (Sigma; Lot. No. MKBP4041V).

Surfactant (Survanta; Abbvie, Chicago, Ill.; Lot No. 1135982) was added to the media in concentrations of 1%, 2.5%, 5%, and 10% (v/v).

Broth Microdilution MIC testing: MIC values were determined using a broth microdilution procedure described by CLSI (1,2). Automated liquid handlers (Biomek 3000 and Biomek FX, Beckman Coulter; Fullerton, Calif.) were used to conduct serial dilutions and liquid transfers.

All wells in column 2 through 12 of a standard 96-well microdilution plate (Costar 3795) were filled with 150 μL of the appropriate diluent. Then, 300 μL of the tested agents were added to the wells of column 1 of the plates at 50.5X the highest final concentration to be tested. Serial two-fold dilutions were made across the rows through column 11 using the Biomek 3000. The wells of column 12 contained no drug and served as the growth control wells. This plate served as the “mother plate” from which MIC assay plates or “daughter plates” were made.

The daughter plates were loaded with 90 μL per well of the appropriate test medium for the tested organism using the Biomek FX or by hand. Row A was filled with media without surfactant, B-E were filled with media containing 1%, 2.5%, 5% and 10% surfactant respectively. The daughter plates were completed on the Biomek FX instrument which transferred 2 LL of drug solution from each well of a mother plate to the corresponding well of each daughter plate in a single step.

A standardized inoculum of each test organism was prepared per CLSI methods (1-2). The inoculum for each organism was dispensed into sterile reservoirs divided by length (Beckman Coulter), and the Biomek 3000 was used to inoculate the plates. Daughter plates were placed on the Biomek 3000 work surface in reverse orientation so that inoculation took place from low to high drug concentration. Daughters were then inoculated with 10 μL of the inoculum resulting in a final cell density of approximately 5×105 CFU/mL.

Plates were stacked 3-4 high, covered with a lid on the top plate, placed in plastic bags, and incubated at 35° C. for approximately 20 hr. Following incubation, the microplates were removed from the incubator and viewed from the bottom using a plate viewer. For CAMHB, an un-inoculated solubility control plate was observed for evidence of drug precipitation. The MIC was read and recorded as the lowest concentration of drug that inhibited visible growth of the organism.

Results and Discussion: BisEDT and comparator MICs were determined in varying concentrations of Survanta against 4 Gram-positive and 6 Gram-negative isolates following CLSI guidelines. Distinct precipitate was observed in all wells containing Survanta that made MIC determination difficult or unreadable if the evaluated isolate only displayed light turbidity in particular at higher concentrations of Survanta (Tables 126 and 126). BisEDT and daptomycin were in QC against all evaluated organisms under standard conditions (Tables 125 and 126). While imipenem was out of QC when testing against S. pneumoniae ATCC 49619, imipenem was in QC against for other evaluated QC organisms thus validating the testing (Table 125).

BisEDT MC values were largely unaffected by the addition of Survanta when testing against the evaluated organisms. BisEDT MIC values were only changed by more than a doubling dilution in the presence of Survanta when testing against A. baumannii CDC 0288, as addition of 1%, 2.5%, and 5% Survanta decreased BisEDT MIC values from 0.5 to 0.12 μg/mL (Table 126). Survanta addition increased the MIC values of daptomycin when testing against S. aureus and S. pneumoniae as expected (Table 125), and imipenem MIC values were unaffected by the addition of Survanta (Tables 125 and 126).

In summary, Survanta addition had little impact on the antibacterial activity of BisEDT, as MIC values were within a doubling dilution for all but one evaluated isolate (Table 125 and 126). Survanta had little impact on imipenem as well; in contrast, the antibacterial activity of daptomycin was adversely affected against Gram-positive organisms as expected (Table 125).

TABLE 125
BisEDT and comparator agents MIC values against Gram-positive
organisms in varying concentrations of Survanta.

| | | MIC (μg/mL) | | | | | |
|---|---|---|---|---|---|---|---|
| | Percent | S. aureus | | S. aureus | S. pneumoniae | | S. pneumoniae |
| Drug | Sirvanta | ATCC 29213 | | ATCC 43300 | ATCC 49619 | | MMX 3162 |
| Pravibismane | 0 | 0.25 | (0.12-1) | ≤0.03 | 0.12 | (0.12-1) | 0.12 |
| | 1 | 0.5 | | ≤0.03 | 0.12 | | 0.06 |
| | 2.5 | 0.12 | | ≤0.03 | 0.12 | | 0.12 |
| | 5 | 0.12 | | ≤0.03 | 0.12 | | 0.12 |
| | 10 | 0.12 | | ≤0.03 | ND | | ND |
| Imipenem | 0 | ≤0.015 | (0.015-0.06) | 0.5 | ≤0.015 | (0.03-0.12) | 0.25 |
| | 1 | ≤0.015 | | ND | 0.03 | | 0.25 |
| | 2.5 | ≤0.015 | | ND | 0.03 | | 0.25 |
| | 5 | ≤0.015 | | ND | ND | | 0.25 |
| | 10 | ≤0.015 | | ND | ND | | ND |
| Daptomycin | 0 | 0.5 | (0.12-1) | 0.25 | 0.06 | (0.06-0.5) | 0.12 |

| | | | | |
|---|---|---|---|---|
| 1 | 16 | 8 | 0.25 | 0.25 |
| 2.5 | 32 | 16 | 1 | 0.25 |
| 5 | >32 | 16 | ND | ND |
| 10 | >32 | >32 | ND | ND |

Parentheses denote the CLSI acceptable MIC QC range (2);
ND, Not determined due to precipitation interfering with determination of MIC endpoint

TABLE 126
BisEDT and comparator agents MIC values against Gram-negative organisms in
varying concentrations of Survanta.

| | | MIC (μg/mL) | | | | | |
|---|---|---|---|---|---|---|---|
| | Percent | P. aeruginosa | P. aeruginosa | A. baumanuii | H. Influenzae | H. Influenzae | B. cepucia |
| Drug | Survanta | ATCC 27853 | CDC 0444 | CSC 0288 | ATCC 49247 | MMX 2795 | MMX9040 |
| Pravibismane | 0 | 0.5 (0.5-4) | 0.5 | 0.5 | 0.06 | 0.03 | 0.25 |
| | | | | | (0.015-0.06) | | |
| | 1 | 0.25 | 0.25 | 0.12 | 0.03 | 0.015 | 0.25 |
| | 2.5 | 0.5 | 0.25 | 0.12 | 0.03 | 0.015 | 0.25 |
| | 5 | 0.25 | 0.25 | 0.12 | NT | NT | 0.25 |
| | 10 | 0.25 | 0.25 | 16 | 0.5 | NT | 0.25 |
| Imspenem | 0 | 2 (1-4) | >16 | 16 | 0.5 | 1 | 8 |
| | 1 | 2 | >16 | 16 | 0.5 | UR | 8 |
| | 2.5 | 2 | >16 | 16 | 0.5 | UR | 8 |
| | 5 | 2 | >16 | 16 | NT | NT | 8 |
| | 10 | 2 | >16 | ND | NT | NT | 8 |

Parentheses denote the CLSI acceptable MIC QC range (2);
ND, not determined due to precipitation intertering with determination of MIC endpoint;
NT, not tested

### Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method of treating, managing or lessening the severity of symptoms associated with a respiratory viral infection in a subject, the method comprising administering to the subject a bismuth-thiol (BT) composition that comprises BisEDT suspended therein, wherein administering the BT composition is via inhalation, orally or nasally, using an aerosol device.

2. The method of claim 1, wherein the infection that is treated, managed or lessened is the viral infection.

3. The method of claim 1, wherein the infection that is treated, managed or lessened is a bacterial and/or fungal infection that is secondary to the viral infection.

4. The method of any one of claims 1-3, wherein the BT composition comprises a plurality of microparticles wherein at least 70% of said microparticles having a volumetric mean diameter (VMD) from about 0.6 μm to about 2.5 μm.

5. The method of claim 4, wherein at least 80% of said microparticles having a VMD from about 0.6 μm to about 2.5 μm.

6. The method of claim 5, wherein at least 90% of said microparticles having a VMD from about 0.6 μm to about 2.5 μm.

7. The method of any one of claims 1-6, wherein when the BT composition is aerosolized, at least 70% of the aerosolized liquid droplets have a mass median aerodynamic diameter (MMAD) from about 0.9 μm to about 3 μm.

8. The method of claim 7, wherein when the BT composition is aerosolized, at least 809% of the aerosolized liquid droplets have a MMAD from about 0.9 μm to about 3 μm.

9. The method of claim 8, wherein when the BT composition is aerosolized, at least 90% of the aerosolized liquid droplets have a MMAD from about 0.9 μm to about 3 μm.

10. The method of any of claims 1-9, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.

11. The method of any of claims 1-10, wherein if deposited to the deep lung region, the BisEDT compounds have an average half-life of about 4 days.

12. The method of any of claims 1-11, wherein the secondary infection is a pulmonary infection comprising one or more bacterial pathogens and/or fungal pathogens.

13. The method of any of claims 1-12, wherein the pulmonary infection is one or more of bronchiectasis infection, pneumonia, valley fever, allergic bronchopulmonary aspergillosis (ABPA), ventilator-acquired pneumonia, hospital acquired pneumonia, community acquired pneumonia, ventilator associated tracheobronchitis, lower respiratory tract infection, non-tuberculous Mycobacteria, anthrax, legionellosis, pertussis, bronchitis, Bronchiolitis, COPD-associated infection, viral pneumonia, viral bronchiolitis, and post-lung transplantation.

14. The method of claim 13, wherein the pulmonary infection is pneumonia or ventilator-acquired pneumonia.

15. The method of any of claims 1-14, wherein the method comprises at least one of: (i) reducing a biofilm (e.g. bacterial and/or fungal), (ii) impairing growth of a biofilm (e.g. bacterial and/or fungal), (iii) preventing initial formation of the biofilm (e.g. bacterial and/or fungal), and/or (iv) preventing reformation of the biofilm (e.g. bacterial and/or fungal).

16. The method of claim 10, wherein the one or more pathogens are selected from Haemophilus influenzae, Pseudomonas aeruginosa, Staphylococcus aureus, Staphylococcus warneri Staphylococcus lugdunensis, Staphylococcus epidermidis, Streptococcus milleri/anginous, Streptococcus pyogenes, non-tuberculosis mycobacteria, Mycobacterium tuberculosis, Burkholderia spp., Achromobacter xylosoxidans, Pandoraea sputorum, Stenotrophomonas maltophilia, Alcaligenes xylosoxidans, Haemophilus pittmaniae, Serratia marcescens, Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, Aspergillus flavus, Morganella morganii, Inquilinus limosus, Ralstonia mannitolilytica, Pandoraea apista, Pandoraea pnomenusa, Pandoraea sputorum, Bdellovibrio bacteriovorus, Bordetella bronchiseptica, Vampirovibrio chlorellavorus, Actinobacter baumanni, Cupriadidus metallidurans, Cupriavidus pauculus, Cupriavidus respiraculi, Delftia acidivordans, Exophilia dermatitidis, Herbaspirillum frisingense, Herbaspirillum seropedicae, Klebsiella pneumoniae, Pandoraea norimbergensis, Pandoraea pulmonicola, Pseudomonas mendocina, Pseudomonas pseudoalcaligenes, Pseudomonas putida, Pseudomonas stutzeri, Ralstonia insidiosa, Ralstonia pickettii, Neisseria gonorrhoeae, NDM-1 positive E. coli, Enterobacter cloaca, Vancomycin-resistant E. faecium, Vancomycin-resistant E. faecalis, E. faecium, E. faecalis, Clindamycin-resistant S. agalactiae, S. agalactiae, Bacteroides fragilis, Clostridium difficile, Streptococcus pneumonia, Moraxella catarrhalis, Haemophilus haemolyticus, Haemophilus parainfluenzae, Chlamydophilia pneumoniae, Mycoplasma pneumoniae, Atopobium, Sphingomonas, Saccharibacteria, Leptotrichia, Capnocytophaga, Oribacterium, Aquabacterium, Lachnoanaerobaculum, Campylobacter, Acinetobacter; Agrobacterium; Bordetella; Brevundimonas; Chryseobacterium; Delftia; Enterobacter; Klebsiella; Pandoraea; Pseudomonas; Ralstonia, Coccidioides, and Prevotella.

17. The method of any of claims 1-16, wherein the respiratory viral infection is one or more of influenza viral infection (e.g., seasonal flu), rhinovirus infection (e.g., common cold), coronavirus infection (e.g., Severe Acute Respiratory Syndrome and common cold), paramyxovirus infection (e.g., measles), and/or acute respiratory distress syndrome.

18. The method of claim 17, wherein the coronavirus infection is selected from Severe Acute Respiratory Syndrome-Corona Virus (SARS-CoV), Middle East Respiratory Syndrome virus (CoV-MERS), human HCoV-229E, HCoV-OC43, HCoV-NL63 and HCoV-HKU1.

19. The method of claim 18, wherein the coronavirus infection is SARS-CoV (e.g. SARS-CoV-1, SARS-CoV-2).

20. The method of claim 18, wherein the SARS-CoV is SARS-CoV-2 (COVID-19).

21. The method of claim 17, wherein the respiratory viral infection is an influenza viral infection selected from the group consisting of Influenza A, Influenza B, and Influenza C viral infections.

22. The method of claim 22, wherein the Influenza A virus comprises H1N1, H2N2, H3N2, H5N1, H7N7, H1N2, H9N2, H7N2, H7N3, or H10N7 subtypes.

23. The method of any of claims 1-21, wherein the BT composition is co-administered with one or more antimicrobial agents.

24. The method of claim 23, wherein at least one of the one or more antimicrobial agents is a broad spectrum antiviral agent.

25. The method of any of claims 1-23, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

26. The method of any one of claims 1-25, wherein the BisEDT is amorphous BisEDT.

27. The method of claim 26, wherein the amorphous BisEDT X-ray powder diffraction pattern does not contain any distinct peaks.

28. The method of claim 26 or claim 27, wherein the amorphous BisEDT X-ray powder diffraction pattern is substantially similar to FIG. 44.

29. The method according to any one of claims 26-28, wherein the amorphous BisEDT differential scanning calorimetry thermogram comprises an exothermic peak at about 168° C.

30. The method according to any one of claims 26-29, wherein the amorphous BisEDT differential scanning calorimetry thermogram further comprises an endotherm at about 64° C. and/or an endotherm peak at about 112° C. and/or an exotherm peak at about 145° C.

31. The method according to any one of claims 26-30, wherein the amorphous BisEDT differential scanning calorimetry thermogram is substantially similar to FIG. 45.

32. The method according to any one of claims 26-31, wherein the amorphous BisEDT has a glass transition at about 101° C.

33. The method according to any one of claims 26-32, wherein the amorphous BisEDT is at least 90% pure.

34. An aerosol comprising a plurality of dispersed liquid droplets in a gas, said liquid droplets comprising a BT composition comprising BisEDT compound suspended therein and at least one antimicrobial agent; and
wherein at least 70% of the liquid droplets have a MMAD from about of from about 0.9 μm to about 3 μm.

35. The aerosol of claim 34, wherein prior to aerosolization, the BT composition comprises a plurality of microparticles wherein at least 70% of said microparticles have a VMD of from about 0.6 μm to about 2.5 μm.

36. The aerosol of any one of claims 34-35, wherein least 90% of the liquid droplets have a MMAD from about of from about 0.9 μm to about 3 μm.

37. The aerosol of any one of claims 34-36, wherein prior to aerosolization, the BT composition comprises a plurality of microparticles wherein at least 90% of said microparticles have a VMD of from about 0.6 μm to about 2.5 μm.

38. The aerosol of any one of claims 34-37, wherein the droplets further comprise Tween 80 (e.g. from about 0.05% to about 1%) and optionally a buffer (e.g. sodium phosphate or sodium citrate) at a pH of about 7.4; and/or sodium chloride.

39. The aerosol of any one of claims 34-38, wherein at least one antimicrobial agents is a broad spectrum antiviral agent.

40. The aerosol of any one of claims 34-39, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

41. A pharmaceutical composition comprising bismuth-thiol (BT) composition that comprises BisEDT suspended therein and at least one antimicrobial agent, wherein the BT composition comprises a plurality of microparticles, wherein the D90 of said microparticles is less than or equal to 1.9 μm.

42. The pharmaceutical composition of claim 41, wherein at least 70% of said microparticles having a volumetric mean diameter of from about 0.6 μm to about 2.5 μm.

43. The pharmaceutical composition of claim 41 or claim 42, wherein at least 90% of said microparticles having a volumetric mean diameter of from about 0.6 μm to about 2.5 μm.

44. The pharmaceutical composition of any one of claims 41-43, wherein at least one antimicrobial agents is a broad spectrum antiviral agent.

45. The pharmaceutical composition of any one of claims 41-44, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Itraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

46. A kit comprising
(1) an aerosol comprising a plurality of dispersed liquid droplets in a gas, said liquid droplets comprising a BT composition comprising BisEDT compound suspended therein; and wherein at least 70% of the liquid droplets have a MMAD from about of from about 0.9 μm to about 3 μm, and
(2) at least one antimicrobial agent.

47. The kit of claim 46, wherein the at least one antimicrobial agent is formulated for administration orally, parenterally, topically, perorally, internally, intranasally, rectally, vaginally, lingually, or transdermally.

48. The kit of claim 46 or claim 47, wherein the at least one antimicrobial agents is a broad spectrum antiviral agent.

49. The kit of any one of claims 46-48, wherein the antimicrobial agents are one or more of Amoxicillin, Nitazoxanide, Favipiravir, Mycophenolic acid, Remdesivir, Cidofovir, Niclosamide, Brincidofovir, Chloroquine, EIPA (amiloride), BCX4430 (Galdecivir), Gemcitabine, ABT-263, Berberine, Cyclosporine, Emetine, Amodiaquine, Brequinar, Obatoclax, Rapamycin (Sirolimus), Luteolin, Glycyrrhizin, Eflomithine, Ribavirin, Sorafenib, Suramin, Monensin, Sunitinib, Labyrinthopeptin A2, Silvetrol, Emodin, Amiodarone, Raloxifene, Azithromycin, Labyrinthopeptin A1, Mitoxantrone, Arbidol (Umifenovir), Ganciclovir, Letermovir, Artesunate, Ivermectin, Foscarnet, Simvastatin, Bortezomib, Camptothecin, Ttraconazole, Leflunomide, CR-31-B (−), Nelfinavir, Valacyclovir, 4-HPR(Fenretinide), Aprotinin, Topotecan, Oritavancin, Novobiocin, Pentosan polysulfate, Ezetimibe, Filociclovir, Dasatinib, Isolanid (lanatoside C), Sofosbuvir, Manidipine, Lovastatin, Metformin, Minocycline, Dalbavancin, Teicoplanin, N-MCT, Roscovitin (Seliciclib), Caffeine, Genistin, Regorafenib, Homoharringtonine, Alisporivir, Lopinavir, Erlotinib, Gefitinib, Hexachlorophene, Imatinib, Hydroxychloroquine, Lobucavir, Veraparnil, Apoptozole, Fluoxetine, Fluvastatin, Posaconazole, Tamoxifen, Aciclovir, Acetylsalicylic acid, Camostat, Memantine, Tenofovir, Dibucaine, Pirlindole, Formoterol, Pleconaril, Indomethacin, Ritonavir, Flavopiridol, Bithionol, Abamectin, Doxycycline, Maribavir, Salinomycin, Bepridil, Bromocriptine, Quinine, Apilimod, Diphyllin, Esomeprazole, Omeprazole, Telavancin, Kasugamycin, Trametinib, Zanamivir, CYT-107, Lamivudine, Thymalfasin, Enoxacin, Famciclovir, Trifluridine, Vidarabine, 6-azauridine, Antimycin A, Azaribine, Mycophenolate mofetil, Pyrazofurin, AVN-944, Camplothecin, Verapamid, Azactinide, Nefamostat, or bioflavonoids (e.g. herbacetin, rhoifilin, pectolinarim).

50. A method of preventing infections in a subject, the method comprising administering to the subject a bismuth-thiol (BT) composition that comprises BisEDT suspended therein, wherein administering the BT composition is via inhalation, orally or nasally, using an aerosol device, wherein the subject is connected to a ventilator.

51. The method of claim 50, wherein the infection is ventilator-acquired pneumonia.

52. The method of claim 50 or 51, further comprising administering the composition to the subject prior to ventilation.

53. The method of claim 50 or 51, further comprising administering the composition to the subject during or after ventilation.

54. The method of any one of claims 50-53, wherein the method comprises at least one of: (i) reducing a biofilm (e.g. bacterial and/or fungal), (ii) impairing growth of a biofilm (e.g. bacterial and/or fungal), (iii) preventing initial formation of the biofilm (e.g. bacterial and/or fungal), and/or (iv) preventing reformation of the biofilm (e.g. bacterial and/or fungal).

55. A method of preventing, treating, managing or lessening the severity of superinfections and/or superantigen production in a subject, the method comprising administering to the subject a bismuth-thiol (BT) composition that comprises BisEDT suspended therein.

56. The method of claim 55, wherein the BT composition is administered via inhalation, orally or nasally, using an aerosol device, or intravenously.

57. The method of claim 56, wherein the BT composition is administered via inhalation, orally or nasally, using an aerosol device.

58. The method of claim any one of claims 55-57, wherein the method is preventing symptoms associated with a later respiratory viral infection in a subject.

59. The method of claim 58, wherein the respiratory infection is a SARS-CoV-2 (COVID-19) infection.

60. The method of any one of claims 55-59, wherein the subject has a pre-existing health condition.

61. The method of claim 56, wherein the pre-existing health condition causes chronic inflammation in the subject.

62. The method of claim 56, wherein the pre-existing health condition is a chronic inflammatory disease.

63. The method of claim 62, wherein the chronic inflammatory disease is related to one of or more conditions selected from one or more of the group consisting of Metabolic Syndrome, Obesity, Vasculitis, Cardiovascular Diseases, Chronic Wounds, Diabetes, hypertension, stroke, inflammatory bowel disease, periodontitis, atherosclerosis, COPD, endocarditis, thrombotic diseases, asthma, advanced age, recurrent infections, autoimmune diseases, chronic rhinosinusitis, inflammatory arthritis, neurodegenerative conditions, device-related infections, osteomyelitis, and depression.

64. The method of any one of claims 55-63, wherein the superantigen is a bacterial superantigen.

65. The method of claim 61, were in the superantigens include potent extotoxins secreted by Staphylococcus aureus and/or Streptococcus pyogenes.

66. The method of any one of claims 55-65, wherein the subject is infected with more than a 1 picomolar concentration of superantigen.

67. The method of any one of claims 55-66, wherein the subject is infected with more than 0.1 micogram (ug) of superantigen.

68. The method of any one of claims 55-61, wherein the subject is infected with more than 0.2 ug of superantigen.

69. The method of any one of claims 55-61, wherein the method further prevents, manages or lessens the severity of a cytokine storm in a subject.

70. The method of any one of claims 55-69, wherein the subject has elevated levels of neutrophil-lymphocyte ratio (NLR), IL-6, IL-17, IL-10, interferon-gamma, TNF-α, IL-2, and/or procalcitonin.

71. The method of any one of claims 55-60, wherein the subject has elevated levels of neutrophil-lymphocyte ratio (NLR), IL-6, IL-17, and/or procalcitonin.

72. The method of claim 62, wherein the NLR ratio is greater than 5.

73. The method of claim 62, wherein the NLR ratio is greater than 7.

74. The method of claim 64, wherein the NLR ratios is between 7 and 100.

75. The method of any one of claims 55-74, wherein the administration to the subject a bismuth-thiol (BT) composition, treats, inhibits or prevents the dispersal of biofilms in the subject.

76. The method of claim 75, wherein the biofilm is a nasopharyngeal biofilm.

77. The method of any one of claims 55-76, wherein the subject is older than 60 years of age, 70 years of age, 80 years of age and/or 90 years of age.

78. The method of any one of claims 55-77, wherein the BT composition comprises a plurality of microparticles wherein at least 70% of said microparticles having a volumetric mean diameter (VMD) from about 0.6 μm to about 2.5 μm.

79. The method of claim 78, wherein at least 80% of said microparticles having a VMD from about 0.6 μm to about 2.5 μm.

80. The method of claim 79, wherein at least 90%/o of said microparticles having a VMD from about 0.6 μm to about 2.5 μm.

81. The method of any one of claims 55-77, wherein when the BT composition is aerosolized, at least 70% of the aerosolized liquid droplets have a mass median aerodynamic diameter (MMAD) from about 0.9 μm to about 3 μm.

82. The method of claim 81, wherein when the BT composition is aerosolized, at least 80% of the aerosolized liquid droplets have a MMAD from about 0.9 μm to about 3 μm.

83. The method of claim 82, wherein when the BT composition is aerosolized, at least 90% of the aerosolized liquid droplets have a MMAD from about 0.9 μm to about 3 μm.

84. The method of any of claims 55-83, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.

85. A method of preventing, treating, managing or lessening the severity of symptoms associated with a respiratory viral infection in a subject in a subject, the method comprising administering to the subject a bismuth-thiol (BT) composition that comprises BisEDT suspended therein, wherein administering the BT composition is via inhalation, orally or nasally, using an aerosol device.

86. The method of claim 85, wherein the respiratory infection is a SARS-CoV-2 (COVID-19) infection.

87. The method of claim any one of claims 85-86, wherein the method is preventing symptoms associated with a respiratory viral infection in a subject.

88. The method of any one of claims 85-87, wherein the subject has a pre-existing health condition.

89. The method of claim 88, wherein the pre-existing health condition causes chronic inflammation in the subject.

90. The method of claim 88, wherein the pre-existing health condition is a chronic inflammatory disease.

91. The method of claim 90, wherein the chronic inflammatory disease is related to one of or more conditions selected from one or more of the group consisting of Metabolic Syndrome, Obesity, Vasculitis, Cardiovascular Diseases, Chronic Wounds, Diabetes, hypertension, stroke, inflammatory bowel disease, periodontitis, atherosclerosis, COPD, endocarditis, thrombotic diseases, asthma, advanced age, recurrent infections, autoimmune diseases, chronic rhinosinusitis, inflammatory arthritis, neurodegenerative conditions, device-related infections, osteomyelitis, and depression.

92. The method of any one of claims 85-91, wherein the patient is infected with a secondary infection.

93. The method of claim 92, wherein the secondary infection in the subject is prior to the respiratory viral infection.

94. The method of any one of claims 92-93, wherein the secondary infection produces a superantigen in the subject.

95. The method of claim 94, wherein the superantigen is a bacterial superantigen.

96. The method of claim 95, were in the superantigens include potent extotoxins secreted by Staphylococcus aureus and/or Streptococcus pyogenes.

97. The method of any one of claims 94-96, wherein the subject is infected with more than a 1 picomolar concentration of bacterial superantigen.

98. The method of any one of claims 94-96, wherein the subject is infected with more than 0.1 ug of superantigen.

99. The method of any one of claims 94-96, wherein the subject is infected with more than 0.2 ug of superantigen.

100. The method of any one of claims 85-99, wherein the method further prevents, manages or lessens the severity of a cytokine storm in a subject.

101. The method of any one of claims 85-100, wherein the subject has elevated levels of neutrophil-lymphocyte ratio (NLR), IL-6, IL-17, IL-10, interferon-gamma, TNF-α, IL-2, and/or procalcitonin.

102. The method of any one of claims 85-101, wherein the subject has elevated levels of neutrophil-lymphocyte ratio (NLR), IL-6, IL-17, and/or procalcitonin.

103. The method of claim 101 or 102, wherein the NLR ratio is greater than 5.

104. The method of claim 101 or 102, wherein the NLR ratio is greater than 7.

105. The method of claim 101 or 102, wherein the NLR ratios is between 7 and 100.

106. The method of any one of claims 85-105, wherein the administration to the subject a bismuth-thiol (BT) composition, treats, inhibits or prevents the dispersal of biofilms in the subject.

107. The method of claim 106, wherein the biofilm is a nasopharyngeal biofilm.

108. The method of any one of claims 85-107, wherein the subject is older than 60 years of age, 70 years of age, 80 years of age and/or 90 years of age.

109. The method of any one of claims 85-108, wherein the BT composition comprises a plurality of microparticles wherein at least 70% of said microparticles having a volumetric mean diameter (VMD) from about 0.6 μm to about 2.5 μm.

110. The method of claim 109, wherein at least 80% of said microparticles having a VMD from about 0.6 μm to about 2.5 μm.

111. The method of claim 109, wherein at least 90% of said microparticles having a VMD from about 0.6 μm to about 2.5 μm.

112. The method of any one of claims 85-110, wherein when the BT composition is aerosolized, at least 70% of the aerosolized liquid droplets have a mass median aerodynamic diameter (MMAD) from about 0.9 μm to about 3 μm.

113. The method of claim 112, wherein when the BT composition is aerosolized, at least 80% of the aerosolized liquid droplets have a MMAD from about 0.9 μm to about 3 μm.

114. The method of claim 112, wherein when the BT composition is aerosolized, at least 90% of the aerosolized liquid droplets have a MMAD from about 0.9 μm to about 3 μm.

115. The method of any of claims 85-114, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.

116. A method of preventing, treating, managing or lessening the severity of Kawasaki disease or Kawasaki syndrome-like illness in a subject, the method comprising administering to the subject a bismuth-thiol (BT) composition that comprises BisEDT suspended therein.

117. The method of claim 116, wherein the subject has been previously infected or is infected with SARS-CoV-2 (COVID-19).

118. The method of claim 116 or 117, wherein the subject is a child.

119. The method of claim 116 or 117, wherein the subject is less than 12 years of age.

120. The method of claim 119, wherein the subject is less than 5 years of age.

121. The method of any one of claims 116-120, wherein the patient is infected with a secondary infection.

122. The method of claim 121, wherein the subject is infected with a bacteria that produces a superantigen.

123. The method of claim 121, were in the superantigens include potent extotoxins secreted by Staphylococcus aureus and/or Streptococcus pyogenes.

124. The method of any one of claims 122-123, wherein the subject is infected with more than a 1 picomolar concentration of superantigen.

125. The method of any one of claims 122-124, wherein the subject is infected with more than 0.1 micogram (ug) of superantigen.

126. The method of any one of claims 122-125, wherein the subject is infected with more than 0.2 ug of superantigen.

127. The method of any one of claims 116-126, wherein the method further prevents, manages or lessens the severity of a cytokine storm in a subject.

128. The method of any one of claims 116-127, wherein the subject has elevated levels of neutrophil-lymphocyte ratio (NLR), IL-6, IL-17, IL-10, interferon-gamma, TNF-α, IL-2, and/or procalcitonin.

129. The method of any one of claim 128, wherein the subject has elevated levels of neutrophil-lymphocyte ratio (NLR), IL-6, IL-17, and/or procalcitonin.

130. The method of any one of claims 116-129, wherein the BT composition comprises a plurality of microparticles wherein at least 70% of said microparticles having a volumetric mean diameter (VMD) from about 0.6 μm to about 2.5 μm.

131. The method of claim 130, wherein at least 80% of said microparticles having a VMD from about 0.6 μm to about 2.5 μm.

132. The method of claim 131, wherein at least 90% of said microparticles having a VMD from about 0.6 μm to about 2.5 μm.

133. A method of preventing, treating, managing or lessening the severity of chronic inflammatory and/or metabolic diseases in a subject, the method comprising administering to the subject a bismuth-thiol (BT) composition that comprises BisEDT suspended therein, wherein administering the BT composition is via inhalation, orally or nasally, using an aerosol device.

134. The method of claim 133, wherein the subject has a SARS-CoV-2 (COVID-19) infection.

135. The method of claim 133-134, wherein the chronic inflammatory disease is related to one of or more conditions selected from one or more of the group consisting of Metabolic Syndrome, Obesity, Vasculitis, Cardiovascular Diseases, Chronic Wounds, Diabetes, hypertension, stroke, inflammatory bowel disease, periodontitis, atherosclerosis, chronic kidney disease, COPD, endocarditis, thrombotic diseases, asthma, aging, recurrent infections, autoimmune diseases, chronic rhinosinusitis, inflammatory arthritis, neurodegenerative conditions (e.g. Alzheimer's Disease), device-related infections, osteomyelitis, depression, chronic respiratory tract infections, chronic otitis media, and/or G.I. tract inflammatory diseases.

136. The method of any one of claims 133-135, wherein the patient is infected with a secondary infection.

137. The method of any one of claim 136, wherein the secondary infection produces a superantigen in the subject.

138. The method of claim 137, wherein the superantigen is a bacterial superantigen.

139. The method of claim 138, were in the superantigens include potent extotoxins secreted by Staphylococcus aureus and/or Streptococcus pyogenes.

140. The method of any one of claims 136-139, wherein the subject is infected with more than a 1 picomolar concentration of bacterial superantigen.

141. The method of any one of claims 136-140, wherein the subject is infected with more than 0.1 ug of superantigen.

142. The method of any one of claims 136-140, wherein the subject is infected with more than 0.2 ug of superantigen.

143. The method of any one of claims 133-142, wherein the method further prevents, manages or lessens the severity of a cytokine storm in a subject.

144. The method of any one of claims 85-100, wherein the subject has elevated levels of IL-1b, IL-2, IL-7, IL-8, IL-9, IL-10, IL-17, G-CSF, GMCSF, IFN-gamma, TNF-α, IP10, MCP1, MIP1A, MIP1B, and/or procalcitonin.

145. The method of any one of claim 144, wherein the subject has elevated levels of neutrophil-lymphocyte ratio (NLR), IL-6, IL-17, and/or procalcitonin.

146. The method of claim 144 or 145, wherein the NLR ratio is greater than 5.

147. The method of claim 144 or 145, wherein the NLR ratio is greater than 7.

148. The method of claim 144 or 145, wherein the NLR ratios is between 7 and 100.

149. The method of any one of claims 133-148, wherein the administration to the subject a bismuth-thiol (BT) composition, treats, inhibits or prevents the dispersal of biofilms in the subject.

150. The method of claim 149, wherein the biofilm is a nasopharyngeal biofilm.

151. The method of any one of claims 133-150, wherein the subject is older than 60 years of age, 70 years of age, 80 years of age and/or 90 years of age.

152. The method of any one of claims 133-151, wherein the BT composition comprises a plurality of microparticles wherein at least 70% of said microparticles having a volumetric mean diameter (VMD) from about 0.6 μm to about 2.5 μm.

153. The method of claim 152, wherein at least 80% of said microparticles having a VMD from about 0.6 μm to about 2.5 μm.

154. The method of claim 152, wherein at least 90% of said microparticles having a VMD from about 0.6 μm to about 2.5 μm.

155. The method of any one of claims 133-154, wherein when the BT composition is aerosolized, at least 70% of the aerosolized liquid droplets have a mass median aerodynamic diameter (MMAD) from about 0.9 μm to about 3 μm.

156. The method of claim 155, wherein when the BT composition is aerosolized, at least 80% of the aerosolized liquid droplets have a MMAD from about 0.9 μm to about 3 μm.

157. The method of claim 155, wherein when the BT composition is aerosolized, at least 90% of the aerosolized liquid droplets have a MMAD from about 0.9 μm to about 3 μm.

158. The method of any of claims 133-157, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.

159. A method of preventing, treating, managing or lessening the severity of lymphopenia in a subject, the method comprising administering to the subject a bismuth-thiol (BT) composition that comprises BisEDT suspended therein, wherein administering the BT composition is via inhalation, orally or nasally, using an aerosol device.

160. The method of claim 159, wherein the BT composition comprises a plurality of microparticles wherein at least 70% of said microparticles having a volumetric mean diameter (VMD) from about 0.6 μm to about 2.5 μm.

161. The method of claim 160, wherein at least 80% of said microparticles having a VMD from about 0.6 μm to about 2.5 μm.

162. The method of claim 160, wherein at least 90% of said microparticles having a VMD from about 0.6 μm to about 2.5 μm.

163. The method of any one of claims 159-162, wherein when the BT composition is aerosolized, at least 70% of the aerosolized liquid droplets have a mass median aerodynamic diameter (MMAD) from about 0.9 μm to about 3 μm.

164. The method of claim 163, wherein when the BT composition is aerosolized, at least 80% of the aerosolized liquid droplets have a MMAD from about 0.9 μm to about 3 μm.

165. The method of claim 163, wherein when the BT composition is aerosolized, at least 90% of the aerosolized liquid droplets have a MMAD from about 0.9 μm to about 3 μm.

166. The method of any of claims 159-165, wherein the BT composition comprises BisEDT at a concentration greater than about 0.1 mg/mL, about 0.05% to about 1.0% Tween 80®, about 0.05 to 40 mM sodium chloride, and optionally about 2 to 20 mM sodium phosphate at about pH. 7.4.
